(19) United States
(12) Patent Application Publication
(10) Pub. No.: US 2019/0177327 A1
(43) Pub. Date: Jun. 13, 2019

(54) SUBSTITUTED METHYL PYRAZOLOPYRIMIDINONE AND METHYL IMIDAZOPYRAZINONE COMPOUNDS AS PDE1 INHIBITORS

(71) Applicant: Dart NeuroScience, LLC, San Diego, CA (US)

(72) Inventors: Brett Bookser, San Diego, CA (US); Iriny Botrous, San Diego, CA (US); Bryan Branstetter, Carlsbad, CA (US); Brian Dyck, San Diego, CA (US); Michael Weinhouse, Lanai City, HI (US)

(21) Appl. No.: 16/159,108

(22) Filed: Oct. 12, 2018

Related U.S. Application Data
(60) Provisional application No. 62/578,315, filed on Oct. 27, 2017.
(60) Provisional application No. 62/571,922, filed on Oct. 13, 2017.

(51) Int. Cl. C07D 487/04

(52) U.S. Cl. CPC C07D 487/04

(57) ABSTRACT

A chemical entity of Formula (I) or Formula (II):

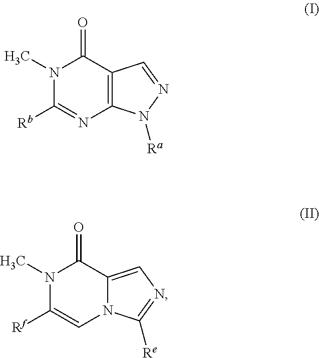

wherein R^{a}, R^{b}, R^{e}, and R^{f} have any of the values described herein, and compositions comprising such chemical entities; methods of making them; and their use in a wide range of methods, including metabolic and reaction kinetic studies; detection and imaging techniques; radioactive treatments; modulating and treating disorders mediated by PDE1 activity or dopaminergic signaling; treating neurological disorders, CNS disorders, dementia, neurodegenerative diseases, and trauma-dependent losses of function; treating stroke, including cognitive and motor deficits during stroke rehabilitation; facilitating neuroprotection and neurorecovery; enhancing the efficiency of cognitive and motor training, including animal skill training protocols; and treating peripheral disorders, including cardiovascular, renal, hematological, gastroenterological, liver, cancer, fertility, and metabolic disorders.

### BACKGROUND

### Field

The present invention relates to certain substituted methyl pyrazolopyrimidinone and methyl imidazopyrazinone compounds and related chemical entities; compositions containing them; processes for making them; and their use in various methods and therapies, including the enhancement of neuroplasticity, and the treatment of neurological, cognitive, cardiovascular, gastrointestinal, renal disorders, and other conditions and diseases involving PDE1, dopaminergic, or cyclic nucleotide signaling.

### Description of the Related Technology

The cyclic nucleotides, adenosine and guanosine 3′,5′-cyclic monophosphate (cAMP and cGMP) are second messengers in cellular signaling cascades activated by diverse transduction pathways, such as those triggered by neurotransmitters and hormones. See, e.g., Kelly and Brandon, 2009, Prog. Brain Res. 179, 67-73; Schmidt, 2010, Curr. Top. Med. Chem. 10, 222-230. Once generated, cAMP and cGMP transmit their signals through various tertiary effectors, such as cAMP dependent protein kinase (PKA), cGMP dependent protein kinase (PKG), and other proteins. In turn, these effectors modulate additional targets in downstream cascades, such as enzymes and transcription factors, ultimately resulting in cellular changes that impact numerous physiological processes, including neuronal plasticity and survival, muscle contraction, sensory transduction, cell division, stress responses, and inflammation.

Cyclic nucleotide levels are subject to tight regulatory controls, including the action of phosphodiesterases (PDEs), a superfamily of intracellular enzymes that hydrolyze cAMP and cGMP to their inactive non-cyclic forms, 5′-AMP and 5′-GMP. See, e.g., Bender and Beavo, 2006, Pharmacol. Rev. 58, 488-520. Mammalian PDEs can be divided into 11 families, PDE1-11, based on structural, biochemical, and pharmacological properties. Some are cAMP-selective hydrolases (PDE4, 7, and 8), some are cGMP-selective hydrolases (PDE5, 6, and 9), and some hydrolyze both cAMP and cGMP (PDE1, 2, 3, 10, and 11). By regulating cAMP and cGMP levels, PDEs play a key role in modulating cyclic nucleotide cascades, and they have become desirable targets for treating various diseases and disorders due to their different tissue distribution and functional properties. See, e.g., Keravis and Lugnier, 2001, Br. J. Pharmacol. 165, 1288-1305. Alterations in cyclic nucleotide concentrations, for example, can impact biochemical and physiological process linked to cognitive function (Kelly and Brandon, 2009, Prog. Brain Res. 179, 67-73; Schmidt, 2010, Curr. Top. Med. Chem. 10, 222-230; Perez-Gonzalez et al., 2013, Neurobiol. Aging. 34, 2133-2145; Lipina et al., 2013, Neuropharmacology 64, 295-214; Morales-Garcia et al., 2016, Stem Cells 35, 458-472).

The PDE1 family, which hydrolyzes both cAMP and cGMP, is distinguished from other PDEs by requiring calcium (Ca²⁺) and calmodulin (CaM) for full activation (Goraya and Cooper, 2005, Cell. Signal. 17, 789-797). The binding of Ca²⁺-CaM complexes at sites near the N-terminus of PDE1 stimulates hydrolysis of cyclic nucleotides. In intact cells, PDE1 is almost exclusively activated by Ca²⁺ entering the cell from the extracellular space. PDE1 is therefore a point of convergence and integration for multiple signaling pathways that regulate numerous downstream targets and cellular events. For review, see Bender and Beavo, 2006, Pharmacol. Rev. 58, 488-520; Sharma et al., 2006, Int. J. Mol. Med. 18, 95-105.

The PDE1 family comprises three members, encoded by separate genes (pde1a, pde1b, and pde1c) that give rise to multiple isoforms via alternative splicing and differential transcription. All PDE1 enzymes appear to hydrolyze both cAMP and cGMP, although they can differ in their relative affinities for each, as well as their relative affinities for calcium and CaM. For review, see Bender and Beavo, 2006, Pharmacol. Rev. 58, 488-520. PDE1 isoforms show distinct but overlapping patterns of expression throughout the body. In the brain, PDE1 is expressed in numerous regions, including the striatum, cerebral cortex, frontal lobe, hippocampus, cerebellum, and amygdala. Brain expression patterns of PDE1B correlate closely with that of dopamine receptors, implicating PDE1 in the modulation of dopamine signaling, a role supported by experiments in PDE1B knockout mice (Reed et al., 2002, J. Neurosci. 22, 5188-5197). Outside the brain, PDE1 is expressed in numerous areas, including muscle, heart, kidney, pancreas, lungs, stomach, and liver. In the cardiovascular system, PDE1 appears to play a central role in organizing cAMP microdomains and mediating hormonal specificity in cardiac cells. See Maurice et al., 2003, Mol. Pharm. 64, 533-546.

Such properties implicate PDE1 in numerous physiological and pathological processes. Alterations in cyclic nucleotide signaling pathways, including those involving PDE1, are implicated in various disorders of the brain, such as depression, schizophrenia and cognitive disorders. See, e.g., Keravis and Lugnier, 2012, Br. J. Pharmacol. 165, 1288-1305. Inhibiting PDE1 activity in the nervous system, for example, can increase cAMP or cGMP levels and consequently induce expression of neuronal plasticity-related genes, neurotrophic factors, and neuroprotective molecules. Similarly, PDE1 enzymes and cyclic nucleotides have been implicated in the etiology of vascular disorders, such as hypertension, myocardial infarction, and heart failure, as well as the development and progression of renal disease. See, e.g., Miller et al., 2011, Basic Res. Cardiol. 106, 1023-1039; Miller et al, 2009, Circ. Res. 105, 956-964; Wang et al., 2010, Kidney Int. 77. 129-140; Cheng et al., 2007, Soc. Exp. Biol. Med. 232, 38-51; Dousa, 1999, Kidney Int. 55, 29-62.

These and other studies highlight the interest in PDE1 as a target for treating numerous disorders and modulating physiological processes, such as cognition. There is a substantial need for PDE1 inhibitors with desirable pharmacological and therapeutic properties, such as effective potency, exposure, selectivity, and safety. The present invention addresses these and other needs in the art by disclosing substituted furanopyrimidine chemical entities as potent, selective, and well-tolerated PDE1 inhibitors.

### SUMMARY

The present disclosure relates to substituted methyl pyrazolopyrimidinone and methyl imidazopyrazinone chemical entities, compositions including such entities, processes for making them, and their use in various methods, including the treatment of central nervous system and peripheral disorders associated with phosphodiesterase 1 (PDE1).

Some embodiments, provide a chemical entity of Formula (I) or Formula (II), or more specifically, a compound or a pharmaceutically acceptable salt of a compound of Formula (I) or Formula (II):

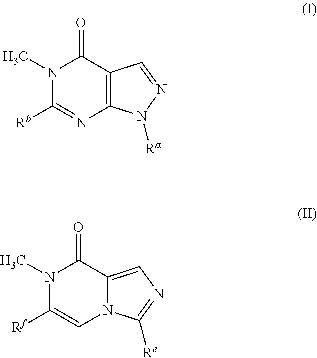

wherein R^{a}, R^{b}, R^{e}, and R^{f} have any of the values described herein.

In some embodiments, a chemical entity of Formula (I) is a chemical entity, or more specifically, a compound or pharmaceutically acceptable salt thereof, of Formula (Ia), Formula (Ib), Formula (Ic), Formula (Id), Formula (Ie), or Formula (If):

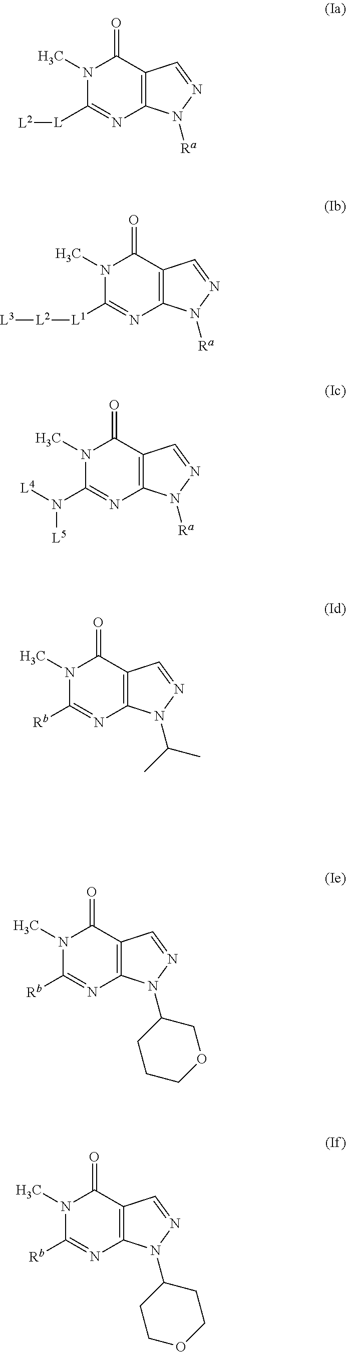

wherein R^{a}, R^{b}, L, L¹, L², L³, L⁴ and L⁵ have any of the values described herein.

In some embodiments, a chemical entity of Formula (Ib) is a chemical entity, or more specifically, a compound or pharmaceutically acceptable salt thereof, of Formula (Iba), Formula (Ibb), or Formula (Ibc):

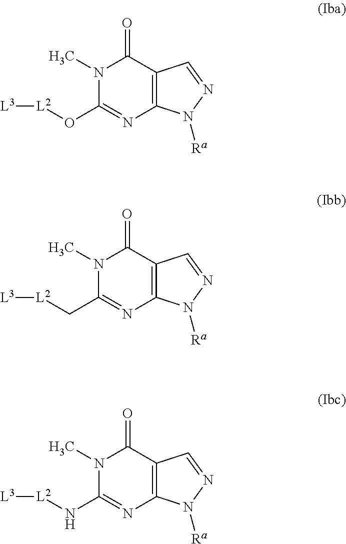

wherein R^{a}, L², and L³ have any of the values described herein.

In some embodiments, a chemical entity of Formula (II) is a chemical entity, or more specifically, a compound or pharmaceutically acceptable salt thereof, of Formula (IIa), Formula (IIb), Formula (IIc), or Formula (IId):

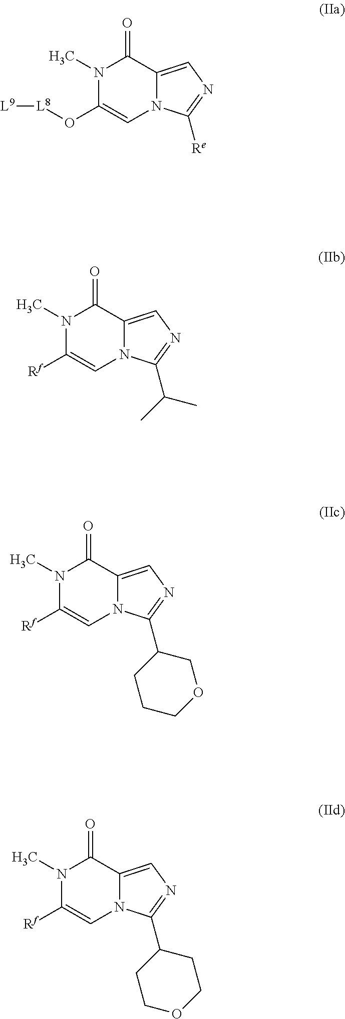

wherein R^{e}, R^{f} L⁸, and L⁹ have any of the values described herein.

In some embodiments, a chemical entity is selected from any of the species described or exemplified herein, and more particularly, is a compound, or pharmaceutically acceptable salt thereof.

In some embodiments, the chemical entities, and compositions including such entities, are used in a wide range of methods, as described herein. In some embodiments, the methods include metabolic and reaction kinetic studies, detection and imaging techniques, and radioactive treatments. In some embodiments, the methods include inhibiting PDE1, treating disorders that are mediated by PDE1, treating disorders characterized by alterations in dopamine signaling, enhancing neuronal plasticity, conferring neuroprotection, and promoting neurogenesis. In some embodiments, the methods include treating neurological disorders, particularly CNS disorders, and more particularly, mental and psychiatric disorders, cognitive disorders, movement disorders, and neurodegenerative disorders. In some embodiments, the methods are directed to treating peripheral disorders, including cardiovascular, renal, hematological, gastrointestinal, liver, fertility, cancer, and metabolic disorders.

In some embodiments, the chemical entities, and compositions including such entities, are useful as augmenting agents to increase the efficiency of cognitive and motor training, including training during post-stroke rehabilitation or post-traumatic brain injury (TBI) rehabilitation; and to increase the efficiency of non-human animal training protocols.

The disclosure is further directed to the general and specific embodiments defined, respectively, and by the independent and dependent claims appended hereto, which are incorporated by reference herein. Additional embodiments, features, and advantages of the disclosure will be apparent from the following detailed description and through practice of the exemplary embodiments.

### DETAILED DESCRIPTION

The invention may be more fully appreciated by reference to the following description, including the Examples. Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. In addition, the materials, methods, and examples are illustrative only and not intended to be limiting.

For the sake of brevity, all publications, including patent applications, patents, and other citations mentioned herein, are incorporated by reference in their entirety. Citation of any such publication, however, shall not be construed as an admission that it is prior art to the present invention.

### Terms and Definitions

The use of headings and subheadings provided in the sections of this specification is solely for convenience of reference and does not limit the various embodiments herein, which are to be construed by reference to the specification as a whole.

General

As used herein, the term “about” or “approximately” means within an acceptable range for a particular value as determined by one skilled in the art, and may depend in part on how the value is measured or determined, e.g., the limitations of the measurement system or technique. For example, “about” can mean a range of up to 20%, up to 10%, up to 5%, or up to 1% or less on either side of a given value. To provide a more concise description, some of the quantitative expressions given herein are not qualified with the term “about.” It is understood that, whether the term “about” is used explicitly or not, every quantity given herein is meant to refer to both the actual given value and the approximation of such given value that would reasonably be inferred based on the ordinary skill in the art, including equivalents and approximations due to the experimental and/or measurement conditions for such given value. Accordingly, for any embodiment of the invention in which a numerical value is prefaced by “about” or “approximately”, the disclosure includes an embodiment in which the exact value is recited. Conversely, for any embodiment of the invention in which a numerical value is not prefaced by “about” or “approximately”, the disclosure includes an embodiment in which the value is prefaced by “about” or “approximately”.

As used herein, the terms “a,” “an,” and “the” are to be understood as meaning both singular and plural, unless explicitly stated otherwise. Thus, “a,” “an,” and “the” (and grammatical variations thereof where appropriate) refer to one or more.

Furthermore, although items, elements or components of the embodiments may be described or claimed in the singular, the plural is contemplated to be within the scope thereof, unless limitation to the singular is explicitly stated.

The terms “comprising” and “including” are used herein in their open, non-limiting sense. Other terms and phrases used in this document, and variations thereof, unless otherwise expressly stated, should be construed as open ended, as opposed to limiting. As examples of the foregoing: the term “example” is used to provide exemplary instances of the item in discussion, not an exhaustive or limiting list thereof; adjectives such as “conventional,” “normal,” “known” and terms of similar meaning should not be construed as limiting the item described to a given time period or to an item available as of a given time, but instead should be read to encompass conventional, or normal technologies that may be available or known now or at any time in the future. Likewise, where this document refers to technologies that would be apparent or known to one of ordinary skill in the art, such technologies encompass those apparent or known to the skilled artisan now or at any time in the future.

As will become apparent to one of ordinary skill in the art after reading this document, the illustrated embodiments and their various alternatives may be implemented without confinement to the illustrated examples.

Chemical Terms

The term “alkyl” refers to a fully saturated aliphatic hydrocarbon group (i.e., contains no double or triple bonds). The alkyl moiety may be a straight- or branched-chain alkyl group having from 1 to 12 carbon atoms in the chain, and more particularly, has 1, 2, 3, 4, 5, 6, 7, 8, 9, 10, 11, or 12 carbons in the chain. Preferably, the alkyl moiety is —C₁₋₆alkyl, and more preferably is C₁₋₄alkyl. Examples of alkyl groups include, but are not limited to, methyl (Me, which also may be structurally depicted by the symbol, “ [symbol] ”), ethyl (Et), n-propyl, isopropyl, butyl, isobutyl, sec-butyl, tert-butyl (tBu), pentyl, isopentyl, tert-pentyl, hexyl, and isohexyl. Alkyl groups may be optionally substituted with one or more substituents including, but not limited to, hydroxyl, alkoxy, thioalkoxy, amino, aminoalkyl, and cyano.

The term “alkenyl” refers to unsaturated acyclic aliphatic moieties having at least one carbon-carbon double bond. The term alkenyl includes all possible geometric isomers including E and Z isomers of said alkenyl moiety unless specifically indicated. Examples of alkenyl radicals include ethenyl, propenyl, butenyl, 1,4-butadienyl, and the like.

The term “alkynyl” refers to optionally substituted unsaturated acyclic aliphatic moieties having at least one carbon-carbon triple bond. Examples of alkynyl radicals include ethynyl, propynyl, butynyl and the like.

The term “haloalkyl” refers to a straight- or branched-chain alkyl group having from 1 to 12 carbon atoms in the chain substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, and —CH₂CF₂CF₃.

The term “alkoxy” includes a straight chain or branched alkyl group with an oxygen atom linking the alkyl group to the rest of the molecule. Examples of alkoxy groups include, but are not limited to, methoxy, ethoxy, propoxy, isopropoxy, butoxy, t-butoxy, and pentoxy. “Aminoalkyl,” “thioalkyl,” and “sulfonylalkyl” are analogous to alkoxy, replacing the terminal oxygen atom of alkoxy with, respectively, NH (or NR), S, and SO₂ where R is selected from hydrogen, C₁₋₆alkyl, C₂₋₆alkenyl, C₂₋₆alkynyl, C₃₋₇cycloalkyl, phenyl, 5-, 6-, 9-, or 10-membered heteroaryl, and 5-10 membered heterocycloalkyl, as defined herein.

The term “haloalkoxy” refers to alkoxy groups substituting one or more hydrogens with halogens. Examples of haloalkoxy groups include, but are not limited to, —OCF₃, —OCHF₂, —OCH₂F, —OCH₂CF₃, —OCH₂CHF₂, —OCH₂CH₂Cl, —OCH₂CF₂CF₃, and —OCH(CH₃)CHF₂.

The term “amino group” refers to an —NH₂ group.

The term “cyano” refers to the group —CN.

The term “aryl” refers to a monocyclic, or fused or spiro polycyclic, aromatic carbocycle (ring structure having ring atoms that are all carbon), having from 3 to 15 ring atoms per ring (carbon atoms in aryl groups are sp2 hybridized). Illustrative examples of aryl groups include the following moieties:

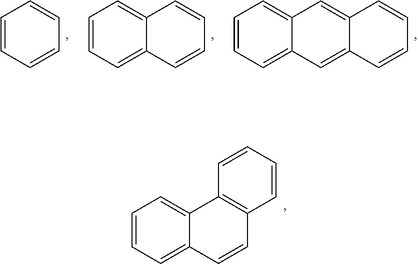

and the like.

The term “phenyl” represents the following moiety:

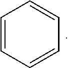

The term “aryloxy” refers to a group having the formula, —O—R, wherein R is an aryl group.

The term “cycloalkyl” refers to a fully saturated or partially saturated carbocycle, such as monocyclic, fused polycyclic, bridged monocyclic, bridged polycyclic, spirocyclic, or spiro polycyclic carbocycle having from 3 to 15 ring atoms per carbocycle. Where the term cycloalkyl is qualified by a specific characterization, such as monocyclic, fused polycyclic, bridged polycyclic, spirocyclic, and spiro polycyclic, then such term cycloalkyl refers only to the carbocycle so characterized. Illustrative examples of cycloalkyl groups include the following entities, in the form of properly bonded moieties:

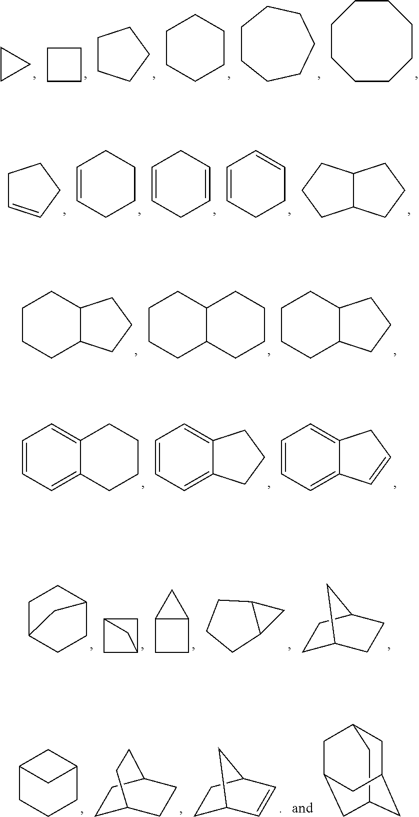

A “heterocycloalkyl” refers to a monocyclic, or fused, bridged, or spiro polycyclic ring structure that is fully saturated or partially saturated and includes at least one heteroatom selected from nitrogen, oxygen, and sulfur in the ring backbone. A heterocycloalkyl may have any degree of saturation provided that at least one ring in a polycyclic ring structure is not aromatic. The heteroatom(s) may be present in either a non-aromatic or aromatic ring in the polycyclic structure. The heterocycloalkyl group may have 3 to 20 ring members (i.e., the number of atoms making up the ring backbone, including carbon atoms and heteroatoms), although the present definition also covers the occurrence of the term “heterocycloalkyl” where no numerical range is designated. The heterocycloalkyl group may be designated as “3-15 membered heterocycloalkyl,” “4-10 membered heterocycloalkyl,” “3-15 membered C₂₋₁₄heterocycloalkyl,” “5-9 membered C₄₋₈heterocycloalkyl,” “5-10 membered C₄₋₉heterocycloalkyl,” “5-membered C₃₋₄heterocycloalkyl,” “6-membered C₄₋₅heterocycloalkyl,” “7-membered C₅₋₆heterocycloalkyl,” or similar designations. The heterocycloalkyl may be a 5-10 membered ring or ring system comprising one to four heteroatoms each independently selected from nitrogen, oxygen, and sulfur. The heterocycloalkyl may be a monocyclic five-membered ring comprising one to three heteroatoms each independently selected from nitrogen, oxygen, and sulfur. The heterocycloalkyl may be a monocyclic six-membered ring comprising one to three heteroatoms each independently selected from nitrogen, oxygen, and sulfur. The heterocycloalkyl may be a bicyclic nine-membered ring comprising one to three heteroatoms each independently selected from nitrogen, oxygen, and sulfur. The heterocycloalkyl may be a bicyclic ten-membered ring comprising one to three heteroatoms each independently selected from nitrogen, oxygen, and sulfur. The heterocycloalkyl may be optionally substituted. Illustrative unsubstituted heterocycloalkyl entities, in the form of properly bonded moieties, include:

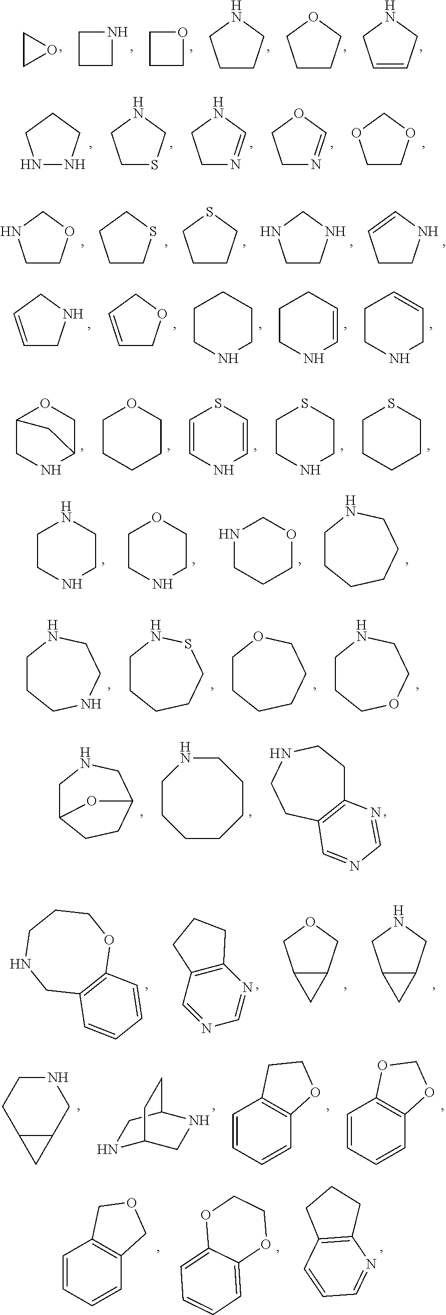

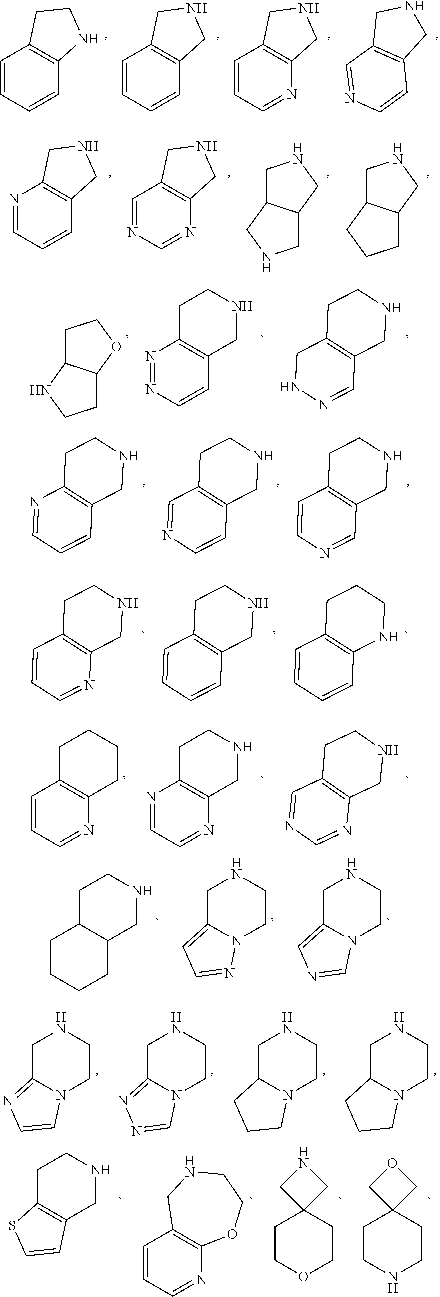

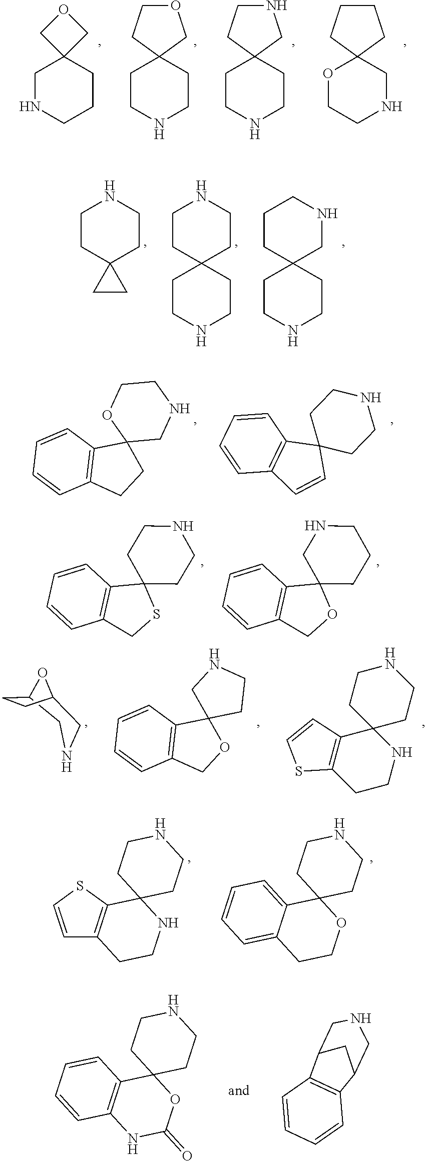

Illustrative carbon or sulfur oxo-substituted heterocycloalkyl entities, in the form of properly bonded moieties, include:

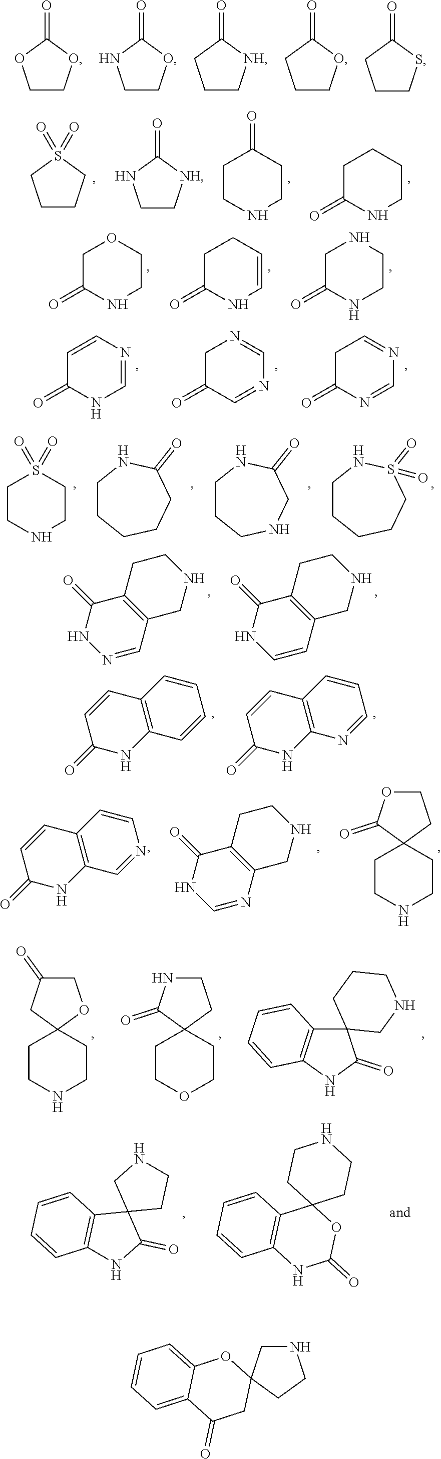

The term “heteroaryl” refers to an aromatic monocyclic, fused bicyclic, or fused polycyclic ring or ring system having one or more heteroatoms selected from nitrogen, oxygen, and sulfur in the ring backbone. When the heteroaryl is a ring system each ring in the ring system is fully unsaturated. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-9 membered heteroaryl,” “5-10 membered heteroaryl,” “5-9 membered C₄₋₈heteroaryl,” “5-10 membered C₄₋₉heteroaryl,” or similar designations. The heteroaryl may be a 5-10 membered ring or ring system comprising one to four heteroatoms each independently selected from nitrogen, oxygen, and sulfur. The heteroaryl may be a monocyclic five-membered ring comprising one to four heteroatoms each independently selected from nitrogen, oxygen, and sulfur. The heteroaryl may be a monocyclic six-membered ring comprising one to four heteroatoms each independently selected from nitrogen, oxygen, and sulfur. The heteroaryl may be a bicyclic nine-membered ring comprising one to four heteroatoms each independently selected from nitrogen, oxygen, and sulfur. The heteroaryl may be a bicyclic ten-membered ring comprising one to four heteroatoms each independently selected from nitrogen, oxygen, and sulfur. In some embodiments, the heteroaryl may be a tautomer of a heterocycloalkyl where the heteroaryl is the predominate form under equilibrium conditions. Illustrative examples of heteroaryl groups include the following entities, in the form of properly bonded moieties:

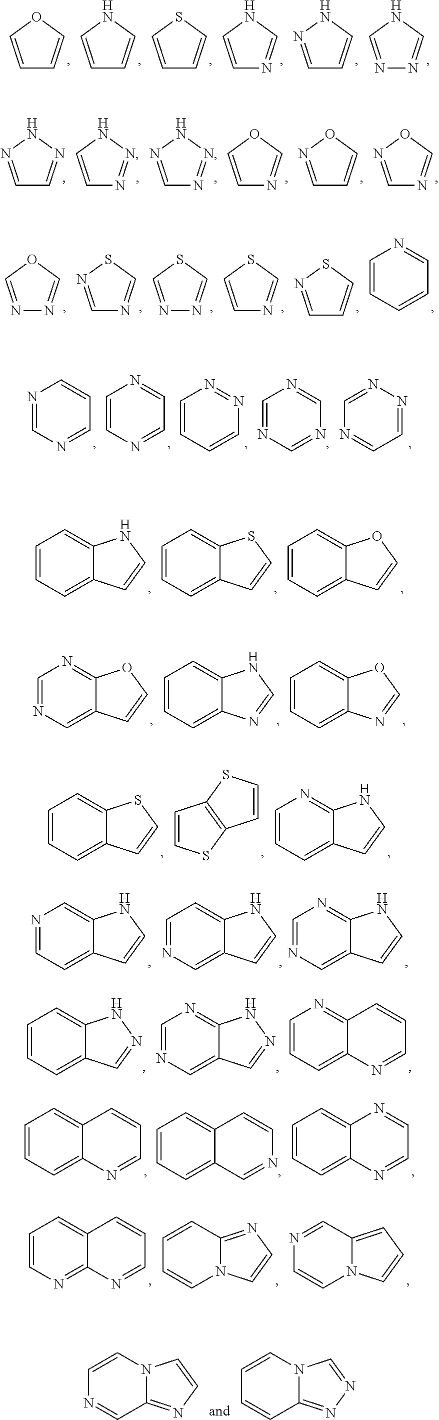

A “cycloalkoxy” refers to a monocyclic, or fused, bridged, or spiro polycyclic ring structure that is fully saturated or partially saturated having at least two carbons and at least one oxygen in the ring backbone. A cycloalkoxy may have any degree of saturation provided that at least one ring in a polycyclic ring structure is not aromatic. The oxygen may be present in the non-aromatic or aromatic ring in the polycyclic structure. The cycloalkoxy group may have 3 to 20 ring members (i.e., the number of atoms making up the ring backbone, including carbon atoms and heteroatoms), although the present definition also covers the occurrence of the term “cycloalkoxy” where no numerical range is designated. The cycloalkoxy group may be designated as “3-15 membered cycloalkoxy,” “4-10 membered cycloalkoxy,” “3-15 membered C₂₋₁₄cycloalkoxy,” “5-9 membered C₄₋₈cycloalkoxy,” “5-10 membered C₄₋₉cycloalkoxy,” “5-membered C₃₋₄cycloalkoxy,” “6-membered C₄₋₅cycloalkoxy,” “7-membered C₅₋₆cycloalkoxy,” or similar designations. The cycloalkoxy may be a 5-10 membered ring or ring system comprising one oxygen and the remainder carbon in the ring backbone. The cycloalkoxy may be optionally substituted. Illustrative unsubstituted cycloalkoxy entities, in the form of properly bonded moieties, include:

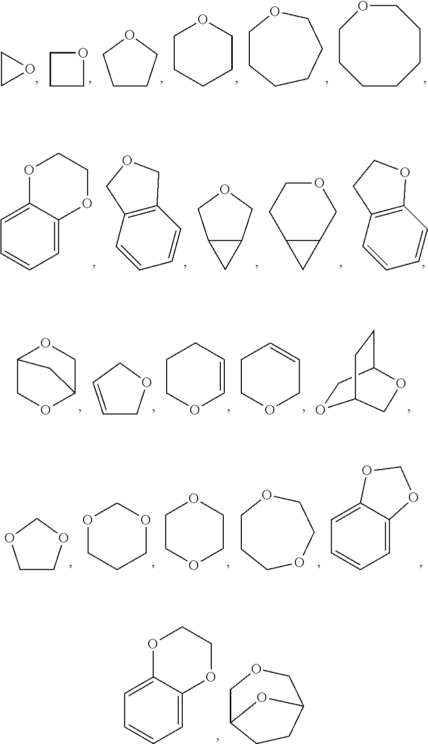

Those skilled in the art will recognize that the species of aryl, cycloalkyl, heterocycloalkyl, heteroaryl and cycloalkoxy groups listed or illustrated above are not exhaustive, and that additional species within the scope of these defined terms may also be selected.

The term “halogen” represents chlorine, fluorine, bromine or iodine. The term “halo” represents chloro, fluoro, bromo or iodo.

The term “heteroatom” used herein refers to, for example, O (oxygen), S (sulfur), or N (nitrogen).

By “optional” or “optionally” is meant that the subsequently described event or circumstance may or may not occur, and that the description includes instances where the event or circumstance occurs and instances or circumstances where it does not. For example, “optionally substituted alkyl” encompasses both “unsubstituted alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

The term “substituted” means that the specified group or moiety bears one or more substituents. A substituted group is derived from the unsubstituted parent group in which there has been an exchange of one or more hydrogen atoms for another atom or group or derived from the unsubstituted parent group in which there has been an addition of one or more atoms or group to a carbon, nitrogen or sulfur. Where the term “substituted” is used to describe a structural system, unless specified otherwise, the substitution is meant to occur at any valency-allowed position on the system. The term “unsubstituted” means that the specified group bears no substituents.

For simplicity, groups described herein that are capable of more than one point of attachment (i.e., divalent, trivalent, polyvalent) may be referred to with a common term. For example, the term “C₃₋₇cycloalkyl” can be used to describe a three to seven membered cycloalkyl group (L²) that is monovalent, as in -L-L², wherein L² has one point of attachment, and that can also be divalent, as in -L¹-L²-L³ (or —O-L²-L³ or —N-L²-L³), wherein L² has two points of attachment. Similarly, L² can refer to an “aryl,” “5-10 membered heteroaryl,” or “3-12 membered heterocycloalkyl” group, each of which can be monovalent, as in -L-L², and divalent, as in -L¹-L²-L³ (or —O-L²-L³ or —N-L²-L³).

As used herein, a substituted group is derived from the unsubstituted parent group in which there has been an exchange of one or more hydrogen atoms for another atom or group.

Unless otherwise indicated, when a group is deemed to be “substituted,” it is meant that the group is substituted with one or more substituents independently selected from C₁-C₆ alkyl, C₂-C₆ alkenyl, C₂-C₆ alkynyl, C₃-C₇ cycloalkyl (optionally substituted with halo, C₁-C₆ alkyl, C₁-C₆ alkoxy, C₁-C₆ haloalkyl, and C₁-C₆ haloalkoxy), C₃-C₇-cycloalkyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy (optionally substituted with halo, C₁-C₆ alkyl, C₁-C₆ alkoxy, C₁-C₆ haloalkyl, and C₁-C₆ haloalkoxy), C₃-C₇ cycloalkyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo, C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-cycloalkyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl (mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents unless the optional substituents are otherwise specifically identified.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers.

As used herein, “tautomer” refers to the migration of protons between adjacent single and double bonds. The tautomerization process is reversible. Compounds described herein can undergo any possible tautomerization that is within the physical characteristics of the compound. The following is an example tautomerization that can occur in compounds described herein:

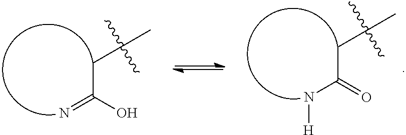 [SMILES: *CC(=N)O ; *CC(N)=O]

The symbols [symbol] and [symbol] are used as meaning the same spatial arrangement in chemical structures shown herein. Analogously, the symbols [symbol] and [symbol] are used as meaning the same spatial arrangement in chemical structures shown herein.

The term “chiral” refers to molecules, which have the property of non-superimposability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

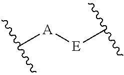

includes the substituent being oriented such that the A is attached at the leftmost attachment point of the molecule as well as the case in which A is attached at the rightmost attachment point of the molecule.

Chemical Entities

As used herein, the term “chemical entity” collectively refers to a compound, along with all pharmaceutically acceptable forms thereof, including pharmaceutically acceptable salts, chelates, solvates, conformers, crystalline forms/polymorphs, tautomers, prodrugs, metabolites, and mixtures thereof. In some embodiments, the chemical entity is selected from the group consisting of a compound and pharmaceutically acceptable salts thereof.

Chelates

The term “chelate” refers to the chemical entity formed by the coordination of a compound to a metal ion at two (or more) points.

Solvates

Additionally, any formula given herein is intended to refer also to hydrates, solvates, and polymorphs of such compounds, and mixtures thereof, even if such forms are not listed explicitly. Some embodiments provide a solvate of a compound of Formula (I) or (II), and the use of such solvates in methods described herein. Certain compounds of Formula (I) or (II) or pharmaceutically acceptable salts of compounds of Formula (I) or (II) may be obtained as solvates. In some embodiments, the solvent is water and the solvates are hydrates.

More particularly, solvates include those formed from the interaction or complexes of compounds of the invention with one or more solvents, either in solution or as a solid or crystalline form. Such solvent molecules are those commonly used in the pharmaceutical art, which are known to be innocuous to the recipient, e.g., water, ethanol, ethylene glycol, and the like. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like. Hydrates include a molecule of a compound associated with water molecules.

Conformers and Crystalline Forms/Polymorphs

Some embodiments provide conformer and crystalline forms of a compound of Formula (I) or (II), and their use in methods of the present disclosure. A conformer is a structure that is a conformational isomer.

Conformational isomerism is the phenomenon of molecules with the same structural formula but different conformations (conformers) of atoms about a rotating bond.

Polymorphs refer to a solid material that can exist in more than one form or crystal structure, where each form or crystal structure is different from the other form(s) or crystal structure(s). Therefore, a single compound may give rise to a variety of polymorphic forms having different and distinct physical properties, such as solubility profiles, melting point temperatures, hygroscopicity, particle shape, density, flowability, compactability and x-ray diffraction peaks. In certain embodiments, compounds of Formula (I) or (II) are obtained in crystalline form. In addition, certain crystalline forms of compounds of Formula (I) or (II) or pharmaceutically acceptable salts of compounds of Formula (I) or (II) may be obtained as co-crystals. In still other embodiments, compounds of Formula (I) or (II) may be obtained in one of several polymorphic forms, as a mixture of crystalline forms, as a polymorphic form, or as an amorphous form.

Compounds

As used herein, a “compound” refers to any one of: (a) the actually recited form of such compound; and (b) any of the forms of such compound in the medium in which the compound is being considered when named. For example, reference herein to a compound such as R—OH encompasses reference to any one of, for example, R—OH(s), R—OH(sol), and R—O-(sol). In this example, R—OH(s) refers to the solid compound, as it could be for example in a tablet or some other solid pharmaceutical composition or preparation; R—OH(sol) refers to the undissociated form of the compound in a solvent; and R—O-(sol) refers to the dissociated form of the compound in a solvent, such as the dissociated form of the compound in an aqueous environment, whether such dissociated form derives from R—OH, from a salt thereof, or from any other entity that yields R—O— upon dissociation in the medium being considered.

In another example, an expression such as “modulate activity of PDE1 or an associated signaling pathway” refers to the exposure of PDE1 to the form, or forms, of the compound R—OH that exists, or exist, in the medium in which such exposure takes place. In this regard, if such compound is, for example, in an aqueous environment, it is understood that the compound R—OH is in the same such medium, and therefore PDE1 is being exposed to the compound as it exists in the medium such as R—OH (aq) and/or R—O— (aq), where the subscript “(aq)” stands for “aqueous” according to its conventional meaning in chemistry and biochemistry. A hydroxyl functional group has been chosen in these nomenclature examples; this choice is not intended, however, as a limitation but is merely an illustration. It is understood that analogous examples can be provided in terms of other functional groups, including, but not limited to, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation and deprotonation. No further examples in this regard are provided herein because these interactions and transformations in a given medium are known by any one of ordinary skill in the art.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of species for the same variable elsewhere in the formula, unless otherwise stated.

Salts

Embodiments include pharmaceutically acceptable salts of the compounds represented by Formula (I) or (II), and methods using such salts.

A “pharmaceutically acceptable salt” is intended to mean a salt of a free acid or base of a compound represented by Formula (I) or (II) that is non-toxic, biologically tolerable, or otherwise biologically suitable for administration to the subject. See, generally, G. S. Paulekuhn et al., 2007, J. Med. Chem. 50, 6665-6672; Berge et al., 1977, J. Pharm. Sci. 66, 1-19; Stahl and Wermuth (eds), Pharmaceutical Salts: Properties, Selection, and Use: 2nd Revised Edition, Wiley-VCS, Zurich, Switzerland (2011). Examples of pharmaceutically acceptable salts are those that are pharmacologically effective and suitable for contact with the tissues of patients without undue toxicity, irritation, or allergic response. A compound of Formula (I) or (II) may possess a sufficiently acidic group, a sufficiently basic group, or both types of functional groups, and accordingly react with a number of inorganic or organic bases, and inorganic and organic acids, to form pharmaceutically acceptable salt bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, borate, nitrate, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycolates, tartrates, methane-sulfonates, propanesulfonates, naphthalene-1-sulfonates, naphthalene-2-sulfonates, besylate, mesylate and mandelates.

When the compound of Formula (I) or (II) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid, glutaric acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

When the compound of Formula (I) or (II) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as N-methyl-O-glucamine, lysine, choline, glycine and arginine, ammonia, carbonates, bicarbonates, primary, secondary, and tertiary amines, and cyclic amines, such as tromethamine, benzylamines, pyrrolidines, piperidine, morpholine, and piperazine, and inorganic salts derived from sodium, calcium, potassium, magnesium, manganese, iron, copper, zinc, aluminum, and lithium.

Prodrugs

Some embodiments provide prodrugs of the compounds of Formula (I) or (II), and the use of such pharmaceutically acceptable prodrugs in methods of the present disclosure, particularly therapeutic methods.

The term “prodrug” means a precursor of a designated compound that is initially inactive or partially inactive, and that following administration to a subject, yields the compound in vivo via a chemical or physiological process such as solvolysis or enzymatic cleavage, or under physiological conditions (e.g., a prodrug on being brought to physiological pH is converted to an active pharmacological compound of Formula (I) or (II)).

A “pharmaceutically acceptable prodrug” is a prodrug that is preferably non-toxic, biologically tolerable, and otherwise biologically suitable for administration to the subject. Prodrugs are often useful because, in some situations, they can be easier to administer than the parent drug. They can, for instance, be bioavailable by oral administration whereas the parent is not. The prodrug can also have improved solubility in pharmaceutical compositions over the parent drug.

Prodrugs may be determined using routine techniques known or available in the art Prodrugs may be produced, for instance, by derivatizing free carboxyl groups, free hydroxy groups, or free amino groups. See, e.g., Bundgaard (ed.), 1985, Design of prodrugs, Elsevier; Krogsgaard-Larsen et al., (eds.), 1991, Design and Application of Prodrugs, Harwood Academic Publishers; Fleisher et al., Adv. Drug Delivery Rev. 1996, 19, 115-130; Robinson et al., 1996, J. Med. Chem. 39, 10-18.

Tautomers

Some embodiments provide tautomers of compounds of Formula (I) or (II), as defined further herein, which may also be used in the methods of the disclosure.

Metabolites

Some embodiments provide pharmaceutically active metabolites of the compounds of Formula (I) or (II), which may also be used in the methods of the disclosure. A “pharmaceutically active metabolite” means a pharmacologically active product of metabolism in the body of a compound of Formula (I) or (II) or salt thereof. Preferably, the metabolite is in an isolated form outside the body.

Active metabolites of a compound may be determined using routine techniques known or available in the art. For example, isolated metabolites can be enzymatically and synthetically produced (e.g., Bertolini et al., J. Med. Chem. 1997, 40, 2011-2016; Shan et al., J. Pharm. Sci. 1997, 86, 765-767; Bagshawe, Drug Dev. Res. 1995, 34, 220-230; and Bodor, Adv. Drug Res. 1984, 13, 224-231).

Isotopes

Isotopes may be present in the compounds described. Each chemical element present in a compound either specifically or generically described herein may include any isotope of the element. Any formula given herein is also intended to represent unlabeled forms as well as isotopically-labeled forms of the compounds. Isotopically-labeled compounds have structures depicted by the formulas given herein except that one or more atoms are replaced by an atom having a selected atomic mass or mass number. Examples of isotopes that can be incorporated into compounds of the embodiments include isotopes of hydrogen, carbon, nitrogen, oxygen, phosphorus, sulfur, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively.

Compositions

The term “composition,” as in pharmaceutical composition, is intended to encompass a product comprising the active ingredient(s) (e.g., one or more of the presently disclosed chemical entities), and the inert ingredient(s) (pharmaceutically acceptable excipients) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a chemical entity of Formula (I) or (II) and a pharmaceutically acceptable excipient.

The term “pharmaceutically acceptable,” as used in connection with compositions of the invention, refers to molecular entities and other ingredients of such compositions that are physiologically tolerable and do not typically produce untoward reactions when administered to an animal (e.g., human). The term “pharmaceutically acceptable” can also mean approved by a regulatory agency of the Federal or a state government or listed in the U.S. Pharmacopeia or other generally recognized pharmacopeia for use in animals (e.g. mammals), and more particularly in humans.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluents to facilitate administration of an agent and that is compatible therewith. Examples of excipients include calcium carbonate, calcium phosphate, various sugars and types of starch, cellulose derivatives, gelatin, vegetable oils, and polyethylene glycols. Suitable pharmaceutical carriers include those described in Remington: The Science and Practice of Pharmacy, 21^{st} Ed., Lippincott Williams & Wilkins (2005).

A “pharmaceutically acceptable salt” is intended to mean a salt of a free acid or base of a compound represented by Formula (I) or (II), as previously defined herein.

The term “carrier” refers to an adjuvant, vehicle, or excipients, with which the compound is administered. In preferred embodiments of this invention, the carrier is a solid carrier. Suitable pharmaceutical carriers include those described in Remington: The Science and Practice of Pharmacy, 21^{st} Ed., Lippincott Williams & Wilkins (2005).

The term “dosage form,” as used herein, is the form in which the dose is to be administered to the subject or patient. The drug is generally administered as part of a formulation that includes nonmedical agents. The dosage form has unique physical and pharmaceutical characteristics. Dosage forms, for example, may be solid, liquid or gaseous. “Dosage forms” may include for example, a capsule, tablet, caplet, gel caplet (gelcap), syrup, a liquid composition, a powder, a concentrated powder, a concentrated powder admixed with a liquid, a chewable form, a swallowable form, a dissolvable form, an effervescent, a granulated form, and an oral liquid solution. In a specific embodiment, the dosage form is a solid dosage form, and more specifically, comprises a tablet or capsule.

As used herein, the term “inert” refer to any inactive ingredient of a described composition. The definition of “inactive ingredient” as used herein follows that of the U.S. Food and Drug Administration, as defined in 21 C.F.R. 201.3(b)(8), which is any component of a drug product other than the active ingredient.

As used herein, “suitable for oral administration” refers to a sterile, pharmaceutical product produced under good manufacturing practices (GMP) that is prepared and presented in a manner such that the composition is not likely to cause any untoward or deleterious effects when orally administered to a subject. Unless specified otherwise, all of the compositions disclosed herein are suitable for oral administration.

Methods and Uses

As used herein, the term “disorder” is used interchangeably with “disease” or “condition”. For example, a CNS disorder also means a CNS disease or a CNS condition.

As used herein, the term “cognitive impairment” is used interchangeably with “cognitive dysfunction” or “cognitive deficit,” all of which are deemed to cover the same therapeutic indications.

The terms “treating,” “treatment,” and “treat” cover therapeutic methods directed to a disease-state in a subject and include: (i) preventing the disease-state from occurring, in particular, when the subject is predisposed to the disease-state but has not yet been diagnosed as having it; (ii) inhibiting the disease-state, e.g., arresting its development (progression) or delaying its onset; and (iii) relieving the disease-state, e.g., causing regression of the disease state until a desired endpoint is reached. Treating also includes ameliorating a symptom of a disease (e.g., reducing the pain, discomfort, or deficit), wherein such amelioration may be directly affecting the disease (e.g., affecting the disease's cause, transmission, or expression) or not directly affecting the disease. Particularly with respect to progressive disease-states or conditions, maintaining the status quo, or arresting the progression of symptoms, is understood to be an amelioration of such symptoms.

As used in the present disclosure, the term “effective amount” is interchangeable with “therapeutically effective amount” and means an amount or dose of a compound or composition effective in treating the particular disease, condition, or disorder disclosed herein, and thus “treating” includes producing a desired preventative, inhibitory, relieving, or ameliorative effect. In methods of treatment according to the invention, “an effective amount” of at least one compound according to the invention is administered to a subject (e.g., a mammal). An “effective amount” also means an amount or dose of a compound or composition effective to modulate activity of PDE1 or an associated signaling pathway. The “effective amount” will vary, depending on the compound, the disease, the type of treatment desired, and its severity, and age, weight, etc.

As used herein, the term “PDE1” refers to all translation products coded by transcripts of any or all three genes, PDE1A, PDE1B, and PDE1C. The amino acid and nucleotide sequences that encode PDE1 of various species are known to those skilled in the art and can be found, for example, in GenBank under accession numbers AJ401610.1, AJ401609.1, and Fiddock et al., 2002, Cell. Signal. 14, 53-60.

The term “animal” is interchangeable with “subject” and may be a vertebrate, in particular, a mammal, and more particularly, a human, and includes a laboratory animal in the context of a clinical trial or screening or activity experiment. Thus, as can be readily understood by one of ordinary skill in the art, the compositions and methods of the present invention are particularly suited to administration to any vertebrate, particularly a mammal, and more particularly, a human.

As used herein, a “control animal” or a “normal animal” is an animal that is of the same species as, and otherwise comparable to (e.g., similar age, sex), the animal that is trained under conditions sufficient to induce transcription-dependent memory formation in that animal.

By “enhance,” “enhancing” or “enhancement” is meant the ability to potentiate, increase, improve or make greater or better, relative to normal, a biochemical or physiological action or effect. For example, enhancing long term memory formation refers to the ability to potentiate or increase long term memory formation in an animal relative to (or “compared to”) the normal long term memory formation of the animal or controls. As a result, long term memory acquisition is faster or better retained. Enhancing performance of a cognitive task refers to the ability to potentiate or improve performance of a specified cognitive task by an animal relative to the normal performance of the cognitive task by the animal or controls.

As used herein, the term “training protocol,” or “training,” refers to either “cognitive training” or “motor training.”

Reference will now be made to the embodiments of the present invention, examples of which are illustrated by and described in conjunction with the accompanying examples. While certain embodiments are described herein, it is understood that the described embodiments are not intended to limit the scope of the invention. On the contrary, the present disclosure is intended to cover alternatives, modifications, and equivalents that can be included within the invention as defined by the appended claims.

### Chemical Entities

Some embodiments provide certain substituted methyl pyrazolopyrimidinone and methyl imidazopyrazinone chemical entities, which are useful, for example, as inhibitors of PDE1 enzymatic activity.

In some embodiments, the chemical entities include the compounds disclosed herein and pharmaceutically acceptable salts, chelates, solvates, conformers, crystalline forms/polymorphs, tautomers, prodrugs, metabolites, and mixtures thereof. In some embodiments, the chemical entities include the compounds disclosed herein and pharmaceutically acceptable salts thereof.

### Formula (I)

Some embodiments provide a chemical entity of Formula (I), or, more specifically, a compound of Formula (I), or a pharmaceutically acceptable salt of a compound of Formula (I):

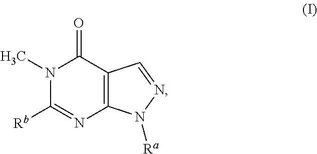

wherein: R^{a} and R^{b}, have any of the values described herein.

In some embodiments of a chemical entity of Formula (I),

R^{a} is a 3-6-membered cycloalkyl ring, a 3-6-membered cycloalkoxy ring, or —CHR^{c}R^{d}, where R^{c} and R^{d} are independently —C₁₋₄alkyl;
R^{b} is -L-L², -L-L²-L³ or —N(L⁴)-L⁵;
L is a member selected from the group consisting of: a bond, —O—, —OCH₂—, —OCH₂CH₂—, and —NH—;
L¹ is a member selected from the group consisting of: a bond, —CH₂—, —CHF—, —CF₂—, —O—, —OCH₂—, —OCH₂CH₂—, and —NH—;
L² is aryl, 5-10-membered heteroaryl, —C₃₋₇cycloalkyl, or 3-12-membered heterocycloalkyl, all optionally substituted with 1 to 3 R^{1A}, where each R^{1A} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
each n is independently 0, 1, or 2;
L³ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1B}, where each R^{1B} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl;
L⁴ and L⁵ are taken together with the nitrogen to which they are attached to form a 3-12-membered heterocycloalkyl ring, optionally substituted with 1 to 3 R^{1C}, where each R^{1C} is independently selected from the group consisting of: L⁶, halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₆alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
L⁶ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, each of said aryl, heteroaryl, cycloalkyl, and heterocycloalkyl groups, as recited above, optionally and independently substituted with 1 to 3 R^{1D}, where each R^{1D} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₄alkyl, —C₁₋₄alkyl-O—C₁₋₄alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In certain embodiments, a chemical entity of Formula (I) is a chemical entity of Formula (Ia), and more particularly, is a compound of Formula (Ia), or a pharmaceutically acceptable salt of a compound of Formula (Ia):

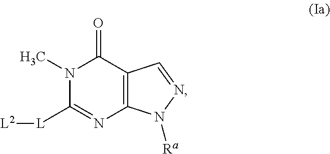

wherein R^{a}, L and L² have any of the values described herein.

In certain embodiments of a chemical entity of Formula (Ia),

R^{a} is a 3-6-membered cycloalkyl ring, a 3-6-membered cycloalkoxy ring, or —CHR^{c}R^{d}, where R^{c} and R^{d} are independently —C₁₋₄alkyl;
L is a member selected from the group consisting of: —O—, —OCH₂—, —OCH₂CH₂—, and —NH—;
L² is aryl, 5-10-membered heteroaryl, —C₃₋₇cycloalkyl, or 3-12-membered heterocycloalkyl, all optionally substituted with 1 to 3 R^{1A}, where each R^{1A} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
and each n is independently 0, 1, or 2.

In certain embodiments, the chemical entity of Formula (I) is a chemical entity of Formula (Ib), and more particularly, is a compound of Formula (Ib), or a pharmaceutically acceptable salt of a compound of Formula (Ib):

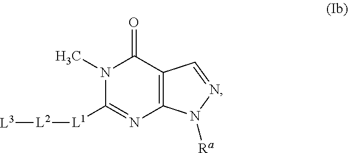

wherein R^{a}, L¹, L² and L³ have any of the values described herein.

In certain embodiments of a chemical entity of Formula (Ib),

R^{a} is a 3-6-membered cycloalkyl ring, a 3-6-membered cycloalkoxy ring, or —CHR^{c}R^{d}, where R^{e} and R^{d} are independently —C₁₋₄alkyl;
L¹ is a member selected from the group consisting of: —CH₂—, —CHF—, —CF₂—, —O—, —OCH₂—, —OCH₂CH₂—, and —NH—;
L² is aryl, 5-10-membered heteroaryl, —C₃₋₇cycloalkyl, or 3-12-membered heterocycloalkyl, all optionally substituted with 1 to 3 R^{1A}, where each R^{1A} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
each n is independently 0, 1, or 2; and
L³ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1B}, where each R^{1B} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In certain embodiments, the chemical entity of Formula (I) is a chemical entity of Formula (Ic), and more particularly, is a compound of Formula (Ic), or a pharmaceutically acceptable salt of a compound of Formula (Ic):

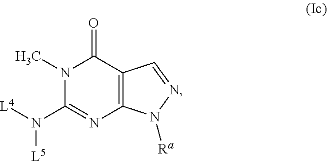

wherein R^{a}, L⁴ and L⁵ have any of the values described herein.

In certain embodiments of a chemical entity of Formula (Ic),

R¹ is a 3-6-membered cycloalkyl ring, a 3-6-membered cycloalkoxy ring, or —CHR^{c}R^{d}, where R^{c} and R^{d} are independently —C₁₋₄alkyl;
L⁴ and L⁵ are taken together with the nitrogen to which they are attached to form a 3-12-membered heterocycloalkyl ring, said heterocycloalkyl ring substituted with 1 L⁶ and optionally substituted with 1 to 3 R^{1C}, where each R^{1C} is independently selected from the group consisting of: L⁶, halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
each n is independently 0, 1, or 2; and
L⁶ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1D}, where each R^{1D} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In certain embodiments, the chemical entity of Formula (I) is a chemical entity of Formula (Iba), and more particularly, is a compound of Formula (Iba), or a pharmaceutically acceptable salt of a compound of Formula (Iba):

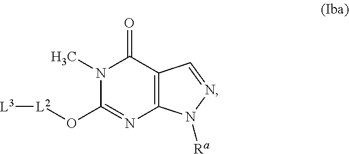

wherein R^{a}, L² and L³ have any of the values described herein.

In certain embodiments of a chemical entity of Formula (Iba),

R^{a} is a 3-6-membered cycloalkyl ring, a 3-6-membered cycloalkoxy ring, or —CHR^{c}R^{d}, where R^{c} and R^{d} are independently —C₁₋₄alkyl;
L² is aryl, 5-10-membered heteroaryl, —C₃₋₇cycloalkyl, or 3-12-membered heterocycloalkyl, all optionally substituted with 1 to 3 R^{1A}, where each R^{1A} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
each n is independently 0, 1, or 2; and
L³ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —CH₂heteroaryl, said aryl, heteroaryl, cycloalkyl and heterocycloalkyl optionally substituted with 1 to 3 R^{1B}, where each R^{1B} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In certain embodiments, the chemical entity of Formula (I) is a chemical entity of Formula (Ibb), and more particularly, is a compound of Formula (Ibb), or a pharmaceutically acceptable salt of a compound of Formula (Ibb):

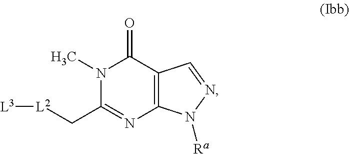

wherein R^{a}, L⁴ and L⁵ have any of the values described herein.

In certain embodiments of a chemical entity of Formula (Ibb),

R^{a} is a 3-6-membered cycloalkyl ring, a 3-6-membered cycloalkoxy ring, or —CHR^{c}R^{d}, where R^{c} and R^{d} are independently —C₁₋₄alkyl;
L² is aryl, 5-10-membered heteroaryl, —C₃₋₇cycloalkyl, or 3-12-membered heterocycloalkyl, all optionally substituted with 1 to 3 R^{1A}, where each R^{1A} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
each n is independently 0, 1, or 2; and
L³ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl and heterocycloalkyl optionally substituted with 1 to 3 R^{1B}, where each R^{1B} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In certain embodiments, the chemical entity of Formula (I) is a chemical entity of Formula (Ibc), and more particularly, is a compound of Formula (Ibc), or a pharmaceutically acceptable salt of a compound of Formula (Ibc):

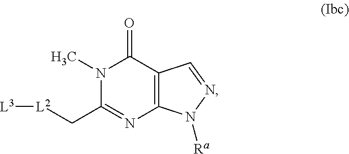

wherein R^{a}, L², and L³ have any of the values described herein.

In certain embodiments of a chemical entity of Formula (Ibc),

R^{a} is a 3-6-membered cycloalkyl ring, a 3-6-membered cycloalkoxy ring, or —CHR^{c}R^{d}, where R^{c} and R^{d} are independently —C₁₋₄alkyl;
L² is aryl, 5-10-membered heteroaryl, —C₃₋₇cycloalkyl, or 3-12-membered heterocycloalkyl, all optionally substituted with 1 to 3 R^{1A}, where each R^{1A} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
each n is independently 0, 1, or 2; and
L³ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1B}, where each R^{1B} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In some embodiments of a chemical entity of Formula (I), (Ia), (Ib), (Ic), (Iba), (Ibb), or (Ibc) disclosed herein: R^{a} is a 3-6-membered cycloalkyl ring, a 3-6-membered cycloalkoxy ring, or —CHR^{c}R^{d}, where R^{c} and R^{d} are independently —C₁₋₄alkyl.

In some embodiments of a chemical entity of Formula (I), (Ia), (Ib), (Ic), (Iba), (Ibb), or (Ibc) disclosed herein: R^{a} is —CHR^{c}R^{d}, where R^{c} and R^{d} are independently —C₁₋₄alkyl.

In some embodiments of a chemical entity of Formula (I), (Ia), (Ib), (Ic), (Iba), (Ibb), or (Ibc) disclosed herein: R^{c} and R^{d} are —CH₃.

In some embodiments of a chemical entity of Formula (I), (Ia), (Ib), (Ic), (Iba), (Ibb), or (Ibc) disclosed herein: R^{a} is isopropyl.

In some embodiments of a chemical entity of Formula (I), (Ia), (Ib), (Ic), (Iba), (Ibb), or (Ibc) disclosed herein: R^{b} is -L¹-L²-L³; L³ is a member selected from the group consisting of: aryl, 5-6-membered heteroaryl, and 3-10-membered heterocycloalkyl, said aryl, heteroaryl, cycloalkyl, and heterocycloalkyl substituted with 1 to 3 R^{1B}, where each R^{1B} is independently selected from the group consisting of: halo, —C₁₋₃alkyl, —C₁₋₃haloalkyl, —C₁₋₃alkoxy, —C₁₋₃haloalkoxy, —C₃₋₅cycloalkyl, —C(O)C₁₋₃alkyl, —C₁₋₃alkyl-O—C₁₋₃alkyl, —C₁₋₃alkyl-CN, —OH, ═O, —O—C₁₋₃alkyl-C₃₋₅cycloalkyl, and —NHC₁₋₃alkyl.

In some embodiments of a chemical entity of Formula (I), (Ia), (Ib), (Ic), (Iba), (Ibb), or (Ibc) disclosed herein: R^{a} is a 3-6-membered cycloalkyl ring, or a 3-6-membered cycloalkoxy ring.

In some embodiments of a chemical entity of Formula (I), (Ia), (Ib), (Ic), (Iba), (Ibb), or (Ibc) disclosed herein: R^{a} is a 3-6-membered cycloalkoxy ring.

In some embodiments of a chemical entity of Formula (I), (Ia), (Ib), (Ic), (Iba), (Ibb), or (Ibc) disclosed herein: R^{a} is a tetrahydropyran or tetrahydrofuran ring.

In some embodiments of a chemical entity of Formula (I), (Ia), (Ib), (Ic), (Iba), (Ibb), or (Ibc) disclosed herein: R^{a} is a tetrahydropyran ring.

In certain embodiments, the chemical entity of Formula (I) is a chemical entity of Formula (Id), and more particularly, is a compound of Formula (Id), or a pharmaceutically acceptable salt of a compound of Formula (Id):

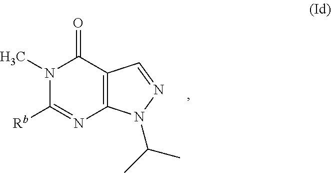

wherein R^{b} has any of the values described herein.

In certain embodiments of a chemical entity of Formula (Id),

R^{b} is -L-L², -L¹-L²-L³ or —N(-L⁴)L⁵;
L is a member selected from the group consisting of: a bond, —O—, —OCH₂—, —OCH₂CH₂—, and —NH—;
L¹ is a member selected from the group consisting of: —CH₂—, —CHF—, —CF₂—, —O—, —OCH₂—, —OCH₂CH₂—, and —NH—;
L² is aryl, 5-10-membered heteroaryl, —C₃₋₇cycloalkyl, or 3-12-membered heterocycloalkyl, all optionally substituted with 1 to 3 R^{1A}, where each R^{1A} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
each n is independently 0, 1, or 2;
L³ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1B}, where each R^{1B} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl;
L⁴ and L⁵ are taken together with the nitrogen to which they are attached to form a 3-12-membered heterocycloalkyl group, said heterocycloalkyl group optionally substituted with 1 to 3 R^{1C}, where each R^{1C} is independently selected from the group consisting of: L⁶, halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
L⁶ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1D}, where each R^{1D} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In certain embodiments, the chemical entity of Formula (I) is a chemical entity of Formula (Ie), and more particularly, is a compound of Formula (Ie), or a pharmaceutically acceptable salt of a compound of Formula (Ie):

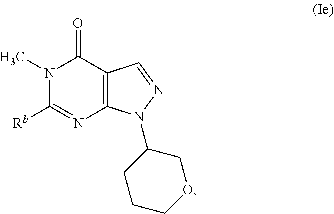

wherein R^{b} has any of the values described herein.

In certain embodiments of a chemical entity of Formula (Ie),

R^{b} is -L-L², -L¹-L²-L³ or —N(-L⁴)L⁵;
L is a member selected from the group consisting of: a bond, —O—, —OCH₂—, —OCH₂CH₂—, and —NH—;
L¹ is a member selected from the group consisting of: —CH₂—, —CHF—, —CF₂—, —O—, —OCH₂—, —OCH₂CH₂—, and —NH—;
L² is aryl, 5-10-membered heteroaryl, —C₃₋₇cycloalkyl, or 3-12-membered heterocycloalkyl, all optionally substituted with 1 to 3 R^{1A}, where each R^{1A} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
each n is independently 0, 1, or 2;
L³ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1B}, where each R^{1B} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl;
L⁴ and L⁵ are taken together to form a 3-12-membered heterocycloalkyl ring, optionally substituted with 1 to 3 R^{1C}, where each R^{1C} is independently selected from the group consisting of: L⁶, halo, —CN, —C₁₋₆-alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
L⁶ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1D}, where each R^{1D} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In certain embodiments, the chemical entity of Formula (I) is a chemical entity of Formula (If), and more particularly, is a compound of Formula (If), or a pharmaceutically acceptable salt of a compound of Formula (If):

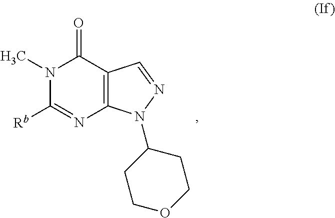

wherein R^{b} has any of the values described herein.

In certain embodiments of a chemical entity of Formula (If),

R^{b} is -L-L², L¹-L²-L³ or —N(-L⁴)L⁵;
L is a member selected from the group consisting of: a bond, —O—, —OCH₂—, —OCH₂CH₂—, and —NH—;
L¹ is a member selected from the group consisting of: —CH₂—, —CHF—, —CF₂—, —O—, —OCH₂—, —OCH₂CH₂—, and —NH—;
L² is aryl, 5-10-membered heteroaryl, —C₃₋₇cycloalkyl, or 3-12-membered heterocycloalkyl, all optionally substituted with 1 to 3 R^{1A}, where each R^{1A} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl; each n is independently 0, 1, or 2;
L³ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1B}, where each R^{1B} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl;
L⁴ and L⁵ are taken together to form a 3-12-membered heterocycloalkyl ring, optionally substituted with 1 to 3 R^{1C}, where each R^{1C} is independently selected from the group consisting of: L⁶, halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
L⁶ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1D}, where each R^{1D} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In some embodiments of a chemical entity of Formula (I), (Ib) (Id), (Ie), or (If) disclosed herein: L¹ is a member selected from the group consisting of: —CH₂—, —CHF—, —CF₂—, —O—, —OCH₂—, —OCH₂CH₂—, and —NH—.

In some embodiments of a chemical entity of Formula (I), (Ib), (Id), (Ie), or (If) disclosed herein: L¹ is a member selected from the group consisting of: —CH₂—, —O—, —OCH₂—, and —NH—.

In some embodiments of a chemical entity of Formula (I), (Ib), (Id), (Ie), or (If) disclosed herein: L¹ is —CH₂—.

In some embodiments of a chemical entity of Formula (I), (Ib), (Id), (Ie), or (If) disclosed herein: L¹ is —O—.

In some embodiments of a chemical entity of Formula (I), (Ib), (Id), (Ie), or (If) disclosed herein: L¹ is —NH—.

In some embodiments of a chemical entity of Formula (I), (Ia), (Id), (Ie), or (If) disclosed herein: L is —OCH₂— or —OCH₂CH₂—.

In some embodiments of a chemical entity of Formula (I), (Ia), (Id), (Ie), or (If) disclosed herein: L is —O—.

In some embodiments of a chemical entity of Formula (I), (Ia), (Id), (Ie), or (If) disclosed herein: L is —NH—.

In some embodiments of a chemical entity of Formula (I), (Ia), (Ib), (Iba), (Ibb), (Ibc), (Id), (Ie), or (If) disclosed herein: L² is aryl, 5-10-membered heteroaryl, —C₃₋₇cycloalkyl, or 3-12-membered heterocycloalkyl, all optionally and independently substituted with 1 to 3 R^{1A}.

In some embodiments of a chemical entity of Formula (I), (Ia), (Ib), (Iba), (Ibb), (Ibc), (Id), (Ie), or (If) disclosed herein: L² is phenyl, optionally substituted with 1 to 3 R^{1A}.

In some embodiments of a chemical entity of Formula (I), (Ia), (Ib), (Iba), (Ibb), (Ibc), (Id), (Ie), or (If) disclosed herein: L² is a 5-10-membered heteroaryl selected from the group consisting of: pyridine, pyrimidine, pyridazine, pyrazine, pyrazole, triazole, imidazole, thiazole, oxazole, isothiazole, isoxazole, indole, indazole, isoquinoline, and furo[2,3-d]pyrimidine, each optionally substituted with 1 to 3 R^{1A}.

In some embodiments of a chemical entity of Formula (I), (Ia), (Ib), (Iba), (Ibb), (Ibc), (Id), (Ie), or (If) disclosed herein: L² is —C₃₋₇cycloalkyl, said —C₃₋₇cycloalkyl optionally substituted with 1 to 3 R^{1A}.

In some embodiments of a chemical entity of Formula (I), (Ia), (Ib), (Ic), (Iba), (Ibb), (Ibc), (Id), (Ie), or (If) disclosed herein: L² is cyclohexyl, optionally substituted with 1 to 3 R^{1A}.

In some embodiments of a chemical entity of Formula (I), (Ia), (Ib), (Iba), (Ibb), (Ibc), (Id), (Ie), or (If) disclosed herein: L² is a 3-12-membered heterocycloalkyl selected from the group consisting of: quinolin-2(1H)-one, indolin-2-one, 1,2,3,4-tetrahydroisoquinoline, 3,4-dihydroisoquinolin-1(2H)-one, tetrahydrofuran, tetrahydropyran, morpholine, pyrrolidine, piperidine, and piperazine, each optionally substituted with 1 to 3 R^{1A}.

In some embodiments of a chemical entity of Formula (I), (Ib), (Iba), (Ibb), (Ibc), (Id), (Ie), or (If) disclosed herein: L³ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, and cycloalkyl, each optionally substituted with 1 to 3 R^{1B}.

In some embodiments of a chemical entity of Formula (I), (Ib), (Iba), (Ibb), (Ibc), (Id), (Ie), or (If) disclosed herein: L³ is phenyl, said phenyl optionally substituted with 1 to 3 R^{1B}.

In some embodiments of a chemical entity of Formula (I), (Ib), (Iba), (Ibb), (Ibc), (Id), (Ie), or (If) disclosed herein: L³ is 5-6-membered heteroaryl selected from the group consisting of: pyridine, pyrimidine, pyridazine, pyrazine, pyrazole, triazole, imidazole, thiazole, oxazole, isothiazole, isoxazole, indole, indazole, isoquinoline, and furo[2,3-d]pyrimidine, said heteroaryl optionally substituted with 1 to 3 R^{1B}.

In some embodiments of a chemical entity of Formula (I), (Ib), (Iba), (Ibb), (Ibc), (Id), (Ie), or (If) disclosed herein: L³ is —C₃₋₇cycloalkyl, said cycloalkyl optionally substituted with 1 to 3 R^{1B}.

In some embodiments of a chemical entity of Formula (I), (Ib), (Iba), (Ibb), (Ibc), (Id), (Ie), or (If) disclosed herein: L³ is a 3-10-membered heterocycloalkyl selected from the group consisting of: quinolin-2(1H)-one, indolin-2-one, 1,2,3,4-tetrahydroisoquinoline, 3,4-dihydroisoquinolin-1(2H)-one, tetrahydrofuran, tetrahydropyran, morpholine, pyrrolidine, piperidine, and piperazine, each optionally substituted with 1 to 3 R^{1B}.

In some embodiments of a chemical entity of Formula (I), (Ib), (Iba), (Ibb), (Ibc), (Id), (Ie), or (If) disclosed herein: L³ is —O(CH₂)ₙ—C₃₋₆cycloalkyl, —O(CH₂)ₙ-heterocycloalkyl, —(CH₂)ₙ-heterocycloalkyl, or —(CH₂)ₙ-heteroaryl.

In some embodiments of a chemical entity of Formula (I), (Ia), (Ib), (Iba), (Ibb), (Ibc), (Id), (Ie), or (If) disclosed herein: each R^{1A} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl.

In some embodiments of a chemical entity of Formula (I), (Ia), (Ib), (Iba), (Ibb), (Ibc), (Id), (Ie), or (If) disclosed herein: R^{b} is -L-L² or -L¹-L²-L³; and each R^{1A} is independently selected from the group consisting of: —F, —Br, —Cl, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₃₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl.

In some embodiments of a chemical entity of Formula (I), (Ib), (Iba), (Ibb), (Ibc), (Id), (Ie), or (If) disclosed herein: each R^{1B} is independently selected from the group consisting of: halo, —CN, —C₁₋₆alkyl, —C₁₋₄haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In some embodiments of a chemical entity of Formula (I), (Ib), (Iba), (Ibb), (Ibc), (Id), (Ie), or (If) disclosed herein: R^{b} is -L-L² or -L¹-L²-L³; and each R^{1B} is independently selected from the group consisting of: —F, —Br, —Cl, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In some embodiments of a chemical entity of Formula (I), (Ic), (Id), (Ie), or (If) disclosed herein: each R^{1C} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl.

In some embodiments of a chemical entity of Formula (I), (Ic), (Id), (Ie), or (If) disclosed herein: R^{b} is —N(L⁴)L⁵; and each R^{1C} is independently selected from the group consisting of: —F, —Br, —Cl, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl.

In some embodiments of a chemical entity of Formula (I), (Ic), (Id), (Ie), or (If) disclosed herein: each R^{1D} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In some embodiments of a chemical entity of Formula (I), (Ic), (Id), (Ie), or (If) disclosed herein: R^{b} is —N(L⁴)L⁵; and each R^{1D} is independently selected from the group consisting of: —F, —Br, —Cl, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In some embodiments of a chemical entity of Formula (I), (Ic), (Id), (Ie), or (If) disclosed herein: L⁴ and L⁵ are taken together with the nitrogen to which they are attached to form a 3-12-membered heterocycloalkyl group, said heterocycloalkyl substituted with one L⁶ and optionally with 1 or 2 additional R^{1C}.

In some embodiments of a chemical entity of Formula (I), (Ic), (Id), (Ie), or (If) disclosed herein: L⁴ and L⁵ are taken together with the nitrogen to which they are attached to form a 3-12-membered heterocycloalkyl selected from the group consisting of: quinolin-2(1H)-one, indolin-2-one, 1,2,3,4-tetrahydroisoquinoline, 3,4-dihydroisoquinolin-1(2H)-one, tetrahydrofuran, tetrahydropyran, morpholine, pyrrolidine, piperidine, and piperazine, each substituted with one L⁶ and optionally substituted with 1 or 2 additional R^{1C}.

In some embodiments of a chemical entity of Formula (I), (Ic), (Id), (Ie), or (If) disclosed herein: L⁶ is a member selected from the group consisting of: aryl, 5-6-membered heteroaryl, —C₃₋₇cycloalkyl, and 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1D}.

In some embodiments of a chemical entity of Formula (I) disclosed herein: L² is pyridine.

In some embodiments of a chemical entity of Formula (I) disclosed herein: L³ is pyrazole.

In some embodiments of a chemical entity of Formula (I) disclosed herein: L³ is pyridine.

In some embodiments of a chemical entity of Formula (I) disclosed herein: L is —O— and L² is phenyl.

In some embodiments of a chemical entity of Formula (I) disclosed herein: L is —O— and L² is pyridine.

In some embodiments of a chemical entity of Formula (I) disclosed herein: L¹ is —O— and L² is phenyl.

In some embodiments of a chemical entity of Formula (I) disclosed herein: L¹ is —O— and L² is pyridine.

In some embodiments of a chemical entity of Formula (I) disclosed herein: L¹ is —O—, L² is phenyl, and L³ is a 5-6-membered heteroaryl selected from the group consisting of: pyridine, pyrimidine, pyrazole, triazole, imidazole, thiazole, oxazole, isothiazole, and isoxazole, said heteroaryl optionally substituted with 1 to 3 R^{1B}.

In some embodiments of a chemical entity of Formula (I) disclosed herein: L¹ is —O—, L² is phenyl, and L³ is pyrazole.

In some embodiments of a chemical entity of Formula (I) disclosed herein: L¹ is —O—, L² is phenyl, and L³ is pyridine.

In some embodiments of a chemical entity of Formula (I) disclosed herein: L¹ is —O—, L² is phenyl, and L³ is pyrimidine.

In some embodiments of a chemical entity of Formula (I) disclosed herein: L¹ is —O—, L² is pyridine, and L³ is pyrazole.

### Formula (II)

Some embodiments provide a chemical entity of Formula (II), or, more specifically, a compound of Formula (II), or a pharmaceutically acceptable salt of a compound of Formula (II):

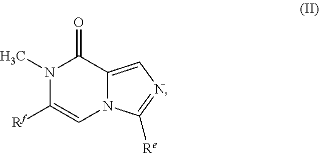

wherein: R^{e} and R^{f} have any of the values described herein.

In some embodiments of Formula (II),

R^{e} is a 3-6-membered cycloalkyl ring, a 3-6-membered cycloalkoxy ring, or —CHR^{g}R^{h}, where R^{g} and R^{h} are independently —C₁₋₄alkyl;
R^{f} is -L⁷-L⁸ or -L⁷-L⁸-L⁹;
L⁷ is —O— or —OCH₂—;
L⁸ is aryl, 5-10-membered heteroaryl, —C₃₋₇cycloalkyl, or 3-12-membered heterocycloalkyl, all optionally substituted with 1 to 3 R^{1E}, where each R^{1E} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —CH₂heteroaryl; each n is independently 0, 1 or 2; and
L⁹ is a member selected from the group consisting of: aryl, 5-6-membered heteroaryl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl and heterocycloalkyl optionally substituted with 1 to 3 R^{1F}, where each R^{1F} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In certain embodiments, a chemical entity of Formula (II) is a chemical entity of Formula (IIa), or, more specifically, a compound of Formula (IIa), or a pharmaceutically acceptable salt of a compound of Formula (IIa):

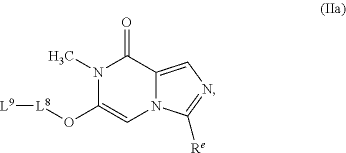

wherein: R^{e}, L⁸ and L⁹ have any of the values described herein.

In certain embodiments of Formula (IIa),

R^{e} is a 3-6-membered cycloalkyl ring, a 3-6-membered cycloalkoxy ring, or —CHR^{g}R^{h}, where R^{g} and R^{h} are independently —C₁₋₄alkyl;
L⁸ is aryl, 5-10-membered heteroaryl, —C₃₋₇cycloalkyl, or 3-12-membered heterocycloalkyl, all optionally substituted with 1 to 3 R^{1E}, where each R^{1E} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —CH₂heteroaryl;
each n is independently 0, 1, or 2; and
L⁹ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —CH₂heteroaryl, said aryl, heteroaryl, cycloalkyl and heterocycloalkyl optionally substituted with 1 to 3 R^{1F}, where each R^{1F} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In some embodiments of a chemical entity of Formula (II) and (IIa), R^{e} is —CHR^{g}R^{h}, where R^{g} and R^{h} are independently —C₁₋₄alkyl, or a 3-6-membered cycloalkyl ring, optionally containing one oxygen atom.

In some embodiments of a chemical entity of Formula (II) and (IIa), R^{e} is —CHR^{g}R^{h}, where R^{g} and R^{h} are independently —C₁₋₄alkyl.

In some embodiments of a chemical entity of Formula (II) and (IIa), R^{g} and R^{h} are —CH₃.

In some embodiments of a chemical entity of Formula (II) and (IIa), R^{f} is -L⁷-L⁸; and L⁸ is 5-10-membered heteroaryl, or 3-12-membered heterocycloalkyl, said heteroaryl, and heterocycloalkyl substituted with 1 to 3 R^{1E}, where each R^{1E} is independently selected from the group consisting of: halo, —C₁₋₃alkyl, —C₁₋₃haloalkyl, —C₁₋₃alkoxy, —C₁₋₃haloalkoxy, —C₃₋₅cycloalkyl, —C(O)C₁₋₃alkyl, —C₁₋₃alkyl-O—C₁₋₃alkyl, —C₁₋₃alkyl-CN, —OH, ═O, and —NHC₁₋₃alkyl.

In some embodiments of a chemical entity of Formula (II) and (IIa), R^{e} is a 3-6-membered cycloalkyl ring or a 3-6-membered cycloalkoxy ring.

In some embodiments of a chemical entity of Formula (II) and (IIa), R^{e} is a 3-6-membered cycloalkoxy ring.

In some embodiments of a chemical entity of Formula (II) and (IIa), R^{e} is a tetrahydropyran or tetrahydrofuran ring.

In some embodiments of a chemical entity of Formula (II) and (IIa), R^{e} is a tetrahydropyran ring.

In certain embodiments, a chemical entity of Formula (II) is a chemical entity of Formula (IIb), or, more specifically, a compound of Formula (IIb), or a pharmaceutically acceptable salt of a compound of Formula (IIb):

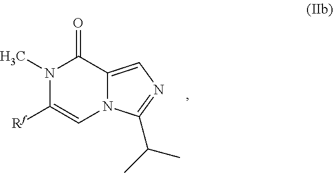

wherein: R^{f} has any of the values described herein.

In certain embodiments of Formula (IIb),

R^{f} is -L-L⁸ or -L⁷-L⁸-L⁹;
L⁷ is —O— or —OCH₂—;
L⁸ is aryl, 5-10-membered heteroaryl, —C₃₋₇cycloalkyl, or 3-12-membered heterocycloalkyl, all optionally substituted with 1 to 3 R^{1E}, where each R^{1E} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —CH₂heteroaryl;
each n is independently 0, 1 or 2; and
L⁹ is a member selected from the group consisting of: aryl, 5-6-membered heteroaryl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl and heterocycloalkyl optionally substituted with 1 to 3 R^{1F}, where each R^{1F} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In certain embodiments, a chemical entity of Formula (II) is a chemical entity of Formula (IIc), or, more specifically, a compound of Formula (IIc), or a pharmaceutically acceptable salt of a compound of Formula (Iic):

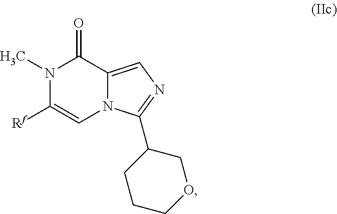

wherein: R^{f} has any of the values described herein.

In certain embodiments of Formula (IIc),

R^{f} is -L-L⁸ or -L⁷-L⁸-L⁹;
L⁷ is —O— or —OCH₂—;
L⁸ is aryl, 5-10-membered heteroaryl, —C₃₋₇cycloalkyl, or 3-12-membered heterocycloalkyl, all optionally substituted with 1 to 3 R^{1E}, where each R^{1E} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —CH₂heteroaryl;
each n is independently 0, 1 or 2; and
L⁹ is a member selected from the group consisting of: aryl, 5-6-membered heteroaryl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl and heterocycloalkyl optionally substituted with 1 to 3 R^{1F}, where each R^{1F} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁-(alkyl)₂, and —SC₁₋₆alkyl.

In certain embodiments, a chemical entity of Formula (II) is a chemical entity of Formula (IId), or, more specifically, a compound of Formula (IId), or a pharmaceutically acceptable salt of a compound of Formula (IId):

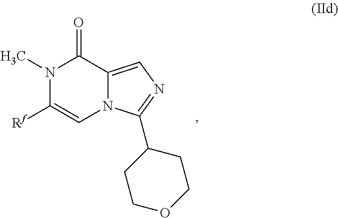

wherein: R^{f} has any of the values described herein.

In certain embodiments of Formula (IId),

R^{f} is -L-L⁸ or -L⁷-L⁸-L⁹;
L⁷ is —O— or —OCH₂—;
L⁸ is aryl, 5-10-membered heteroaryl, —C₃₋₇cycloalkyl, or 3-12-membered heterocycloalkyl, all optionally substituted with 1 to 3 R^{1E}, where each R^{1E} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —CH₂heteroaryl;
each n is independently 0, 1 or 2; and
L⁹ is a member selected from the group consisting of: aryl, 5-6-membered heteroaryl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl and heterocycloalkyl optionally substituted with 1 to 3 R^{1F}, where each R^{1F} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In some embodiments of a chemical entity of Formula (II), (IIa), (IIb), (IIc), and (IId), L⁷ is a —O— or —OCH₂—.

In some embodiments of a chemical entity of Formula (II), (IIa), (IIb), (IIc), and (IId), L⁷ is —OCH₂—.

In some embodiments of a chemical entity of Formula (II), (IIa), (IIb), (IIc), and (IId), L⁷ is a —O—.

In some embodiments of a chemical entity of Formula (II), (IIa), (IIb), (IIc), and (IId), L⁸ is aryl, 5-10-membered heteroaryl, —C₃₋₇cycloalkyl, or 3-12-membered heterocycloalkyl, all optionally substituted with 1 to 3 R^{1E}, where each R^{1E} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —CH₂heteroaryl.

In some embodiments of a chemical entity of Formula (II), (IIa), (IIb), (IIc), and (IId), L⁸ is phenyl, optionally substituted with 1 to 3 R^{1E}.

In some embodiments of a chemical entity of Formula (II), (IIa), (IIb), (IIc), and (IId), L⁸ is a 5-10-membered heteroaryl selected from the group consisting of: pyridine, pyrimidine, pyridazine, pyrazine, pyrazole, triazole, imidazole, thiazole, oxazole, isothiazole, isoxazole, indole, indazole, isoquinoline, furo[2,3-d]pyrimidine, optionally substituted with 1 to 3 R^{1E}.

In some embodiments of a chemical entity of Formula (II), (IIa), (IIb), (IIc), and (IId), L⁸ is —C₃₋₇cycloalkyl, optionally substituted with 1 to 3 R^{1E}.

In some embodiments of a chemical entity of Formula (II), (IIa), (IIb), (IIc), and (IId), La is -cyclohexyl, optionally substituted with 1 to 3 R^{1E}.

In some embodiments of a chemical entity of Formula (II), (IIa), (IIb), (IIc), and (IId), L⁸ is a 3-12-membered heterocycloalkyl selected from the group consisting of: quinolin-2(1H)-one, indolin-2-one, 1,2,3,4-tetrahydroisoquinoline, 3,4-dihydroisoquinolin-1(2H)-one, tetrahydrofuran, tetrahydropyran, morpholine, pyrrolidine, piperidine, and piperazine, optionally substituted with 1 to 3 R^{1E}.

In some embodiments of a chemical entity of Formula (II), (IIa), (IIb), (IIc), and (IId), R^{f} is -L⁷-L⁸-L⁹ and; L⁹ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1F}, where each R^{1F} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₄alkyl.

In some embodiments of a chemical entity of Formula (II), (IIa), (IIb), (IIc), and (IId), R is -L⁷-L⁸-L⁹ and; L⁹ is phenyl, optionally substituted with 1 to 3 R^{1F}.

In some embodiments of a chemical entity of Formula (II), (IIa), (IIb), (IIc), and (IId), R^{f} is -L⁷-L⁸-L⁹ and; L⁹ is a 5-6-membered heteroaryl selected from the group consisting of: pyridine, pyrimidine, pyridazine, pyrazine, pyrazole, triazole, imidazole, thiazole, oxazole, isothiazole, isoxazole, indole, indazole, isoquinoline, and furo[2,3-d]pyrimidine, optionally substituted with 1 to 3 R^{1F}.

In some embodiments of a chemical entity of Formula (II), (IIa), (IIb), (IIc), and (IId), R^{f} is -L⁷-L⁸-L⁹ and; L⁹ is —C₃₋₇cycloalkyl, optionally substituted with 1 to 3 R^{1F}.

In some embodiments of a chemical entity of Formula (II), (IIa), (IIb), (IIc), and (IId), R^{f} is -L⁷-L⁸-L⁹ and; L⁹ is a 3-10-membered heterocycloalkyl selected from the group consisting of: quinolin-2(1H)-one, indolin-2-one, 1,2,3,4-tetrahydroisoquinoline, 3,4-dihydroisoquinolin-1(2H)-one, tetrahydrofuran, tetrahydropyran, morpholine, pyrrolidine, piperidine, and piperazine, optionally substituted with 1 to 3 R^{1F}.

In some embodiments of a chemical entity of Formula (II), (IIa), (IIb), (IIc), and (IId), R^{f} is -L⁷-L⁸-L⁹ and; L⁹ is —O(CH₂)ₙ—C₃₋₆cycloalkyl, —O(CH₂)ₙ-heterocycloalkyl, —(CH₂)ₙ-heterocycloalkyl, or —(CH₂)ₙ-heteroaryl.

In some embodiments of a chemical entity of Formula (II), (IIa), (IIb), (IIc), and (IId), R^{1E} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl.

In some embodiments of a chemical entity of Formula (II), (IIa), (IIb), (IIc), and (IId), R^{f} is -L⁷-L⁸-L⁹ and; R^{1F} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

In some embodiments of a chemical entity of Formula (II) disclosed herein: L⁸ is pyridine.

In some embodiments of a chemical entity of Formula (II) disclosed herein: L⁹ is pyrazole.

In some embodiments of a chemical entity of Formula (II) disclosed herein: L⁹ is pyridine.

In some embodiments of a chemical entity of Formula (II) disclosed herein: L⁷ is —O— and L⁸ is phenyl.

In some embodiments of a chemical entity of Formula (II) disclosed herein: L⁷ is —O— and L⁸ is pyridine.

In some embodiments of a chemical entity of Formula (II) disclosed herein: L⁷ is —O—, La is phenyl, and L⁹ is a 5-6-membered heteroaryl selected from the group consisting of: pyridine, pyrimidine, pyrazole, triazole, imidazole, thiazole, oxazole, isothiazole, and isoxazole, said heteroaryl optionally substituted with 1 to 3 R^{1B}.

In some embodiments of a chemical entity of Formula (II) disclosed herein: L⁷ is —O—, L⁸ is phenyl, and L⁹ is pyrazole.

In some embodiments of a chemical entity of Formula (II) disclosed herein: L⁷ is —O—, L⁸ is phenyl, and L⁹ is pyridine.

In some embodiments of a chemical entity of Formula (II) disclosed herein: L⁷ is —O—, L⁸ is phenyl, and L⁹ is pyrimidine.

In some embodiments of a chemical entity of Formula (II) disclosed herein: L⁷ is —O—, L⁸ is pyridine, and L⁹ is pyrazole.

In some embodiments, a chemical entity is selected from compounds of Examples 1-202, and all pharmaceutically acceptable forms thereof, including pharmaceutically acceptable chelates, solvates, conformers, crystalline forms/polymorphs, salts, prodrugs, and pharmaceutically active metabolites. In other embodiments, a chemical entity is selected from compounds of Examples 1-202 and pharmaceutically acceptable salts thereof. In still other embodiments, a chemical entity is a compound selected from Examples 1-202.

In some embodiments, a chemical entity is selected from compounds of Examples 1-19 or Examples 26-94, and all pharmaceutically acceptable forms thereof, including pharmaceutically acceptable chelates, solvates, conformers, crystalline forms/polymorphs, salts, prodrugs, and pharmaceutically active metabolites. In other embodiments, a chemical entity is selected from compounds of Examples 1-19 or Examples 26-94 and pharmaceutically acceptable salts thereof. In still other embodiments, a chemical entity is a compound selected from Examples 1-19 or Examples 26-94.

In some embodiments, a chemical entity is selected from compounds of Examples 20-25 or Examples 95-202, and all pharmaceutically acceptable forms thereof, including pharmaceutically acceptable chelates, solvates, conformers, crystalline forms/polymorphs, salts, prodrugs, and pharmaceutically active metabolites. In other embodiments, a chemical entity is selected from compounds of Examples 20-25 or Examples 95-202 and pharmaceutically acceptable salts thereof. In still other embodiments, a chemical entity is a compound selected from Examples 20-25 or Examples 95-202.

Further embodiments are provided by pharmaceutically acceptable salts of compounds of Formula (I) and (II), tautomers of compounds of Formula (I) and (II), pharmaceutically acceptable prodrugs of compounds of Formula (I) and (II), and pharmaceutically active metabolites of compounds of Formula (I) and (II).

Isotopically-Labeled Compounds

Compounds of Formula (I) and (II) may include any isotope where one or more atoms are replaced by an atom having an atomic mass or mass number different from the atomic mass or mass number usually found in nature. For example, the isotopes may be isotopes of carbon, chlorine, fluorine, hydrogen, iodine, nitrogen, oxygen, phosphorous, sulfur, and technetium, including ¹¹C, ¹³C, ¹⁴C, ³⁶Cl, ¹⁸F, ²H, ³H, ¹²³I, ¹²⁵I, ¹³N, ¹⁵N, ¹⁵O, ¹⁷O, ¹⁸O, ³¹P, ³²P, ³⁵S, and ^{99m}Tc.

Compounds of the present invention (and all forms of such compounds, such as pharmaceutically acceptable salts) that contain the aforementioned isotopes or other isotopes of other atoms are within the scope of the invention. Isotopically-labeled compounds of the present embodiments are useful in binding affinity studies, as well as drug and substrate tissue distribution and target occupancy assays. For example, isotopically labeled compounds are particularly useful in SPECT (single photon emission computed tomography) and in PET (positron emission tomography), as discussed further herein. In addition, isotopically labelled compounds are useful for improving the absorption, distribution, metabolism and/or excretion (ADME) properties of drugs. For instance, replacement of one or more hydrogen atoms with deuterium (²H) can modify the metabolism of a drug and improve the metabolic profile by decreasing the metabolic clearance in vivo, extending the half-life, reducing Cₘₐₓ or reducing levels of potentially toxic metabolites.

### Compositions

In some embodiments, the chemical entities disclosed herein, and more particularly, compounds and pharmaceutically acceptable salts thereof, are used, alone or in combination with one or more additional active ingredients, to formulate pharmaceutical compositions.

In some embodiments, a pharmaceutical composition can comprise: (a) an effective amount of at least one chemical entity of the present disclosure; and (b) a pharmaceutically acceptable carrier.

In some embodiments, a pharmaceutical composition comprises a compound, or pharmaceutically acceptable salt thereof, of any of the embodiments and examples disclosed herein; and a pharmaceutically acceptable carrier. In specific embodiments, a pharmaceutical composition comprises a compound of any one of preparative examples 1-202; and a pharmaceutically acceptable carrier.

Formulations and Administration

Numerous standard references are available that describe procedures for preparing various formulations suitable for administering the compounds according to the invention.

Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Osol, ed.), 1980, 1553-1593.

Any suitable route of administration may be employed for providing an animal, especially a human, with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent, or excipient used will depend upon the means and purpose for which the compound of the present invention is being applied. Solvents are generally selected based on solvents recognized by persons skilled in the art as safe (GRAS) to be administered to an animal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., a compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable and appropriate dosage of the drug.

The pharmaceutical composition (or formulation) for application may be packaged in a variety of ways, depending upon the method used to administer the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Dosage Forms

The present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be in a range from 1% to 65% or 2 to 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are typically prepared by incorporating the active compound in the required amount in the appropriate solvent with a variety of the other ingredients enumerated above, as required, followed by filter sterilization. In the case of sterile powders for the preparation of sterile injectable solutions, common methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid. These compositions and formulations can be prepared according to ordinary skill in the art.

Useful solid carriers include finely divided solids such as talc, clay, microcrystalline cellulose, silica, alumina, and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Dosages

Useful dosages of the chemical entities and compounds (active agents) of the present disclosure can be determined by comparing their in vitro activity and in vivo activity in animal models. Methods for the extrapolation of effective dosages in mice, and other animals, to humans are known to the art. Useful dosages of the compounds of formula I can be determined by comparing their in vitro activity, and in vivo activity in animal models. Methods for the extrapolation of effective dosages in mice, and other animals, to humans are known to the art (e.g., U.S. Pat. No. 4,938,949).

Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, concomitant medications, and the judgment of the treating physician. An exemplary dose can be in the range from 0.0001 to 200 mg of active agent per day, from 0.001 to 200 mg per day, from 0.05 to 100 mg per day, from 0.1 to 10 mg/day, from 1 to 200 mg/day, or from 5 to 50 mg/day.

In some embodiments, the desired dose may be presented in a unit dosage form; for example, a composition containing from 0.01 to 1000 mg, from 0.1 to 200 mg, from 0.5 to 100 mg, or from 1 to 50 mg, of active ingredient per unit dosage form.

In other embodiments, the desired dose may be presented in divided doses administered at appropriate intervals, for example, as two, three, four, or more sub-doses per day. (e.g., BID, TID, QID). The sub-dose itself may be further divided, e.g., into a number of temporally-distinct administrations used according to the compositions and methods of the present invention.

### Methods and Uses

Uses of Isotopically-Labeled Compounds

In some embodiments, the present disclosure provides methods of using isotopically labeled compounds the present invention in: (i) metabolic studies (with, for example, ¹⁴C) and reaction kinetic studies (with, for example ²H or ³H); (ii) detection or imaging techniques [such as positron emission tomography (PET) or single-photon emission computed tomography (SPECT)] including drug or substrate tissue distribution assays; or (iii) radioactive treatment of patients.

Isotopically labeled compounds and related chemical entities of Formula (I) and (II) can generally be prepared by carrying out the procedures disclosed in the schemes or in the examples and preparations described below by substituting a readily available isotopically labeled reagent for a non-isotopically labeled reagent. Compounds labeled with ¹⁸F or ¹¹C may be particularly preferred for PET, and an ¹²³I-labeled compound may be particularly preferred for SPECT studies. Further substitution of compounds of Formula (I) and (II) with heavier isotopes such as deuterium (i.e., ²H) may afford certain therapeutic advantages resulting from greater metabolic stability, for example increased in vivo half-life or reduced dosage requirements.

Therapeutic Methods

Generally Chemical entities and compositions of the present disclosure are useful in various therapeutic methods (or in the manufacture of a medicament for use in such methods), comprising administering to a subject in need thereof a chemical entity or composition herein. In a specific aspect, the chemical entity is a compound of Formula (I) or (II) or a pharmaceutically acceptable salt thereof. Such therapeutic methods can be directed to a wide range of indications, as described further herein, including cognitive or motor deficits associated with neurological disorders, neurodegenerative disorders, immunological and inflammatory disorders, and numerous peripheral disorders.

In some embodiments, chemical entities and compositions herein are useful in methods of inhibiting PDE1 activity, comprising exposing PDE1 to an effective amount of a chemical entity or composition of any one of the embodiments disclosed herein. In some embodiments, the PDE1 is in an animal, and more particularly, is in a human subject. In some embodiments, chemical entities and compositions herein show selectivity for PDE1 enzymes versus enzyme isoforms from many, if not all, other PDE families. More particularly, in some embodiments, chemical entities and compositions herein show greater specificity for the PDE1B enzyme subtype compared to the PDE1A and PDE1C subtypes.

In some embodiments, chemical entities and compositions herein are useful in methods of treating a subject suffering from or diagnosed with a disorder mediated by PDE1 activity, comprising administering to a subject in need thereof an effective amount of a chemical entity or composition of any one of the embodiments herein. In one aspect, the subject is diagnosed with a disorder mediated by PDE1 activity. In another aspect, the subject is suffering from a disorder mediated by PDE1 activity.

In some embodiments, chemical entities and compositions herein are useful in methods of enhancing neuronal plasticity, an essential property of the brain that can be impaired in numerous CNS disorders and augmented in healthy animals. Without being limited by mechanism, such chemical entities can enhance cyclic adenosine monophosphate (cAMP) response element binding protein (CREB) pathway function in cells, modulating transcription of multiple genes involved in synaptic plasticity (see, e.g., Tully et al., 2003, Nat. Rev. Drug Discov. 2, 267-277; Alberini, 2009, Physiol. Rev. 89, 121-145; Medina, 2011, Front. Neurosci. 5, 21). Accordingly, in some embodiments, the present disclosure provides methods of enhancing neuronal plasticity, comprising administering to a subject in need thereof an effective amount of a chemical entity or composition of any one of the embodiments herein. In specific embodiments, chemical entities of the present disclosure are useful in methods of enhancing cognitive or motor function, comprising administering to a subject in need thereof an effective amount of a chemical entity or composition of any one of the embodiments disclosed herein.

In some embodiments, chemical entities and compositions herein are used as neuroprotective agents, for example, by enhancing neuronal growth and survival. Accordingly, the present disclosure provides methods of conferring neuroprotection, comprising administering to a subject in need thereof an effective amount of a chemical entity of Formula (I) or (II).

In some embodiments, chemical entities and compositions herein are used as agents to promote neurogenesis, which may be applicable to treating neurological disorders, as described further herein. PDE1B is highly expressed in the dentate gyrus and olfactory bulb, the two areas where neurogenesis occurs in the adult nervous system. Neurogenesis in the hippocampus has been implicated in memory formation in depression, and in cognitive deficits underlying neuropsychiatric disease, including, but not limited to, PTSD and other anxiety disorders. See, e.g., Shors et al., 2001, Nature 410, 372-376; Shors et al., 2004, Trends Neurosci. 27, 250-256; Ming and Song, 2011, Neuron 70, 687-702; Hill et al., 2015, Neuropsychopharmacology 40, 2368-2378; Kheirbek et al., 2012, Nat. Neurosci. 15, 1613-1620.

In some embodiments, chemical entities and compositions herein are used as treating disorders that include aberrant or dysregulated signaling pathways mediated by PDE1. Such PDE1-related signaling pathways include, but are not limited to, those involving nitric oxide, natriuretic peptides (e.g., ANP, BNP, CNP), dopamine, noradrenalin, neurotensin, cholecystokinin (CCK), vasoactive intestinal peptide (VIP), serotonin, glutamate (e.g., NMDA receptor, AMPA receptor), GABA, acetylcholine, adenosine (e.g., A2A receptor), cannabinoids, natriuretic peptides (e.g., ANP, BNP, CNP), and endorphins.

In a specific aspect, they are useful in modulating dopaminergic signaling or treating disorders characterized by alterations in dopamine signaling, particularly dopaminergic signaling mediated by the dopamine receptor D1, which in humans is encoded by the DRD1 gene. See, e.g., Nishi and Snyder, 2010, J. Pharmacol. Sci. 114, 6-16.

In some embodiments, chemical entities and compositions are used as “agents” (or “augmenting agents”) to increase the efficiency of training protocols that facilitate functional reorganization in targeted “domains” (or “functions”) in the brain.

In some embodiments, chemical entities and compositions are used in combination with other therapies or with other active agents, as described further herein.

Neurological Disorders

In some embodiments the present disclosure provides methods of treating neurological disorders, comprising administering to a subject in need thereof a chemical entity or composition described herein. In a specific aspect, the chemical entity is a compound of Formula (I) or (II), or pharmaceutically acceptable salt thereof.

In some embodiments, the method is directed to a neurological impairment (“neurological deficit”) associated with the neurological disorder, including a cognitive impairment (“cognitive deficit”) or a motor impairment (“motor deficit”) associated with the pathology of the neurological disorder.

A cognitive impairment can manifest, for example, as a deficit in: attention (e.g., sustained attention, divided attention, selective attention, processing speed); executive function (e.g., planning, decision, and working memory); memory (e.g., immediate memory; recent memory, including free recall, cued recall, and recognition memory; and long-term memory, which can be divided into explicit memory (declarative memory), such as episodic, semantic, and autobiographical memory, and into implicit memory (e.g., procedural memory)); expressive language, including naming, word recall, fluency, grammar, and syntax; understanding speech or writing (e.g., aphasia); perceptual-motor functions (e.g., abilities encompassed under visual perception, visual-constructional, perceptual-motor praxis, and gnosis); and social cognition (e.g., recognition of emotions, theory of mind). In certain embodiments, the cognitive deficit is a deficit in memory and more particularly, a deficit in long-term memory.

A motor impairment can manifest, for example, as weakness or paralysis, deficits in upper and lower extremity function, problems with balance or coordination, impairments of gross motor skills, and deficits in fine motor skills.

A neurological disorder (or condition or disease) is any disorder of the body's nervous system. Neurological disorders can be categorized according to the primary location affected, the primary type of dysfunction involved, and the primary type of cause. The broadest division is between disorders of the central nervous system (CNS), which comprises the nerves in the brain and spinal cord, and disorders of the peripheral nervous system (PNS), which comprises the nerves outside the brain and spinal cord.

Many CNS disorders are amenable for treatment with chemical entities and compositions, including those discussed herein. As used herein, the terms “Neurodevelopment disorders,” “Schizophrenia spectrum and other psychotic disorders,” “Bipolar and related disorders,” “Depressive disorders,” “Anxiety disorders,” “Obsessive-compulsive and related disorders,” “Dissociative disorders,” “Disruptive, impulse-control, and conduct disorders,” “Trauma- and stressor-related disorders,” “Feeding and eating disorders,” “Sleep disorders,” “Sexual disorders,” “Substance-related and addictive disorders,” “Personality disorders,” “Somatic symptom disorders,” “Neurodegenerative disorders,” “Neurocognitive disorders,” “Delirium,” “Dementias,” and “Age-associated cognitive deficits,” include the diagnosis and classification of these CNS conditions and disorders (and related CNS conditions and disorders) as described in the Diagnostic and Statistical Manual of Mental Disorders (DSM-5; 5^{th} ed., 2013, American Psychiatric Association). The skilled artisan will recognize that there are alternative nomenclature and classification systems for these CNS disorders, and that these systems evolve with medical and scientific progress. Thus, these terms in this paragraph are intended to include like disorders that are described in other diagnostic sources.

Mental and Psychiatric Disorders:

In certain embodiments, chemical entities and compositions herein are useful in treating mental or psychiatric disorders, and more particularly, a cognitive impairment associated with the pathology of such disorders. In a specific aspect, the chemical entity is a compound of Formula (I) or (II), or pharmaceutically acceptable salt thereof.

Mental and psychiatric disorders are well known in the art, and include, but are not limited to, one or more of the following:
Neurodevelopmental (or “developmental” disorders), such as intellectual disability disorders (e.g., Rubinstein-Taybi syndrome, Down syndrome, and Fragile X syndrome); communication disorders; autism-spectrum disorders; attention-deficit/hyperactivity disorders; specific learning, language, or reading (e.g., dyslexia) disorders; motor disorders; fetal alcohol spectrum disorders (FASD); and other neurodevelopmental disorders;
Schizophrenia spectrum and other psychotic disorders, such as schizophrenia, schizotypal (personality) disorder, delusional disorder, brief psychotic disorder, schizoaffective disorder, substance/medication-induced psychotic disorder, psychotic disorder due to another medical condition, catatonia, catatonia associated with another mental disorder (catatonia specifier), catatonic disorder due to another medical condition, unspecified catatonia, schizophreniform disorder, and other schizophrenia spectrum and psychotic disorders;
Bipolar and related disorders, such as Bipolar I and II disorders, cyclothymic disorders, and other bipolar and related disorders;
Depressive disorders, such as major depressive disorder, persistent depressive disorder (dysthymia), a major depressive episode of the mild, moderate, or severe type, a depressive episode with melancholic features, a depressive episode with catatonic features, seasonal depression (seasonal affective disorder), disruptive mood dysregulation disorder, premenstrual dysphoric disorder, substance/medication-induced depressive disorder, depressive disorder due to another medical condition, mood disorders due to a general medical conditions, and other depressive disorder;
Anxiety disorders, such as specific phobia, agoraphobia, social anxiety disorder (social phobia), panic attack, panic disorder, acute stress disorder, generalized anxiety disorder, posttraumatic stress disorder (PTSD), and other anxiety disorders;
Obsessive-compulsive and related disorders, such as obsessive-compulsive disorder (OCD), body dysmorphic disorder, hoarding disorder, trichotillomania (hair-pulling disorder), excoriation (skin-picking) disorder, substance/medication-induced obsessive-compulsive and related disorder, obsessive-compulsive and related disorder due to another medical condition, and other specified obsessive-compulsive and related disorder and unspecified obsessive-compulsive and related disorder (e.g., body-focused repetitive behavior disorder, obsessional jealousy), and other obsessive-compulsive and related disorders;
Dissociative disorders, such as dissociative identity disorder, dissociative amnesia, depersonalization/derealization disorder, dissociative subtypes (in conjunction with other disorders), and other dissociative disorders;
Disruptive, impulse-control, and conduct disorders, such as conduct disorder, antisocial personality disorder, pyromania, kleptomania, and other disruptive, impulse-control, and conduct disorders;
Trauma- and stressor-related disorders, such as reactive attachment disorder, disinhibited social engagement disorder, posttraumatic stress disorder, acute stress disorder, adjustment disorders, and other trauma- and stressor-related disorders;
Feeding and eating disorders, such as pica, rumination disorder, avoidant/restrictive food intake disorder, anorexia, bulimia, binge-eating disorder, and other feeding and eating disorders;
Sleep disorders, such as sleep-wake disorders, insomnia disorder, hypersomnolence disorder, narcolepsy, breathing-related sleep disorders, sleep apnea, circadian rhythm sleep-wake disorders, non-rapid eye movement (NREM) sleep arousal disorders, nightmare disorder, rapid eye movement (REM) sleep behavior disorder, restless legs syndrome, and substance/medication-induced sleep disorder, parasomnias, and other sleep-wake disorders;
Sexual disorders, such as arousal disorders, desire disorders, dysfunctions, substance- and medication-induced dysfunctions, impotence and other sexual disorders;
Substance-related and addictive disorders, such as those involving alcohol, drugs, stimulants, opioids, tobacco, and non-substance-related addictive disorders; and other substance-related and addictive disorders;
Personality disorders, such as antisocial personality disorder, borderline personality disorder, histrionic personality disorder, narcissistic personality disorder, avoidant personality disorder, dependent personality disorder, obsessive-compulsive personality disorder, paranoid personality disorder, schizoid personality disorder, schizotypal personality disorder, personality change due to another medical condition, and other personality disorders; and
Somatic symptom and related disorders, such as somatic symptom disorder, illness anxiety disorder (hypochondriasis), factitious disorder, factitious disorder imposed on another, pain disorders, conversion disorder, and other somatic symptom and related disorders.

Schizophrenia:

In specific embodiments, the mental or psychiatric disorder is a schizophrenia spectrum or psychotic disorder, and, in particular, is schizophrenia. Schizophrenia is a devastating neurological disorder, characterized by a combination of symptoms, which may include negative, positive, or cognitive symptoms. Negative symptoms can include flat affect (lack or decline in emotional response), alogia (lack or decline in speech), avolition (lack or decline in motivation), anhedonia (the inability to experience pleasure from activities usually found enjoyable), and asociality (lack of motivation to engage in social interaction, or a preference for solitary activities). Positive symptoms include paranoia, hallucinations, and delusions. Cognitive symptoms can include impairments in such functions as attention, memory, reasoning, and processing speed. See, e.g., Keefe and Harvey, 2012, Handb. Exp. Pharmacol. 213, 11-23. Intracellular signaling of dopamine D1 and various serotonin receptors, which signal through cyclic nucleotides, is known to be defective in schizophrenia, as well as depression and other cognitive disorders. More generally, PDEs, include PDE1, have been implicated at the interface between cognitive deficits and neuropsychiatric disorders. See, e.g., Wang et al., 2015, Curr. Pharm. Des. 21, 303-316.

Accordingly, the present disclosure provides a method of treating schizophrenia, comprising administering to a subject in need thereof an effective amount of a chemical entity or composition herein. In a specific aspect, the chemical entity is a compound of Formula (I) or (II), or pharmaceutically acceptable salt thereof. In some embodiments, the treatment is directed to a positive symptom of schizophrenia. In some embodiments, treatment is directed to a negative symptom of schizophrenia. In some embodiments, treatment is directed to cognitive impairment associated with schizophrenia (CIAS). In some embodiments, the treatment also include a cognitive training protocol.

Addictive Disorders:

In specific embodiments, the mental or psychiatric disorder is an addictive disorder.

In one aspect, the subject is addicted to an addictive agent selected from the group consisting of alcohol, nicotine, marijuana, a marijuana derivative, an opioid agonist (such as morphine, methadone, fentanyl, sufentanil, or heroin), a benzodiazepine, a barbiturate, and a psychostimulant, such as cocaine or amphetamine. In another aspect, the addiction is associated with an obsessive-compulsive disorder. In another aspect, the disorder is associated with a primary impulse-control disorder, such as binge eating, pathological gambling, addiction to pornography, sex addiction, compulsive spending, anorexia, bulimia, kleptomania, pyromania, trichotillomania, compulsive over-exercising, or compulsive overworking.

Accordingly, the present disclosure provides a method of treating an addictive disorder, comprising administering to a subject in need thereof an effective amount of a chemical entity or composition herein. In a specific embodiment, the chemical entity is a compound of Formula (I) or (II), or pharmaceutically acceptable salt thereof.

Cognitive Disorders:

In specific embodiments, the present disclosure provides a method of treating a cognitive disorder, and more particularly, a neurological impairment associated with the disorder, comprising administering to a subject in need thereof an effective amount of a chemical entity or composition described herein. In a specific aspect, the chemical entity is a compound of Formula (I) or (II), or pharmaceutically acceptable salt thereof.

A “cognitive disorder” (or “neurocognitive disorder”) is one in which the primary clinical feature is impaired cognition, i.e., a disorder in which the primary cognitive deficit has not been present since birth or very early life and therefore represents a decline from a previously attained level of functioning. Such disorders, include one or more of the following:
Delirium, such as substance-intoxication (or withdrawal) delirium, medication-induced delirium, and other forms of delirium;
Dementias and other cognitive impairments due to acquired diseases, such as HIV infection, or transmissible encephalopathies; or due to neurodegenerative or progressive nervous system diseases, such as Alzheimer's disease, Parkinson's disease (in particular Parkinson's Disease Dementia (PDD)), Huntington's disease, Lewy body disease, Pick's disease, a prion disease (e.g., Creutzfeldt-Jakob disease), Amyotrophic lateral sclerosis (ALS), multiple sclerosis (MS), frontotemporal lobar degeneration (FTLD), and corticobasal degeneration;
dementia due to a vascular disease (“vascular disease”); autoimmune disorders; and other dementias and neurodegenerative diseases.
Age-associated cognitive decline, including age-associated memory impairment (AAMI), also referred to as age-related memory impairment (AMI) (see, e.g., Crook et al., 1986, Devel. Neuropsychol. 2, 261-276); and cognitive decline affecting patients in early stages of cognitive decline, as in Mild Cognitive Impairment (MCI) (see, e.g., Arnmiz and Almkvist, 2003, Acta Neurol. Scand. Suppl. 179, 34-41);
Trauma-dependent losses of function, including vascular diseases, such as stroke (e.g., ischemic or hemorrhagic stroke) or ischemia; infarction, including cerebral and myocardial; microvascular or macrovascular disease arising from diabetes or arthrosclerosis; traumatic brain injury (TBI), such as brain trauma including subdural hematoma and brain tumor; head trauma (closed and penetrating); head injury; tumors, such as nervous system cancers, including cerebral tumors affecting the thalamic or temporal lobe; hypoxia, and viral, fungal, or bacterial infection (e.g., encephalitis, or meningitis); excitotoxicity; and seizures; and
Cognitive impairments due to chemotherapy, such as post-chemotherapy cognitive impairments (PCCI); chemotherapy-induced cognitive dysfunction or impairments; chemo brain; or chemo fog.

Such cognitive disorders can include neurological impairments other than cognitive impairments. For example, trauma-dependent losses of function, such as stroke, traumatic brain injury, head trauma, and head injury, can include impairments in multiple neurological functions, such as impairments in motor functions.

Age Associated Cognitive Decline:

In specific embodiments, the cognitive disorder is age-associated cognitive decline.

In one aspect, the age-related cognitive decline is age-associated memory impairment (AAMI). AAMI is a decline in various cognitive abilities, in particular memory abilities, associated with normal aging. For example, AAMI subjects show a decline in the ability to encode new memories of events or facts, as well as in working memory (Hedden and Gabrieli, 2004, Nat. Rev. Neurosci. 5, 87-96). In addition, AAMI subjects, when compared with age-matched controls, appeared to be impaired in tests of executive functions associated with frontal lobe function. These and other studies suggest an important role for frontal lobe dysfunction in the memory loss of elderly people (Nilsson, 2003, Acta Scand. Suppl. 179, 7-13). In general, an AAMI diagnosis identifies persons with subjectively and objectively evidenced memory loss without cognitive decline impaired enough to warrant the diagnosis of dementia. For example, the NIH working group has established multiple criteria for a diagnosis of AAMI in a person aged 50 or older, including the presence of subjective memory decline, objective evidence of memory loss, evidence of adequate intellectual function, and the absence of dementia (or other memory-affecting disease) (Crook et al., 1986, Devel. Neuropsychol. 2, 261-276). Individuals with AAMI have been shown to have a three-fold greater risk for development of dementia than individuals who do not meet AAMI criteria (Goldman and Morris, 2002, Alzheimer Dis. Assoc. Disord. 75, 72-79).

In another aspect, the age-associated cognitive decline is Mild Cognitive Impairment, which may be diagnosed when an individual's memory declines below the level considered normal for that age group. In other words, MCI is a condition in which people face memory problems more often than that of the average person their age. Symptoms often include misplacing items, forgetting events or appointments, and having trouble thinking of desired words (e.g., Arnaiz and Almkvist, 2003, Acta Neurol. Scand. Suppl. 179, 34-41). MCI can represent a transitional state between cognitive changes of normal aging and Alzheimer's disease (AD). Many people who experience mild cognitive impairment are at a high risk of developing Alzheimer's disease. About 12% of people aged 65 or older diagnosed with MCI go on to develop Alzheimer's disease within a year, and about 40% develop Alzheimer's within three years. This is a much higher rate than in the general population, in which only about 1% of people aged 65 or older develop Alzheimer's each year. Thus, people with MCI are considered at heightened risk to develop Alzheimer's disease. Some patients with MCI, however, never progress to AD.

Accordingly, the disclosure includes methods of treating age-associated cognitive decline, and more particularly, age-related memory impairment or mild cognitive impairment, comprising administering to a subject in need thereof an effective amount of a chemical entity or composition disclosed herein. In a specific aspect, the chemical entity is a compound of Formula (I) or (II), or pharmaceutically acceptable salt thereof.

Trauma-Dependent Loss of Function:

In specific embodiments, the cognitive disorder is a trauma-dependent loss of function, and more particularly, stroke or TBI. Accordingly, the disclosure includes methods of treating a trauma-dependent loss of function, and more particularly, stroke or TBI, comprising administering to a subject in need thereof an effective amount of a chemical entity or composition disclosed herein.

Movement Disorders:

In certain embodiments, the present disclosure provides methods of treating movement and motor disorders, and more particularly, a movement or motor impairment associated with the pathology of such disorders, comprising administering to a subject in need thereof an effective amount of a chemical entity or composition described herein. In a specific aspect, the chemical entity is a compound of Formula (I) or (II), or pharmaceutically acceptable salt thereof.

Loss of dopaminergic neurotransmission in striatum is a central cause of neurodegenerative diseases leading to movement disorders, such as Parkinson's disease and Huntington's disease. See, e.g., Sasaki et al., 2004, J. Neurochem. 89, 474-483; Morales-Garcia et al., 2014, Neurobiol. Aging. 36, 1160-1173; Banerjee et al., 2012, Bioorg. Med. Chem. Lett. 22, 6286-6291. PDE1 is highly expressed in the striatum, and growing amount of evidence suggest that phosphodiesterases play a critical role in modulating dopamine signaling in the brain (Ramirez and Smith, 2014, Cent. Nerv. Syst. Agents Med. Chem. 14, 72-82).

Movement disorders include, but are not limited to, basal ganglia disorders, Parkinson's disease, Post-Encephalitic Parkinsonism, Dopamine-Responsive Dystonia, Hallervorden-Spatz Syndrome (HSS), Restless Leg Syndromes, Wilson's Disease, Shy-Drager Syndrome, Periodic Limb Movement Disorder (PLMD), Periodic Limb Movements in Sleep (PLMS), Tourette's Syndrome, Restless Leg(s) Syndrome (RLS); chorea, such as that in Huntington's disease; myoclonus (including generalized myoclonus and focal myoclonus); tics (including simple tics, complex tics and symptomatic tics); and hyperkinetic, hypokinetic, and dyskinetic disorders; movement disorders induced by drugs, diseases associated with striatal hypofunction; and other movement and motor disorders.

In specific embodiments, the dyskinetic disorder is a drug-induced dyskinesia. More particularly, the dyskinetic disorder is levodopa induced dyskinesia (LID) or tardive dyskinesia (TD), which represent the most common forms of drug-induced dyskinesias. For example, uncontrolled stimulation of supersensitized dopamine D1 receptors in the direct striatonigral pathway are thought to mediate LIDs. In addition, long-term blockade of dopamine D2 receptors in the basal ganglia by dopamine D2 antagonists (e.g., neuroleptics) may produce compensatory supersensitivity of dopamine receptors and TD. Accordingly, in specific embodiments, then present disclosure provides methods of treating LID (or TD), comprising administering to a subject in need therefor an effective amount of a chemical entity of any of the embodiments disclosed herein.

In certain embodiments, the movement disorder is a basal ganglia disorder.

In other embodiments, the movement disorder includes kinesias and akinetic-rigid syndromes, such as Parkinson's disease or corticobasal degeneration; Tourette's syndrome, epilepsy, muscular spasms, and disorders associated with muscular spasticity or weakness; dyskinesias, including tremors, such as rest tremor, postural tremor and intention tremor.

In specific embodiments, the movement disorder is Parkinson's disease or Huntington's disease, as discussed further herein.

In some embodiments, the methods are directed to a specific movement abnormality associated with the pathology of a movement or motor disorder. Movement abnormalities include, but are not limited to, tremors, resting tremors, rigidity, bradykinesia, and deficient postural reflexes.

Neurodegenerative Disorders:

In specific embodiments, the disclosure provides methods of treating a neurodegenerative disorder, and more particularly treating a neurological impairment associated with the pathology of a neurodegenerative disorder, comprising administering to a subject in need thereof an effective amount of a chemical entity or composition described herein.

Neurodegenerative disorders can result from a primary nervous system disease or a primary nervous system injury. Chronic neuroinflammation is a hallmark of neurodegenerative disorders, and in animal and cellular models, PDE1 inhibition shows neuroprotective and anti-inflammatory effects that are expected to be beneficial in treating neuroinflammation and other hallmarks of such disorders.

Accordingly, in some embodiments, the therapeutic methods are directed to neurodegenerative disorders resulting from a primary nervous system disease. Such diseases include, but are not limited to, Parkinson's disease, Alzheimer's disease, Huntington's disease, Lewy body disease, Pick's disease, a prion disease (e.g., Creutzfeldt-Jakob disease), Amyotrophic lateral sclerosis (ALS), multiple sclerosis (MS), frontotemporal lobar degeneration (FTLD), and corticobasal degeneration.

In other embodiments, the therapeutic methods are directed to a neurodegenerative disorder resulting from a primary nervous system injury. Such primary injuries can include, but are not limited to, stroke, including hemorrhagic stroke and ischemic stroke; a traumatic brain injury (TBI), which can include closed head injuries and blunt trauma, including those caused by participation in sports, and penetrating trauma, such as gunshot wounds; spinal cord injuries; glaucoma, cerebral ischemia, or damages caused by surgery such as tumor excision.

Parkinson's Disease:

In specific embodiments, the present disclosure provides methods of treating Parkinson's disease, comprising administering to a subject in need thereof an effective amount of a chemical entity or composition described herein. Parkinson's disease (PD), also known as Parkinson's, idiopathic Parkinsonism, or primary Parkinsonism, is a degenerative disorder of the CNS estimated to afflict more than 5 million people worldwide. It is a slowly progressive neurological condition, characterized by tremors, stiffness, slowness of movement (bradykinesia) and impaired balance. Altered cAMP/cGMP levels are associated with Parkinson's disease, and PDE1B activity is increased in models of Parkinson' disease. Sancesario et al., 2004, Eur. J. Neurosci. 20, 989-1000.

While Parkinson's disease has been defined by its motor hallmarks, non-motor features such as cognitive impairment and dementia have been increasingly recognized. For example, MCI is common in a significant fraction (with estimates ranging from 20%-50%) of non-demented PD patients. See, e.g., Broeders et al., 2013, Neurology 81, 346-352. While diagnostic criteria are not completely uniform, PD patients with MCI (PD-MCI patients) typically exhibit non-amnestic deficits in cognitive domains such as executive function, attention, and visuospatial function (Litvan et al., 2012, Mov. Disord. 27, 349-356). The cognitive phenotype of PD-MCI is heterogeneous, however, with some patients demonstrating amnestic deficits. Certain PD-MCI patients may be at high risk for developing dementia. (e.g., Goldman and Litvan, 2011, Minerva Med. 102, 441-459).

Thus, in specific embodiments, chemical entities and compositions herein can be used to treat motor deficits associated with PD, and in other embodiments to treat cognitive impairments associated with PD, including in PD-MCI subjects. In a specific aspect, the chemical entity is a compound of Formula (I) or (II), or pharmaceutically acceptable salt thereof.

Alzheimer's Disease:

In specific embodiments, the present disclosure provides methods of treating Alzheimer's disease (AD), comprising administering to an animal in need thereof an effective amount of a chemical entity or composition disclosed herein. In a specific aspect, the chemical entity is a compound of Formula (I) or (II), or pharmaceutically acceptable salt thereof. Alzheimer's disease is a neurodegenerative disorder that involves the progressive loss of memory and other cognitive functions. Although the pathogenesis of AD is not well known, its etiology is associated with the presence of β-amyloid (or senile) plaques; deficiencies in neurotransmission; loss of neurons, especially in the cortex and hippocampus; neurofibrillary tangles; and the hyperphosphorylation and intraneuronal deposition of the microtubule-associated protein tau in the form of filaments; intraneuronal deposition of aggregated tau filaments. In Alzheimer's accumulation of the amyloid-β protein may lead to a reduction on CREB phosphorylation, which may be related to the cognitive deficits seen in this condition, and more generally, increasing cAMP or cGMP levels by PDE4 inhibition can restore neuronal plasticity in Alzheimer models (Vitolo et al., 2002, Proc. Natl. Acad. Sci. U.S.A. 99, 13217-13221; Medina, 2011, Front. Neurosci. 5, 21).

Huntington's Disease:

In specific embodiments, the disclosure provides a method of treating Huntington's disease (or “Huntington's chorea”), comprising administering to a subject in need thereof an effective amount of a chemical entity or composition disclosed herein. In a specific aspect, the chemical entity is a compound of Formula (I) or (II), or pharmaceutically acceptable salt thereof. There are two forms of Huntington's disease: adult-onset Huntington's disease, which is the most common form and usually begins in subjects aged in the mid 30's and 40's, and early-onset Huntington's disease, which accounts for a small number of cases and begins in childhood or adolescence. Symptoms of Huntington's disease include behavioral changes, abnormal and unusual movements, and worsening dementia (e.g., Dumas et al., 2013, Front. Biosci. (Schol. Ed) 5, 1-18). Huntington's disease (HD, or Huntington chorea) is a genetic disorder, whose pathology includes degeneration of striatal neurons in the basal ganglia responsible for movement and coordination. PDE1 is highly expressed in the striatum, and PDE1 inhibition has been shown to confer protection against behavioral and biochemical toxicities in an experimental models of Huntington's disease. Gupta and Sharma, 2014, Eur. J. Pharmacol. 732, 111-122. A detailed set of criteria for the diagnosis of Huntington's disease is set forth in the Diagnostic and Statistical Manual of Mental Disorders (DSM-5; 5^{th} ed., 2013, American Psychiatric Association).

Augmented Training

In some embodiments, chemical entities, and compositions thereof, of the present disclosure are used as augmenting agents in methods to increase the efficiency of training protocols for enhancing a neurological function or treating a neurological impairment associated with a neurological disorder. Such methods are known as “augmented training,” and more particularly, in the case of cognitive impairments, “augmented cognitive training,” and in the case of motor impairments, “augmented motor training.” Augmenting agents can act by shortening the time that methods of rehabilitating (or enhancing) a cognitive or motor function result in improved performance or a functional gain. Such augmented training therefore comprises a specific training protocol for a particular brain function, such as that underlying declarative memory, performance of a fine motor skill, a specific locomotor function, language acquisition, executive function, etc.; and a general administration of an augmenting agent of the present disclosure.

Training (or a “training protocol”) generally requires many sessions to attain the desired benefits, for example, to rehabilitate a motor deficit or language deficit following stroke. This can be costly and time-consuming, deterring subject compliance and the realization of real world benefits that endure over time. The efficiency of such training protocols can be improved by administering certain agents (known as augmenting agents) in conjunction with the training protocol (see, e.g., U.S. Pat. Nos. 7,868,015; 7,947,731; U.S. 2008-0188525). When administered in combination with training protocols (or “training”), augmenting agents enhance functional reorganization in targeted domains (or “functions”) in the brain.

Cognitive domains (or “functions”) that can be targeted by training protocols include, but are not limited to, the following: attention (e.g., sustained attention, divided attention, selective attention, processing speed); executive function (e.g., planning, decision, and working memory); learning and memory (e.g., immediate memory; recent memory, including free recall, cued recall, and recognition memory; and long-term memory, which can be divided into explicit memory (declarative memory) memory, such as episodic, semantic, and autobiographical memory, and into implicit memory (e.g., procedural memory)); language (e.g., expressive language, including naming, word recall, fluency, grammar, and syntax; and receptive language); perceptual-motor functions (e.g., abilities encompassed under visual perception, visuo-constructional, perceptual-motor praxis, and gnosis); and social cognition (e.g., recognition of emotions, theory of mind). In specific embodiments, the cognitive function is learning and memory, and more particularly, long term memory.

Motor domains (or functions) that can be targeted by training protocols include, but are not limited to, those involved in gross body control, coordination, posture, and balance; bilateral coordination; upper and lower limb coordination; muscle strength and agility; locomotion and movement; motor planning and integration; manual coordination and dexterity; gross and fine motor skills; and eye-hand coordination.

Training Protocols:

Training protocols (or “modules”) include cognitive training and motor training protocols. Training protocols are well-known in the art and typically comprise a set of distinct exercises that can be process-specific or skill-based: See, e.g., Kim et al., 2014, J. Phys. Ther. Sci. 26, 1-6; Allen et al., 2012, Parkinson's Dis. 1-15; Jaeggi et al., 2011, Proc. Natl. Acad. Sci. USA 108, 10081-10086; Chein et al., 2010, Psychon. Bull. Rev. 17, 193-199; Klingberg, 2010, Trends Cogn. Sci. 14, 317-324; Owen et al., 2010, Nature 465, 775-778; Tsao et al., 2010, J. Pain 11, 1120-1128; Lustig et al., 2009, Neuropsychol. Rev. 19, 504-522; Park and Reuter-Lorenz, 2009, Ann. Rev. Psych. 60, 173-196; Oujamaa et al., 2009, Ann. Phys. Rehabil. Med. 52, 269-293; Frazzitta et al., 2009, Mov. Disord. 8, 1139-1143; Jaeggi et al., 2008, Proc. Natl. Acad. Sci. USA 105, 6829-6833; Volpe et al., 2008, Neurorehabil. Neural Repair 22, 305-310; Fischer et al., 2007, Top. Stroke Rehab. 14, 1-12; Jonsdottir et al., 2007, Neurorehabil. Neural Repair 21, 191-194; Stewart et al., 2006, J. Neurol. Sci. 244, 89-95; Krakauer, 2006, Curr. Opin. Neurol. 19, 84-90; Belleville et al., 2006, Dement. Geriatr. Cogn. Disord. 22, 486-499; Klingberg et al., 2005, J. Am. Acad. Child. Adolesc. Psychiatry 44, 177-186; Dean et al., 2000, Arch. Phys. Med. Rehabil. 81, 409-417; Whitall et al., 2000, Stroke 31, 2390-2395; Hummelsheim and Eickhof, 1999, Scand. J. Rehabil. Med. 31, 250-256; Merzenich et al., 1996, Science 271, 77-81; Merzenich et al., 1996, Cold Spring Harb. Symp. Quant. Biol. 61, 1-8; Rider and Abdulahad, 1991, Percept. Mot. Skills 73, 219-224.

Process-specific training focuses on improving a particular domain such as attention, memory, language, executive function, or motor function. Here the goal of training is to obtain a general improvement that transfers from the trained activities to untrained activities based on the same cognitive or motor function or domain.

Skill-based training is aimed at improving performance of a particular activity or ability, such as learning a new language, performing a musical instrument, improving memory, or learning a fine motor skill. The different exercises within such a protocol will focus on core components within one or more domains underlying the skill. Modules for increasing memory, for example, may include tasks directed to specific domains involved in memory processing, e.g., the recognition and use of facts, and the acquisition and comprehension of explicit knowledge rules.

In some embodiments, the battery of exercises is administered as part of a single training session. In one aspect, the training protocol comprises multiple training sessions, each separated by a discrete interval. In another aspect, the number of training sessions sufficient to improve performance is reduced compared to that produced by training alone.

In a further aspect, the augmenting agent is a PDE1 inhibitor, and more particularly, is a chemical entity of the present disclosure, and is administered in conjunction with training. The phrase “in conjunction with” means that the augmenting agent enhances CREB pathway function during training. In some embodiments, the deficit is a motor deficit. In other embodiments, the deficit is a cognitive deficit. In still other embodiments, the deficit may include both a cognitive and motor deficit. In other aspects, the compound is administered before and during each training session. In one aspect, the subject is a human. In some embodiments, the subject is a non-human, and more particularly, is a primate or a canine.

In one aspect, a chemical entity or composition of the present disclosure can be used as an augmenting agent in conjunction with any psychotherapeutic approach intended to modulate cognitive function in the brain, thereby enhancing the efficacy of such therapy by reducing the number of sessions necessary to attain benefits. In a specific aspect, the chemical entity is a compound of Formula (I) or (II), or pharmaceutically acceptable salt thereof.

Accordingly, in some embodiments, the disclosure provides the use of a chemical entity or composition herein in a method of augmented training to treat a neurological disorder, the method comprising: (a) providing training to an animal in need of treatment of a neurological impairment associated with the neurological disorder under conditions sufficient to produce an improvement in performance by said animal of a neurological function whose deficit is associated with said neurological impairment; (b) administering the chemical entity or composition to the animal in conjunction with said training; (c) repeating said providing and administering steps one or more times; and (d) reducing the number of training sessions sufficient to produce the improvement in performance, relative to the improvement in performance produced by training alone. In some aspects, the animal is a human subject. In some aspects, the augmented training is augmented cognitive training. In some aspects, the neurological impairment is a cognitive impairment. In some aspects, the neurological impairment is a motor impairment. In a specific aspect, the neurological disorder is stroke or traumatic brain injury. In some aspects, the augmented training is provided to a stroke patient during post-stroke rehabilitation, as described further herein. In a specific aspect, the chemical entity is a compound of Formula (I) or (II), or pharmaceutically acceptable salt thereof. In some embodiments, training comprises spaced training sessions. In other embodiments, training comprises massed training sessions.

Animal Skill Protocols:

In some embodiments, chemical entities of the present invention are used to enhance the efficiency of training protocols directed to cognitive and motor skills in an animal. Such augmented training (augmenting agent and training) reduces the time necessary to acquire a cognitive or motor skill, and/or enhance function or cognitive ability beyond what would be possible by training alone in the non-human animal.

In particular embodiments, the animal is a non-human animal, and more particularly, is a service animal, a category that includes, but is not limited to, dogs, miniature horses, and capuchin monkeys. Service animals may be involved in public service or private service, and the training protocols will be appropriately matched to these objections. For example, training protocols directed to public service include public order maintenance, search and rescue, and contraband detection, and training protocols directed to private service include private security, handicap assistance, health care, psychiatric assistance, and pest control.

The training protocol may be directed to a single skill, such as the detection of a specific contraband category by a service animal. In other embodiments, the training protocol may be directed to a complex set of skills, such as those underlying search and rescue training of a service animal; for a complex set of skills, training will therefore comprise more than one tasks.

Accordingly, in some embodiments, the present invention provides a method of teaching a non-human animal one or more skills, comprising (a) administering to a non-human animal in need thereof a PDE1 inhibitor; (b) providing training to the animal under conditions sufficient to improve performance of the one or more skills; and (c) repeating steps (a) and (b) one or more times, whereby the amount of training sufficient to improve the performance is reduced compared to that produced by training alone.

Stroke

In certain embodiments, chemical entities and compositions of the present disclosure are useful in methods of treating a trauma-dependent loss of function, and more particularly, stroke. Stroke is a leading cause of serious long-term disability in adults and is the second leading cause of death worldwide (e.g., Go et al., 2014, Circulation 129, e28-e92.). Stroke is comprises two main types: 1) ischemic stroke which occurs when blood vessels supplying the brain are blocked by clot formation (85% of all strokes) and 2) hemorrhagic stroke which occurs when blood vessels rupture within the brain (13-15% of all strokes). Stroke care is a temporal continuum that includes medical intervention during the acute phase of stroke and subsequent rehabilitative therapy directed to restoring function during the post-stroke phase of stroke.

Acute Treatments:

Treatments following the onset of stroke directly target the initial damage triggered by ischemic or hemorrhagic stroke. Acute treatment options for ischemic stroke include pharmacotherapy with intravenous recombinant tissue plasminogen activator (r-tPA) to thrombolyze the clot, or the use of endovascular procedures or mechanical thrombectomy to physically remove the clot. Acute treatment options for hemorrhagic stroke typically involve endovascular or surgical procedures to physically repair the rupture.

Post-Stroke Rehabilitation:

Following the acute phase of stroke—and typically after the patient has been medically stabilized—the focus of stroke treatment shifts to restoring function by rehabilitation. Depending on the severity and location of the stroke as well as the timing and effectiveness of acute interventions, post-stroke symptoms may persist and can include motor deficits (e.g., hemiparesis, apraxia), speech impairment (e.g., aphasia), visual impairments (e.g., visual field loss), emotional and behavioral changes (e.g., depression, anxiety), and mental and cognitive changes (e.g., confusion, apathy, cognitive impairment) (Winstein et al., 2016, Stroke 47, e98-e169). Rehabilitation (also referred to as “stroke rehabilitation” or “post-stroke rehabilitation”) is directed to post-stroke deficits, such as cognitive and motor deficits that persist after the initial stroke injury. The goal is to restore and recover neurological functions, e.g., physical, intellectual, psychological, and social functions, as much as possible to compensate for the permanent tissue loss (e.g., 1995 Clinical Guideline by the Department of Health and Human Services on Post-Stroke Rehabilitation).

Stroke rehabilitation is typically a comprehensive program coordinated by a team of medical professionals, which may include occupational, speech, and physical therapists. A physical therapist on the team, for example, may focus on maintaining and restoring range of motion and strength in affected limbs, maximizing mobility in walking, improving manual dexterity, and rehabilitating other motor and sensorimotor functions. A mental health professional may be involved in the treatment of loss of cognitive skills. Rehabilitation services can occur in multiple environments, such as a rehabilitation hospital, long-term care facility, outpatient clinic, or at home.

Neurological functions impacted by stroke (and which can be targeted during rehabilitation) include impairments in cognitive and motor functions. Cognitive function impairments, for example, can manifest as deficits in understanding speech or writing (aphasia); knowing the right words but having trouble saying them clearly (dysarthria); as well as deficits in other cognitive functions, such as attention, reasoning, planning, execution, and learning and memory. Motor function impairments, for example, can manifest as weakness (hemiparesis) or paralysis (hemiplegia) on one side of the body that may affect the whole side or just the arm or leg; as problems with balance or coordination; as deficits in gross motor skills such as gait and walking speed; as deficits in fine motor skills or manual dexterity; and as deficits in upper and lower extremity function.

In the United States, more than 700,000 people suffer a stroke each year, two-thirds of these survive and require rehabilitation. Unfortunately, recovery is generally only partial and considerable deficits persist in many patients (e.g., Gordon et al., 2004, Stroke 35, 1230-1240). For example, after standard rehabilitation, approximately 30% to 60% of patients are left without functional use of their paretic/plegic arm (Gowland, 1982, Physiother. Can. 34, 77-84; Kwakkel et al., 1996, Age Ageing 25, 479-489), and despite intensive rehabilitation efforts, only approximately 5% to 20% reach complete functional recovery of their arm (Nakayama et al., 1994, Arch. Phys. Med. Rehabil. 75, 394-398).

As discussed herein, chemical entities, and compositions thereof, of the present disclosure are used as augmenting agents to increase the efficiency of training protocols for treating a neurological impairment, which encompasses impairments due to traumatic events such as stroke. Accordingly, in some embodiments, the present disclosure provides methods of treating a neurological deficit during post-stroke rehabilitation comprising: (a) administering to a subject in need thereof a PDE1 inhibitor disclosed herein during recovery of the subject from stroke; (b) providing training to the subject under conditions sufficient to improve performance of a neurological function whose impairment is due to the deficit; and (c) repeating steps (a) and (b) one or more times, whereby the amount of training sufficient to improve the performance is reduced compared to that produced by training alone.

In some embodiments, administration can begin during the acute stage. In other embodiments, the PDE1 inhibitor is administered only after the acute stage, i.e., during post-stroke rehabilitation, which may include sub-acute and chronic stages. In some embodiments, administration occurs during the acute stage and post-stroke stage. In some embodiments, the PDE1 inhibitor is administered chronically, meaning that it is indicated for long-term use after the acute stage of the stroke has ended and the patient has been medically stabilized.

In other embodiments, the subject is a post-stroke patient, and PDE1 inhibitors are administered during stroke rehabilitation to treat stroke deficits (or “post-stroke deficits”) resulting from impaired neurological functions. In some embodiments, the deficit is a motor deficit, including upper or lower extremity motor deficit. In other embodiments, the deficit is a cognitive deficit, such as such as aphasia, apraxia, and mental and cognitive changes, particularly, a deficit in memory formation, and more specifically, a deficit in long-term memory formation. In still other embodiments, the deficit may include a cognitive and motor deficit. In another aspect, training comprises a battery of tasks directed to the neurological function. In a specific aspect, the reduction in the amount of training is a reduction in the number of training sessions.

In a further embodiment, the administering step (a) is in conjunction with the training step (b). In one aspect, the subject is a human. In another aspect, the subject has undergone neuronal stem cell manipulation. In other aspects, the compound is administered before and during each training session.

Traumatic Brain Injury

In some embodiments, chemical entities and compositions are useful in methods of treating traumatic brain injury (TBI), and in more specific embodiments, treating motor or cognitive impairments during rehabilitation of TBI after the initial trauma.

TBI, also known as intracranial injury, occurs when an external force injures the brain. TBI can be classified based on severity, mechanism (closed or penetrating head injury), or other features (e.g., occurring in a specific location or over a widespread area). TBI can result in physical, cognitive, social, emotional, and behavioral symptoms. Causes include falls, vehicle collisions, gunshot injuries, and explosives. Outcomes can range from complete recovery to permanent disability or death.

Like stroke care, TBI case is a temporal continuum that includes acute (or sub-acute) treatments directed to the injury itself and subsequent rehabilitative therapy directed to restoring function.

Accordingly, in some embodiments, the chemical entities and compositions of the present disclosure are useful during the acute (or sub-acute) stage of TBI, during which their administration can treat neuroinflammatory and neurodegenerative events following the primary injury.

Some embodiments provide the use of a PDE1 inhibitor disclosed during TBI rehabilitation to treat TBI deficits (or “post-TBI deficits”) resulting from impaired neurological functions. Some embodiments provide methods of treating a neurological deficit during post-TBI rehabilitation comprising: (a) administering to a subject in need thereof a PDE1 inhibitor during recovery of the subject from TBI; (b) providing training to the subject under conditions sufficient to improve performance of a neurological function whose impairment is due to the deficit; and (c) repeating steps (a) and (b) one or more times, whereby the amount of training sufficient to improve the performance is reduced compared to that produced by training alone.

In one aspect, the PDE1 inhibitor is a chemical entity of the present disclosure, and more specifically, is a compound, or pharmaceutically acceptable salt thereof, of Formula (I) or (II). In some embodiments, the deficit is a motor deficit. In other embodiments, the deficit is a cognitive deficit, particularly, a deficit in memory formation, and more specifically, a deficit in long-term memory formation. In still other embodiments, the deficit may include a cognitive and motor deficit. In another aspect, training comprises a battery of tasks directed to the neurological function. In a specific aspect, the reduction in the amount of training is a reduction in the number of training sessions.

In a further embodiment, the administering step (a) is in conjunction with the training step (b). In one aspect, the subject is a human. In another aspect, the subject has undergone neuronal stem cell manipulation. In other aspects, the compound is administered before and during each training session.

Peripheral Disorders

In some embodiments, the present disclosure provides methods of treating a peripheral disorder (i.e., a disorder other than a primary neurological disorder), comprising administering to a subject in need thereof an effective amount of a chemical entity or composition disclosed herein. In one embodiment of these methods, the chemical entity is a compound, or pharmaceutically acceptable salt thereof, of Formula (I) or (II). Peripheral disorders involving PDE1 include a wide variety of diseases, based on numerous biological studies and the expression of PDE1 subtypes in peripheral tissues, such as the heart, lungs, veins and arteries, smooth muscle, skeletal muscle, skin, adrenal gland, thyroid, pancreas, esophagus, stomach, small intestine, colon, liver, leukocytes, testis, ovary, bladder, and kidney. See, e.g., Bender and Beavo, 2006, Pharmacol. Rev. 58, 488-520. Accordingly, peripheral disorders that can be treated by compounds and compositions of the present invention include, but are not limited to, cardiovascular disorders, renal disorders, hematological disorders, gastrointestinal and liver disorders, cancer disorders, fertility disorders, and metabolic diseases, such as diabetes or obesity.

Peripheral disorders also include, in certain embodiments, diseases and conditions (other than primary neurological disorders) characterized by low levels of cAMP or cGMP in cells expressed PDE1, by inhibition of cAMP or cGMP signaling pathways in cells expressing PDE1, and by reduced dopamine D1 receptor signaling activity.

Cardiovascular Disorders:

In certain embodiments, the peripheral disorder is a cardiovascular disorder. PDE1 enzymes and cyclic nucleotides are emerging as key mediators of pathological processes that underlie many vascular disorders, including hypertension and myocardial infarction. All three PDE1 isoforms are expressed in the human pulmonary artery, as well as the aorta and small mesenteric arteries. Schermuly et al., 2007, Circulation 115, 2331-2339; Murray et al., 2007, Am. J. Physiol. Lung Cell. Mol. Physiol., 292, L294-L303. In addition, selective PDE1 inhibition induces vasodilation and lower blood pressure in rats. Laursen et al., 2017, Br. J. Pharmacol. 174, 2563-2575. Moreover, PDE1 enzymes constitute the majority of cAMP- and cGMP-hydrolytic activity in human myocardium, implicating them in the modulation of signaling pathways involved in heart failure.

Accordingly, the present invention includes the use of a compound or composition herein in a method of treating a cardiovascular disorder, comprising administration of an effective amount of the chemical entity or composition to a patient in need thereof.

Cardiovascular diseases within the scope of the present invention encompass, but are not limited to, angina pectoris, coronary artery disease, hypertension, congestive heart failure, myocardial infarction, ischemic diseases of the heart, atrial and ventricular arrhythmias, hypertensive vascular diseases, peripheral vascular diseases, pulmonary hypertension (PH) (or pulmonary arterial hypertension (PAH)), atherosclerosis, and other pulmonary and respiratory disorders.

In some embodiments, methods of treating a cardiovascular disorder in accord with the present invention comprise increasing cGMP concentration, cAMP concentration, or both, in any part of the heart muscle of a subject, the method comprising administering to the subject a chemical entity or composition described herein.

In other embodiments, chemical entities and compositions of the present invention may be useful in lowering the heart rate or blood pressure in an animal.

Renal Disorders

In certain embodiments, the peripheral disorder is a renal disease. PDE1 inhibitors are emerging therapeutic agents for progressive renal disease. See, e.g., Cheng et al., 2007, Soc. Exp. Biol. Med. 232, 38-51. Consistent with these findings, recent studies indicate that cAMP and cGMP regulate a variety of signaling pathways involved in the development and progression of renal disease, including pathways that modulate mitogenesis, inflammation, and extracellular matrix synthesis. See e.g., Wang et al., 2010, Kidney Int. 77. 129-140; Wang et al., 2017, PLoS One 12, e0181087.

Accordingly, the present invention provides chemical entities or compositions in methods for treating a renal disorder, comprising administering an effective amount of the chemical entity or composition to a patient in need thereof. In a particular aspect, the renal disorder is selected from one or more of the group comprising renal artery stenosis, pyelonephritis, glomerulonephritis, kidney tumors, polycystic kidney disease, injury to the kidney, and damage resulting from radiation of the kidney, and autosomal dominant polycystic kidney disease (ADPKD).

Hematological Disorders

In certain embodiments, the peripheral disorder is a hematological disorder. PDE1B is highly expressed in the hematological system, including leukocytes (peripheral blood), bone marrow stromal cells, bone marrow CD33+ cells, cord blood CD34+ cells, neutrophils cord blood, neutrophils peripheral blood, spleen, spleen liver cirrhosis. Accordingly, the present invention includes methods to treat a hematological disorder, comprising administering a chemical entity or composition herein to a patient in need thereof. Hematological diseases within the scope of the present invention comprises disorders of the blood and all its constituents, including, but not limited to anemias, myeloproliferative disorders, hemorrhagic disorders, leukopenia, eosinophilic disorders, leukemias, lymphomas, plasma cell dyscrasias, and disorders of the spleen.

Gastrointestinal and Liver Diseases

In certain embodiments, the peripheral disorder is a gastrointestinal or liver disease. PDE1B shows differential expression between diseased (e.g., cancerous) and healthy stomach tissue, diseased (e.g., cancerous) versus healthy ileum tissue, diseased (cirrhotic) versus and healthy liver. Accordingly, the present invention includes methods to treat a gastrointestinal of liver disorder, comprising administering a compound or composition herein to a patient in need thereof. Gastrointestinal and liver diseases within the scope of the present invention comprise, but are not limited to, disorders of the esophagus, stomach, duodenum, pancreas, bowel, and liver.

Cancer Disorders

In certain embodiments, the peripheral disorder is a cancer disorder. PDE1B shows high expression in numerous cancer tissues, including tumors of the stomach, ileum, ovary, breast, and kidney, as well as differential expression between cancerous and healthy stomach, ileum, lung, ovary, breast, and kidney. Accordingly, the present invention includes methods to treat a cancer disorder, comprising administering a compound or composition herein to a patient in need thereof. Cancer disorders within the scope of the present invention comprise, but are not limited to, neoplasms, dysplasias, hyperplasias, and neoplasms, including cancers of the stomach, ileum, ovary, breast, and kidney.

Fertility Disorders

In certain embodiments, the peripheral disorder is a fertility disorder. PDE1 inhibitors, for example, have been implicated in the enhancement of progesterone signaling (e.g., WO 2008/070095). Accordingly, the present invention includes methods to treat a fertility disorder, comprising administering a compound or composition herein to a patient in need thereof. Fertility disorders within the scope of the present invention comprise female sexual dysfunction and disorders involving impairments in progesterone signaling, which include, but are not limited to, exercise-induced amenorrhea, anovulation, menopause, menopausal symptoms, hypothyroidism, pre-menstrual syndrome, premature labor, infertility, irregular menstrual cycles, abnormal uterine bleeding, osteoporosis, autoimmune disease, multiple sclerosis, estrogen-induced endometrial hyperplasia and estrogen-induced endometrial carcinoma.

Treatment Combinations

Chemical entities and compositions of the present disclosure can be administered as a monotherapy or as part of a combination therapy. “Monotherapy” refers to a treatment regimen based on the delivery of one (e.g., one and only one) therapeutically effective chemical entity or composition thereof.

In a combination therapy, one or more chemical entities or compositions of the present invention can be co-administered or used in combination with one or more additional agents (or therapies), such as additional agents (or therapies) known in the art. Such administration may be simultaneous, sequential, or staggered. In certain embodiments, the additional agent (or therapies) is based on a different target or modality (e.g., is not a PDE1 inhibitor).

In some embodiments, the combination is administered as part of an adjunct (or adjunctive) therapy, in which one agent is given in addition to a primary agent to assist or maximize the effectiveness of the primary agent.

In specific embodiments, the combination is administered to treat schizophrenia, Parkinson's disease, Alzheimer's disease, Huntington's disease, anxiety and depressive disorders, or stroke. In some embodiments, a chemical entity or composition disclosed herein is administered as an adjunct therapy in conjunction with a dopamine precursor, such as levodopa, to treat Parkinson's disease or a related disorder.

Exemplary agents for treating schizophrenia include, but are not limited to, clozapine, aripiprazole, brexpiprazole, cariprazine, lurasidone, paliperidone, quetiapine, risperidone, olanzapine, ziprasidone, and iloperidone.

Exemplary agents for treating Parkinson's disease include, but are not limited to, dopamine preparations (including dopamine precursors such as levodopa), dopamine agonists, or COMT agents (drugs that inhibit the action of catechol-methyl transferase).

Exemplary agents for treating Alzheimer's disease include, but are not limited to, donepezil, rivastigmine, galantamine, marijuana-like cannabinoids, and memantine.

Exemplary agents for treating Huntington's disease (or other motor disorders) may include, but are not limited to, tetrabenazine, as well as antipsychotic drugs such as haloperidol, chlorpromazine, risperidone, and quetiapine, and anti-epileptic drugs such as levetiracetam and clonazepam, which may be beneficial in treating chorea or related motor disorders.

Exemplary agents for treating anxiety or depression include, but are not limited to, benzodiazepines and other anxiolytics; serotonin reuptake inhibitors (SSRIs), such as sertraline, fluoxetine, citalopram, escitalopram, paroxetine, fluvoxamine, and trazodone; serotonin and norepinephrine reuptake inhibitors (SNRIs), such as desvenlafaxine, duloxetine, levomilnacipran, and venlafaxine; tricyclic antidepressants (TCAs), such as amitriptyline, amoxapine, clomipramine, desipramine, doxepin, imipramine, nortriptyline, protriptyline, and trimipramine; monoamine oxidase inhibitors (MAOIs), such as isocarboxazid, phenelzine, selegiline, and tranylcypromine; and other classes of drugs, such as maprotiline, bupropion, vilazodone, nefazodone, trazodone, vortioxetine, and mirtazapine

Exemplary agents for treating stroke include, but are not limited to, a thrombolytic agent (e.g., streptokinase, acylated plasminogen-streptokinase activator complex (APSAC), urokinase, single-chain urokinase-plasminogen activator (scu-PA), anti-inflammatory agents, thrombin-like enzymes, tissue plasminogen activator (t-PA); an anticoagulant (e.g., warfarin or heparin); an antiplatelet drug (e.g., aspirin); a glycoprotein IIb/IIIa inhibitor; a glycosaminoglycan; coumarin; GCSF; melatonin; an apoptosis inhibitor (e.g., caspase inhibitor), an anti-oxidant (e.g., NXY-059); and a neuroprotectant (e.g., an NMDA receptor antagonists or a cannabinoid antagonist).

The preceding list of additional active agents is meant to be exemplary rather than fully inclusive. Additional active agents not included in the above list may be administered in combination with a compound of Formula (I) or (II), such as those know for treating peripheral disorders described herein. The additional active agent will be dosed according to its approved prescribing information, though in some embodiments the additional active agent may be dosed at less the typically prescribed dose.

### EXAMPLES

The present disclosure will be further illustrated by the following non-limiting Examples. These Examples are understood to be exemplary only, and they are not to be construed as limiting the scope of the one or more embodiments, and as defined by the appended claims.

### PREPARATIVE EXAMPLES

Exemplary compounds will now be described by reference to the illustrative synthetic schemes for their general preparation below and the specific examples to follow.

One skilled in the art will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I) or (II). Reactions may be performed between −100° C. and the reflux temperature of the solvent. Reactions may be heated employing conventional heating or microwave heating. Reactions may also be conducted in sealed pressure vessels above the normal reflux temperature of the solvent.

Abbreviations

The specification includes numerous abbreviations, whose meanings are listed in the following Table:

TABLE 1

| Abbreviation | Definition |
|---|---|
| AcCl | Acetyl chloride |
| ACN | Acetonitrile |
| BBr₃ | Boron tribromide |
| BF₃•Et₂O | Boron trifluoride diethyl etherate |
| Boc | tert-Butyloxycarbonyl |
| Br₂ | Bromine |
| n-BuLi | n-Butyl lithium |
| t-BuONO | tert-Butyl nitrite |
| CAS | Chemical abstracts service |
| CDCl₃ | Deuterated chloroform |
| Celite ® | Diatomaceous earth |
| CH₂Cl₂ | Chloroform |
| ClPO₃Et₂ | Diethyl chlorophosphate |
| CO | Carbon monoxide |
| CO₂ | Carbon dioxide |
| mCPBA | meta-Chloroperoxybenzoic acid |
| Cs₂CO₃ | Cesium carbonate |
| CuBr₂ | Copper(II) bromide |
| DBU | 1,8-Diazabicyclo[5.4.0]undec-7-ene |
| DCM | Dichloromethane |
| DIPEA, DIEA | N,N-ethyldiisopropylamine or |
| | N,N-Diisopropylethyl amine |
| DMA | N,N-Dimethylacetamide |
| DMF | N,N-Dimethylformamide |
| DMF-DMA | N,N-Dimethylformamide dimethyl acetal |
| DMSO | Dimethylsulfoxide |
| dppf | 1,1'-Bis(diphenylphosphino)ferrocene |
| EtOAc, or EA | Ethyl acetate |
| EtOH | Ethanol |
| FCC | Flash column chromatography |
| Ghaffar-Parkins | Hydrido(dimethylphosphinous acid-kP)[hydrogen |
| catalyst | bis(dimethylphosphinito-kP)] |
| H₂ | Hydrogen |
| HCl | Hydrochloric acid |
| H₂O | Water |
| H₂O₂ | Hydrogen peroxide |
| HPLC | High-performance liquid chromatography |
| I₂ | Iodine |
| KHCO₃ | Potassium bicarbonate |
| K₂CO₃ | Potassium carbonate |
| KOH | Potassium hydroxide |
| K₃PO₄ | Tripotassium phosphate |
| KOt-Bu | Potassium tert-butoxide |
| LaCl₃•2LiCl | Lanthanum(III) chloride bis(lithium chloride) |
| | complex |
| LCMS, LC/MS | Liquid chromatography-mass spectrometry |
| LDA | Lithium diisopropylamide |
| MeI | Iodomethane, methyl iodide |
| MeNHNH₂ | Methyl hydrazine |
| MeOH | Methanol |
| Me₂SO₄ | Dimethyl sulfate |
| MgSO₄ | Magnesium sulfate |
| N₂ | Nitrogen |
| NaCl | Sodium chloride, brine |
| Na₂CO₃ | Sodium carbonate |
| NaHCO₃ | Sodium bicarbonate |
| NaHMDS | Sodium hexamethyldisilazane, |
| | Sodium bis(trimethylsilyl)amide |
| Na₂SO₃ | Sodium sulfite |
| Na₂SO₄ | Sodium sulfate |
| NBS | N-Bromosuccinimide |
| NCS | N-Chlorosuccinimide |
| NFSI | N-Fluorobenzenesulfonimide, |
| | N-Fluorodi(benzenesulfonyl)amine |
| NH₄Cl | Ammonium chloride |
| NH₂OH•HCl | Hydroxylamine hydrochloride |
| NH₄OH | Ammonium hydroxide |
| NIS | N-Iodosuccinimide |
| Pd/C | Palladium on carbon, 10% |
| Pd(dppf)Cl₂ | [1,11-Bis(diphenylphosphino)ferrocene] |
| | dichloropalladium(II) |
| Pd(dppf)Cl₂•CH₂Cl₂ | [1,11-Bis(diphenylphosphino)ferrocene] |
| | dichloropalladium(II), complex with |
| | dichloromethane |
| Pd(OAc)₂ | Palladium(II) acetate |
| i-PrMgBr | i-Propyl magnesiumbromide |
| i-PrMgCl | i-Propyl magnesium chloride |
| PTFE | Polytetrafluoroethylene |
| PtO₂ | Platinum(I) oxide |
| Raney Ni | Raney ®-Nickel, W. R. Grace and Co. Raney ® |
| | 2800, slurry, in H₂O, active catalyst |
| RT, rt | Room temperature |
| SFC | Supercritical fluid chromatography |
| SiO₂ | Silica |
| TBAF | Tetrabutylammonium fluoride |
| TBSCl, TBDMSCl | tert-Butyldimethylsilyl chloride, |
| | tert-Butyldimethylchlorosilane |
| TEA, Et₃N | Triethylamine |
| THF | Tetrahydrofuran |
| TMSBr | Trimethylsilyl bromide |
| TsCN | p-Tolylsulfonyl cyanide, Tosyl cyanide |

Synthetic Schemes

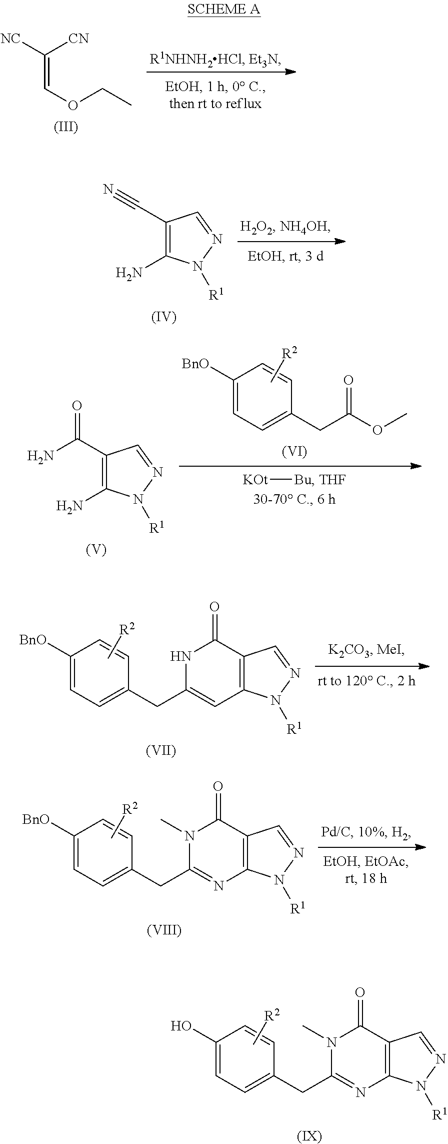

1,6-Substituted pyrazolopyrimidinone compounds, where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl, are synthetically accessible in 5 steps starting from 2-(ethoxymethylene)malononitrile and a commercially available substituted hydrazine or hydrazine hydrochloride salt. Initially, a pyrazole ring is formed by combining a substituted hydrazine and a substituted malononitrile. For example, treatment of a hydrazine compound with a base, such as trimethylamine or the like, in a solvent, such as ethanol, at an initial temperature of 0° C., for an hour or more, followed by slow addition of 2-(ethoxymethylene)malononitrile, followed by stirring at a temperature ranging from rt to reflux for several hours, ideally stirring at rt for many hours then heating at reflux for several hours, provides a substituted pyrazole compound of formula (IV), where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl. Subsequent treatment with hydrogen peroxide and aqueous ammonia, in a solvent such as ethanol, or the like, at room temperature for a time period of many hours to several days, provides a primary amide substituted compound of formula (V). Formation of a pyrazolopyrimidinone is achieved by treatment of a pyrazole of formula (V) with a substituted methyl 2-phenylacetate in the presence of a base, under conditions known to one of skill in the art. For example, treatment of a pyrazole of formula (IV) with a substituted methyl 2-phenylacetate of formula (V), where R² is independently 1 or more halogens, C₁₋₆alkyl, C₁₋₆haloalkyl, C₁₋₆alkoxy, or C₁₋₆haloalkoxy, in the presence of 3 Å molecular sieves, in a solvent such as THF, or the like, followed by addition of a base, such as potassium tert-butoxide or sodium tert-butoxide, or the like, at a temperature ranging from 30° C. to 70° C., for several hours, provides a pyrazolopyrimidinone of formula (VII), where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl, and R² is independently 1 or more halogens, C₁₋₆alkyl, C₁₋₆haloalkyl, C₁₋₆alkoxy, or C₁₋₆haloalkoxy groups. Methylation of the nitrogen of a pyrazolopyrimidinone of formula (VII) is achieved by treatment with a methylating agent, such as a halo methane, in the presence of a base, under conditions known to one of skill in the art. For instance, treatment of a compound of formula (VII) with iodomethane, in the presence of a base, such as potassium carbonate or the like, in a solvent such as DMF or DMA, or the like, at a temperature ranging from rt to 120° C., provides a compound of formula (VIII). Removal of the benzyl protecting group under typical reductive conditions known to one of skill in the art, provides the final phenolic intermediate of formula (IX). For instance, treatment of a compound of formula (VIII) with a catalyst, such as palladium on carbon (10%), or the like, under an atmosphere of hydrogen gas, in a solvent such as ethanol, or the like, or a solvent mixture such as ethyl acetate and ethanol, provides a phenol compound of formula (IX), where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl, and R² is independently 1 or more halogens, C₁₋₆alkyl, C₁₋₆haloalkyl, C₁₋₆alkoxy, or C₁₋₆haloalkoxy groups.

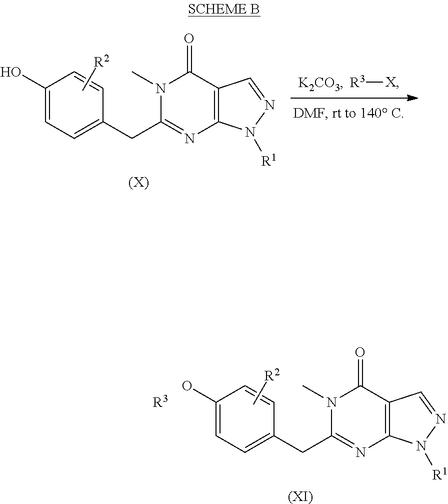

A nucleophilic substitution reaction of an alkyl halide with a phenol, under conditions known to one of skill in the art, in the presence of a base, provides a compound of formula (XI), where R³ is C₁₋₆alkyl, C₁₋₆haloalkyl, or C₃₋₆cycloalkyl. For example, treatment of a phenol of formula (X) with a base, such as potassium carbonate or cesium carbonate or the like, and an alkyl halide, in a solvent such as DMF or DMA or the like, at a temperature ranging from rt to 140° C., sometimes 60° C., for a time period ranging from several hours to several days, provides a compound for formula (XI), where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl, R² is independently 1 or more halogen, C₁₋₆alkyl, C₁₋₆haloalkyl, C₁₋₆alkoxy, or C₁₋₆haloalkoxy, and R³ is C₁₋₆ alkyl, C₁₋₆haloalkyl, or C₃₋₆cycloalkyl.

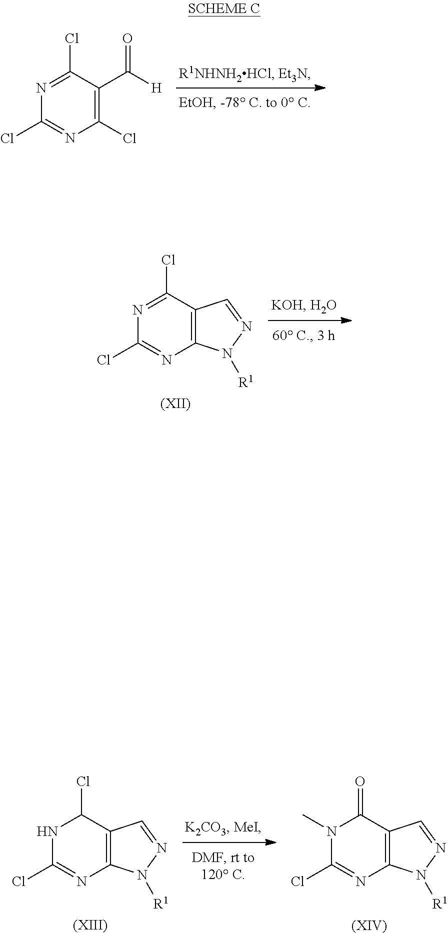

According to Scheme C, 6-chloro-pyrazolopyrimidinone compounds of formula (XIV), where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl, are synthetically accessible in 3 steps starting from 2,4,6-trichloropyrimidine-5-carbaldehyde. Initially, formation of the pyrazole ring is achieved by combining a commercially available substituted hydrazine or hydrazine hydrochloride salt and 2,4,6-trichloropyrimidine-5-carbaldehyde. For example, treatment of a solution of 2,4,6-trichloropyrimidine-5-carbaldehyde in a solvent, such as ethanol or the like, with a hydrazine compound, at an initial temperature of −78° C., followed by slow addition of a base, such as trimethylamine or the like, followed by stirring at −78° C. for several hours, then stirring at 0° C. for several hours, provides a substituted pyrazolopyrimidine compound of formula (XII). Subsequent treatment with a base, such as potassium hydroxide or sodium hydroxide or the like, in water, at a temperature ranging from 40° C. to 80° C., sometimes 60° C., provides a 6-chloropyrazolopyrimidinone of formula (XIII). Methylation of the nitrogen of a pyrazolopyrimidinone of formula (XIII) is achieved by treatment with a halo methane, in the presence of a base, under conditions known to one of skill in the art. For instance, treatment of a compound of formula (XIII) with iodomethane, in the presence of a base, such as potassium carbonate or the like, in a solvent such as DMF or DMA, or the like, at a temperature ranging from rt to 120° C., for several hours, provides a compound of formula (XIV).

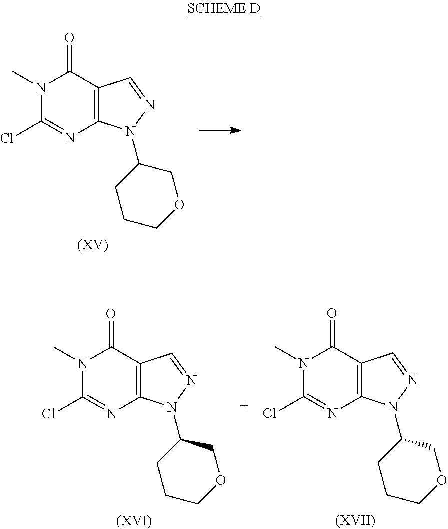

Isolation of two pure enantiomers of formula (XVI) and formula (XVII) is achieved by chiral separation of the corresponding racemate of formula (XV), using chiral SFC.

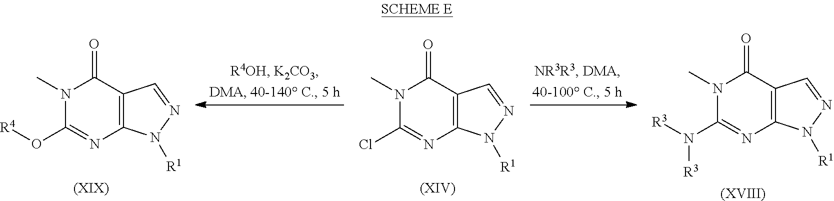

Nucleophilic substitution reactions of 6-chloropyrazolopyrimidinones of formula (XIV) with either a substituted amine or alcohol, with or without a base, under conditions known to one of skill in the art, provide 6-aminopyrazolopyrimidinone and 6-oxopyrazolopyrimidinones, respectively. For example, treatment of a 6-chloropyrazolopyrimidinone with an amine, in a solvent such as DMA or DMF, or the like, at a temperature ranging from 40° C. to 140° C., sometimes 100° C., for several hours, provides a compound of formula (XVIII) where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl and both R³ are independently H, C₁₋₆alkyl, aryl, or heteroaryl, or both R³ can come together to form a heterocyclic ring including piperidine, piperazine, pyrrolidine or 1,2,3,4-tetrahydroisoquinoline. Similarly, treatment of 6-chloropyrazolopyrimidinone with an alcohol, in a solvent such as DMA or DMF, or the like, in the presence of a base, such as potassium carbonate, or the like, at a temperature ranging from 40° C. to 140° C., sometimes 100° C., for several hours, provides a compound of formula (XIX) where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl, and R⁴ is phenyl, pyridyl, pyrimidine, pyrazine, pyridazine, indole, indazole, quinolin-2(1H)-one, isoquinoline, indolin-2-one, or 3,4-dihydroisoquinoline-1(2H)-one.

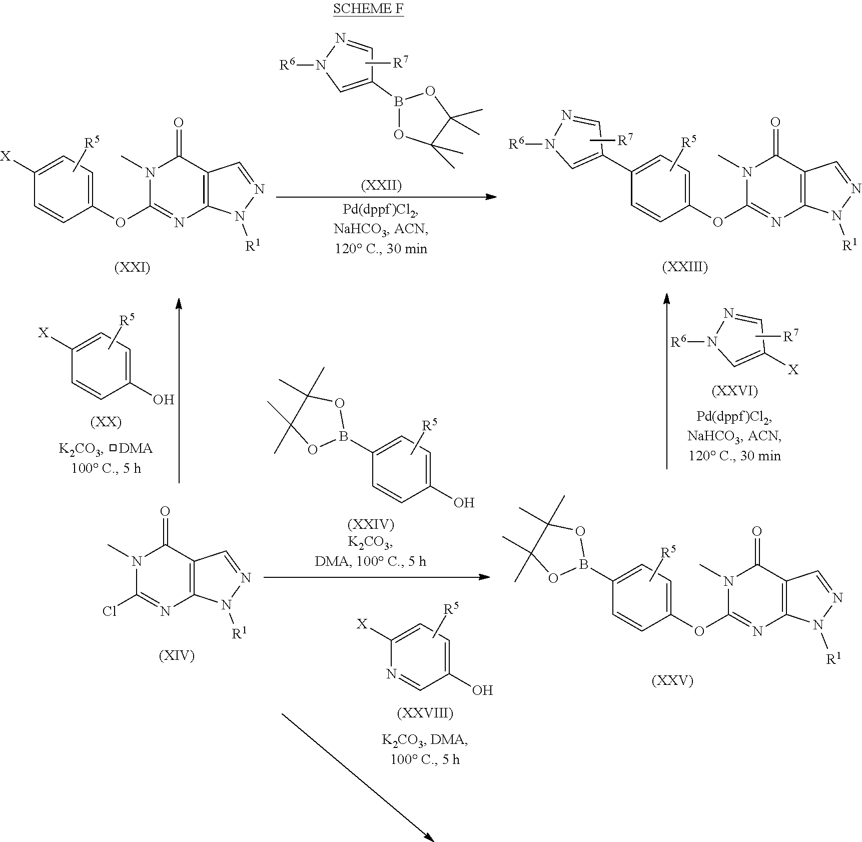

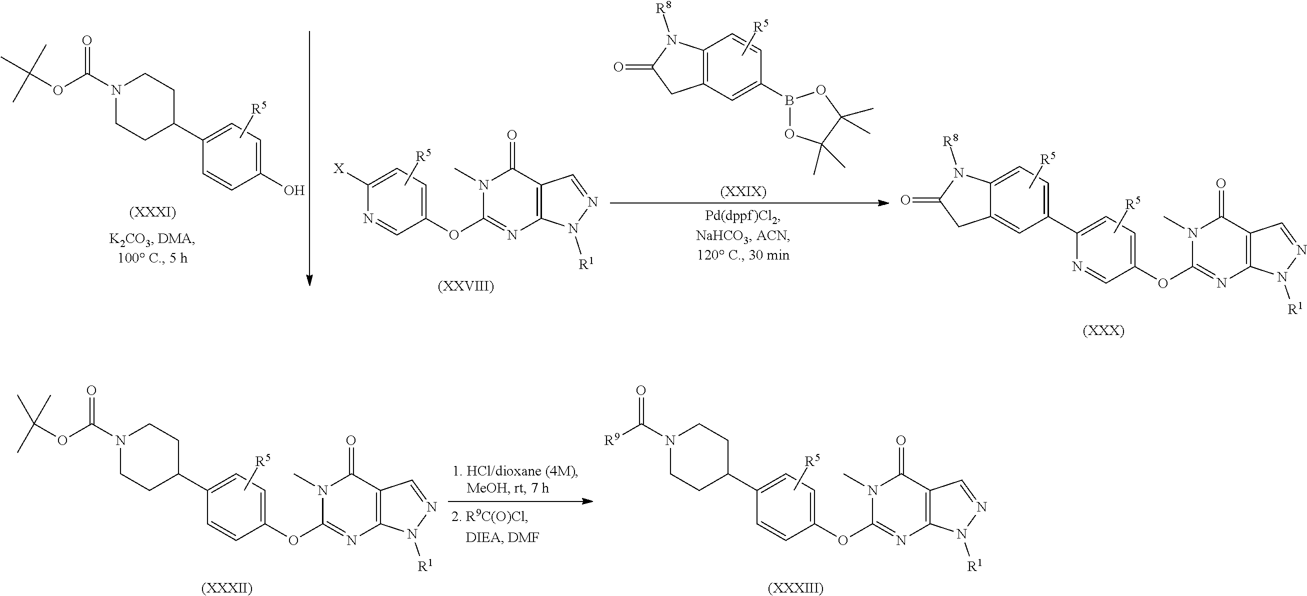

An SN_{Ar} coupling of a 6-chloropyrazolopyrimidinone and a substituted phenol or a substituted hydroxyheteroaryl compound, using conditions known to one of skill in the art, followed by a Suzuki coupling with an heteroaryl or heterocycloalkyl group, gives compounds of formula (XXIII) and formula (XXX). For example, treatment of a 6-chloropyrazolopyrimidinone with a substituted 4-(4,4,5,5-tetramethyl-1,3,2-dioxaborolan-2-yl)phenol, in the presence of a base, such as potassium carbonate or the like, in a solvent such as DMA or DMF, or the like, heated to a temperature ranging from 40° C. to 140° C., for several hours, provides a compound of formula (XXV), where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl, and R⁷ is 1 or 2 C₁₋₆alkyl, halo, or cyclopropyl. Further treatment with a 1-substituted-4-halopyrazole, provides a compound of formula (XXIII). For example, treatment of a compound of formula (XXV) with 1-substituted-4-halopyrazole in the presence of a catalyst, such as Pd(dppf)Cl₂, or the like, and a base, such as NaHCO₃, or the like, in a solvent like ACN, heated to a temperature ranging from 60° C. to 120° C. in a microwave reactor, provides a compound of formula (XXIII), where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl, and R⁵ is 1 or more C₁₋₆alkyl, C₁₋₆haloalkyl, C₁₋₆alkoxy, C₁₋₆haloalkoxy, or halogen, and R⁶ is C₁₋₆alkyl, C₁₋₆haloalkyl, C₃₋₆cycloalkyl, C₃₋₆halocycloalkyl and R⁷ is 1 or 2 C₁₋₆alkyl groups. Alternatively, treatment of a 6-chloropyrazolopyrimidinone with a substituted 4-halophenol, using the conditions described above, provides a compound of formula (XXI), where R¹ is C₁₋₄alkyl, C₁₋₆haloalkyl, C₃₋₆cycloalkyl, C₃₋₆halocycloalkyl, oxetanyl, tetrahydropyranyl, or tetrahydrofuranyl, R⁵ is 1 or more C₁₋₆alkyl, C₁₋₆haloalkyl, C₁₋₆alkoxy, C₁₋₆haloalkoxy, or halogen, and X is Cl, Br or I. Further treatment with a boronate ester or boronic acid substituted heteroaryl group, for instance with 1-alkyl-4-(4,4,5,5-tetramethyl-1,3,2-dioxaborolan-2-yl)-1H-pyrazole, also provides a compound of formula (XXIII). For example, treatment of a compound of formula (XXI) with 1-alkyl-4-(4,4,5,5-tetramethyl-1,3,2-dioxaborolan-2-yl)-1H-pyrazole in the presence of a catalyst, such as Pd(dppf)Cl₂, or the like, and a base, such as NaHCO₃, or the like, in a solvent like ACN, heated to a temperature ranging from 60° C. to 120° C. in a microwave reactor, provides a compound of formula (XXIII), where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl, and R⁵ is 1 or more C₁₋₆alkyl, C₁₋₆haloalkyl, C₁₋₆alkoxy, C₁₋₆haloalkoxy, or halogen, and R⁶ is C₁₋₆alkyl, C₁₋₆haloalkyl, C₃₋₆cycloalkyl, and C₃₋₆halocycloalkyl.

In an alternate method, treatment of a 6-chloropyrazolopyrimidinone with a substituted 6-halopyridin-3-ol of formula (XXVII), according to the procedure described above, provides an intermediate of formula (XXVIII), where X is Cl, Br or I. Further treatment with substituted 5-(4,4,5,5-tetramethyl-1,3,2-dioxaborolan-2-yl)indolin-2-one, using the conditions described above, affords a compound for formula (XXX), where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl, R⁵ is 1 or more C₁₋₆alkyl, C₁₋₆haloalkyl, C₁₋₆alkoxy, C₁₋₆haloalkoxy, or halogen, and R⁸ is H, C₁₋₆alkyl, C₃₋₆cycloalkyl, C₁₋₆haloalkyl or C₃₋₆halocycloalkyl.

In addition, acylpiperidine analogs can be synthesized from intermediates of formula (XIV) in two steps. Initially, a nucleophilic substitution reaction of a compound of formula (XIV) with a 4-phenyl piperidine compound, under conditions described above, provides a tert-butoxycarbonyl protected compound of formula (XXXII). Subsequent deprotection followed by acetylation provides an amide of formula (XXXIII). For example, removal of the tert-butyloxycarbonyl protecting group under acidic conditions known to one of skill in the art, for instance addition of a 4M solution of HCl in dioxane, in a solvent such as methanol or the like, at rt for several hours, provides the free piperidine analog. Acetylation, using acetyl chloride, in the presence of a base, such as DIEA, in a solvent such as DMF or DMA or the like, provides a compound of formula (XXXIII), where R¹ is C₁₋₆alkyl, C₁₋₆haloalkyl, C₃₋₆cycloalkyl, C₃₋₆halocycloalkyl, oxetanyl, tetrahydropyranyl, or tetrahydrofuranyl, R⁵ is 1 or more C₁₋₆alkyl, C₁₋₆haloalkyl, C₁₋₆alkoxy, C₁₋₆haloalkoxy, or halogen, and R⁹ is CH₃. Additional amide analogs, where R⁹ is C₁₋₆alkyl, C₁₋₆haloalkyl, C₃₋₆cycloalkyl, or C₃₋₆halocycloalkyl can be synthesized in a similar fashion using the appropriate acid chloride.

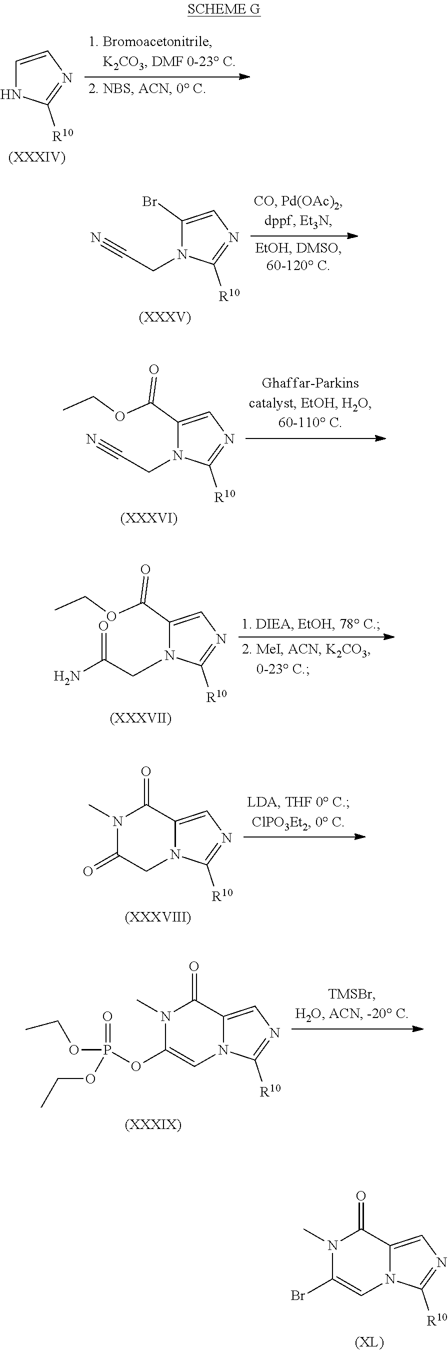

According to Scheme G, 3-substituted-6-bromo-7-methylimidazo[1,5-a]pyrazin-8(7H)-one compounds of formula (XL) can be synthesized from a 2-substituted imidazole of formula (XXXIV) in 7 steps. Initially, treatment of an imidazole analog with a haloacetonitrile, such as bromoacetonitrile, in the presence of a base, such as potassium carbonate, in a solvent such as DMF or DMA, or the like, at a temperature ranging from 0° C. to 23° C., provides a 2-(1H-imidazol-1-yl)acetonitrile. Subsequent halogenation, under conditions known to one of skill in the art, for example bromination, using NBS, in a solvent such as ACN, or the like, at 0° C., provides a compound of formula (XXXV), where R¹⁰ is C₁₋₆alkyl, C₁₋₆haloalkyl, C₃₋₆cycloalkyl, or C₃₋₆halocycloalkyl. Further, treatment with carbon monoxide, in the presence of a catalyst, such as palladium (II) acetate, or the like, and a ligand, such as 1,1′ bis(diphenylphosphino)ferrocene, and a base, such as triethylamine or the like, in a solvent mixture such as ethanol and DMSO, under an atmosphere of carbon monoxide (80 psi) and at a temperature ranging from 60° C. to 120° C., sometimes 80° C., provides an ester of formula (XXXVI). Hydrolysis of the acetonitrile to a primary amide can be achieved in the presence of a catalyst, such as Ghaffar-Parkins catalyst, or the like, in a solvent mixture such as ethanol and water, at a temperature ranging from 60° C. to 110° C., provides an amide of formula (XXXVII). Cyclization to an imidazopyrazinedione is achieved by treatment of a compound of formula (XXXVII) with a base, such as DIEA, in a solvent such as ethanol, at reflux for a period of 10-24 h. Subsequent methylation of the pyrazinedione nitrogen is achieved by addition of a methylating agent, under conditions known to one of skill in the art, such as addition of iodomethane, in the presence of a base, such as potassium carbonate, or the like, in a solvent such as acetonitrile, at a temperature ranging from 0° C. to rt, to provide a substituted bicycle of formula (XXXVIII). Treatment of the imidazopyrazinedione with LDA, which is formed by treatment of diisopropyl amine with n-BuLi in a solvent such as THF, or the like, followed by addition of diethyl chlorophosphate, at a temperature of 0° C., affords a diethyl phosphonate derivative of formula (XXXIX), where R¹⁰ is C₁₋₆alkyl, C₁₋₆haloalkyl, C₃₋₆cycloalkyl, or C₃₋₆halocycloalkyl. Finally, formation of a substituted 6-halo-7-methylimidazo[1,5-a]pyrazin-8(7H)-one of formula (XL) is achieved by a halogenation reaction, for example treatment of a compound of formula (XXXIX) with trimethylsilylbromide, in a solvent mixture such as water and acetonitrile, cooled to a temperature ranging from −30° C. to 0° C.

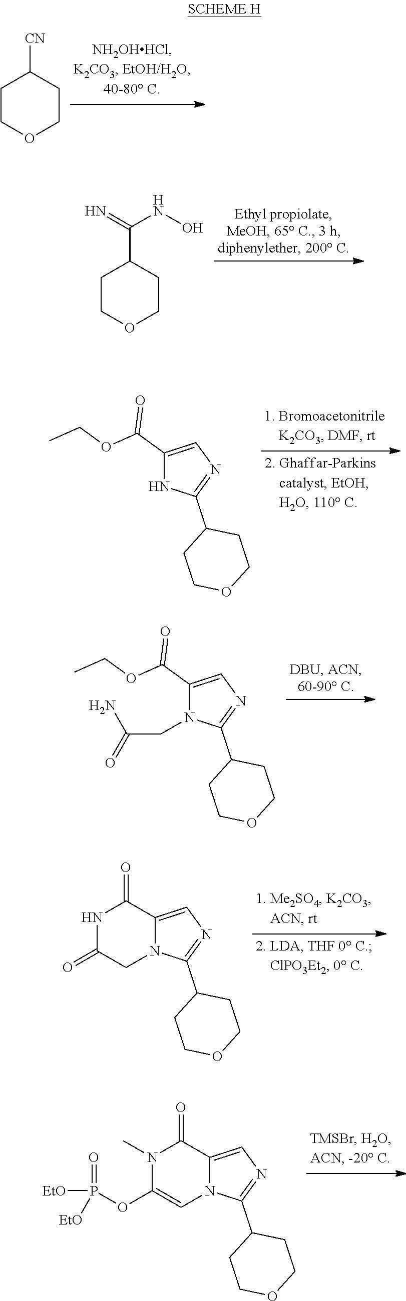 [SMILES: N#CC1CCOCC1 ; N=C(NO)C1CCOCC1 ; CCOC(=O)c1cnc(C2CCOCC2)[nH]1 ; CCOC(=O)c1cnc(C2CCOCC2)n1CC(N)=O ; O=C1Cn2c(cnc2C2CCOCC2)C(=O)N1 ; CCOP(=O)(OCC)Oc1cn2c(C3CCOCC3)ncc2c(=O)n1C]

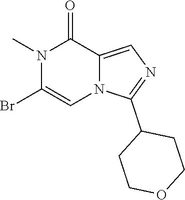 [SMILES: Cn1c(Br)cn2c(C3CCOCC3)ncc2c1=O]

6-Bromo-7-methyl-3-(tetrahydro-2H-pyran-4-yl)imidazo[1,5-a]pyrazin-8(7H)-one can be prepared from tetrahydropyran in 8 steps, according to Scheme H.

N-hydroxytetrahydro-2H-pyran-4-carboximidamide can be synthesized from tetrahydro-2H-pyran-4-carbonitrile and hydroxylamine in the presence of a base. For example, treatment of tetrahydro-2H-pyran-4-carbonitrile with hydroxylamine hydrochloride, in the presence of a base, such as potassium carbonate or the like, in a solvent mixture such as ethanol and water, heated to a temperature ranging from 40° C. to 80° C., sometimes 70° C., provides N-hydroxytetrahydro-2H-pyran-4-carboximidamide. Subsequent treatment with ethyl propiolate, in a solvent such as methanol, at refluxing temperature, followed by removal of solvent and replacement with diphenylether, then heating to a temperature ranging from 140° C. to 250° C., preferably 200° C., for a time period of 8-24 h, provides ethyl 2-(tetrahydro-2H-pyran-4-yl)-1H-imidazole-5-carboxylate. The synthesis of ethyl 1-(2-amino-2-oxoethyl)-2-(tetrahydro-2H-pyran-4-yl)-1H-imidazole-5-carboxylate is achieved by treatment of ethyl 2-(tetrahydro-2H-pyran-4-yl)-1H-imidazole-5-carboxylate with bromoacetonitrile, followed by Ghaffar-Parkins catalyst, according to the methods described above in Scheme G. Then, addition of DBU in a solvent, such as ACN, or the like, at a temperature ranging from 60° C. to 90° C., provides the cyclized compound 3-(tetrahydro-2H-pyran-4-yl)imidazo[1,5-a]pyrazine-6,8(5H,7H)-dione. Methylation of the pyrazinedione nitrogen is achieved using dimethyl sulfate, in the presence of a base, such as potassium carbonate or the like, in a solvent, such as ACN or the like, to provide 6-bromo-7-methyl-3-(tetrahydro-2H-pyran-4-yl)imidazo[1,5-a]pyrazin-8(7H)-one. Finally, formation of the diethylphosphonate ester, and subsequent bromination, according to the conditions previously described in Scheme G, afford 6-bromo-7-methyl-3-(tetrahydro-2H-pyran-4-yl)imidazo[1,5-a]pyrazin-8(7H)-one.

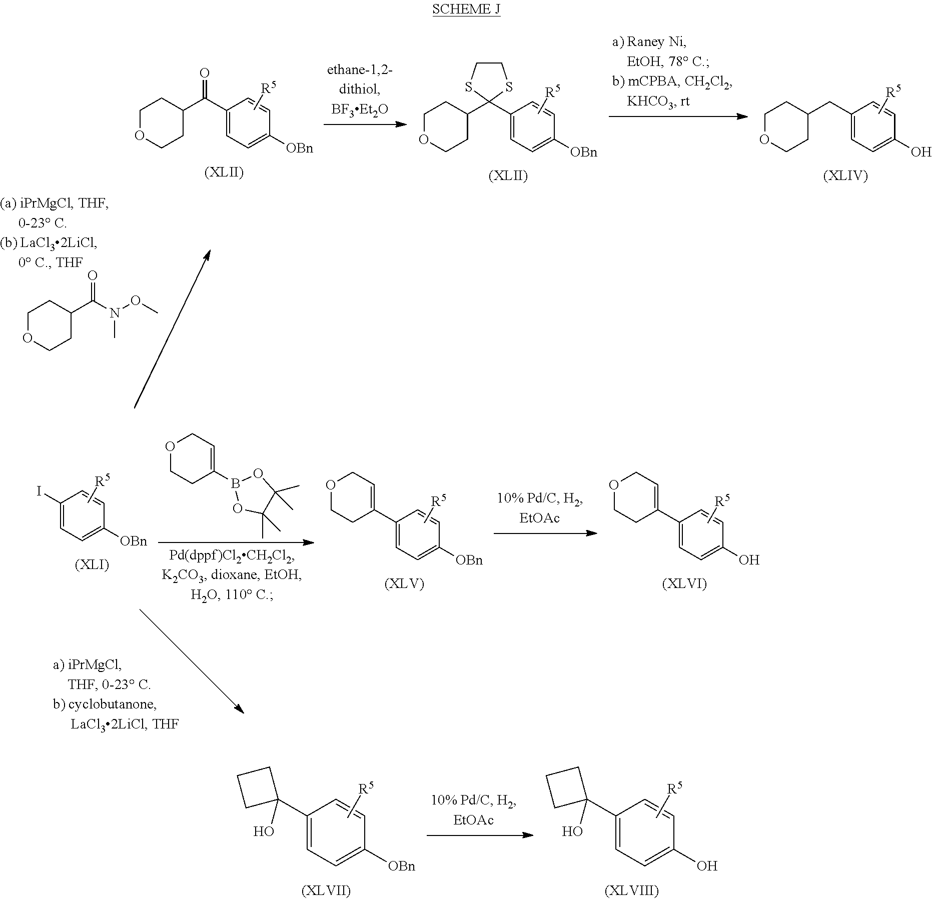

Several substituted phenol analogs can be synthesized according to Scheme J. In one instance, substituted 4-((tetrahydro-2H-pyran-4-yl)methyl)phenol can be synthesized from a substituted 1-(benzyloxy)-4-iodobenzene of formula (XLI). Initially, treatment of an aryl iodide analog with an isopropylmagnesium halide, such as isopropylmagnesium chloride, in a solvent such as THF or the like, at a temperature ranging from 0° C. to rt, for several hours provides the corresponding aryl Grignard reagent. Subsequent addition of this Grignard reagent to a solution of N-methoxy-N-methyltetrahydro-2H-pyran-4-carboxamide and LaCl₃.2LiCl in THF, cooled to a temperature of 0° C. for several hours provides a compound of formula (XLII), where R⁵ is 1 or more C₁₋₆alkyl, C₁₋₆haloalkyl, C₁₋₆alkoxy, C₁₋₆haloalkoxy, or halogen. Formation of a dithiane of formula (XLIII) is achieved by treatment of the ketone with ethane-1,2-dithiol and diethyl etherate, at 0° C., followed by stirring at rt for several hours. Reduction of the dithiane, using a catalyst such as Raney nickel, in a solvent, such as ethanol or the like, cooled to a temperature of −78° C. for several hours provides the desired compound, which can be contaminated with a partially reduced alkene intermediate. Treatment of the mixture with mCPBA, in a solvent such as DCM, in the presence of a base such as KHCO₃, or the like, provides the epoxide of the alkene byproduct, which can easily be separated from the desired product of formula (XLIV).

In another instance, substituted 4-(tetrahydro-2H-pyran-4-yl)phenol can be synthesized from a substituted 1-(benzyloxy)-4-iodobenzene of formula (XLI). In a Suzuki coupling reaction, treatment of a substituted aryl iodide with a boronate ester or boronic acid substituted 3,6-dihydro-2H-pyran, such as 2-(3,6-dihydro-2H-pyran-4-yl)-4,4,5,5-tetramethyl-1,3,2-dioxaborolane, using a catalyst, such as [1,1′-bis(diphenylphosphino)-ferrocene]dichloropalladium(II), in the presence of a base, such as potassium carbonate, or the like, in a solvent mixture, such as dioxane, ethanol and water, at a temperature ranging from 80° C. to 110° C., provides a compound of formula (XLV). Subsequent reduction of the double bond and removal of the benzyl protecting group using hydrogenation conditions, known to one of skill in the art, such as using a catalyst, such as Pd/C, 10%, in a solvent such as ethyl acetate or the like, in the presence of hydrogen gas, provides a substituted 4-(tetrahydro-2H-pyran-4-yl)phenol of formula (XLVI).

In a third instance, substituted 1-(4-(benzyloxy)phenyl)cyclobutan-1-ol can be synthesized from a substituted 1-(benzyloxy)-4-iodobenzene of formula (XLI) in a Grignard addition with cyclobutanone, using the conditions described above. Subsequent hydrogenation to remove the benzyl protecting group, using the conditions described above, provides a compound of formula (XLVIII), where R⁵ is 1 or more C₁₋₆alkyl, C₁₋₆haloalkyl, C₁₋₆alkoxy, C₁₋₆haloalkoxy, or halogen.

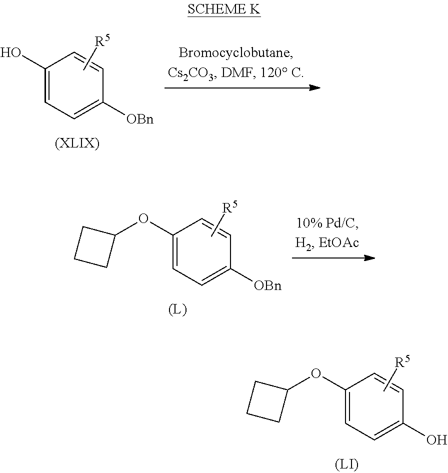

According to Scheme K, a substituted 4-cyclobutoxyphenol compound can be synthesized from substituted 4-(benzyloxy)phenol in two steps by a nucleophilic substitution reaction followed by a hydrogenation reaction. Treatment of the phenol with bromocyclobutane, in the presence of a base such as cesium carbonate, or the like, in a solvent such as DMF or DMA or the like, heated to a temperature ranging from 80° C. to 140° C. provides an ether of formula (L). Subsequent hydrogenation to remove the benzyl protecting group, using conditions described above, provides a phenol of formula (LI), where R⁵ is 1 or more C₁₋₆alkyl, C₁₋₆haloalkyl, C₁₋₆alkoxy, C₁₋₆haloalkoxy, or halogen.

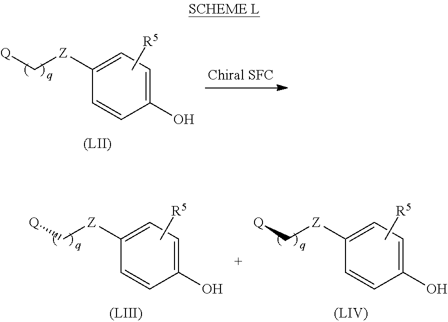

Isolation of two pure enantiomers of formula (LIII) and formula (LIV) is achieved by chiral separation of the corresponding racemate of formula (LII), using chiral SFC, where Q is heterocycloalkyl or cycloalkyl, Z is O or NH, R⁵ is 1 or more C₁₋₆alkyl, C₁₋₆haloalkyl, C₁₋₆alkoxy, C₁₋₆haloalkoxy, or halogen and q is 0 or 1.

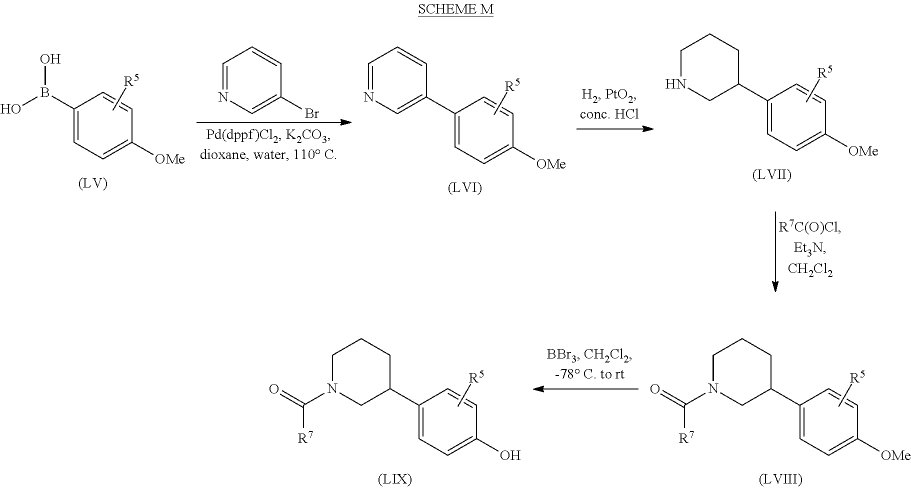

According to Scheme M, substituted 4-piperidinephenols can be synthesized from a substituted aryl boronic acid or aryl boronate ester in 4 steps. Coupling of substituted 3-(4-methoxyphenyl)pyridine and a bromopyridine in a Suzuki coupling, using conditions known to one of skill in the art, provides a compound of formula (LVI), where R⁵ is 1 or more C₁₋₆alkyl, C₁₋₆haloalkyl, C₁₋₆alkoxy, C₁₋₆haloalkoxy, or halogen. For example, treatment of a boronic acid of formula (LV) with 2-bromopyridine, in the presence of a catalyst such as Pd(dppf)Cl₂, in the presence of a base, such as potassium carbonate or the like, in a solvent mixture such as dioxane and water, heated to a temperature ranging from 80° C. to 110° C., provides a substituted 3-(4-methoxyphenyl)pyridine of formula (LVI). Subsequent reduction of the pyridine using hydrogenation conditions, known to one of skill in the art, for instance using a catalyst such as platinum(IV) oxide, in the presence of an acid, such as HCl, under a hydrogen environment, provides a compound of formula (LVII). Acylation, using an acyl chloride, such as acetyl chloride, in the presence of a base, such as triethylamine or DIEA, in a solvent such as DMF or DMA or the like, provides a compound of formula (LVIII), where and R⁵ is 1 or more C₁₋₆alkyl, C₁₋₆haloalkyl, C₁₋₆alkoxy, C₁₋₆haloalkoxy, or halogen and R⁷ is CH₃. Additional amide analogs, where R⁷ is C₁₋₆alkyl, C₁₋₆haloalkyl, C₃₋₆cycloalkyl, or C₃₋₆halocycloalkyl, can be synthesized in a similar fashion using the appropriate acid chloride. Finally, cleavage of the methyl ether to provide the free phenol is achieved by treatment of the phenol analog with boron tribromide, in a solvent such as DCM, or the like, cooled to a temperature ranging from −78° C. to room temperature, sometimes ranging from −40° C. to 0° C., provides a compound of formula (LIX).

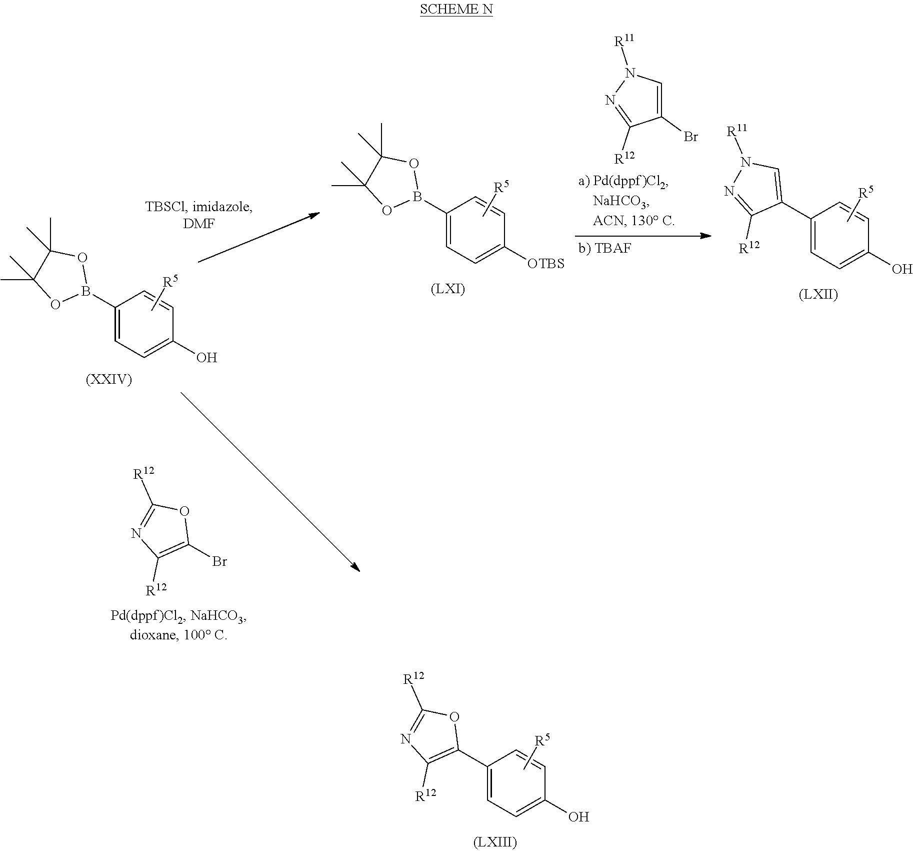

According to Scheme N, 4-heteroaryl substituted phenols can be synthesized in 1 to 3 steps from a substituted 4-hydroxyphenyl boronic acid or substituted 4-hydroxyphenyl boronate ester. In one instance, protection of the phenol oxygen with a TBS group, followed by a Suzuki coupling, provides a 4-heteroaryl substituted phenol after removal of the silyl protecting group. For instance, TBS protection, under conditions known to one of skill in the art, such as treatment of a phenol of formula (XXIV) with TBSCl, in the presence of a base, such as imidazole or the like, in a solvent such as DMF or DMA, or the like, provides a tert-butyldimethylsilyl ether of formula (LX). Next, Suzuki coupling with a substituted 4-bromopyrazole of formula (LXI), under conditions known to one of skill in the art, for example using a catalyst, such as Pd(dppf)Cl₂, in the presence of a base, such as sodium bicarbonate or the like, in a solvent such as ACN or dioxane, or the like, at a temperature ranging from 80° C. to 140° C., then TBS removal, provides an aryl pyrazole of formula (LXII), where R⁵ is 1 or more C₁₋₆alkyl, C₁₋₆haloalkyl, C₁₋₆alkoxy, C₁₋₆haloalkoxy, or halogen, R¹¹ is C₁₋₆alkyl, C₁₋₆haloalkyl, C₃₋₆cycloalkyl, or C₃₋₆halocycloalkyl, and R¹² is C₁₋₆alkyl, C₁₋₆haloalkyl. In another instance, treatment of a boronate ester of formula (XXIV) with 5-bromo-2,4-dimethyloxazole in a Suzuki coupling, using the conditions described above, provides a substituted 4-oxazolophenol of formula (LXIII), where R⁵ is 1 or more C₁₋₆alkyl, C₁₋₆haloalkyl, C₁₋₆alkoxy, C₁₋₆haloalkoxy, or halogen.

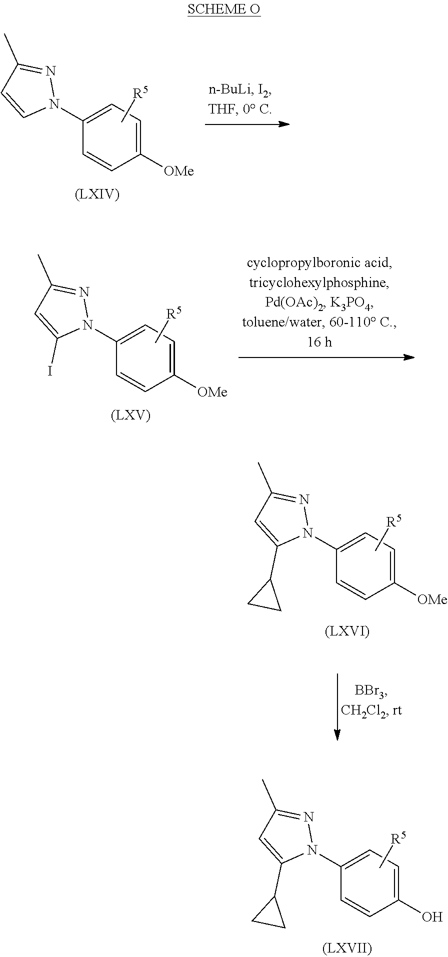

Formation of a cycloalkyl-substituted pyrazole compound of formula (LXVII) can be synthesized in 3 steps, according to Scheme O. Iodination of the pyrazole, under conditions known to one of skill in the art, for instance, treatment with n-butyllithium and iodine, in a solvent such as THF or the like, at 0° C. provides an iodopyrazole of formula (LXV). A Suzuki coupling of the iodide compound and cyclopropyl boronic acid, under conditions known to one of skill in the art, provides a cyclopropyl-substituted pyrazole. For example, treatment of the iodopyrazole of formula (LXV) with cyclopropyl boronic acid, using a catalyst, such as palladium(II) acetate, and a ligand, such as tricyclohexylphosphine, in the presence of a base, such as potassium phosphate or the like, in a solvent mixture such as toluene and water, at a temperature ranging from 60° C. to 110° C., sometimes 80° C., provides a compound of formula (LXVI). Cleavage of the methyl ether to provide the free phenol is achieved by treatment of the ether analog with boron tribromide, under conditions described in Scheme M, provides a compound of formula (LXVII).

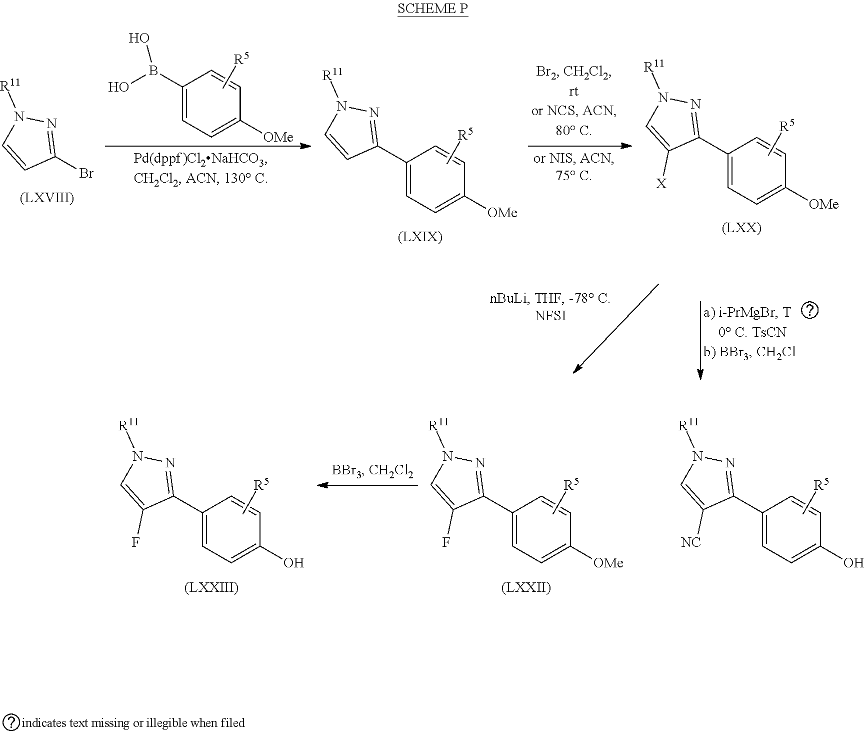

According to Scheme P, several methods can be employed to synthesize various substituted 4-pyrazole substituted phenol analogs. Starting from a substituted 3-bromopyrazole, a compound of formula (LXX) can be synthesized in two steps, where X is Br, Cl or I and R⁵ is 1 or more C₁₋₆alkyl, C₁₋₆haloalkyl, C₁₋₆alkoxy, C₁₋₆haloalkoxy, or halogen. For example, a Suzuki coupling of an aryl bromide and an aryl boronic acid, under conditions similar to those described in Scheme N, using a solvent mixture of DCM and ACN, provides a compound of formula (LXIX). Halogenation, under conditions know to one of skill in the art, for example bromination using brominating reagent, such as bromine, in a solvent such as DCM provides a 4-bromopyrazole compound for formula (LXX). Alternatively, treatment of a compound of formula (LXIX) with a chlorinating or iodinating reagent, such as NCS or NIS, in a solvent such as ACN, heated to a temperature ranging from 60° C. to 100° C. for several hours, provides a compound of formula (LXX), where X is Cl or I, respectively.

Substitution of the halide with a nitrile group is achieved by treatment with isopropyl magnesium bromide, in a solvent such as THF or the like, followed by addition of TsCN, at a temperature of 0° C. Subsequent removal of the methyl ether, using BBr₃, under conditions described in Scheme M, affords a 5-cyanopyrazole analogs of formula (LXXI), where R⁵ is 1 or more C₁₋₆alkyl, C₁₋₆haloalkyl, C₁₋₆alkoxy, C₁₋₆haloalkoxy, or halogen.

Alternatively, conversion of the 4-halopyrazole analog to a 4-fluoropyrazole analog of formula (LXXIII) can be achieved in 2 steps, including fluorination, followed by ether cleavage. For example, treatment of a compound of formula (LXX) with a base, such as n-BuLi, in a solvent such as THF, or the like, at a temperature of −78° C., followed by addition of NFSI, provides a 4-fluoropyrazole compound of formula (LXXII). Cleavage of the methyl ether using BBr₃, under conditions described in Scheme M, affords a phenol analog of formula (LXXIII), where R⁵ is 1 or more C₁₋₆alkyl, C₁₋₆haloalkyl, C₁₋₆alkoxy, C₁₋₆haloalkoxy, or halogen.

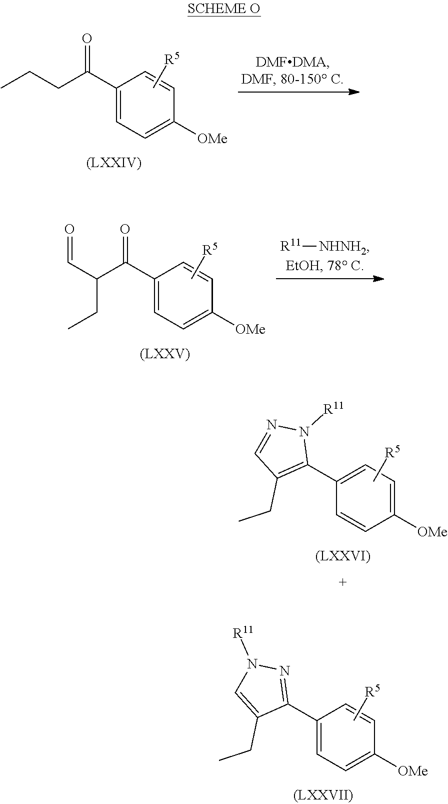

The synthesis of 4-alkyl substituted 5-aryl pyrazoles can be achieved in two steps, from a substituted 1-(4-methoxyphenyl)butan-1-one compound, according to Scheme Q. Formation of a substituted 2-benzoylbutanal of formula (LXXV) is achieved by treatment of the ketone compound with N,N-dimethylformamide dimethyl acetal, in a solvent such as DMF, at a temperature ranging from 80° C. to 150° C., sometimes 120° C. Subsequent treatment of the aldehyde intermediate with an alkyl hydrazine, in a solvent such as ethanol, with heat, provides both pyrazole regioisomers of formula (LXXVI) and (LXXVII), which can be isolated by separation using flash chromatography.

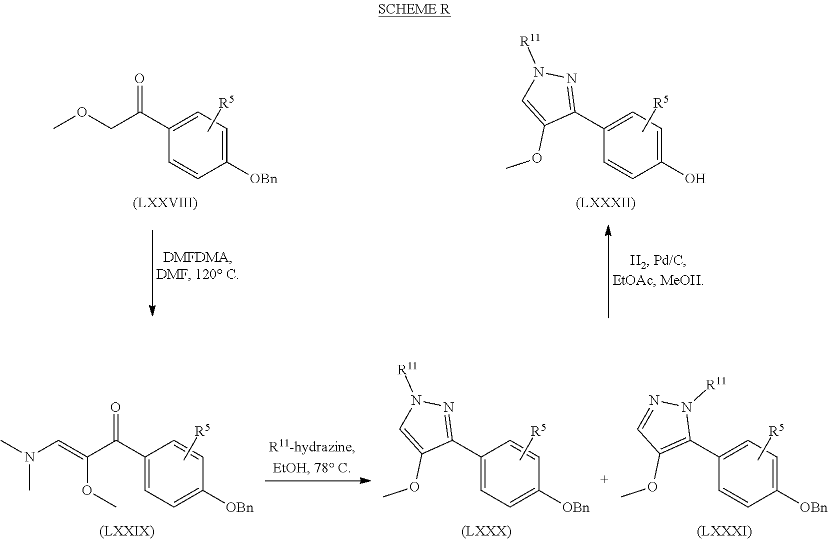

Aryl, alkoxy substituted pyrazole compounds can be synthesized in three steps from a compound of formula (LXXVIII), as depicted in Scheme R. Treatment of substituted 2-methoxy-1-phenylethan-1-one with N,N-dimethylformamide dimethyl acetal, in a solvent such as DMF or DMA or the like, heated to a temperature ranging from 60° C. to 140° C. provides a dimethylamino compound of formula (LXXIX), where R⁵ is 1 or more C₁₋₆alkyl, C₁₋₆haloalkyl, C₁₋₆alkoxy, C₁₋₆haloalkoxy, or halogen. Subsequent pyrazole formation using a substituted hydrazine, in a solvent such as ethanol, or the like, at reflux, provides both pyrazole regioisomers of formula (LXXX) and (LXXXI). Removal of the benzyl protecting group using hydrogenation conditions, as previously described in Scheme J, using a solvent mixture of ethyl acetate and methanol, followed by separation using flash chromatography, provides the desired 3-aryl-4-alkyoxy substituted pyrazole of formula (LXXXII).

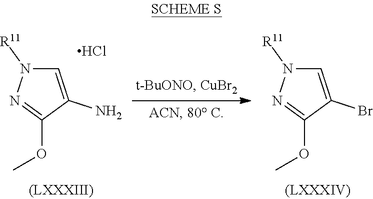

An amino-substituted pyrazole can be converted in a bromo-substituted pyrazole using tert-butyl nitrite and copper(II) bromide, under conditions known to one of skill in the art. For example, treatment of an aminopyrazole of formula (LXXXIII) with tert-butyl nitrite and copper(II) bromide, in a solvent such as acetonitrile or the like, at 80° C., provides a bromopyrazole of formula (LXXXIV).

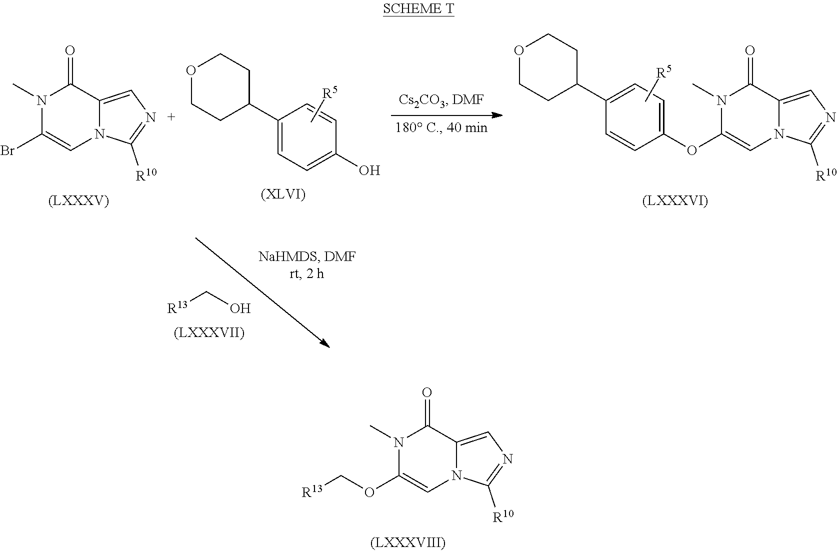

According to Scheme T, a substituted 6-alkoxyimidazopyrazinone or 6-aryloxyimidazopyrazinone can be synthesized in a single step from an alcohol or phenol and a substituted 6-bromomidazopyrazinone of formula (LXXXV). In one embodiment, treatment of a 6-bromomidazopyrazinone with a phenol of formula (XLVI), in the presence of a base, such as cesium carbonate or the like, in a solvent such as DMF or DMA, or the like, heated to a temperature ranging from 80° C. to 200° C., using traditional or microwave heating, provides a compound of formula (LXXXVI), where R⁵ is 1 or more C₁₋₆alkyl, C₁₋₆haloalkyl, C₁₋₆alkoxy, C₁₋₆haloalkoxy, or halogen, and R¹⁴ is C₁₋₆alkyl, C₁₋₆haloalkyl, C₃₋₆cycloalkyl, C₃₋₆halocycloalkyl, oxetanyl, tetrahydropyranyl, or tetrahydrofuranyl. In another embodiment, treatment of a 6-bromomidazopyrazinone with an alcohol of formula (LXXXVII), in the presence of a base such as sodium hexamethyldisilazane or the like, in a solvent such as DMF or DMA or the like, at room temperature, for several hours, provides a compound for formula (LXXXVIII), where R¹³ is C₃₋₆cycloalkyl, or C₄₋₆heterocycloalkyl and R¹⁴ is C₁₋₆alkyl, C₁₋₆haloalkyl, C₃₋₆cycloalkyl, C₃₋₆halocycloalkyl, oxetanyl, tetrahydropyranyl, or tetrahydrofuranyl.

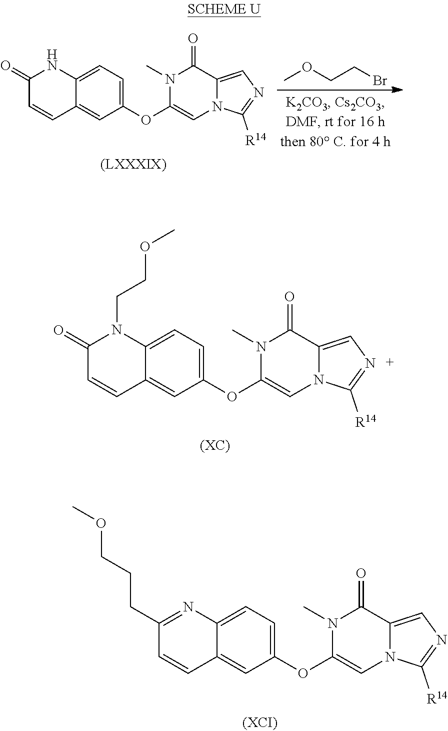

Alkylation of a quinolinone compound of formula (LXXXIX) using an alkyl bromide, in the presence of a base, under conditions known to one of skill in the art, provides both the N-alkylated compound of formula (XC) and the O-alkylated compound of formula (XCI). For example, treatment of a compound of formula (LXXXIX) with an alkyl bromide, in the presence of a base such as potassium carbonate or cesium carbonate, or the like, in a solvent such as DMF, at a temperature ranging from rt to 140° C., provides compounds of formula (XC) and (XCI), where R¹⁴ is C₁₋₆alkyl, C₁₋₆haloalkyl, C₃₋₆cycloalkyl, C₃₋₆halocycloalkyl, oxetanyl, tetrahydropyranyl, or tetrahydrofuranyl.

### EXAMPLES

Chemistry:

In obtaining the compounds described in the examples below, and the corresponding analytical data, the following experimental and analytical protocols were followed unless otherwise indicated.

Unless otherwise stated, reaction mixtures were magnetically stirred at room temperature (rt) under an atmosphere of nitrogen. Where solutions were “dried,” they were generally dried over a drying agent such as Na₂SO₄ or MgSO₄. Where mixtures, solutions, and extracts were “concentrated,” they were typically concentrated on a rotary evaporator under reduced pressure.

Reactions under microwave irradiation conditions were carried out in a CEM Discover-SP with Activent microwave reaction apparatus, model number 909150, or Biotage Initiator, model number 355302.

Normal-phase flash column chromatography (FCC) was performed on Silica (SiO₂) using packed or prepackaged cartridges, eluting with the indicated solvents.

Analytical LC/MS were obtained on a Waters 2695 Separations Unit, 2487 Dual Absorbance Detector, Micromass ZQ fitted with ESI Probe, or a Waters Acquity™ Ultra performance LC (UPLC) with PDA eλ and SQ detectors. Alternatively, LC-MS was performed on a Waters Acquity UPLC-MS instrument equipped with a Acquity UPLC BEH C18 column (1.7 μm, 2.1×50 mm) and the solvent system A: 0.1% HCOOH in H₂O and B: 0.1% HCOOH in ACN. Column temperature was 45° C. All compounds were run using the same elution gradient, i.e., 5% to 95% solvent B in 0.75 min with a flow rate of 1 mL/min.

Analytical SFC-MS was performed on a Waters UPC²-MS instrument equipped with a Acquity UPC² BEH 2-ethylpyridine column (1.7 μm, 2.1×50 mm) and the solvent system A: CO₂ and B: 0.1% NH₄OH in MeOH. Column temperature was 55° C. All compounds were run using the same elution gradient, i.e., 3% to 35% solvent B in 0.75 min with a flow rate of 2.5 mL/min.

Preparative HPLC was performed on a Shimadzu SIL-10AP system using a Waters SunFire™ OBD (5 m, 30×100 mm) C18 column with a 15-minute gradient of 10-100% acetonitrile in water and 0.05% trifluoroacetic acid added as a modifier to both phases. Elution profiles were monitored by UV at 254 and 220 nm.

Some compounds were purified using a Waters Fractionlynx system equipped with a XBridge Prep C18 OBD column (5 μm, 19×50 mm) and the solvent system: H₂O:AcCN and 2% TFA in H₂O. Specific elution gradients were based on retention times obtained with an analytical UPLC-MS, however, in general all elution gradients of H₂O and ACN were run over a 5.9 min run time with a flow rate of 40 mL/min. An autoblend method was used to ensure a concentration of 0.1% TFA throughout each run.

Some compounds were purified using a Waters Fractionlynx system equipped with a XBridge Prep C18 OBD column (5 μm, 30×100 mm) and the solvent system: H₂O:AcCN and 2% TFA in H₂O. Specific elution gradients were based on retention times obtained with an analytical UPLC-MS, however, in general all elution gradients of H₂O and ACN were run over a 9 min run time with a flow rate of 60 mL/min. An autoblend method was used to ensure a concentration of 0.1% TFA throughout each run.

Preparative SFC-MS was run on a Waters Prep100 SFC-MS system equipped with a Viridis 2-ethylpyridine OBD column (5 μm, 30×100 mm) and the solvent system: CO₂:MeOH and 0.2% NH₄OH in MeOH as a co-solvent. Specific elution gradients were based on retention times obtained with an analytical UPC²-MS, however, in general all elution gradients of CO₂ and MeOH were run over a 3.6 min run time with a flow rate of 100 mL/min and a column temperature of 55° C. An autoblend method was used to ensure a concentration of 0.2% NH₄OH throughout each run.

Nuclear magnetic resonance (NMR) spectra were obtained in an Agilent 300 MHz VNMR (Varian 300 MHz NMR), a Varian 400 MHz or Bruker 400 MHz NMR. Samples were analyzed in either deuterated acetone ((CD₃)₂CO), chloroform (CDCl₃), MeOH-d₄ (CD₃OD), N,N-dimethylformamide-d₇ (DMF-d₇) or dimethyl sulfoxide-d₆ (DMSO-d₆). For (CD₃)₂CO samples, the residual central resonance peak at 2.05 for ¹H was used for chemical shift assignment for ¹H NMR spectra. For CDCl₃ samples, the residual central resonance peak at 7.26 for ¹H was used for chemical shift assignment for ¹H NMR spectra. For CD₃OD the residual central resonance peak at 3.31 for ¹H was used for chemical shift assignment and for DMF-d₇ the residual central resonance peaks at 2.92 or 2.75 for ¹H were used for chemical shift assignment. For DMSO-d₆ the residual central resonance peak at 2.50 ppm for ¹H was used for chemical shift assignment. The format of the ¹H NMR data below is: chemical shift in ppm downfield the tetramethylsilane reference (multiplicity, coupling constant J in Hz, integration), using conventional abbreviations for designation of major peaks: e.g. s, singlet; d, doublet; t, triplet; q, quartet; p, pentet; m, multiplet; br, broad.

Chemical names were generated using ChemDraw Ultra 12.0 (CambridgeSoft Corp., Cambridge, Mass.), ChemDraw Professional 15.1 (CambridgeSoft Corp., Cambridge, Mass.) or ChemAxon.

### Intermediate 1. 6-(4-hydroxybenzyl)-1-isopropyl-5-methyl-1,5-dihydro-4H-pyrazolo[3,4-d]pyrimidin-4-one

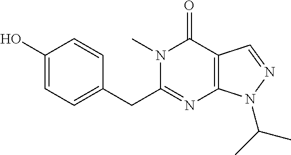 [SMILES: CC(C)n1ncc2c(=O)n(C)c(Cc3ccc(O)cc3)nc21]

Step 1. 5-Amino-1-isopropyl-1H-pyrazole-4-carbonitrile. Triethylamine (77 mL, 550 mmol) was added to an ice-cooled stirred solution of isopropyl hydrazine hydrochloride (25 g, 183 mmol) in ethanol (300 mL) and the resulting white slurry was stirred for 1 hour. 2-(ethoxymethylene)malononitrile (22.4 g, 183 mmol) in ethanol (200 mL) was warmed gently to effect dissolution and then was added over 30 minutes without allowing the temperature to go above 12° C. The reaction was stirred at room temperature for 18 h and reflux for 3 h and then concentrated under vacuum. The residue was taken up in water (300 mL) and extracted with ethyl acetate (1×200 mL and 3×100 mL). The combined extracts were dried (MgSO₄) and concentrated to afford 27 g (90%) of the title compound as a yellow semi-solid. ¹H NMR (400 MHz, CD₃OD) δ 7.50 (s, 1H), 4.39 (td, J=6.6, 13.2 Hz, 1H), 1.38 (d, J=6.7 Hz, 6H). [M+H]=151.1.

Step 2. 5-Amino-1-isopropyl-1H-pyrazole-4-carboxamide. Hydrogen peroxide (78.7 mL, 35% w/w, 0.90 mol) and aqueous ammonia (236 mL, 14.5 mol/L, 3.42 mol) were added to a stirred solution of 5-amino-1-isopropyl-1H-pyrazole-4-carbonitrile (27 g, 0.18 mol) in ethanol (236 mL) and the mixture was stirred for 3 days at room temperature. It was treated with a solution of saturated sodium thiosulfate (800 mL) over 4 hours with ice-cooling (CAUTION!). The mixture was concentrated under vacuum to remove the ethanol. The resulting precipitate was isolated by filtration, washed with water and dried under suction to afford the title compound (17.6 g, 58%) as yellow crystals. On standing, the mother liquor produced a second crop of the title compound as yellow crystals (3.12 g, 10%). ¹H NMR (400 MHz, DMSO-d₆) δ 7.63 (s, 1H), 7.15 (br s, 1H), 6.61 (br s, 1H), 6.14 (s, 2H), 4.45-4.30 (m, 1H), 1.28 (d, J=6.7 Hz, 6H). [M+H]=169.1.

Step 3. 6-(4-(Benzyloxy)benzyl)-1-isopropyl-1H-pyrazolo[3,4-d]pyrimidin-4(5H)-one. 5-Amino-1-isopropyl-1H-pyrazole-4-carboxamide (0.40 g, 2.38 mmol), methyl 2-(4-(benzyloxy)phenyl)acetate (1.22 g, 4.76 mmol), tetrahydrofuran (20 mL) and three 4A molecular sieve pellets were combined. Potassium tert-butoxide in THF (2.38 mL, 2.00 mol/1, 4.76 mmol) was added and the mixture was heated at 60° C. under nitrogen for 16 h. The mixture was filtered, diluted with ethyl acetate (100 mL) and washed with water (50 mL). The organic layer was dried (MgSO₄) and concentrated under vacuum to afford the title compound as a crude yellow solid (0.89 g, 90%), which was used in the subsequent methylation without further purification. ¹H NMR (400 MHz, CDCl₃) δ 11.41 (br s, 1H), 8.10 (s, 1H), 7.46-7.30 (m, 7H), 6.98-6.92 (m, 2H), 5.10 (quin, J=6.7 Hz, 1H), 5.05 (s, 2H), 4.03 (s, 2H), 1.58 (d, J=6.7 Hz, 6H). [M+H]=375.0.

Step 4. 6-(4-(Benzyloxy)benzyl)-1-isopropyl-5-methyl-1H-pyrazolo[3,4-d]pyrimidin-4(5H)-one. 6-(4-(Benzyloxy)benzyl)-1-isopropyl-1H-pyrazolo[3,4-d]pyrimidin-4(5H)-one (0.89 g, 2.38 mmol), DMF (9 mL), potassium carbonate (0.99 g, 7.14 mmol) and iodomethane (0.30 mL, 4.8 mmol) were combined and heated at 60° C. for 2 hours. The mixture was poured into water (50 mL) and extracted with DCM (3×25 mL). The combined extracts were concentrated under vacuum and the residue was purified by flash LC (elution with 5-100% ethyl acetate in hexanes) to afford the title compound (0.52 g, 53%). ¹H NMR (400 MHz, CDCl₃) δ 8.08 (s, 1H), 7.46-7.31 (m, 5H), 7.15 (d, J=8.7 Hz, 2H), 6.96 (d, J=8.7 Hz, 2H), 5.11-5.03 (m, 3H), 4.17 (s, 2H), 3.49 (s, 3H), 1.57 (d, J=6.7 Hz, 6H). [M+H]=389.0.

Step 5. 6-(4-Hydroxybenzyl)-1-isopropyl-1H-pyrazolo[3,4-d]pyrimidin-4(5H)-one. Palladium on carbon (10%, 109 mg, 0.10 mmol) was suspended in ethanol (2 mL) and added to a stirred solution of 6-(4-(benzyloxy)benzyl)-1-isopropyl-5-methyl-1H-pyrazolo[3,4-d]pyrimidin-4(5H)-one (515 mg, 1.02 mmol) in ethanol (10 mL) and ethyl acetate (5 mL). The mixture was purged with nitrogen and stirred under a balloon of hydrogen for 18 h. It was filtered through a plug of Celite®, and the Celite® was washed with ethyl acetate. The combined organics were concentrated under vacuum to afford the title compound (320 mg, 94%) as a white foam. ¹H NMR (400 MHz, DMSO-d₆) δ 9.40 (br s, 1H), 8.04 (s, 1H), 7.07 (d, J=8.5 Hz, 2H), 6.73 (d, J=8.4 Hz, 2H), 4.93 (td, J=6.7, 13.3 Hz, 1H), 4.15 (s, 2H), 3.39 (s, 3H), 1.48-1.40 (m, 6H). [M+H]=299.2.

### Intermediate 2. 6-Chloro-1-isopropyl-5-methyl-1H-pyrazolo[3,4-d]pyrimidin-4(5H)-one

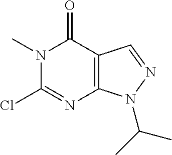

Step 1. 4,6-Dichloro-1-isopropyl-1H-pyrazolo[3,4-d]pyrimidine. To a stirred solution of 2,4,6-trichloropyrimidine-5-carboxylic acid (34.4 g, 163 mmol) in ethanol (430 mL) under a nitrogen atmosphere at −78° C. was added isopropyl hydrazine hydrochloride (18 g, 162.8 mmol), followed slowly by TEA (101.4 mL, 733 mmol). The mixture was stirred for 2 h at −78° C. and 3 h at 0° C. The reaction mixture was quenched with ice and the product was extracted into petroleum ether. The organic solvent was removed under reduced pressure below 35° C. to afford the crude compound as a dark yellow solid (27 g, 72%). [M+H]=231.0.

Step 2. 6-Chloro-1-isopropyl-1H-pyrazolo[3,4-d]pyrimidin-4(5H)-one. To a stirred suspension of 4,6-dichloro-1-isopropyl-1H-pyrazolo[3,4-d]pyrimidine in water (270 mL) was added potassium hydroxide (20 g, 351 mmol) and the resulting mixture was heated to 60° C. for 3 h. The reaction mixture was cooled to room temperature and the pH was adjusted to 2.0 using 2 N hydrochloric acid. The resulting solid isolated by filtration, washed with water and dried under vacuum to afford the title compound (19 g, 77%) as a pale yellow solid. ¹H NMR (400 MHz, DMSO-d₆) δ 13.16 (s, 1H), 8.08 (s, 1H), 4.89 (hept, J=6.7 Hz, 1H), 1.43 (d, J=6.6 Hz, 6H). [M+H]=213.0.

Step 3. 6-Chloro-1-isopropyl-5-methyl-1H-pyrazolo[3,4-d]pyrimidin-4(5H)-one. Iodomethane (17.6 mL, 282 mmol, 1.2 eq.) was added to a stirred suspension of 6-chloro-1-isopropyl-1H-pyrazolo[3,4-d]pyrimidin-4(5H)-one (50 g, 235 mmol) and potassium carbonate (65 g, 470 mmol) in DMF (470 mL) and the resulting mixture was stirred for 1 h. The mixture was filtered and the frit was washed with ethyl acetate. This solution was washed twice with a mixture of water (125 mL) and brine (25 mL). The potassium carbonate isolated above was dissolved in water and added to the aqueous layer from the above extraction. This solution was further extracted with ethyl acetate (5×125 mL). All organic extracts were combined, dried (MgSO₄) and concentrated. The resulting solid was triturated in water (500 mL) for 2 hours. The solid was isolated by filtration, washed with water and dried under vacuum to afford the title compound as a yellow solid (48.1 g, 90%). ¹H NMR (400 MHz, CDCl₃) δ 8.07 (s, 1H), 4.99 (spt, J=6.7 Hz, 1H), 3.74 (s, 3H), 1.55 (d, J=6.7 Hz, 6H). [M+H]=227.2.

### Intermediate 3. 6-Chloro-5-methyl-1-(tetrahydro-2H-pyran-4-yl)-1H-pyrazolo[3,4-d]pyrimidin-4(5H)-one

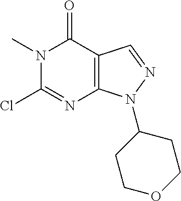 [SMILES: Cn1c(Cl)nc2c(cnn2C2CCOCC2)c1=O]

The title compound was prepared in a manner similar to Intermediate 2, steps 1-3, using the appropriate starting material substitutions. ¹H NMR (400 MHz, CD₃OD) δ 8.06 (s, 1H), 4.95-4.79 (m, 1H), 4.10 (dd, J=4.5, 11.5 Hz, 2H), 3.73 (s, 2H), 3.63 (dt, J=2.0, 12.0 Hz, 2H), 2.29 (dd, J=4.4, 12.4 Hz, 2H), 2.02-1.81 (m, 2H). [M+H]=269.3.

### Intermediate 4. 6-Chloro-5-methyl-1-(tetrahydro-2H-pyran-3-yl)-1H-pyrazolo[3,4-d]pyrimidin-4(5H)-one

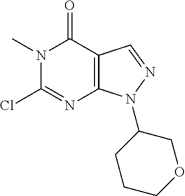 [SMILES: Cn1c(Cl)nc2c(cnn2C2CCCOC2)c1=O]

The title compound was prepared analogously to Intermediate 2, steps 1-3, using the appropriate starting material substitutions. ¹H NMR (400 MHz, CDCl₃) δ 8.07 (s, 1H), 4.82-4.74 (m, 1H), 4.06-3.97 (m, 2H), 3.86-3.76 (m, 1H), 3.74 (s, 3H), 3.57-3.46 (m, 1H), 2.35-2.23 (m, 1H), 2.23-2.12 (m, 1H), 1.95-1.82 (m, 2H). [M+H]=269.1.

### Intermediate 5 and 6. (R)-6-Chloro-5-methyl-1-(tetrahydro-2H-pyran-3-yl)-1H-pyrazolo[3,4-d]pyrimidin-4(5H)-one and (S)-6-chloro-5-methyl-1-(tetrahydro-2H-pyran-3-yl)-1H-pyrazolo[3,4-d]pyrimidin-4(5H)-one

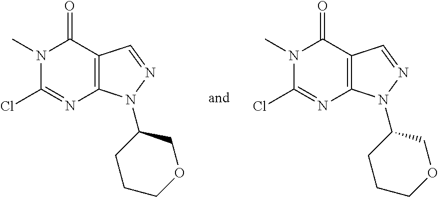 [SMILES: Cn1c(Cl)nc2c(cnn2[C@@H]2CCCOC2)c1=O ; Cn1c(Cl)nc2c(cnn2[C@H]2CCCOC2)c1=O]

6-Chloro-5-methyl-1-(tetrahydro-2H-pyran-3-yl)-1H-pyrazolo[3,4-d]pyrimidin-4(5H)-one (Intermediate 4) was resolved into constituent enantiomers using SFC chromatography. Separation was effected on a Chiralpak IC, 30×150 mm, 5-μm column using 25% methanol in CO₂ at 1500 psi with a flow rate of 100 mL over a six-minute run. X-ray crystallography confirmed that the (R)-enantiomer eluted first, followed by the (S)-enantiomer.

### Intermediate 7. 6-((6-Chloropyridin-3-yl)oxy)-1-isopropyl-5-methyl-1,5-dihydro-4H-pyrazolo[3,4-d]pyrimidin-4-one

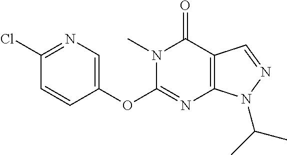 [SMILES: CC(C)n1ncc2c(=O)n(C)c(Oc3ccc(Cl)nc3)nc21]

Intermediate 2 (4.45 g, 19.6 mmol) and 6-chloropyridin-3-ol (3.31 g, 25.5 mmol, 1.3 eq.) were combined in DMF (89 ml) and treated with DIEA (8.6 mL, 49 mmol, 2.50 eq.). The mixture was heated to 85° C. for 18 h. It was cooled, concentrated under vacuum and purified by flash LC (elution with 0-70% ethyl acetate in hexanes) to afford 6.04 g (96%) of the title compound as a beige solid. ¹H NMR (400 MHz, CDCl₃) δ 8.42 (d, J=2.81 Hz, 1H) 8.02 (s, 1H) 7.61 (dd, J=8.68, 2.93 Hz, 1H) 7.40-7.54 (m, 1H) 4.61 (spt, J=6.72 Hz, 1H) 3.66 (s, 3H) 1.41 (d, J=6.85 Hz, 6H). [M+H]=320.1.

### Intermediate 8. 6-(4-Iodophenoxy)-1-isopropyl-5-methyl-1,5-dihydro-4H-pyrazolo[3,4-d]pyrimidin-4-one

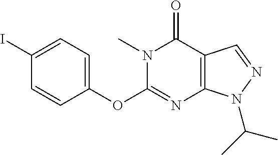 [SMILES: CC(C)n1ncc2c(=O)n(C)c(Oc3ccc(I)cc3)nc21]

This title compound was prepared in a manner analogous to Intermediate 7, using the appropriate starting material substitutions. ¹H NMR (400 MHz, CDCl₃) δ 8.00 (s, 1H), 7.78 (d, J=8.9 Hz, 2H), 7.02 (d, J=8.9 Hz, 2H), 4.62 (td, J=6.7, 13.4 Hz, 1H), 3.64 (s, 3H), 1.40 (d, J=6.7 Hz, 6H). [M+H]=411.0.

### Intermediate 9. 1-Isopropyl-5-methyl-6-(4-(4,4,5,5-tetramethyl-1,3,2-dioxaborolan-2-yl)phenoxy)-1,5-dihydro-4H-pyrazolo[3,4-d]pyrimidin-4-one

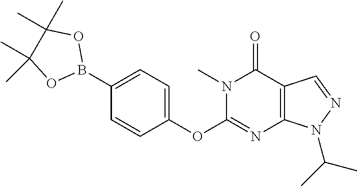 [SMILES: CC(C)n1ncc2c(=O)n(C)c(Oc3ccc(B4OC(C)(C)C(C)(C)O4)cc3)nc21]

The title compound was synthesized in a manner analogous to Intermediate 7, using the appropriate starting material substitutions. ¹H NMR (400 MHz, CDCl₃) δ 8.00 (s, 1H), 7.91 (d, J=8.4 Hz, 2H), 7.25 (d, J=8.4 Hz, 2H), 4.60 (spt, J=6.7 Hz, 1H), 3.65 (s, 3H), 1.44-1.32 (m, 18H). [M+H]=411.3.

### Intermediate 10. 6-(4-Iodophenoxy)-5-methyl-1-(tetrahydro-2H-pyran-4-yl)-1,5-dihydro-4H-pyrazolo[3,4-d]pyrimidin-4-one

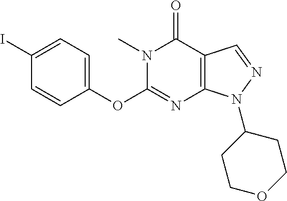 [SMILES: Cn1c(Oc2ccc(I)cc2)nc2c(cnn2C2CCOCC2)c1=O]

The title compound was synthesized in a manner analogous to Intermediate 7, using the appropriate starting material substitutions. ¹H NMR (400 MHz, CDCl₃) δ 8.03 (s, 1H), 7.80 (d, J=8.4 Hz, 2H), 7.03 (d, J=8.6 Hz, 2H), 4.52-4.36 (m, 1H), 4.06 (d, J=12.0 Hz, 2H), 3.66 (s, 3H), 3.50 (t, J=11.5 Hz, 2H), 2.25 (dq, J=4.3, 12.1 Hz, 2H), 1.81 (d, J=11.5 Hz, 2H). [M+H]=453.1.

### Intermediate 11. 5-Methyl-1-(tetrahydro-2H-pyran-4-yl)-6-(4-(4,4,5,5-tetramethyl-1,3,2-dioxaborolan-2-yl)phenoxy)-1,5-dihydro-4H-pyrazolo[3,4-d]pyrimidin-4-one

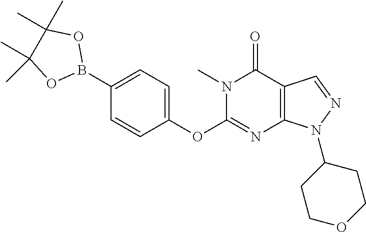 [SMILES: Cn1c(Oc2ccc(B3OC(C)(C)C(C)(C)O3)cc2)nc2c(cnn2C2CCOCC2)c1=O]

The title compound was synthesized in a manner analogous to Intermediate 7, using the appropriate starting material substitutions. ¹H NMR (400 MHz, CDCl₃) δ 8.01 (s, 1H), 7.91 (d, J=8.4 Hz, 2H), 7.24 (d, J=8.4 Hz, 2H), 4.42 (tt, J=4.2, 11.4 Hz, 1H), 4.03 (dd, J=2.6, 11.9 Hz, 2H), 3.65 (s, 3H), 3.47 (dt, J=1.8, 11.9 Hz, 2H), 2.22 (dq, J=4.5, 12.2 Hz, 2H), 1.78 (dd, J=2.2, 12.7 Hz, 2H), 1.43-1.35 (m, 12H). [M+H]=453.3.

### Intermediate 12. 6-(4-Bromo-2-fluorophenoxy)-1-isopropyl-5-methyl-1,5-dihydro-4H-pyrazolo[3,4-d]pyrimidin-4-one

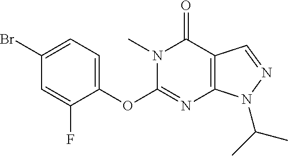 [SMILES: CC(C)n1ncc2c(=O)n(C)c(Oc3ccc(Br)cc3F)nc21]

The title compound was synthesized in a manner analogous to Intermediate 7, using the appropriate starting material substitutions. ¹H NMR (400 MHz, CDCl₃) δ 8.00 (s, 1H), 7.46-7.36 (m, 2H), 7.20 (t, J=8.3 Hz, 1H), 4.65-4.50 (m, 1H), 3.66 (s, 3H), 1.39 (d, J=6.7 Hz, 6H). [M+H]=381.3.

### Intermediate 13. 6-(4-Bromo-3-fluorophenoxy)-1-isopropyl-5-methyl-1,5-dihydro-4H-pyrazolo[3,4-d]pyrimidin-4-one

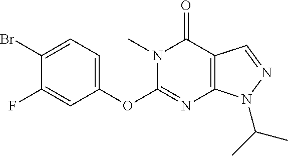 [SMILES: CC(C)n1ncc2c(=O)n(C)c(Oc3ccc(Br)c(F)c3)nc21]

The title compound was synthesized in a manner analogous to Intermediate 7, using the appropriate starting material substitutions. ¹H NMR (400 MHz, CDCl₃) δ 8.01 (s, 1H), 7.86 (t, J=8.4 Hz, 1H), 7.61 (dd, J=2.6, 9.7 Hz, 1H), 7.28 (ddd, J=1.1, 2.6, 8.8 Hz, 1H), 4.50 (quin, J=6.7 Hz, 1H), 3.50 (s, 3H), 1.30 (d, J=6.7 Hz, 6H). [M+H]=381.2.

### Intermediate 14. 6-(4-Bromo-2,3-difluorophenoxy)-1-isopropyl-5-methyl-1,5-dihydro-4H-pyrazolo[3,4-d]pyrimidin-4-one

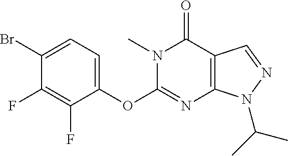 [SMILES: CC(C)n1ncc2c(=O)n(C)c(Oc3ccc(Br)c(F)c3F)nc21]

The title compound was synthesized in a manner analogous to Intermediate 7, using the appropriate starting material substitutions. ¹H NMR (400 MHz, CDCl₃) δ 8.01 (s, 1H), 7.44 (ddd, J=2.4, 6.7, 9.0 Hz, 1H), 7.05 (ddd, J=2.2, 6.9, 9.0 Hz, 1H), 4.59 (spt, J=6.7 Hz, 1H), 3.67 (s, 3H), 1.40 (d, J=6.7 Hz, 6H). [M+H]=399.2.

### Intermediate 15. 6-(4-Bromo-2,6-difluorophenoxy)-1-isopropyl-5-methyl-1,5-dihydro-4H-pyrazolo[3,4-d]pyrimidin-4-one

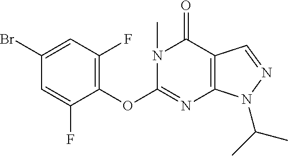 [SMILES: CC(C)n1ncc2c(=O)n(C)c(Oc3c(F)cc(Br)cc3F)nc21]

The title compound was synthesized in a manner analogous to Intermediate 7, using the appropriate starting material substitutions. ¹H NMR (400 MHz, CDCl₃) δ 8.01 (s, 1H), 7.28 (d, J=6.8 Hz, 2H), 4.58 (spt, J=6.7 Hz, 1H), 3.68 (s, 3H), 1.40 (d, J=6.7 Hz, 6H). [M+H]=399.3.

### Intermediate 16. 6-(4-Bromo-2,5-difluorophenoxy)-1-isopropyl-5-methyl-1,5-dihydro-4H-pyrazolo[3,4-d]pyrimidin-4-one

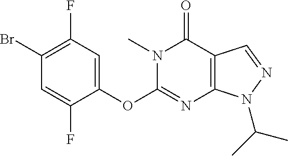 [SMILES: CC(C)n1ncc2c(=O)n(C)c(Oc3cc(F)c(Br)cc3F)nc21]

The title compound was synthesized in a manner analogous to Intermediate 7, using the appropriate starting material substitutions. ¹H NMR (400 MHz, CDCl₃) δ 8.01 (s, 1H), 7.50 (dd, J=6.2, 9.0 Hz, 1H), 7.19 (dd, J=6.5, 8.0 Hz, 1H), 4.60 (spt, J=6.7 Hz, 1H), 3.68-3.62 (m, 3H), 1.41 (d, J=6.7 Hz, 6H). [M+H]=399.2.

### Intermediate 17. 1-Isopropyl-5-methyl-6-(4-(piperidin-4-yl)phenoxy)-1,5-dihydro-4H-pyrazolo[3,4-d]pyrimidin-4-one

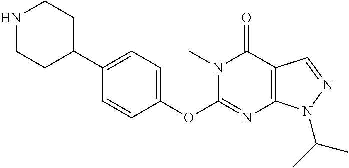 [SMILES: CC(C)n1ncc2c(=O)n(C)c(Oc3ccc(C4CCNCC4)cc3)nc21]

Step 1. tert-Butyl 4-(4-((1-isopropyl-5-methyl-4-oxo-4,5-dihydro-1H-pyrazolo[3,4-d]pyrimidin-6-yl)oxy)phenyl)piperidine-1-carboxylate. The title compound was synthesized in a manner analogous to Intermediate 7, using the appropriate starting material substitutions. ¹H NMR (400 MHz, DMSO-d₆) δ 7.99 (s, 1H), 7.34-7.39 (m, 2H), 7.27-7.33 (m, 2H), 4.43-4.55 (m, 1H), 4.05-4.16 (m, 2H), 3.51 (s, 3H), 2.70-2.86 (m, 3H), 1.94-1.99 (m, 1H), 1.76-1.84 (m, 2H), 1.48-1.59 (m, 2H), 1.43 (s, 9H), 1.29 (d, J=6.72 Hz, 6H). [M+H]=468.4.

Step 2. 1-Isopropyl-5-methyl-6-(4-(piperidin-4-yl)phenoxy)-1,5-dihydro-4H-pyrazolo[3,4-d]pyrimidin-4-one. Hydrogen chloride in dioxane (4 M, 12.6 mL, 50.3 mmol) was added to a stirred solution of tert-Butyl 4-(4-((1-isopropyl-5-methyl-4-oxo-4,5-dihydro-1H-pyrazolo[3,4-d]pyrimidin-6-yl)oxy)phenyl)piperidine-1-carboxylate (0.94 g, 2.0 mmol) in methanol (12.6 mL), and the resulting mixture was stirred at room temperature for 7 h. The reaction was then concentrated under vacuum to afford the hydrochloride salt of the title compound (0.80 g, 99%) as a colorless foam. ¹H NMR (400 MHz, CD₃OD) δ 7.95 (s, 1H), 7.47-7.36 (m, J=8.6 Hz, 2H), 7.30 (d, J=8.6 Hz, 2H), 4.58 (spt, J=6.7 Hz, 1H), 3.64 (s, 3H), 3.53 (d, J=12.5 Hz, 2H), 3.18 (t, J=12.1 Hz, 2H), 3.07-2.95 (m, 1H), 2.14 (d, J=14.2 Hz, 2H), 2.02-1.88 (m, 2H), 1.34 (d, J=6.7 Hz, 6H). [M+H]=368.2.

### Intermediate 18. 6-Bromo-3-isopropyl-7-methylimidazo[1,5-a]pyrazin-8(7H)-one

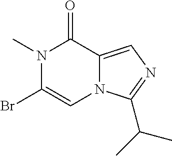

Step 1. 2-(2-Isopropyl-1H-imidazol-1-yl)acetonitrile. To a stirred solution of 2-isopropyl-1H-imidazole (80.0 g, 0.73 mol) in DMF (800 mL) at 0° C. was added K₂CO₃(301 g, 2.2 mol) followed by bromoacetonitrile (77 mL, 1.1 mol). Then the reaction mixture was allowed to stir at rt for 16 h. The mixture was diluted with EtOAc (800 mL) and water (1 L), the organic layer was separated and the aqueous layer was extracted with EtOAc (2×1 L). The combined organic extracts were dried over Na₂SO₄ and the solvent was removed by evaporation. Purification (FCC, SiO₂, 30-40% EtOAc/hexanes) afforded the title compound (58.3 g, 54%) as a dark brown oil. ¹H NMR (400 MHz, CDCl₃) δ 7.00 (d, 1H, J=1.6 Hz), 6.94 (d, 1H, J=1.6 Hz), 4.89 (s, 2H), 3.05-3.01 (m, 1H), 1.37-1.35 (d, 6H, J=6.8 Hz).

Step 2. 2-(5-Bromo-2-isopropyl-1H-imidazol-1-yl)acetonitrile. To a stirred solution of 2-(2-isopropyl-1H-imidazol-1-yl)acetonitrile (58.0 g, 0.39 mol) in acetonitrile (600 mL) at 0° C. was slowly added a solution of NBS (55.4 g, 0.31 mol) in acetonitrile (300 mL) over a period of 30 minutes. The mixture was allowed to stir at 0° C. for 30 min. Then the mixture was poured into ice-cold water (1 L) and extracted with EtOAc (3×1 L), then the combined organic extract was dried (Na₂SO₄) and the solvent was removed by evaporation. Purification (FCC, SiO₂, 0-20% EtOAc/hexanes) afforded the title compound (38 g, 43%) as a pale beige solid. ¹H NMR (300 MHz, CDCl₃) δ 7.00 (s, 1H), 4.89 (s, 2H), 3.07-2.97 (m, 1H), 1.38-1.36 (d, 6H, J=6.9 Hz). [M+H]=228.2.

Step 3. Ethyl 1-(cyanomethyl)-2-isopropyl-1H-imidazole-5-carboxylate. To a stirred solution of 2-(5-bromo-2-isopropyl-1H-imidazol-1-yl)acetonitrile (40 g, 0.18 mol) in EtOH (2.8 L) and DMSO (400 mL, degassed with argon) at rt was added 1,1′-bis(diphenylphosphino)ferrocene (19.5 g, 36 mmol) and triethylamine (56.9 mL, 0.40 mol) followed by Pd(OAc)₂ (7.89 g, 36 mmol). The reaction was then heated to 80° C. in an autoclave under a carbon monoxide atmosphere (80 psi) for 16 h. The resulting mixture was dried under reduced pressure. The residue was diluted with water (1 L), extracted with EtOAc (2×1 L) and the organic extracts were dried over Na₂SO₄ and the solvent was removed in vacuo. Purification (FCC, SiO₂, 0-20% EtOAc/hexanes) afforded the title compound (32.8 g, 84%) as a pale brown solid. ¹H NMR (300 MHz, CDCl₃) δ 7.73 (s, 1H), 5.34 (s, 2H), 4.36 (q, 2H), 3.10-3.03 (m, 1H), 1.41-1.36 (m, 9H). [M+H]=222.2.

Step 4. Ethyl 1-(2-amino-2-oxoethyl)-2-isopropyl-1H-imidazole-5-carboxylate. To a stirred solution of ethyl 1-(cyanomethyl)-2-isopropyl-1H-imidazole-5-carboxylate (26 g, 120 mmol) in EtOH (270 mL) and water (30 mL) at rt was added hydrido(dimethylphosphinous acid-kP)[hydrogen bis(dimethylphosphinito-kP)]platinum(II) (1.0 g, 2.3 mmol), then the reaction was heated to 110° C. for 2 h. The mixture was cooled to rt and diluted with DCM (2 L) and passed through a bed of SiO₂. The filtrate was removed by evaporation to afford the title compound (22 g, 78%) as a pale yellow solid. ¹H NMR (400 MHz, CDCl3) δ 7.76 (s, 1H), 6.02 (br s, 1H), 5.78 (br s, 1H), 4.95 (s, 2H), 4.32-4.27 (m, 2H), 3.13-3.06 (m, 1H), 1.37-1.33 (m, 9H). [M+H]=240.0.

Steps 5 and 6. 3-isopropyl-7-methylimidazo[1,5-a]pyrazine-6,8(5H,7H)-dione. To a stirred solution of ethyl 1-(2-amino-2-oxoethyl)-2-isopropyl-1H-imidazole-5-carboxylate (10.0 g, 42 mmol) in ethanol (100 mL) was added DIEA (21.8 mL, 125 mmol) at rt and then the mixture was heated to reflux for 16 h. The reaction mixture was cooled to rt and dried under reduced pressure to afford crude 3-isopropylimidazo[1,5-a]pyrazine-6,8(5H,7H)-dione (12.3 g). A small sample was purified (FCC, SiO₂, CH₂Cl₂) to afford 3-isopropylimidazo[1,5-a]pyrazine-6,8(5H,7H)-dione. ¹H NMR (400 MHz, DMSO-d₆) δ 11.46 (br s, 1H), 7.66 (s, 1H), 4.97 (s, 2H), 3.06 (spt, 1H, J=6.7 Hz), 1.22 (d, 6H, J=6.8 Hz).] To the crude 3-isopropylimidazo[1,5-a]pyrazine-6,8(5H,7H)-dione (42 mmol, assuming 100% yield from above) in acetonitrile (100 mL) was added K₂CO₃ (11.4 g, 84 mmol) followed by methyl iodide (2.58 mL, 42 mmol) at 0° C. and the mixture stirred at rt for 16 h. The resulting mixture was diluted with acetonitrile (150 mL) and filtered, then the filtrate was evaporated and diluted with THF (250 mL). The resulting solid was filtered and the solvent was removed by evaporation to afford the title compound (3.6 g, 41% for 2 steps) as a dark brown solid. ¹H NMR (300 MHz, CDCl₃) δ 7.89 (s, 1H), 4.88 (s, 2H), 3.33 (s, 3H), 2.99-2.94 (m, 1H), 1.38 (d, 6H, J=6.8 Hz). [M+H]=208.3.

Step 7. Diethyl (3-isopropyl-7-methyl-8-oxo-7,8-dihydroimidazo[1,5-a]pyrazin-6-yl) phosphate. To a stirred solution of diisopropylamine (9.6 mL, 72 mmol) in THF (160 mL) was added n-BuLi (1.6 M in hexanes, 45.2 mL, 72 mmol) at 0° C. and the mixture stirred for 30 min, then a solution of 3-isopropyl-7-methylimidazo[1,5-a]pyrazine-6,8(5H,7H)-dione (6 g, 29 mmol) in THF (50 mL) was added and the mixture stirred at 0° C. for one hour, upon which time a tan solid formed. Then a solution of diethyl chlorophosphate (10.47 mL, 72 mmol) in THF (50 mL) was added at 0° C. was added and the mixture was stirred for 1 h at 0° C. The mixture was diluted with a 1 M aqueous NH₄Cl solution and CH₂Cl₂ (200 mL), the organic layer was separated and the aqueous layer was extracted with CH₂Cl₂ (2×100 mL). The combined organic extracts were washed with a sat. NaHCO₃ solution and then the solvent was removed by evaporation to afford the title compound (11.8 g crude) as dark brown oil. This was used directly in the next reaction. A small sample was purified (FCC, SiO₂, 10-66% acetone/heptanes) and afforded the pure title compound. ¹H NMR (400 MHz, CDCl₃) δ 7.90 (s, 1H), 7.15 (d, 1H, J=2.2 Hz), 4.37-4.27 (m, 4H), 3.46 (s, 3H), 3.16 (spt, 1H, J=6.8 Hz), 1.47-1.39 (m, 12H). [M+H]=344.3.

Step 8. 6-Bromo-3-isopropyl-7-methylimidazo[1,5-a]pyrazin-8(7H)-one. To a stirred solution of diethyl (3-isopropyl-7-methyl-8-oxo-7,8-dihydroimidazo[1,5-a]pyrazin-6-yl) phosphate (11.8 g, 34 mmol) in acetonitrile (200 mL) and water (2 mL) was added TMSBr (20.4 mL, 155 mmol) at −20 to −30° C. and the reaction stirred at this temperature for 1 h. The mixture was diluted with CH₂Cl₂ (250 mL) and washed with a 1M aqueous solution of NaHCO₃ (2×250 mL), a sat. solution of Na₂SO₃, and brine. The organic extract was dried (MgSO₄) and purified (FCC, SiO₂, 0-2% MeOH/CH₂Cl₂) to afford the title compound (3.1 g, 40% for 2 steps) as a pale brown solid. ¹H NMR (300 MHz, CDCl₃) δ 7.87 (s, 1H), 7.16 (s, 1H), 3.62 (s, 3H), 3.16-3.11 (m, 1H), 1.41 (d, 6H, J=6.8 Hz). [M+H]=270.1/272.1.

### Intermediate 19. 6-Bromo-7-methyl-3-(tetrahydro-2H-pyran-4-yl)imidazo[1,5-a]pyrazin-8(7H)-one

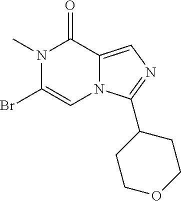 [SMILES: Cn1c(Br)cn2c(C3CCOCC3)ncc2c1=O]

Step 1. N-hydroxytetrahydro-2H-pyran-4-carboximidamide. To a stirred solution of tetrahydro-2H-pyran-4-carbonitrile (100 g, 0.9 mol) in ethanol (500 mL) and water (500 mL) at rt, was added hydroxylamine hydrochloride (144 g, 2.1 mol) followed by K₂CO₃ (136.7 g, 1.0 mol) and the mixture was heated to 70° C. for 16 h. The mixture was cooled to rt and the solvent was removed by evaporation under reduced pressure. The residue was dissolved in 10% MeOH in DCM and filtered. The solvent was removed by evaporation under reduced pressure to afford the title compound (91 g, 70%) as an off-white solid. ¹H NMR (300 MHz, DMSO-d₆) δ 1.56-1.63 (m, 4H), 2.23 (m, 1H), 3.24-3.32 (m, 2H), 3.81-3.97 (m, 2H), 5.40 (br s, 2H), 8.82 (s, 1H). [M+H]=145.3.

Step 2. Ethyl 2-(tetrahydro-2H-pyran-4-yl)-1H-imidazole-5-carboxylate. To a stirred solution of N-hydroxytetrahydro-2H-pyran-4-carboximidamide (95 g, 0.66 mol) in methanol (1 L) at rt, was added ethyl propiolate (67.3 mL, 0.66 mol) and then the reaction was heated to reflux for 3 h. The mixture was evaporated under reduced pressure and the residue was dissolved in diphenyl ether (1 L). The mixture was heated to 200° C. for 12 h, then the mixture was cooled to rt and diluted with hexanes (1 L). The solvent was decanted from the resulting solid and the solid suspended in hexanes (1 L), then decanted (3×). The resulting solid was filtered and triturated with hexanes to provide the title compound (48 g, 33%) as a dark brown solid. [M+H]=225.3.

Step 3. Ethyl 1-(cyanomethyl)-2-(tetrahydro-2H-pyran-4-yl)-1H-imidazole-5-carboxylate. To a stirred solution of ethyl 2-(tetrahydro-2H-pyran-4-yl)-1H-imidazole-5-carboxylate (95 g, 0.42 mol) in DMF (1 L) at rt was added K₂CO₃ (175.5 g, 1.3 mol) and bromoacetonitrile (35.9 mL, 0.50 mol) and the mixture stirred at rt for 20 h, then the solvent was removed by evaporation. Then, the residue was diluted with water (1 L) and extracted with EtOAc (3×1 L). The combined organic layers were dried (Na₂SO₄) and the solvent was removed by evaporation to afford the crude product. Purification (FCC, SiO₂, 30-40% EtOAc/hexanes) afforded the title compound (48 g, 43%) as a pale yellow oil. ¹H NMR (300 MHz, CDCl₃) δ 7.73 (s, 1H), 5.37 (s, 2H), 4.35 (m, 2H), 4.10 (m, 2H), 3.51-3.59 (m, 2H), 2.89 (m, 1H), 1.81-2.17 (m, 4H), 1.38 (m, 3H). [M+H]=264.1.

Step 4. Ethyl 1-(2-amino-2-oxoethyl)-2-(tetrahydro-2H-pyran-4-yl)-1H-imidazole-5-carboxylate. To a stirred suspension of ethyl 1-(cyanomethyl)-2-(tetrahydro-2H-pyran-4-yl)-1H-imidazole-5-carboxylate (20.0 g, 76 mmol) in EtOH (180 mL) and water (20 mL) was added hydrido(dimethylphosphinous acid-kP)[hydrogen bis(dimethylphosphinito-kP)]platinum(II) (649 mg, 1.5 mmol) at rt and then the reaction was heated to 110° C. for 2 h. The resulting mixture was cooled to rt and diluted with CH₂Cl₂ (2.5 L) and filtered through a pad of Celite®. The filtrate was evaporated under reduced pressure and the resulting residue was triturated with diethyl ether (2×250 mL) to afford the title compound (17 g, 82%) as an off-white solid. ¹H NMR (300 MHz, DMSO-d₆) δ 1.25 (t, 3H, J=7.2 Hz), 1.62-1.83 (m, 4H), 2.96 (m, 1H), 3.31 (dt, 2H, J=11.7, 1.8 Hz), 3.91 (dd, 2H, J=10.8, 1.8 Hz), 1.25 (q, 2H, J=7.2 Hz), 4.95 (s, 2H), 7.18 (br s, 1H), 7.56 (s, 1H), 7.60 (br s, 1H). [M+H]=282.4.

Step 5. 3-(Tetrahydro-2H-pyran-4-yl)imidazo[1,5-a]pyrazine-6,8(5H,7H)-dione. DBU (13.9 mL, 92 mmol) was added to a suspension of ethyl 1-(2-amino-2-oxoethyl)-2-(tetrahydro-2H-pyran-4-yl)-1H-imidazole-5-carboxylate (20.0 g, 71 mmol) in acetonitrile (250 mL) at rt and heated to 90° C. for 2 h, then the mixture was concentrated under reduced pressure and the resulting residue was purified (FCC, SiO₂, 8% MeOH/CH₂Cl₂) to provide the title compound (5.9 g, 35%) as an off-white solid. ¹H NMR (400 MHz, DMSO-d₆) δ 11.46 (br s, 1H), 7.68 (s, 1H), 5.00 (s, 2H), 3.90 (m, 2H), 3.45 (m, 2H), 3.03 (m, 1H), 1.77 (m, 4H). [M+H]=236.1.

Step 6. 7-Methyl-3-(tetrahydro-2H-pyran-4-yl)imidazo[1,5-a]pyrazine-6,8(5H,7H)-dione. To a stirred suspension of 3-(tetrahydro-2H-pyran-4-yl)imidazo[1,5-a]pyrazine-6,8(5H,7H)-dione (5.9 g, 25 mmol) in acetonitrile (150 mL) was added K₂CO₃ (6.92 g, 50 mmol) followed by Me₂SO₄ (2.61 mL, 28 mmol) at 0° C. and the mixture was allowed to stir at rt for 12 h. Then, the mixture was diluted with acetonitrile and filtered. The filtrate was evaporated under reduced pressure and the resulting residue was purified (FCC, SiO₂, 6-7% MeOH/CH₂Cl₂) to provide the title compound (4.1 g, 66% yield) as an off-white solid. ¹H NMR (400 MHz, DMSO-d₆) δ 1.75 (m, 4H), 3.03 (m, 1H), 3.13 (s, 3H), 3.45 (m, 2H), 3.92 (m, 2H), 5.12 (s, 2H), 7.72 (s, 1H). [M+H]=250.1.

Step 7. Diethyl 7-methyl-8-oxo-3-(tetrahydro-2H-pyran-4-yl)-7,8-dihydroimidazo[1,5-a]pyrazin-6-yl phosphate. To a stirred solution of diisopropylamine (5.4 mL, 40 mmol) in THF (20 mL) at −10° C. was added n-BuLi (2.5 M in hexanes, 16.4 mL, 40 mmol) and, after stirring for 30 min, a solution of 7-methyl-3-(tetrahydro-2H-pyran-4-yl)imidazo[1,5-a]pyrazine-6,8(5H,7H)-dione (4.1 g, 16 mmol) in THF (50 mL) was added. After stirring at −10° C. for 1 h, a solution of diethyl chlorophosphate (5.92 mL, 40 mmol) in THF (50 mL) was added at −10° C. After stirring at 0° C. for 1 h, the mixture was diluted with a sat. aqueous solution of NH₄Cl and extracted with EtOAc (3×200 mL). The combined organic extracts were dried (Na₂SO₄) and concentrated in vacuo to afford the title compound (4.8 g crude) as a dark brown oil, which was used directly in the next reaction.

Step 8. 6-Bromo-7-methyl-3-(tetrahydro-2H-pyran-4-yl)imidazo[1,5-a] pyrazin-8(7H)-one. To a stirred solution of diethyl 7-methyl-8-oxo-3-(tetrahydro-2H-pyran-4-yl)-7,8-dihydroimidazo[1,5-a]pyrazin-6-yl phosphate (4.8 g, 12 mmol) in acetonitrile (80 mL) and water (0.8 mL) at −20 to −40° C. was added TMSBr (7.38 mL, 54 mmol) and the reaction was stirred at the same temperature for 1 h. Then the mixture was diluted with CH₂Cl₂ (150 mL) and washed with a sat. aq. NaHCO₃ solution. The organic extracts were dried (Na₂SO₄) and the solvent was removed by evaporation to afford a residue, which, on trituration with diethyl ether, yielded the title compound (3.0 g, 59% yield over 2 steps) as a pale yellow solid. ¹H NMR (400 MHz, CDCl₃) δ 1.86 (m, 2H), 2.10 (m, 2H), 3.06 (m, 1H), 3.58 (m, 5H), 4.11 (m, 2H), 7.17 (s, 1H), 7.88 (s, 1H). [M+H]=311.8/313.8.

### Intermediate 20. 4-(Tetrahydro-2H-pyran-4-yl)phenol

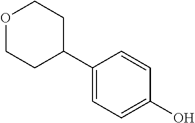 [SMILES: Oc1ccc(C2CCOCC2)cc1]

Step 1. 4-(4-(Benzyloxy)phenyl)-3,6-dihydro-2H-pyran. A mixture of 1-(benzyloxy)-4-iodobenzene (2.50 g, 8.1 mmol), 2-(3,6-dihydro-2H-pyran-4-yl)-4,4,5,5-tetramethyl-1,3,2-dioxaborolane (2.20 g, 10.5 mmol), [1,1′-bis(diphenylphosphino)-ferrocene]dichloropalladium(II) (295 mg, 0.4 mmol), K₂CO₃ (2.8 g, 20 mmol), dioxane (20 mL), ethanol (13 mL), and water (7 mL) was bubbled with N₂ for 1 min then heated to 110° C. for 16 h. After cooling to rt, the mixture was diluted with EtOAc, filtered through Celite®, and the filtrate was washed with water, brine, and dried (MgSO₄). The solvent was removed by evaporation and the resulting residue was purified (FCC, SiO₂, 5-20% EtOAc/heptanes) to provide 4-(4-(benzyloxy)phenyl)-3,6-dihydro-2H-pyran (1.68 g, 78%) as an off-white solid. ¹H NMR (400 MHz, CDCl₃) δ 7.49-7.44 (m, 2H), 7.41 (t, J=7.3 Hz, 2H), 7.38-7.32 (m, 3H), 6.97 (d, J=8.9 Hz, 2H), 6.10-6.01 (m, 1H), 5.10 (s, 2H), 4.39-4.31 (m, 2H), 3.95 (t, J=5.5 Hz, 2H), 2.52 (dd, J=2.6, 4.3 Hz, 2H). [M+H]=267.2.

Step 2. 4-(Tetrahydro-2H-pyran-4-yl)phenol. A mixture of 4-(4-(benzyloxy)phenyl)-3,6-dihydro-2H-pyran (1.65 g, 6.2 mmol), and 10% Pd/C (659 mg, 0.62 mmol) in of EtOAc (62 mL) was stirred in a stainless steel vessel under 180 psi H₂ at rt for 16 h. Then the mixture was filtered through Celite® and the solvent was removed by evaporation. The residue was purified (FCC, SiO₂, 10-40% EtOAc/heptanes) to provide the title compound (838 mg, 76%) as a white solid. ¹H NMR (400 MHz, CDCl₃) δ 7.12 (d, J=8.4 Hz, 2H), 6.81 (d, J=8.6 Hz, 2H), 5.32 (br s, 1H), 4.19-4.06 (m, 2H), 3.56 (dt, J=3.4, 11.1 Hz, 2H), 2.72 (tt, J=5.2, 10.6 Hz, 1H), 1.95-1.73 (m, 4H). [M+H]=179.1.

### Intermediate 21. 4-(1-Hydroxycyclobutyl)phenol

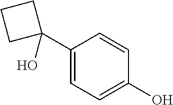 [SMILES: Oc1ccc(C2(O)CCC2)cc1]

Step 1. 1-(4-(Benzyloxy)phenyl)cyclobutan-1-ol. To a solution of 1-(benzyloxy)-4-iodobenzene (6.00 g, 19.4 mmol) in THF (39 mL) at 0° C. was added a 2 M solution of i-PrMgCl in THF (9.7 mL, 19.4 mmol) and the mixture stirred at rt for 1 h. From the resulting solution, 17 mL (5.9 mmol) was added to a premixed solution of cyclobutanone (0.40 mL, 5.4 mmol) in a 0.6 M solution of LaCl₃.2LiCl in THF (9 mL, 5.4 mmol) at 0° C. and the reaction was stirred at 0° C. for 2 h. Then, a solution of 1 M NH₄Cl was added, followed by EtOAc, and the organic layer was separated and washed with brine, dried (MgSO₄) and the solvent was removed under reduced pressure. The resulting residue was purified (FCC, SiO₂, 10-100% EtOAc/heptanes) to provide the title compound (639 mg, 47% yield) as an oil. ¹H NMR (400 MHz, CDCl₃) δ 7.53-7.32 (m, 7H), 7.00 (d, J=8.7 Hz, 2H), 5.10 (s, 2H), 2.68-2.52 (m, 2H), 2.46-2.30 (m, 2H), 2.14-1.96 (m, 1H), 1.95 (s, 1H), 1.67 (quind, J=8.3, 10.9 Hz, 1H). [M+H]=237.1.

Step 2. 4-(1-Hydroxycyclobutyl)phenol. The title compound was prepared from 1-(4-(benzyloxy)phenyl)cyclobutan-1-ol in a manner analogous to Intermediate 20, Step 2, using any appropriate starting material substitutions. ¹H NMR (400 MHz, CDCl₃) δ 7.12 (d, J=8.3 Hz, 2H), 6.79 (d, J=8.4 Hz, 2H), 4.59 (s, 1H), 3.50 (quin, J=8.7 Hz, 1H), 2.41-2.27 (m, 2H), 2.19-2.06 (m, 2H), 2.06-1.94 (m, 1H), 1.90-1.80 (m, 1H). [M+H]=149.0.

### Intermediate 22. 4-Cyclobutoxyphenol

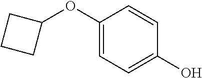 [SMILES: Oc1ccc(OC2CCC2)cc1]

Step 1. 1-(Benzyloxy)-4-cyclobutoxybenzene. A mixture of 4-(benzyloxy)phenol (600 mg, 3.0 mmol), bromocyclobutane (0.34 mL, 3.6 mmol), and Cs₂CO₃ (1.17 g, 3.6 mmol) in DMF was stirred at 120° C. for 16 h, then cooled and diluted with EtOAc and water. The organic layer was separated and washed with brine, dried (MgSO₄) and the solvent was removed under reduced pressure. The residue was purified (FCC, SiO₂, 10-100% EtOAc/heptanes) to provide the title compound (409 mg, 54%) as an oil. [M+H]=255.2.

Step 2. 4-Cyclobutoxyphenol. The title compound was prepared from 1-(benzyloxy)-4-cyclobutoxybenzene in a manner analogous to Intermediate 20, Step 2, using any appropriate starting material substitutions. ¹H NMR (400 MHz, CDCl₃) δ 6.79-6.74 (m, 2H), 6.74-6.70 (m, 2H), 4.57 (quin, J=7.1 Hz, 1H), 4.46 (s, 1H), 2.50-2.35 (m, 2H), 2.25-2.09 (m, 2H), 1.97-1.80 (m, 1H), 1.79-1.63 (m, 1H). [M+H]=164.1.

### Intermediate 23. 4-((Tetrahydro-2H-pyran-4-yl)methyl)phenol

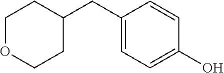 [SMILES: Oc1ccc(CC2CCOCC2)cc1]

Step 1. (4-(Benzyloxy)phenyl)(tetrahydro-2H-pyran-4-yl)methanone. To a solution of 1-(benzyloxy)-4-iodobenzene (3.35 g, 10.8 mmol) in 24 mL THF at 0° was added a 1.3 M solution of i-PrMgCl.LiCl in THF (8.3 mL, 10.8 mmol) and the mixture stirred at rt for 1 h. The resulting solution was added to a premixed solution of N-methoxy-N-methyltetrahydro-2H-pyran-4-carboxamide (1.63 g, 9.4 mmol) in 18 mL of 0.6 M LaCl₃.2LiCl (10.8 mmol) at −78° C. and stirred at 23° C. for 3 h, then 1 M NH₄Cl and EtOAc were added, the organic layer separated and washed with brine, dried (MgSO₄) and evaporated. The residue was purified (FCC, SiO₂, 10-40% EtOAc/heptanes) to provide the title compound (885 mg, 32% yield) as an oil. ¹H NMR (400 MHz, CDCl₃) δ 7.96 (d, J=8.8 Hz, 2H), 7.51-7.35 (m, 5H), 7.05 (d, J=8.8 Hz, 2H), 5.16 (s, 2H), 4.15-4.04 (m, 2H), 3.58 (dt, J=2.2, 11.6 Hz, 2H), 3.48 (tt, J=3.9, 11.1 Hz, 1H), 2.04-1.85 (m, 2H), 1.81-1.72 (m, 2H). [M+H]=297.2.

Step 2. 4-(2-(Tetrahydro-2H-pyran-4-yl)-1,3-dithiolan-2-yl)phenol. To a mixture of (4-(benzyloxy)phenyl)(tetrahydro-2H-pyran-4-yl)methanone (875 mg, 3.0 mmol) and ethane-1,2-dithiol (2.5 mL, 29.5 mmol) at 0° C., was added BF₃.Et₂O (2.9 mL, 23.6 mmol) and the mixture was stirred at rt for 30 min, then diluted with CH₂Cl₂, washed with a 1 M solution of NaHCO₃, dried (MgSO₄) and the solvent removed by evaporation. The residue was purified (FCC, SiO₂, 0-15% MeOH/CH₂Cl₂) to provide the title compound (637 mg, 76% yield) as an oil. ¹H NMR (400 MHz, CDCl₃) δ 7.59 (d, J=8.6 Hz, 2H), 6.76 (d, J=8.6 Hz, 2H), 5.18 (s, 1H), 3.99 (dd, J=3.9, 11.0 Hz, 2H), 3.49-3.26 (m, 4H), 3.22-3.04 (m, 2H), 2.39-2.21 (m, 1H), 1.76 (d, J=12.7 Hz, 2H), 1.53 (dq, J=4.4, 12.5 Hz, 2H). [M+H]=283.1.

Step 3. 4-((Tetrahydro-2H-pyran-4-yl)methyl)phenol. A mixture of 4-(2-(tetrahydro-2H-pyran-4-yl)-1,3-dithiolan-2-yl)phenol (100 mg, 0.35 mmol) and Raney nickel (0.61 g, 7.1 mmol) was stirred at 78° C. for 2 h. The mixture was filtered and the solvent was removed by evaporation. The residue was purified (FCC, SiO₂, 1-5% MeOH/CH₂Cl₂) to provide of the title compound (31 mg) that was 85% pure, presumably contaminated by an alkene byproduct. The residue was dissolved in CH₂Cl₂ (2 mL) then mCPBA (37 mg, 0.21 mmol) and KHCO₃ (18 mg, 0.18 mmol) were added. The mixture was stirred at rt for 2 h, diluted with CH₂Cl₂, washed with a 1 M aqueous solution of Na₂SO₃, dried (MgSO₄) and the solvent was removed by evaporation. The residue was purified (FCC, SiO₂, 5-30% acetone/heptanes) to provide the title compound (18 mg, 26% yield) as an oil. ¹H NMR (400 MHz, CDCl₃) δ 7.02 (d, J=8.4 Hz, 2H), 6.82-6.69 (m, 2H), 5.61 (s, 1H), 3.99 (dd, J=3.9, 11.0 Hz, 2H), 3.38 (dt, J=2.0, 11.8 Hz, 2H), 2.50 (d, J=7.2 Hz, 2H), 1.72 (dtt, J=3.6, 7.4, 11.2 Hz, 1H), 1.58 (dd, J=1.7, 13.1 Hz, 2H), 1.35 (ddd, J=5.3, 12.0, 21.2 Hz, 2H).

### Intermediate 24 and 25. (R)-4-(Oxetan-2-ylmethoxy)phenol and (S)-4-(oxetan-2-ylmethoxy)phenol

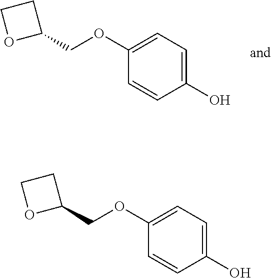 [SMILES: Oc1ccc(OC[C@H]2CCO2)cc1 ; Oc1ccc(OC[C@@H]2CCO2)cc1]

Racemic 4-(oxetan-2-ylmethoxy)phenol was prepared in a manner analogous to Intermediate 22, using any appropriate starting material substitutions. ¹H NMR (400 MHz, CDCl₃) δ 6.88-6.82 (m, 2H), 6.80-6.73 (m, 2H), 5.20-5.06 (m, 1H), 4.96 (br s, 1H), 4.80-4.63 (m, 2H), 4.09 (d, J=4.3 Hz, 2H), 2.85-2.64 (m, 2H). [M+H]=181.0.

Racemic 4-(oxetan-2-ylmethoxy)phenol (300 mg, 1.7 mmol) was dissolved in MeOH (10 mL). Separately, twenty 0.5 mL samples of this solution were injected onto a 30×150 mm Chiralpak OJ (5 μm) column and eluted at 100 mL/min at 50° C. with an eluent of 10% MeOH in CO₂ (1500 psi) for 8 min. This provided as the first eluent (R)-4-(oxetan-2-ylmethoxy)phenol (100 mg, 33%) as a white solid. Analytical SFC was performed with a 2.1×100 mm Chiralpak OJ-3 (3 μm) column and eluted at 1.5 mL/min at 50° C. with an eluent of 10% MeOH (with 0.1% NH₄OH v/v) in CO₂ (1500 psi) for 5 min. Preparative SFC Rₜ=5.05 min. Analytical Rₜ=1.19 min (100% ee). ¹H NMR (CDCl₃) and [M+H] values matched those for the previously described racemic 4-(oxetan-2-ylmethoxy)phenol. A 50 mg sample of (R)-4-(oxetan-2-ylmethoxy)phenol was dissolved in THF (0.5 mL) in a 1.5-mL vial. This vial was placed inside a 20-mL vial containing pentane (5 mL). The larger vial was sealed. After sitting at rt for 16 h, rod shaped crystals resulted. These crystals were subjected to X-ray structural absolute configuration determination at the UCSD Crystallography lab and the results confirmed that this enantiomer was of the (R) configuration.

Continued elution provided the second enantiomer (S)-4-(oxetan-2-ylmethoxy)phenol (85 mg, 28%) as a white solid. Preparative SFC Rₜ=5.88 min. Analytical Rₜ=1.33 min (93% ee). ¹H NMR (CDCl₃) and [M+H] values matched those for the previously described racemic 4-(oxetan-2-ylmethoxy)phenol.

### Intermediate 26 and 27. (R)-4-((Tetrahydrofuran-3-yl)oxy)phenol and (S)-4-((tetrahydrofuran-3-yl)oxy)phenol

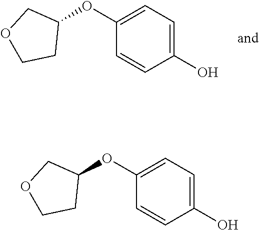 [SMILES: Oc1ccc(O[C@@H]2CCOC2)cc1 ; Oc1ccc(O[C@H]2CCOC2)cc1]

Racemic 4-((tetrahydrofuran-3-yl)oxy)phenol was prepared in a manner analogous to Intermediate 22, using any appropriate starting material substitutions. ¹H NMR (400 MHz, CDCl₃) δ 6.78 (s, 4H), 4.92-4.80 (m, 1H), 4.69 (br s, 1H), 4.06-3.96 (m, 3H), 3.95-3.88 (m, 1H), 2.22-2.13 (m, 2H). [M+H]=181.1.

Then, racemic 4-((tetrahydrofuran-3-yl)oxy)phenol (640 mg, 3.5 mmol) was dissolved in MeOH (13 mL). Separately, twenty-six 0.5 mL samples of this were injected onto a 30×150 mm Chiralpak IF (5 μm) column and eluted at 100 mL/min at 50° C. with an eluent of 10% MeOH in CO₂ (1500 psi) for 7 min. This provided, as the first eluent, (R)-4-((tetrahydrofuran-3-yl)oxy)phenol (203 mg, 32% yield) as a white solid. Analytical SFC was performed with a 2.1×100 mm Chiralpak IF-3 (3 μm) column and eluted at 1.5 mL/min at 50° C. with an eluent of 10% MeOH (with 0.1% NH₄OH v/v) in CO₂ (1500 psi) for 2.5 min. Preparative SFC Rₜ=4.18 min. Analytical Rₜ=1.00 min (100% ee). ¹H NMR (CDCl₃) and [M+H] values matched those previously described for racemic 4-((tetrahydrofuran-3-yl)oxy)phenol. A 40 mg sample of (R)-4-((tetrahydrofuran-3-yl)oxy)phenol was dissolved in THF (0.5 mL) in a 1.5-mL vial. This vial was placed inside a 20-mL vial containing pentane (5 mL). The larger vial was sealed. After sitting at rt for 16 h, rod shaped crystals resulted. These crystals were subjected to X-ray structural absolute configuration determination at the UCSD Crystallography lab and the results confirmed that this enantiomer was of the (R) configuration.

Continued elution provided the second enantiomer (S)-4-((tetrahydrofuran-3-yl)oxy)phenol (225 mg, 35% yield) as a white solid. Preparative SFC Rₜ=4.90 min. Analytical Rₜ=1.14 min (90% ee). ¹H NMR (CDCl₃) and [M+H] values matched the previously described for racemic 4-((tetrahydrofuran-3-yl)oxy)phenol.

### Intermediate 28 and 29. (R)-4-((Tetrahydrofuran-2-yl)methoxy)phenol and (S)-4-((tetrahydrofuran-2-yl)methoxy)phenol

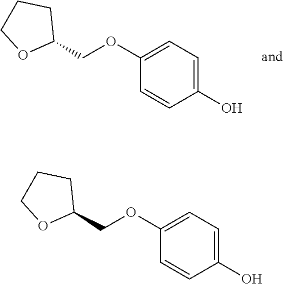 [SMILES: Oc1ccc(OC[C@H]2CCCO2)cc1 ; Oc1ccc(OC[C@@H]2CCCO2)cc1]

Racemic 4-((tetrahydrofuran-2-yl)methoxy)phenol was prepared in a manner analogous to Intermediate 22, using any appropriate starting material substitutions. ¹H NMR (400 MHz, CDCl₃) δ 6.89-6.79 (m, 2H), 6.78-6.71 (m, 2H), 4.86 (br s, 1H), 4.36-4.19 (m, 1H), 4.04-3.78 (m, 4H), 2.32-1.79 (m, 4H). [M+H]=195.1.

Racemic 4-((tetrahydrofuran-2-yl)methoxy)phenol (735 mg, 3.9 mmol) was dissolved in MeOH (16 mL). Separately, thirty-two 0.5 mL samples of this were injected onto a 30×150 mm Chiralpak IB (5 μm) column and eluted at 100 mL/min at 50° C. with an eluent of 10% MeOH in CO₂ (1500 psi) for 8 min. This provided as the first eluent (R)-4-((tetrahydrofuran-2-yl)methoxy)phenol (291 mg, 40% yield) as a white solid. Analytical SFC was performed with a 2.1×100 mm Chiralpak IB-3 (3 μm) column and eluted at 1.5 mL/min at 50° C. with an eluent of 10% MeOH (with 0.1% NH₄OH v/v) in CO₂ (1500 psi) for 2.5 min. Preparative SFC Rₜ=4.68 min. Analytical Rₜ=0.97 min (100% ee). ¹H NMR (CDCl₃) and [M+H] values matched the previously described for racemic 4-((tetrahydrofuran-2-yl)methoxy)phenol. A 40 mg sample of (R)-4-((tetrahydrofuran-2-yl)methoxy)phenol was dissolved in THF (0.5 mL) in a 1.5-mL vial. This vial was placed inside a 20-mL vial containing pentane (5 mL). The larger vial was sealed. After sitting at rt for 16 h, rod shaped crystals resulted. These crystals were subjected to X-ray structural absolute configuration determination at the UCSD Crystallography lab and the results confirmed that the enantiomer was of the (R) configuration.

Continued elution provided the second enantiomer (S)-4-((tetrahydrofuran-2-yl)methoxy)phenol (290 mg, 40% yield) as a white solid. Preparative SFC Rₜ=6.13 min. Analytical Rₜ=1.10 min (99% ee). ¹H NMR (CDCl₃) and [M+H] values matched the previously described for racemic 4-((tetrahydrofuran-2-yl)methoxy)phenol.

### Intermediate 30 and 31. (R)-4-(Tetrahydrofuran-3-yl)phenol and (S)-4-(tetrahydrofuran-3-yl)phenol

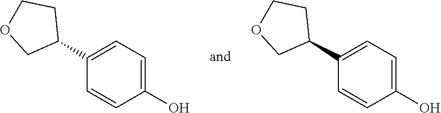 [SMILES: Oc1ccc([C@H]2CCOC2)cc1 ; Oc1ccc([C@@H]2CCOC2)cc1]

Racemic 4-(tetrahydrofuran-3-yl)phenol was prepared in a manner analogous to Intermediate 21, using any appropriate starting material substitutions. ¹H NMR (400 MHz, CDCl₃) δ 7.14 (d, J=8.4 Hz, 2H), 6.81 (d, J=8.6 Hz, 2H), 5.24 (s, 1H), 4.15 (t, J=7.9 Hz, 1H), 4.09 (dt, J=4.5, 8.3 Hz, 1H), 3.95 (q, J=8.0 Hz, 1H), 3.70 (t, J=8.1 Hz, 1H), 3.38 (quin, J=7.9 Hz, 1H), 2.42-2.31 (m, 1H), 2.00 (qd, J=8.2, 12.3 Hz, 1H). [M+H]=165.1.

Racemic 4-(tetrahydrofuran-3-yl)phenol (840 mg, 5.12 mmol) was dissolved in MeOH (16 mL). Separately, thirty-two 0.5 mL samples of this were injected onto a 30×150 mm Chiralpak IF (5 μm) column and eluted at 100 mL/min at 50° C. with an eluent of 10% MeOH in CO₂ (1500 psi) for 14 min. This provided as the first eluent (R)-4-(tetrahydrofuran-3-yl)phenol (230 mg, 27%) as a white solid. Analytical SFC was performed with a 2.1×100 mm Chiralpak IF-3 (3 μm) column and eluted at 1.5 mL/min at 50° C. with an eluent of 10% MeOH (with 0.1% NH₄OH v/v) in CO₂ (1500 psi) for 2.5 min. Preparative SFC Rₜ=10.28 min. Analytical Rₜ=0.90 min (99% ee). ¹H NMR (CDCl₃) and [M+H] values matched the previously described for racemic 4-(tetrahydrofuran-3-yl)phenol. A 40 mg sample of (R)-4-(tetrahydrofuran-3-yl)phenol was dissolved in THF (0.5 mL) in a 1.5-mL vial. This vial was placed inside a 20-mL vial containing pentane (5 mL). The larger vial was sealed. After sitting at rt for 16 h, rod shaped crystals resulted. These crystals were subjected to X-ray structural absolute configuration determination at the UCSD Crystallography lab and the results confirmed that this enantiomer was of the (R) configuration.

Continued elution provided the second enantiomer (S)-4-(tetrahydrofuran-3-yl)phenol (238 mg, 28%) as a white solid. Preparative SFC Rₜ=11.48 min. Analytical Rₜ=0.97 min (94% ee). ¹H NMR (CDCl₃) and [M+H] values matched the previously described for racemic 4-(tetrahydrofuran-3-yl)phenol.

### Intermediate 32. 1-(3-(4-Hydroxyphenyl)piperidin-1-yl)ethan-1-one

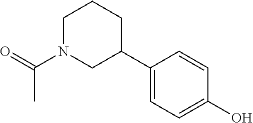 [SMILES: CC(=O)N1CCCC(c2ccc(O)cc2)C1]

Step 1. 3-(4-Methoxyphenyl)pyridine. A mixture of 3-bromopyridine (0.38 mL, 4.0 mmol), (4-methoxyphenyl)boronic acid (500 mg, 3.3 mmol), Pd(dppf)Cl₂ (69 mg, 0.094 mmol), K₂CO₃ (720 mg, 5.2 mmol), dioxane (10 mL), and water (5 mL), was bubbled with N₂ for 1 min and then stirred at 110° C. for 12 h. After cooling to rt, the mixture was diluted with EtOAc, filtered through Celite®, the filtrate washed with water, brine, dried (MgSO₄), the solvent was removed by evaporation and the residue was purified (FCC, SiO₂, 0-100% EtOAc/heptanes) to provide the title compound (350 mg, 57%) as an off-white solid. ¹H NMR (300 MHz, DMSO-d₆) δ 8.8 (d, 1H, J=2.1 Hz), 8.5 (q, 1H, J=1.2, 3.3 Hz), 7.85-7.81 (m, 1H), 7.55-7.50 (m, 2H), 7.35-7.26 (m, 1H), 7.04-6.99 (m, 2H), 3.86 (s, 1H). [M+H]=186.1.

Step 2. 3-(4-Methoxyphenyl)piperidine. To a solution of 3-(4-methoxyphenyl)pyridine (35 g, 0.19 mol) in MeOH (350 mL) and conc. HCl (70 mL) was added PtO₂ (3.5 g, 15.4 mmol) and the mixture stirred under 50 psi H₂ at rt for 12 h. The mixture was filtered through Celite® and the filtrate was concentrated by evaporation and made basic with a 1 M NaOH solution. The mixture was extracted with 10% MeOH in CH₂Cl₂. The combined organic layers were dried (Na₂SO₄) and evaporated under reduced pressure to afford the title compound (30 g, 83%) as a brown solid. ¹H NMR (300 MHz, DMSO-d₆) δ 8.86 (br s, 2H), 7.19 (d, 2H, J=8.7 Hz), 6.90 (d, 2H, J=8.7 Hz), 3.32-3.18 (m, 5H), 2.98-2.82 (m, 3H), 1.83-1.63 (m, 5H). [M+H]=192.2.

Step 3. 1-(3-(4-Methoxyphenyl)piperidin-1-yl)ethan-1-one. To a solution of 3-(4-methoxyphenyl)piperidine (300 mg, 1.6 mmol) in CH₂Cl₂ (6 mL) was added triethylamine (0.66 mL, 4.6 mmol) and the solution was cooled to 0° C., then acetyl chloride (0.12 mL, 1.8 mmol) was added dropwise and the reaction was stirred at rt for 4 h. Water was added and the mixture was extracted with CH₂Cl₂ (3×50 mL). The combined organic layers were washed with a saturated solution of NaHCO₃ and brine, dried (Na₂SO₄), and evaporated to provide the title compound (340 mg, 91%) as a brown liquid. ¹H NMR (400 MHz, CDCl₃) δ 7.14 (d, 1H), 6.86 (q, 1H, J=8.8 Hz, 3.6 Hz), 4.7 (t, 1H, J=12.4, 16.4 Hz), 3.85-3.79 (m, 2H), 3.07-3.00 (m, 1H), 2.66-2.50 (m, 1H), 2.12-2.02 (m, 3H), 1.86-1.79 (m, 1H), 1.73-1.65 (m, 1H), 1.39 (s, 1H), 1.22-1.11 (m, 1H). [M+H]=234.3.

Step 4. 1-(3-(4-Hydroxyphenyl)piperidin-1-yl)ethan-1-one. To a solution of 3-(4-methoxyphenyl) piperidine (2.0 g, 9 mmol) in CH₂Cl₂ (20 mL) at −40° C. was added BBr₃ (1.71 ml, 18 mmol) and reaction mixture was allowed to stir at room temperature for 4 h. The resulting mixture was diluted slowly with water and extracted with DCM (3×50 mL). The combined organic layers were washed with a saturated solution of NaHCO₃ and brine, dried (Na₂SO₄), and evaporated. The residue was purified (FCC, SiO₂, 2% MeOH/CH₂Cl₂) to provide the title compound (1.60 g, 81% yield) as an off-white solid. ¹H NMR (300 MHz, DMSO-d₆) δ 9.20 (d, 1H, J=4.2 Hz) 7.06 (q, 2H, J=8.4, 7.8 Hz) 6.69 (q, 2H, J=2.1, 6.3 Hz), 4.39 (t, 1H, J=18, 11.7 Hz) 3.76 (q, 1H, J=11.7, 15.3 Hz), 3.31-2.95 (m, 1H), 2.56-2.40 (m, 2H), 2.00 (d, 1H, J=3.6 Hz), 1.85 (d, 1H, J=11.7 Hz), 1.75-1.37 (m, 3H). [M+H]=220.2.

### Intermediate 33 and 34. (R)-1-(3-(4-Hydroxyphenyl)piperidin-1-yl)ethan-1-one and (S)-1-(3-(4-hydroxyphenyl)piperidin-1-yl)ethan-1-one

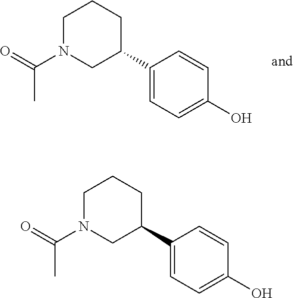 [SMILES: CC(=O)N1CCC[C@H](c2ccc(O)cc2)C1 ; CC(=O)N1CCC[C@@H](c2ccc(O)cc2)C1]

Racemic 1-(3-(4-hydroxyphenyl)piperidin-1-yl)ethan-1-one (Intermediate 32, 1.42 g, 6.5 mmol) was dissolved in MeOH (28 mL). Separately, fifty-six 0.5 mL samples of this were injected onto a 30×150 mm Chiralpak IC (5-μm) column and eluted at 100 mL/min at 50° C. with an eluent of 20% MeOH in CO₂ (1500 psi) for 8 min. This provided, as the first eluent, (R)-1-(3-(4-hydroxyphenyl)piperidin-1-yl)ethan-1-one (437 mg, 31%) as a white solid. Analytical SFC was performed with a 2.1×100 mm Chiralpak IC-3 (3 μm) column and eluted at 1.5 mL/min at 50° C. with an eluent of 15% MeOH (with 0.1% NH₄OH v/v) in CO₂ (1500 psi) for 5 min. Preparative SFC Rₜ=4.83 min. Analytical Rₜ=2.03 min (100% ee). A 50 mg sample of (R)-1-(3-(4-hydroxyphenyl)piperidin-1-yl)ethan-1-one was dissolved in THF (0.5 mL) in a 1.5 mL vial. This vial was placed inside a 20-mL vial containing pentane (5 mL). The larger vial was sealed. After sitting at rt for 16 h, spiny shaped crystals resulted. These crystals were subjected to X-ray structural absolute configuration determination at the UCSD Crystallography lab and the results confirmed that this enantiomer was of the (R) configuration.

Continued elution provided the second enantiomer (S)-1-(3-(4-hydroxyphenyl)piperidin-1-yl)ethan-1-one (470 mg, 33%) as a white solid. Preparative SFC Rₜ=5.88 min. Analytical Rₜ=2.55 min (96.2% ee).

### Intermediate 35. 4-(1-Methyl-3-(trifluoromethyl)-1H-pyrazol-4-yl)phenol

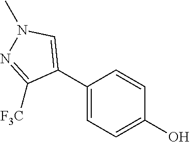 [SMILES: Cn1cc(-c2ccc(O)cc2)c(C(F)(F)F)n1]

Step 1. tert-Butyldimethyl(4-(4,4,5,5-tetramethyl-1,3,2-dioxaborolan-2-yl)phenoxy)silane. A mixture of 4-(4,4,5,5-tetramethyl-1,3,2-dioxaborolan-2-yl)phenol (10 g, 45 mmol), imidazole (3.25 g, 48 mmol), and TBSCl (7.2 g, 48 mmol) in DMF (45 mL) was stirred at rt for 16 h, then diluted with ether, washed with water (2×100 mL), brine, dried (MgSO₄) and evaporated to provide the title compound (17.7 g, 117%) as an amorphous solid. ¹H NMR (400 MHz, CDCl₃) δ 7.73 (d, J=8.4 Hz, 2H), 6.84 (d, J=8.4 Hz, 2H), 5.32 (d, J=2.1 Hz, 1H), 1.36 (s, 12H), 1.00 (s, 9H), 0.22 (s, 6H). [M+H]=335.4.

Step 2. 4-(1-Methyl-3-(trifluoromethyl)-1H-pyrazol-4-yl)phenol. A mixture of tert-butyldimethyl(4-(4,4,5,5-tetramethyl-1,3,2-dioxaborolan-2-yl)phenoxy)silane (1.0 g, 3.0 mmol), 4-bromo-1-methyl-3-(trifluoromethyl)-1H-pyrazole (822 mg, 3.6 mmol), and Pd(dppf)Cl₂ (110 mg, 0.15 mmol), in a mixture of acetonitrile (12 mL) and a 1 M aqueous solution of NaHCO₃ (5.4 mL) was bubbled with N₂ for 1 min and then heated in a microwave reactor to 130° C. for 15 min. After cooling to rt, a 1 M solution of TBAF in THF (3 mL, 3 mmol) was added and the mixture was stirred at rt for 5 h, then diluted with CH₂Cl₂ and washed with water, brine, dried (MgSO₄) and evaporated. The residue was purified (FCC, SiO₂, 10-60% EtOAc/heptanes) to provide the title compound (445 mg, 61% yield) as an off-white solid. ¹H NMR (300 MHz, CDCl₃) δ 7.45 (d, J=1.00 Hz, 1H), 7.28 (d, J=1.00 Hz, 2H), 6.88 (d, J=8.56 Hz, 2H), 5.16 (br s, 1H), 3.99 (s, 3H). [M+H]=243.2.

### Intermediate 36. 4-Bromo-3-methoxy-1-methyl-1H-pyrazole

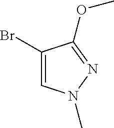

A mixture of 3-methoxy-1-methyl-1H-pyrazol-4-amine hydrochloride salt (500 mg, 3.1 mmol), t-butyl nitrite (0.54 mL, 4.6 mmol) and CuBr₂ (819 mg, 3.7 mmol) in acetonitrile (12 mL) was stirred at 80° C. for 16 h, then cooled to rt, diluted with ether and washed with 4 M HCl, washed with brine, dried (MgSO₄) and evaporated. The residue was purified (FCC, SiO₂, 25-100% EtOAc/heptanes) to provide the title compound (240 mg, 41% yield) as a white solid. ¹H NMR (300 MHz, CDCl₃) δ 7.20 (s, 1H), 3.96 (s, 3H), 3.75 (s, 3H).

### Intermediate 37. 4-(2,4-Dimethyloxazol-5-yl)phenol

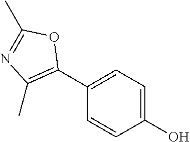

A mixture of 4-(4,4,5,5-tetramethyl-1,3,2-dioxaborolan-2-yl)phenol (2.92 g, 21 mmol), 5-bromo-2,4-dimethyloxazole (3.11 g, 17.7 mmol), Pd(dppf)Cl₂ (646 mg, 0.88 mmol), dioxane (135 mL) and a 1 M aqueous solution of NaHCO₃ (47 mL) was bubbled with N₂ for 1 min and then heated to 100° C. for 1 h. After cooling to rt, the mixture was filtered and diluted with EtOAc and washed with brine, dried (MgSO₄) and evaporated. The residue was purified (FCC, SiO₂, 0-100% EtOAc/heptanes) to provide the title compound (2.95 g, 88% yield) as an off-white solid. ¹H NMR (400 MHz, DMSO-d₆) δ 9.70 (s, 1H), 7.37 (d, J=8.7 Hz, 2H), 6.85 (d, J=8.7 Hz, 2H), 2.37 (s, 3H), 2.22 (s, 3H). [M+H]=190.1.

### Intermediate 38. 4-(5-Cyclopropyl-3-methyl-1H-pyrazol-1-yl)phenol

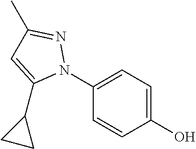 [SMILES: Cc1cc(C2CC2)n(-c2ccc(O)cc2)n1]

Step 1. 5-Iodo-1-(4-methoxyphenyl)-3-methyl-1H-pyrazole. To a solution of 1-(4-methoxyphenyl)-3-methyl-1H-pyrazole (4.00 g, 21.2 mmol) in THF (85 mL) at 0° C. was added a 2 M solution of n-BuLi in hexanes (11.7 mL, 23 mmol) and the mixture was stirred at 0° C. for 30 min. To this mixture was added a solution of 12 (5.93 g, 23.4 mmol) in THF (35 mL) and the resulting mixture stirred at 0° C. for 1 h. Then, the mixture was diluted with EtOAc, washed with a 1 M aqueous solution of Na₂SO₃, dried (MgSO₄) and evaporated. The residue was purified (FCC, SiO₂, 5-20% EtOAc/heptanes) to provide the title compound (729 mg, 11%) as a white solid. ¹H NMR (400 MHz, CDCl₃) δ 7.37-7.44 (m, 2H), 6.96-7.03 (m, 2H), 6.40 (s, 1H), 3.88 (s, 3H), 2.35 (s, 3H). [M+H]=314.9.

Step 2. 5-Cyclopropyl-1-(4-methoxyphenyl)-3-methyl-1H-pyrazole. A mixture of 5-iodo-1-(4-methoxyphenyl)-3-methyl-1H-pyrazole (630 mg, 2.0 mmol), cyclopropylboronic acid (345 mg, 4.0 mmol), Pd(Oac)₂ (45 mg, 0.20 mmol), tricyclohexylphosphine (112 mg, 0.40 mmol), K₃PO₄ (1.5 g, 7.02 mmol), in toluene (9 mL) and water (0.5 mL) was bubbled with N₂ for 1 min, then heated to 80° C. for 16 h, cooled, diluted with EtOAc, washed with brine, dried (MgSO₄) and evaporated. The residue was purified (FCC, SiO₂, 5-50% ether/heptanes) to provide the title compound (280 mg, 61%) as a white solid. ¹H NMR (400 MHz, CDCl₃) δ 7.47-7.54 (m, 2H), 6.95-7.02 (m, 2H), 5.74 (s, 1H), 3.87 (s, 3H), 2.29 (s, 3H), 1.74 (tt, J=5.07, 8.38 Hz, 1H), 0.91-0.99 (m, 2H), 0.70-0.76 (m, 2H). [M+H]=229.0.

Step 3. 4-(5-Cyclopropyl-3-methyl-1H-pyrazol-1-yl)phenol. To a mixture of 5-cyclopropyl-1-(4-methoxyphenyl)-3-methyl-1H-pyrazole (278 mg, 1.22 mmol) in CH₂Cl₂ (6 mL) at −40° C. was added a 1 M solution of BBr₃ in CH₂Cl₂ (2.4 mL, 2.4 mmol) and the mixture stirred at rt for 2 h, then diluted with CH₂Cl₂ and water, washed with a 1 M aq. solution of NaHCO₃, dried (MgSO₄) and evaporated. The residue was purified (FCC, SiO₂, 10-40% EtOAc/heptanes) to provide the title compound (159 mg, 61%) as a white solid. ¹H NMR (400 MHz, CDCl₃) δ 7.26-7.29 (m, 2H), 6.71-6.79 (m, 2H), 5.74 (s, 1H), 2.33 (s, 3H), 1.81-2.12 (m, 1H), 1.71 (tt, J=5.12, 8.39 Hz, 1H), 0.93-1.00 (m, 2H), 0.70-0.77 (m, 2H). [M+H]=215.1.

### Intermediate 39. 4-Bromo-3-(4-methoxyphenyl)-1-methyl-1H-pyrazole

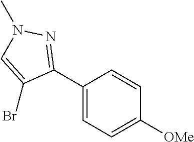

To a solution of 3-(4-methoxyphenyl)-1-methyl-1H-pyrazole (1.2 g, 6.4 mmol) in CH₂Cl₂ (11 mL) was added Br₂ (0.40 mL, 7.7 mmol) and the mixture stirred at rt for 15 min, then diluted with CH₂Cl₂, washed with 1 M aq Na₂SO₃, dried (MgSO₄) and evaporated to provide the title compound (1.82 g, 107%) as an amorphous solid. [M+H]=267.0/269.0.

### Intermediate 40. 4-Chloro-3-(4-methoxyphenyl)-1-methyl-1H-pyrazole

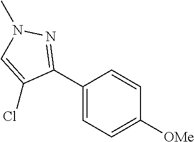

To a solution of 3-(4-methoxyphenyl)-1-methyl-1H-pyrazole (700 mg, 3.7 mmol) in acetonitrile (7 mL) was added NCS (600 mg, 4.5 mmol) and the mixture was stirred at 80° C. for 3 h, then cooled to rt, diluted with EtOAc, washed with a 1 M aq solution of Na₂SO₃, dried (MgSO₄) and evaporated. The residue was purified (FCC, SiO₂, 10-30% EtOAc/heptanes) to provide the title compound (576 mg, 74%) as a white solid. ¹H NMR (400 MHz, CDCl₃) δ 7.85 (d, J=8.93 Hz, 2H), 7.42 (s, 1H), 6.98 (d, J=8.93 Hz, 2H), 3.91 (s, 3H), 3.86 (s, 3H). [M+H]=223.0.

### Intermediate 41 and 42. 4-Ethyl-5-(4-methoxyphenyl)-1-methyl-1H-pyrazole and 4-Ethyl-3-(4-methoxyphenyl)-1-methyl-1H-pyrazole

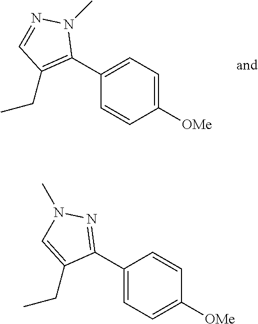 [SMILES: CCc1cnn(C)c1-c1ccc(OC)cc1 ; CCc1cn(C)nc1-c1ccc(OC)cc1]

Step 1. 2-(4-Methoxybenzoyl)butanal. A mixture of 1-(4-methoxyphenyl)butan-1-one (5.0 g, 28 mmol) and DMF-DMA (7.4 mL, 56 mmol) in DMF (56 mL) was stirred at 120° C. for 72 h, then cooled, diluted with ether, washed with water, brine, dried (MgSO₄) and evaporated. The residue was purified (FCC, SiO₂, 10-30% EtOAc/heptanes) to provide the title compound (2.90 g, 50%) as a white solid. ¹H NMR (400 MHz, CDCl₃) δ 9.71 (d, J=3.30 Hz, 1H), 7.98 (d, J=9.05 Hz, 2H), 6.99 (d, J=8.93 Hz, 2H), 4.21 (dt, J=3.30, 6.91 Hz, 1H), 3.91 (s, 3H), 1.96-2.23 (m, 2H), 1.00 (t, J=7.46 Hz, 3H). [M+H]=206.9.

Step 2. 4-Ethyl-5-(4-methoxyphenyl)-1-methyl-1H-pyrazole and 4-Ethyl-3-(4-methoxyphenyl)-1-methyl-1H-pyrazole. A mixture of 2-(4-methoxybenzoyl)butanal (1.45 g, 7.0 mmol), and methylhydrazine (1.8 mL, 35 mmol) in EtOH (14 mL) was stirred at 78° C. for 4 h, then cooled to rt, diluted with EtOAc, washed with water, brine, dried (MgSO₄) and evaporated. The residue was purified (FCC, SiO₂, 10-40% EtOAc/heptanes) to first provide 4-ethyl-5-(4-methoxyphenyl)-1-methyl-1H-pyrazole (276 mg, 18%) as an oil. ¹H NMR (400 MHz, CDCl₃) δ 7.43 (s, 1H), 7.24 (d, J=8.93 Hz, 2H), 7.02 (d, J=8.80 Hz, 2H), 3.89 (s, 3H), 3.75 (s, 3H), 2.40 (q, J=7.58 Hz, 2H), 1.13 (t, J=7.52 Hz, 3H). [M+H]=217.0. Continued elution provided a 60/40 mixture of 4-ethyl-5-(4-methoxyphenyl)-1-methyl-1H-pyrazole and 4-ethyl-3-(4-methoxyphenyl)-1-methyl-1H-pyrazole (556 mg, 37%). Final elution provided pure 4-ethyl-3-(4-methoxyphenyl)-1-methyl-1H-pyrazole (534 mg, 36%) as an oil. ¹H NMR (400 MHz, CDCl₃) δ 7.59 (d, J=8.80 Hz, 2H), 7.22 (s, 1H), 6.97 (d, J=8.93 Hz, 2H), 3.91 (s, 3H), 3.86 (s, 3H), 2.60-2.70 (m, 2H), 1.23 (t, J=7.46 Hz, 3H). [M+H]=217.0.

### Intermediate 43. 4-(4-Methoxy-1-methyl-1H-pyrazol-3-yl)phenol

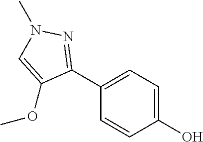

Step 1. 1-(4-(Benzyloxy)phenyl)-3-(dimethylamino)-2-methoxyprop-2-en-1-one. A mixture of 1-(4-(benzyloxy)phenyl)-2-methoxyethan-1-one (2.50 g, 9.8 mmol) and DMF-DMA (1.9 mL, 15 mmol) in DMF (20 mL) was stirred at 90° C. for 16 h, then cooled, diluted with ether, and washed with water and brine. Then, EtOAc was added to the organic layer, the organic layer was dried (MgSO₄) and the solvent was evaporated. The resulting residue was purified (FCC, SiO₂, 10-100% EtOAc/heptanes) to provide the title compound (1.81 g, 60%) as a white solid. ¹H NMR (400 MHz, CDCl₃) 7.67 (d, J=8.80 Hz, 2H), 7.36-7.53 (m, 5H), 6.98 (d, J=8.80 Hz, 2H), 6.74 (s, 1H), 5.13 (s, 2H), 3.57 (s, 3H), 3.12 (s, 6H). [M+H]=312.1.

Step 2. 3-(4-(Benzyloxy)phenyl)-4-methoxy-1-methyl-1H-pyrazole and 5-(4-(benzyloxy)phenyl)-4-methoxy-1-methyl-1H-pyrazole. A mixture of 1-(4-(benzyloxy)phenyl)-3-(dimethylamino)-2-methoxyprop-2-en-1-one (1.50 g, 4.8 mmol) and methylhydrazine (1.3 mL, 24 mmol) in EtOH (19 mL) was stirred at 78° C. for 4 h, then cooled to rt, diluted with EtOAc, washed with water, washed with brine, dried (MgSO₄) and evaporated. The residue was purified (FCC, SiO₂, 10-50% EtOAc/heptanes) to first provide an early fraction of pure 3-(4-(benzyloxy)phenyl)-4-methoxy-1-methyl-1H-pyrazole (12 mg, 1%) and then a 4:1 mixture of 3-(4-(benzyloxy)phenyl)-4-methoxy-1-methyl-1H-pyrazole and 5-(4-(benzyloxy)phenyl)-4-methoxy-1-methyl-1H-pyrazole, respectively (1.4 g, 98%) as an oil. The pure isomer 3-(4-(benzyloxy)phenyl)-4-methoxy-1-methyl-1H-pyrazole was analyzed. ¹H NMR (400 MHz, CDCl₃) δ 7.86-7.94 (m, 2H), 7.45-7.50 (m, 2H), 7.37-7.43 (m, 2H), 7.30-7.36 (m, 1H), 7.09 (s, 1H), 7.03 (d, J=8.93 Hz, 2H), 5.12 (s, 2H), 3.88 (s, 3H), 3.82 (s, 3H). [M+H]=295.1. The 4:1 mixture of 3-(4-(benzyloxy)phenyl)-4-methoxy-1-methyl-1H-pyrazole and 5-(4-(benzyloxy)phenyl)-4-methoxy-1-methyl-1H-pyrazole was used in the next reaction without further purification.

Step 3. 4-(4-Methoxy-1-methyl-1H-pyrazol-3-yl)phenol. The 4:1 mixture of 3-(4-(benzyloxy)phenyl)-4-methoxy-1-methyl-1H-pyrazole and 5-(4-(benzyloxy)phenyl)-4-methoxy-1-methyl-1H-pyrazole (1.4 g, 4.8 mmol) was combined with 10% Pd/C (506 mg, 0.5 mmol) in a mixture of EtOAc (32 mL) and MeOH (32 mL) and stirred under 170 psi H₂ at rt for 12 h. Then, the mixture was filtered through Celite®, concentrated to ca. 30 mL and then passed through a 0.2-μm PTFE filter. The filtrate was evaporated and the white solid that was obtained was recrystallized from MeOH (10 mL) to provide the title compound (478 mg, 49%) as a white crystalline solid. ¹H NMR (400 MHz, DMSO-d₆) δ 9.37 (s, 1H), 7.65 (d, J=8.68 Hz, 2H), 7.51 (s, 1H), 6.76 (d, J=8.80 Hz, 2H), 3.76 (s, 3H), 3.73 (s, 3H). [M+H]=204.9. These crystals were subjected to X-ray structure determination at the UCSD Crystallography lab to confirm the structure of the title compound.

### Intermediate 44. 3-(4-Hydroxyphenyl)-1-methyl-1H-pyrazole-4-carbonitrile

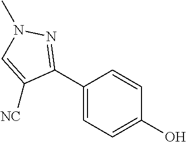

Step 1. 4-Iodo-3-(4-methoxyphenyl)-1-methyl-1H-pyrazole. To a solution of 3-(4-methoxyphenyl)-1-methyl-1H-pyrazole (1.25 g, 6.6 mmol) in acetonitrile (13 mL) was added NIS (1.64 g, 7.3 mmol) and the mixture stirred at 80° C. for 90 min, then cooled to rt, diluted with EtOAc, washed with a 1 M aq solution of Na₂SO₃, dried (MgSO₄) and evaporated. The residue was purified (FCC, SiO₂, 10-30% EtOAc/heptanes) to provide the title compound (1.84 g, 88%) as a white solid. ¹H NMR (400 MHz, CDCl₃) δ 7.78 (d, J=8.80 Hz, 2H), 7.48 (s, 1H), 6.98 (d, J=8.80 Hz, 2H), 3.96 (s, 3H), 3.87 (s, 3H). [M+H]=314.9.

Step 2. 3-(4-Methoxyphenyl)-1-methyl-1H-pyrazole-4-carbonitrile. To a solution of 4-iodo-3-(4-methoxyphenyl)-1-methyl-1H-pyrazole (1.00 g, 3.2 mmol) in THF (32 mL) at 0° C. was added a 2.9 M solution of i-PrMgBr in THF (1.7 mL, 4.9 mmol) and the mixture was stirred at 0° C. for 30 min, then TsCN (870 mg, 4.8 mmol) was added as a solid and the resulting mixture stirred at 0° C. for 2 h. Then, the mixture was diluted with a 1 M aq solution of NH₄Cl and EtOAc, the layers were separated and the organic layer was dried (MgSO₄) and the solvent was evaporated. The residue was purified (FCC, SiO₂, 10-40% EtOAc/heptanes) to provide the title compound (493 mg, 72%) as a white solid. ¹H NMR (400 MHz, CDCl₃) δ 7.93 (d, J=8.80 Hz, 2H), 7.83 (s, 1H), 7.00 (d, J=8.93 Hz, 2H), 3.98 (s, 3H), 3.88 (s, 3H). [M+H]=213.9.

Step 3. 3-(4-Hydroxyphenyl)-1-methyl-1H-pyrazole-4-carbonitrile. To a solution of 3-(4-methoxyphenyl)-1-methyl-1H-pyrazole-4-carbonitrile (493 mg, 2.3 mmol) in CH₂Cl₂ (12 mL) was added a 1 M solution of BBr₃ in CH₂Cl₂ (4.6 mL, 4.6 mmol) at −40° C. and the reaction mixture was allowed to stir at room temperature for 4 h. To the mixture was added an additional CH₂Cl₂ (12 mL) and additional 1 M solution of BBr₃ in CH₂Cl₂ (4.6 mL, 4.6 mmol) at 0° C. The heterogeneous mixture was sonicated for 8 h then was stirred at rt for 16 h. The resulting mixture was diluted slowly with water, then diluted with a 1 M aq solution of NaHCO₃ until the pH was 8, the organic layer was separated and the aqueous layer was extracted with a 9:1 mixture of CH₂Cl₂ and MeOH (6×50 mL). These organic layers were combined. A 4 N aqueous solution of HCl was added to the aqueous layer until a pH of 4 was obtained, then the aqueous solution was extracted with a 9:1 mixture of CH₂Cl₂ and MeOH (3×50 mL). All organic layers were combined, dried (MgSO₄) and evaporated. The residue was purified (FCC, SiO₂, 10-60% EtOAc/heptanes) to provide the title compound (210 mg, 46%) as a white solid. ¹H NMR (300 MHz, DMSO-d₆) δ 9.82 (s, 1H), 8.53 (s, 1H), 7.69 (d, J=8.68 Hz, 2H), 6.88 (d, J=8.68 Hz, 2H), 3.91 (s, 3H). [M+H]=199.9.

### Intermediate 45. 4-(1-Ethyl-4-fluoro-1H-pyrazol-3-yl)phenol

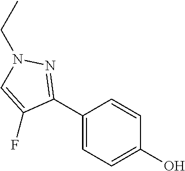

Step 1. 1-Ethyl-3-(4-methoxyphenyl)-1H-pyrazole. A mixture of 3-bromo-1-ethyl-1H-pyrazole (3.0 g, 17 mmol), (4-methoxyphenyl)boronic acid (5.73 g, 37 mmol), Pd(dppf)Cl₂—CH₂Cl₂ (700 mg, 0.86 mmol), a 1 M aq. solution of NaHCO₃ (43 mL), and acetonitrile (86 mL) was bubbled with N₂ for 1 min and then stirred at 80° C. for 16 h. After cooling to rt, the mixture was diluted with EtOAc, washed with brine, dried (MgSO₄), and the solvent was removed by evaporation. The residue was purified (FCC, SiO₂, 0-25% EtOAc/heptanes) to provide the title compound (2.55 g, 74%) as an amorphous solid. ¹H NMR (400 MHz, CDCl₃) δ 7.76 (d, J=8.93 Hz, 2H), 7.42 (d, J=2.20 Hz, 1H), 6.95 (d, J=8.93 Hz, 2H), 6.49 (d, J=2.32 Hz, 1H), 4.24 (q, J=7.34 Hz, 2H), 3.86 (s, 3H), 1.55 (t, J=7.27 Hz, 3H). [M+H]=203.1.

Step 2. 1-Ethyl-4-iodo-3-(4-methoxyphenyl)-1H-pyrazole. To a solution of 1-Ethyl-3-(4-methoxyphenyl)-1H-pyrazole (2.55 g, 12.6 mmol) in 25 mL acetonitrile was added NIS (3.12 g, 13.9 mmol) and the mixture stirred at 80° C. for 90 min, then cooled to rt and diluted with EtOAc, washed with a 1 M aq solution of Na₂SO₃, dried (MgSO₄) and the solvent was removed by evaporation. The residue was purified (FCC, SiO₂, 10-30% EtOAc/heptanes) to provide the title compound (3.87 g, 93%) as an amorphous solid. ¹H NMR (400 MHz, CDCl₃) δ 7.79 (d, J=8.80 Hz, 2H), 7.52 (s, 1H), 6.99 (d, J=8.93 Hz, 2H), 4.23 (q, J=7.34 Hz, 2H), 3.87 (s, 3H), 1.54 (t, J=7.34 Hz, 3H). [M+H]=328.8.

Step 3. 1-Ethyl-4-fluoro-3-(4-methoxyphenyl)-1H-pyrazole. To a solution of 1-ethyl-4-iodo-3-(4-methoxyphenyl)-1H-pyrazole (1.40 g, 4.27 mmol) in THF (28 mL) at −78° C. was added a 1.6 M n-BuLi solution in THF (3.22 mL, 5.12 mmol). After stirring at −78° C. for 20 min, a solution of NFSI (1.61 g, 5.12 mmol) in THF (14 mL) was added and the resulting mixture stirred at −78° C. for 1 h. The reaction was diluted with a 1 M aq solution of NH₄Cl and EtOAc, the layers were separated and the aqueous layer extracted with EtOAc. The combined organic layers were washed with a 1 M aq solution of NaHCO₃, brine, dried (MgSO₄) and the solvent was removed by evaporation. The residue was purified (FCC, SiO₂, 0-25% EtOAc/heptanes) to provide the title compound (373 mg, 40%) as an amorphous solid. ¹H NMR (400 MHz, CDCl₃) δ 7.81 (d, J=8.31 Hz, 2H), 7.35 (d, J=4.77 Hz, 1H), 6.97 (d, J=9.05 Hz, 2H), 4.15 (q, J=7.25 Hz, 2H), 3.86 (s, 3H), 1.52 (t, J=7.34 Hz, 3H). [M+H]=221.1.

Step 4. 4-(1-Ethyl-4-fluoro-1H-pyrazol-3-yl)phenol. To a solution of 1-ethyl-4-fluoro-3-(4-methoxyphenyl)-1H-pyrazole (373 mg, 1.7 mmol) in CH₂Cl₂ (9 mL) was added a 1 M solution of BBr₃ in CH₂Cl₂ (4.2 mL, 4.2 mmol) at 0° C. and the reaction mixture was allowed to stir at room temperature for 2 h. To the mixture was added additional CH₂Cl₂ (12 mL) and a 1 M solution of BBr₃ in CH₂Cl₂ (4.6 mL, 4.6 mmol) at 0° C. and the heterogeneous mixture was sonicated for 8 h then let stir at rt for 16 h. The resulting mixture was diluted with CH₂Cl₂ and stirred with a 1 M aq solution of NaHCO₃. Then, a 4 N aq solution of HCl was added until a pH of 7 was obtained. The solution was then extracted with a 9:1 mixture of CH₂Cl₂ and MeOH (2×50 mL). The combined organic layers were dried (MgSO₄) and concentrated to a volume of approximately 5 mL and heptane (10 mL) was added. The solvent was removed by evaporation to provide the title compound (347 mg, 99%) as a white solid. ¹H NMR (300 MHz, DMSO-d₆) δ 9.55 (br s, 1H), 7.92 (d, J=4.65 Hz, 1H), 7.55 (d, J=8.31 Hz, 2H), 6.82 (d, J=8.68 Hz, 2H), 4.06 (q, J=7.21 Hz, 2H), 1.37 (t, J=7.27 Hz, 3H). [M+H]=207.1.

### Intermediate 46. 1-(4-(4-Hydroxyphenyl)piperidin-1-yl)ethan-1-one

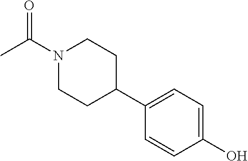 [SMILES: CC(=O)N1CCC(c2ccc(O)cc2)CC1]

The title compound was made in a manner analogous to Intermediate 20, using the appropriate starting material substitutions. ¹H NMR (400 MHz, CDCl₃) δ 7.06 (d, J=8.4 Hz, 2H), 6.82 (d, J=8.6 Hz, 2H), 6.11 (br s, 1H), 4.85-4.69 (m, 1H), 3.94 (td, J=2.0, 13.6 Hz, 1H), 3.18 (dt, J=2.6, 13.1 Hz, 1H), 2.81-2.57 (m, 2H), 2.16 (s, 3H), 1.97-1.82 (m, 2H), 1.60 (tq, J=3.9, 12.6 Hz, 2H). [M+H]=220.1.

### Intermediate 47. 6-(1,3-Dimethyl-1H-pyrazol-4-yl)pyridin-3-ol

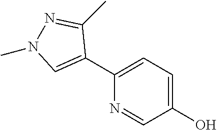

The title compound was made in a manner analogous to Intermediate 20, using the appropriate starting material substitutions. ¹H NMR (400 MHz, DMSO-d₆) δ 9.72 (s, 1H), 8.10 (d, J=2.4 Hz, 1H), 7.96 (s, 1H), 7.35 (d, J=8.6 Hz, 1H), 7.15 (dd, J=2.9, 8.6 Hz, 1H), 3.76 (s, 3H), 2.36 (s, 3H). [M+H]=190.1.

### Intermediate 48. 4-(Cyclobutylmethoxy)phenol

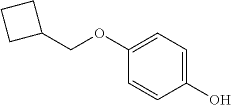 [SMILES: Oc1ccc(OCC2CCC2)cc1]

The title compound was made in a manner analogous to Intermediate 22, using the appropriate starting material substitutions. ¹H NMR (400 MHz, CDCl₃) δ 6.84-6.80 (m, 2H), 6.79-6.75 (m, 2H), 4.47 (br s, 1H), 3.89 (d, J=6.7 Hz, 2H), 2.84-2.67 (m, 1H), 2.26-2.09 (m, 2H), 2.03-1.78 (m, 4H). [M+H]=178.2.

### Intermediate 49. 4-(Oxetan-2-ylmethoxy)phenol

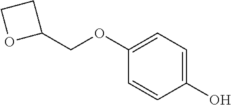 [SMILES: Oc1ccc(OCC2CCO2)cc1]

The title compound was made in a manner analogous to Intermediate 22, using the appropriate starting material substitutions. ¹H NMR (400 MHz, CDCl₃) δ 6.88-6.82 (m, 2H), 6.80-6.73 (m, 2H), 5.20-5.06 (m, 1H), 4.96 (br s, 1H), 4.80-4.63 (m, 2H), 4.09 (d, J=4.3 Hz, 2H), 2.85-2.64 (m, 2H). [M+H]=181.

### Intermediate 50. 4-(Oxetan-3-yloxy)phenol

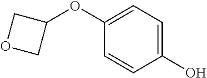 [SMILES: Oc1ccc(OC2COC2)cc1]

The title compound was made in a manner analogous to Intermediate 22, using the appropriate starting material substitutions. [M+H]=166.1.

### Intermediate 51. 4-((Tetrahydrofuran-3-yl)oxy)phenol

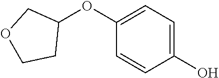 [SMILES: Oc1ccc(OC2CCOC2)cc1]

The title compound was made in a manner analogous to Intermediate 22, using the appropriate starting material substitutions. ¹H NMR (400 MHz, CDCl₃) δ 6.78 (s, 4H), 4.92-4.80 (m, 1H), 4.69 (br s, 1H), 4.06-3.96 (m, 3H), 3.95-3.88 (m, 1H), 2.22-2.13 (m, 2H). [M+H]=181.1.

### Intermediate 52. 4-(Oxetan-3-ylmethoxy)phenol

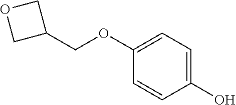 [SMILES: Oc1ccc(OCC2COC2)cc1]

The title compound was made in a manner analogous to Intermediate 22, using the appropriate starting material substitutions. ¹H NMR (400 MHz, CDCl₃) δ 6.86-6.80 (m, 2H), 6.80-6.75 (m, 2H), 5.21 (s, 1H), 4.91 (dd, J=6.4, 7.7 Hz, 2H), 4.61 (t, J=6.1 Hz, 2H), 4.16 (d, J=6.7 Hz, 2H), 3.44 (quind, J=6.8, 13.6 Hz, 1H). [M+H]=181.1.

### Intermediate 53. 4-(Tetrahydrofuran-3-yl)phenol

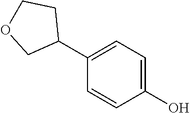 [SMILES: Oc1ccc(C2CCOC2)cc1]

The title compound was made in a manner analogous to Intermediate 20, using the appropriate starting material substitutions. ¹H NMR (400 MHz, CDCl₃) δ 7.14 (d, J=8.4 Hz, 2H), 6.81 (d, J=8.6 Hz, 2H), 5.24 (s, 1H), 4.15 (t, J=7.9 Hz, 1H), 4.09 (dt, J=4.5, 8.3 Hz, 1H), 3.95 (q, J=8.0 Hz, 1H), 3.70 (t, J=8.1 Hz, 1H), 3.38 (quin, J=7.9 Hz, 1H), 2.42-2.31 (m, 1H), 2.00 (qd, J=8.2, 12.3 Hz, 1H). [M+H]=165.1.

### Intermediate 54. 4-(4-Hydroxyphenyl)tetrahydro-2H-pyran-4-ol

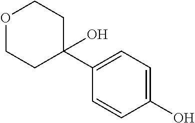 [SMILES: Oc1ccc(C2(O)CCOCC2)cc1]

The title compound was made in a manner analogous to Intermediate 21, using the appropriate starting material substitutions. ¹H NMR (400 MHz, CDCl₃) δ 7.40 (d, J=8.7 Hz, 2H), 6.86 (d, J=8.7 Hz, 2H), 4.90 (br s, 1H), 4.00-3.92 (m, 2H), 3.92-3.86 (m, 2H), 2.17 (dt, J=5.3, 12.7 Hz, 2H), 1.73 (d, J=12.3 Hz, 2H), 1.53 (s, 1H). [M+H]=177.1.

### Intermediate 55. 4-Cyclopropoxyphenol

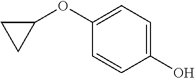 [SMILES: Oc1ccc(OC2CC2)cc1]

The title compound was made in a manner analogous to Intermediate 22, using the appropriate starting material substitutions. ¹H NMR (400 MHz, CDCl₃) δ 6.95 (d, J=9.0 Hz, 2H), 6.79 (d, J=9.0 Hz, 2H), 4.54 (br s, 1H), 3.76-3.64 (m, 1H), 0.83-0.71 (m, 4H). [M+H]=150.1.

### Intermediate 56. 4-((Tetrahydro-2H-pyran-4-yl)oxy)phenol

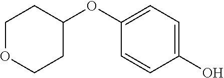 [SMILES: Oc1ccc(OC2CCOCC2)cc1]

The title compound was made in a manner analogous to Intermediate 22, using the appropriate starting material substitutions. [M+H]=195.1.

### Intermediate 57. 4-((Tetrahydrofuran-2-yl)methoxy)phenol

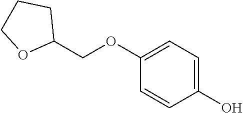 [SMILES: Oc1ccc(OCC2CCCO2)cc1]

The title compound was made in a manner analogous to Intermediate 22, using the appropriate starting material substitutions. ¹H NMR (400 MHz, CDCl₃) δ 6.89-6.79 (m, 2H), 6.78-6.71 (m, 2H), 4.86 (br s, 1H), 4.36-4.19 (m, 1H), 4.04-3.78 (m, 4H), 2.32-1.79 (m, 4H). [M+H]=195.1.

### Intermediate 58. 6-(1-Isopropyl-1H-pyrazol-4-yl)pyridin-3-ol

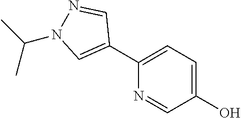

The title compound was made in a manner analogous to Intermediate 20, using the appropriate starting material substitutions. ¹H NMR (400 MHz, CDCl₃) δ 10.73 (br s, 1H), 8.94 (br s, 1H), 8.88 (s, 1H), 8.03 (s, 1H), 7.88-7.71 (m, 2H), 4.61 (td, J=6.6, 13.3 Hz, 1H), 1.60 (d, J=6.7 Hz, 6H). [M+H]=204.1.

### Intermediate 59. 6-(1-Methyl-3-(trifluoromethyl)-1H-pyrazol-4-yl)pyridin-3-ol

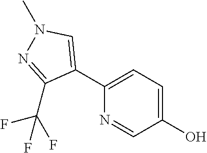 [SMILES: Cn1cc(-c2ccc(O)cn2)c(C(F)(F)F)n1]

The title compound was made in a manner analogous to Intermediate 35, using the appropriate starting material substitutions. ¹H NMR (400 MHz, CDCl₃) δ 8.28 (br s, 1H), 7.86 (s, 1H), 7.49 (d, J=7.34 Hz, 1H), 7.16-7.28 (m, 1H), 4.00 (s, 3H). [M+H]=244.2.

### Intermediate 60. 4-(3-Cyclopropyl-1-methyl-1H-pyrazol-4-yl)phenol

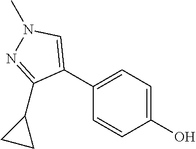 [SMILES: Cn1cc(-c2ccc(O)cc2)c(C2CC2)n1]

The title compound was made in a manner analogous to Intermediate 35, using the appropriate starting material substitutions. ¹H NMR (400 MHz, CDCl₃) δ 7.84 (s, 1H), 7.40 (d, J=8.68 Hz, 2H), 7.02 (d, J=8.68 Hz, 2H), 5.48 (br s, 1H), 3.91 (s, 3H). [M+H]=215.2.

### Intermediate 61. 5-(1-Methyl-3-(trifluoromethyl)-1H-pyrazol-4-yl)pyridin-2-ol

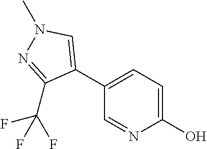 [SMILES: Cn1cc(-c2ccc(O)nc2)c(C(F)(F)F)n1]

The title compound was made in a manner analogous to Intermediate 20, using the appropriate starting material substitutions. ¹H NMR (400 MHz, CDCl₃) δ 7.54 (d, J=9.17 Hz, 1H), 7.41-7.48 (m, 2H), 6.67 (d, J=9.29 Hz, 1H), 4.01 (s, 3H). [M+H]=244.

### Intermediate 62. 4-(3-Methoxy-1-methyl-1H-pyrazol-4-yl)phenol

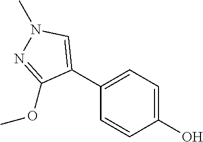

The title compound was made in a manner analogous to Intermediate 35, using the appropriate starting material substitutions. ¹H NMR (400 MHz, CDCl₃) δ 7.48 (d, J=8.68 Hz, 2H), 7.36 (s, 1H), 6.85 (d, J=8.80 Hz, 2H), 5.52 (br s, 1H), 4.01 (s, 3H), 3.79 (s, 3H). [M+H]=205.

### Intermediate 63. 4-(1,2-Dimethyl-1H-imidazol-4-yl)phenol

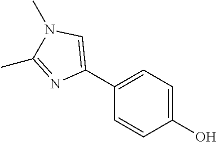

The title compound was made in a manner analogous to Intermediate 37, using the appropriate starting material substitutions. ¹H NMR (400 MHz, DMSO-d₆) δ 9.26 (s, 1H), 7.48 (d, J=7.8 Hz, 2H), 7.25 (s, 1H), 6.71 (d, J=7.9 Hz, 2H), 3.62-3.46 (m, 3H), 2.35-2.21 (m, 3H). [M+H]=189.2.

### Intermediate 64. 4-(4-Methoxypyrimidin-2-yl)phenol

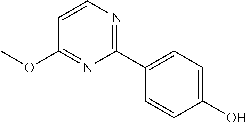

The title compound was made in a manner analogous to Intermediate 35, using the appropriate starting material substitutions. ¹H NMR (400 MHz, CDCl₃) δ 8.48 (d, J=5.7 Hz, 1H), 8.36 (d, J=8.7 Hz, 2H), 6.93 (d, J=8.7 Hz, 2H), 6.61 (d, J=5.9 Hz, 1H), 6.23 (br s, 1H), 4.10 (s, 3H). [M+H]=203.2.

### Intermediate 65. 4-(4-(Methylthio)pyrimidin-2-yl)phenol

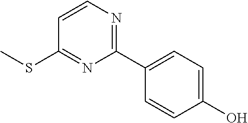

The title compound was made in a manner analogous to Intermediate 35, using the appropriate starting material substitutions. ¹H NMR (400 MHz, CDCl₃) δ 8.41-8.34 (m, 3H), 7.03 (d, J=5.4 Hz, 1H), 6.93 (d, J=8.8 Hz, 2H), 5.95 (br s, 1H), 2.68 (s, 3H). [M+H]=219.2.

### Intermediate 66. 4-(4-(Trifluoromethyl)pyridin-2-yl)phenol

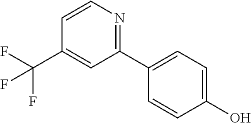 [SMILES: Oc1ccc(-c2cc(C(F)(F)F)ccn2)cc1]

¹H NMR (400 MHz, CDCl₃) δ 8.83 (d, J=5.13 Hz, 1H), 7.92 (d, J=8.68 Hz, 2H), 7.88 (s, 1H), 7.43 (d, J=5.01 Hz, 1H), 6.93 (d, J=8.68 Hz, 2H), 6.39 (br s, 1H). [M+H]=240.2.

### Intermediate 67. 4-(5-(Trifluoromethyl)pyridin-3-yl)phenol

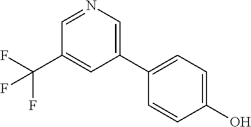 [SMILES: Oc1ccc(-c2cncc(C(F)(F)F)c2)cc1]

The title compound was made in a manner analogous to Intermediate 35, using the appropriate starting material substitutions. ¹H NMR (400 MHz, CDCl₃) δ 9.01 (s, 1H), 8.84 (s, 1H), 8.09 (s, 1H), 7.53 (d, J=8.56 Hz, 2H), 7.02 (d, J=8.68 Hz, 2H), 6.13 (br s, 1H). [M+H]=240.2.

### Intermediate 68. 4-(4-Bromo-1-methyl-1H-pyrazol-3-yl)phenol

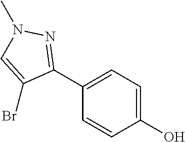

The title compound was made in a manner analogous to Intermediate 38, using the appropriate starting material substitutions. ¹H NMR (400 MHz, CDCl₃) δ 7.78 (d, J=8.80 Hz, 2H), 7.46 (s, 1H), 6.91 (d, J=8.80 Hz, 2H), 3.94 (s, 3H). [M+H]=252.9/254.9.

### Intermediate 69. 4-(4-Chloro-1-methyl-1H-pyrazol-3-yl)phenol

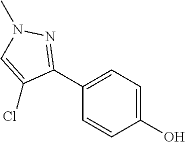

The title compound was made in a manner analogous to Intermediate 38, using the appropriate starting material substitutions. ¹H NMR (400 MHz, CDCl₃) δ 7.79 (d, J=8.80 Hz, 2H), 7.43 (s, 1H), 6.90 (d, J=8.80 Hz, 2H), 3.92 (s, 3H). [M+H]=209.

### Intermediate 70. 4-(4-Cyclopropyl-1-methyl-1H-pyrazol-3-yl)phenol

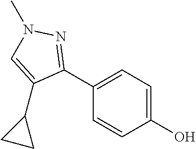 [SMILES: Cn1cc(C2CC2)c(-c2ccc(O)cc2)n1]

The title compound was made in a manner analogous to Intermediate 38, using the appropriate starting material substitutions. ¹H NMR (400 MHz, CDCl₃) δ 7.74 (d, J=8.68 Hz, 2H), 7.06 (s, 1H), 6.86 (d, J=8.68 Hz, 2H), 6.02 (br s, 1H), 3.88 (s, 3H), 1.77 (tt, J=5.21, 8.24 Hz, 1H), 0.87-0.97 (m, 2H), 0.51-0.58 (m, 2H). [M+H]=215.1.

### Intermediate 71. 4-(4-Ethyl-1-methyl-1H-pyrazol-5-yl)phenol

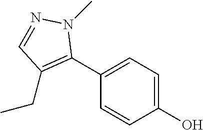

The title compound was made from Intermediate 41 in a manner analogous to Step 3 in the preparation of Intermediate 38. ¹H NMR (400 MHz, DMSO-d₆) δ 7.44 (s, 1H), 7.19 (d, J=8.56 Hz, 2H), 6.89 (d, J=8.56 Hz, 2H), 3.66 (s, 3H), 2.32 (q, J=7.58 Hz, 2H), 1.05 (t, J=7.52 Hz, 3H). [M+H]=202.9.

### Intermediate 72. 4-(4-Ethyl-1-methyl-1H-pyrazol-3-yl)phenol

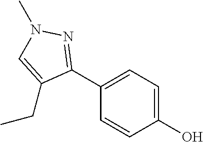

The title compound was made from Intermediate 42 in a manner analogous to Step 3 in the preparation of Intermediate 38. ¹H NMR (400 MHz, DMSO-d₆) δ 7.50 (s, 1H), 7.39 (d, J=8.80 Hz, 2H), 6.79 (d, J=8.68 Hz, 2H), 3.79 (s, 3H), 2.52-2.59 (m, 2H), 1.14 (t, J=7.52 Hz, 3H). [M+H]=202.9.

### Intermediate 73. 4-(1-Ethyl-1H-pyrazol-3-yl)phenol

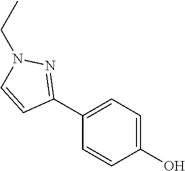

The title compound was made in a manner analogous to Intermediate 37, then Intermediate 38, step 3, using the appropriate starting material substitutions. ¹H NMR (400 MHz, DMSO-d₆) δ 1.39 (t, J=7.3 Hz, 3H), 4.14 (q, J=7.2 Hz, 2H), 5.76 (s, 1H), 6.54 (d, J=2.2 Hz, 1H), 6.78 (d, J=8.7 Hz, 2H), 7.59 (d, J=8.7 Hz, 2H), 7.73 (d, J=2.2 Hz, 1H). [M+H]=189.

### Intermediate 74. 4-(4-Chloro-1-ethyl-1H-pyrazol-3-yl)phenol

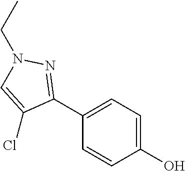

The title compound was made in a manner analogous to Intermediate 37, then Intermediate 40, then Intermediate 38, step 3, using the appropriate starting material substitutions. ¹H NMR (400 MHz, DMSO-d₆) δ 1.39 (t, J=7.3 Hz, 3H), 4.12 (q, J=7.21 Hz, 2H), 6.83 (d, J=8.7 Hz, 2H), 7.62 (d, J=8.7 Hz, 2H), 8.04 (s, 1H). [M+H]=223.

### Intermediate 75. 4-(1-Propyl-1H-pyrazol-3-yl)phenol

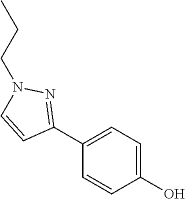

The title compound was made in a manner analogous to Intermediate 37, then Intermediate 38, step 3, using the appropriate starting material substitutions. ¹H NMR (400 MHz, DMSO-d₆) δ 0.85 (t, J=7.2 Hz, 3H), 1.77 (sext, J=7.2 Hz, 2H), 4.06 (t, J=7.2 Hz, 2H), 6.48 (d, J=2.2 Hz, 1H), 6.75 (d, J=8.7 Hz, 2H), 7.57 (d, J=8.7 Hz, 2H), 7.72 (d, J=2.2 Hz, 1H), 9.41 (s, 1H). [M+H]=203.

### Intermediate 76. 4-(4-Chloro-1-propyl-1H-pyrazol-3-yl)phenol

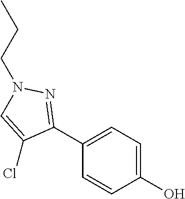 [SMILES: CCCn1cc(Cl)c(-c2ccc(O)cc2)n1]

The title compound was made in a manner analogous to Intermediate 37, then Intermediate 40, then Intermediate 38, step 3, using the appropriate starting material substitutions. ¹H NMR (400 MHz, DMSO-d₆) δ 0.85 (t, J=7.2 Hz, 3H), 1.80 (sext, J=7.2 Hz, 2H), 4.04 (t, J=7.2 Hz, 2H), 6.83 (d, J=8.7 Hz, 2H), 7.63 (d, J=8.7 Hz, 2H), 8.02 (s, 1H), 9.59 (s, 1H). [M+H]=237.

### Intermediate 77. 4-(1-Isopropyl-1H-pyrazol-3-yl)phenol

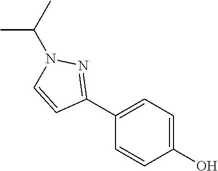

The title compound was made in a manner analogous to Intermediate 37, then Intermediate 38, step 3, using the appropriate starting material substitutions. ¹H NMR (400 MHz, DMSO-d₆) δ 1.44 (d, J=6.6 Hz, 6H), 4.50 (sept, J=6.6 Hz, 1H), 6.51 (d, J=2.2 Hz, 1H), 6.76 (d, J=8.7 Hz, 2H), 7.60 (d, J=8.7 Hz, 2H), 7.72 (d, J=2.2 Hz, 1H), 9.40 (s, 1H). [M+H]=203.

### Intermediate 78. 4-(4-Chloro-1-isopropyl-1H-pyrazol-3-yl)phenol

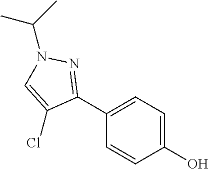 [SMILES: CC(C)n1cc(Cl)c(-c2ccc(O)cc2)n1]

The title compound was made in a manner analogous to Intermediate 37, then Intermediate 40, then Intermediate 38, step 3, using the appropriate starting material substitutions. ¹H NMR (400 MHz, DMSO-d₆) δ 9.59 (br s, 1H), 8.05 (s, 1H), 7.63 (d, J=8.68 Hz, 2H), 6.83 (d, J=8.68 Hz, 2H), 4.46 (spt, J=6.66 Hz, 1H), 1.43 (d, J=6.60 Hz, 6H). [M+H]=237.

### Intermediate 79. 4-(4-Iodo-1-methyl-1H-pyrazol-3-yl)phenol

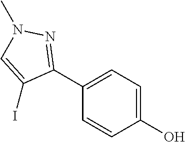

The title compound was made in a manner analogous Intermediate 38, using the appropriate starting material substitutions. ¹H NMR (400 MHz, DMSO-d₆) δ 9.57 (br s, 1H), 7.90 (s, 1H), 7.58 (d, J=8.68 Hz, 2H), 6.82 (d, J=8.68 Hz, 2H), 3.86 (s, 3H). [M+H]=300.9.

### Intermediate 80. 4-(1-Ethyl-4-methoxy-1H-pyrazol-3-yl)phenol

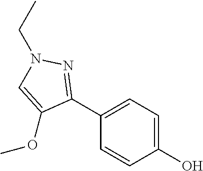 [SMILES: CCn1cc(OC)c(-c2ccc(O)cc2)n1]

The title compound was made in a manner analogous Intermediate 43, using the appropriate starting material substitutions. ¹H NMR (400 MHz, DMSO-d₆) δ 9.41 (br s, 1H), 7.65 (d, J=8.80 Hz, 2H), 7.56 (s, 1H), 6.76 (d, J=8.68 Hz, 2H), 4.03 (q, J=7.34 Hz, 2H), 3.74 (s, 3H), 1.37 (t, J=7.27 Hz, 3H). [M+H]=219.

### Intermediate 81. 4-(1-Ethyl-4-methoxy-1H-pyrazol-5-yl)phenol

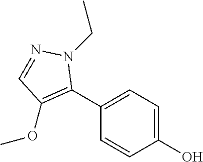 [SMILES: CCn1ncc(OC)c1-c1ccc(O)cc1]

The title compound was made in a manner analogous Intermediate 43, using the appropriate starting material substitutions. ¹H NMR (400 MHz, DMSO-d₆) δ 9.71 (br s, 1H), 7.38 (s, 1H), 7.20 (d, J=8.56 Hz, 2H), 6.87 (d, J=8.56 Hz, 2H), 3.97 (q, J=7.21 Hz, 2H), 3.66 (s, 3H), 1.22 (t, J=7.15 Hz, 3H). [M+H]=219.

### Intermediate 82. 4-(1-Isopropyl-4-methoxy-1H-pyrazol-3-yl)phenol

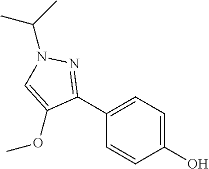 [SMILES: COc1cn(C(C)C)nc1-c1ccc(O)cc1]

The title compound was made in a manner analogous Intermediate 43, using the appropriate starting material substitutions. ¹H NMR (400 MHz, DMSO-d₆) δ 9.40 (br s, 1H), 7.65 (d, J=8.80 Hz, 2H), 7.59 (s, 1H), 6.76 (d, J=8.68 Hz, 2H), 4.37 (spt, J=6.68 Hz, 1H), 3.74 (s, 3H), 1.41 (d, J=6.60 Hz, 6H). [M+H]=233.

### Intermediate 83. 4-(1-Isopropyl-4-methoxy-1H-pyrazol-5-yl)phenol

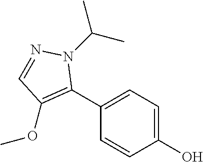 [SMILES: COc1cnn(C(C)C)c1-c1ccc(O)cc1]

The title compound was made in a manner analogous Intermediate 43, using the appropriate starting material substitutions. ¹H NMR (400 MHz, DMSO-d₆) δ 9.70 (br s, 1H), 7.39 (s, 1H), 7.16 (d, J=8.68 Hz, 2H), 6.87 (d, J=8.56 Hz, 2H), 4.35 (spt, J=6.54 Hz, 1H), 3.65 (s, 3H), 1.31 (d, J=6.48 Hz, 6H). [M+H]=233.

### Intermediate 84. 4-(1,4-Dimethyl-1H-pyrazol-5-yl)phenol

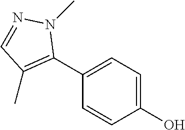

The title compound was made in a manner analogous to Intermediate 41, then Intermediate 38, step 3, using the appropriate starting material substitutions. ¹H NMR (400 MHz, CDCl₃) δ 7.44 (s, 1H), 7.21 (d, J=8.56 Hz, 2H), 7.00 (d, J=8.56 Hz, 2H), 3.81 (s, 3H), 2.03 (s, 3H). [M+H]=188.9.

### Intermediate 85. 4-(1,4-Dimethyl-1H-pyrazol-3-yl)phenol

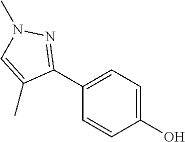

The title compound was made in a manner analogous to Intermediate 42, then Intermediate 38, step 3, using the appropriate starting material substitutions. ¹H NMR (400 MHz, CDCl₃) δ 7.53 (d, J=8.68 Hz, 2H), 7.21 (s, 1H), 6.86 (d, J=8.68 Hz, 2H), 5.94 (br s, 1H), 3.91 (s, 3H), 2.21 (s, 3H). [M+H]=188.9.

### Intermediate 86. 4-(4-Fluoro-1-methyl-1H-pyrazol-3-yl)phenol

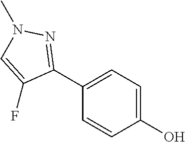

The title compound was made in a manner analogous to Intermediate 43, steps 1 and 2, then Intermediate 44, step 3, using the appropriate starting material substitutions. ¹H NMR (400 MHz, DMSO-d₆) δ 9.56 (s, 1H), 7.86 (d, J=4.65 Hz, 1H), 7.54 (d, J=8.31 Hz, 2H), 6.82 (d, J=8.68 Hz, 2H), 3.78 (s, 3H). [M+H]=192.9.

### Intermediate 87. 4-(4-Fluoro-1-isopropyl-1H-pyrazol-3-yl)phenol

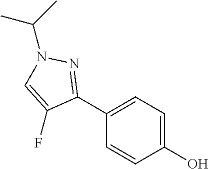 [SMILES: CC(C)n1cc(F)c(-c2ccc(O)cc2)n1]

The title compound was made in a manner analogous to Intermediate 45, using the appropriate starting material substitutions. ¹H NMR (400 MHz, DMSO-d₆) δ 9.55 (br s, 1H), 7.96 (d, J=4.52 Hz, 1H), 7.55 (d, J=8.44 Hz, 2H), 6.82 (d, J=8.56 Hz, 2H), 4.40 (spt, J=6.68 Hz, 1H), 1.41 (d, J=6.72 Hz, 6H). [M+H]=221.1.

### Intermediate 88. 5-(4-Hydroxyphenyl)-1-methyl-1H-pyrazole-4-carbonitrile

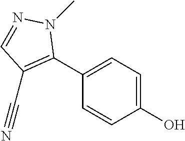

The title compound was made in a manner analogous to Intermediate 35, using the appropriate starting material substitutions. ¹H NMR (400 MHz, CDCl₃) δ 7.84 (s, 1H), 7.40 (d, J=8.68 Hz, 2H), 7.02 (d, J=8.68 Hz, 2H), 5.48 (br s, 1H), 3.91 (s, 3H).

### Intermediate 89. 4-(1-Isopropyl-1H-pyrazol-5-yl)phenol

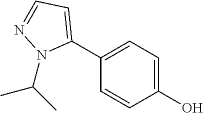

The title compound was made in a manner analogous to Intermediate 35, using the appropriate starting material substitutions. ¹H NMR (400 MHz, CDCl₃) δ 7.41 (d, J=8.93 Hz, 2H), 7.28 (s, 1H), 6.99 (d, J=8.93 Hz, 2H), 6.40 (s, 1H), 3.88 (s, 3H), 2.35 (s, 3H). [M+H]=203.

### Example 1. 6-(4-(Allyloxy)benzyl)-1-isopropyl-5-methyl-1H-pyrazolo[3,4-d]pyrimidin-4(5H)-one (or 5-methyl-6-{[4-(prop-2-en-1-yloxy)phenyl]methyl}-1-(propan-2-yl)-1H,4H,5H-pyrazolo[3,4-d]pyrimidin-4-one)

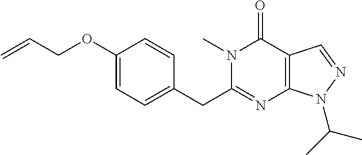 [SMILES: C=CCOc1ccc(Cc2nc3c(cnn3C(C)C)c(=O)n2C)cc1]

6-(4-Hydroxybenzyl)-1-isopropyl-1H-pyrazolo[3,4-d]pyrimidin-4(5H)-one (Intermediate 1, 30 mg, 0.10 mmol), potassium carbonate (42 mg, 0.30 mmol), DMF (1 mL) and allyl bromide (0.0017 mL, 0.020 mmol) were combined and heated at 60° C. for 2 days. The mixture was filtered and purified by prep HPLC (elution with 30-100% ACN in water) to afford the title compound (11 mg, 32%) as a white powder. ¹H NMR (400 MHz, CDCl₃) δ 8.08 (s, 1H), 7.15 (d, J=8.7 Hz, 2H), 6.93-6.88 (m, 2H), 6.11-6.01 (m, 1H), 5.42 (qd, J=1.6, 17.3 Hz, 1H), 5.31 (dd, J=1.4, 10.5 Hz, 1H), 5.07 (quin, J=6.7 Hz, 1H), 4.54 (td, J=1.4, 5.3 Hz, 2H), 4.17 (s, 2H), 3.49 (s, 3H), 1.58 (d, J=6.8 Hz, 6H). [M+H]=339.0.

### Example 2. 1-Isopropyl-6-(6-methoxy-1-methyl-3,4-dihydroisoquinolin-2(1H)-yl)-5-methyl-1H-pyrazolo[3,4-d]pyrimidin-4(5H)-one (or 6-(6-methoxy-1-methyl-1,2,3,4-tetrahydroisoquinolin-2-yl)-5-methyl-1-(propan-2-yl)-1H,4H,5H-pyrazolo[3,4-d]pyrimidin-4-one)

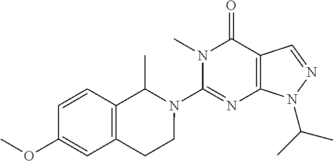 [SMILES: COc1ccc2c(c1)CCN(c1nc3c(cnn3C(C)C)c(=O)n1C)C2C]

6-Chloro-1-isopropyl-5-methyl-1H-pyrazolo[3,4-d]pyrimidin-4(5H)-one (Intermediate 2, 50 mg, 0.22 mmol) was dissolved in DMA (1 mL) and 6-methoxy-1-methyl-1,2,3,4-tetrahydroisoquinoline (59 mg, 0.33 mmol) was added. The mixture was stirred at 100° C. for 5 h. The solution was cooled, filtered and purified by preparative HPLC (elution with 20-85% ACN in water). Fractions containing product were lyophilized to afford 28 mg (35%) of the title compound as a white powder. ¹H NMR (400 MHz, CDCl₃) δ 8.02 (s, 1H), 7.05 (d, J=8.5 Hz, 1H), 6.79 (dd, J=2.6, 8.5 Hz, 1H), 6.69 (d, J=2.4 Hz, 1H), 4.98-4.77 (m, 2H), 3.86-3.75 (m, 3H), 3.75-3.66 (m, 1H), 3.66-3.49 (m, 4H), 3.24 (ddd, J=6.1, 10.8, 16.7 Hz, 1H), 2.85 (td, J=3.2, 16.4 Hz, 1H), 1.57-1.46 (m, 9H). [M+H]=368.21.

### Example 3. 1-Isopropyl-6-((1-(2-methoxyethyl)-1H-indol-5-yl)oxy)-5-methyl-1H-pyrazolo[3,4-d]pyrimidin-4(5H)-one (or 6-{[1-(2-methoxyethyl)-1H-indol-5-yl]oxy}-5-methyl-1-(propan-2-yl)-1H,4H,5H-pyrazolo[3,4-d]pyrimidin-4-one)

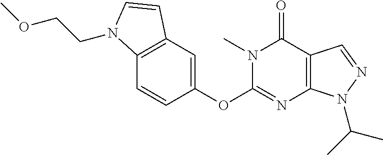 [SMILES: COCCn1ccc2cc(Oc3nc4c(cnn4C(C)C)c(=O)n3C)ccc21]

6-Chloro-1-isopropyl-5-methyl-1H-pyrazolo[3,4-d]pyrimidin-4(5H)-one (Intermediate 2, 50 mg, 0.22 mmol), potassium carbonate (61 mg, 0.44 mmol), and DMA (1 mL) were combined, then 1-(2-methoxyethyl)-1H-indol-5-ol (63 mg, 0.33 mmol) was added and the mixture was stirred at 100° C. for 5 h. The solution was cooled, filtered and purified by preparative HPLC (elution with 20-85% ACN in water). Fractions containing product were lyophilized to afford the title compound as a white powder. ¹H NMR (400 MHz, CDCl₃) δ 8.02 (s, 1H), 7.50-7.34 (m, 2H), 7.28-7.26 (m, 1H), 7.06 (dd, J=2.2, 8.9 Hz, 1H), 6.55 (d, J=2.8 Hz, 1H), 4.59 (spt, J=6.7 Hz, 1H), 4.34 (t, J=5.4 Hz, 2H), 3.77 (t, J=5.5 Hz, 2H), 3.69 (s, 3H), 3.36 (s, 3H), 1.35 (d, J=6.7 Hz, 6H). [M+H]=382.24.

### Example 4. 6-(4-(Cyclopropylmethoxy)phenoxy)-5-methyl-1-(tetrahydro-2H-pyran-4-yl)-1H-pyrazolo[3,4-d]pyrimidin-4(5H)-one (or 6-[4-(cyclopropylmethoxy)phenoxy]-5-methyl-1-(oxan-4-yl)-1H,4H,5H-pyrazolo[3,4-d]pyrimidin-4-one)

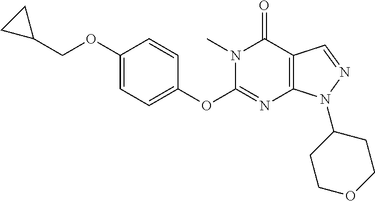 [SMILES: Cn1c(Oc2ccc(OCC3CC3)cc2)nc2c(cnn2C2CCOCC2)c1=O]

The title compound was prepared in a manner analogous to Example 3 using the appropriate starting material substitutions. ¹H NMR (400 MHz, DMSO-d₆) δ 8.02 (s, 1H), 7.28 (d, J=9.16 Hz, 2H), 7.01 (d, J=9.16 Hz, 2H), 4.29-4.41 (m, 1H), 3.82-3.93 (m, 4H), 3.50 (s, 3H), 3.33-3.41 (m, 2H), 1.94-2.06 (m, 2H), 1.70-1.78 (m, 2H), 1.20-1.30 (m, 1H), 0.56-0.62 (m, 2H), 0.31-0.37 (m, 2H). [M+H]=397.19.

### Example 5. 6-(4-(3-Methoxyphenyl)piperazin-1-yl)-5-methyl-1-(tetrahydro-2H-pyran-4-yl)-1H-pyrazolo[3,4-d]pyrimidin-4(5H)-one (or 6-[4-(3-methoxyphenyl)piperazin-1-yl]-5-methyl-1-(oxan-4-yl)-1H,4H,5H-pyrazolo[3,4-d]pyrimidin-4-one)

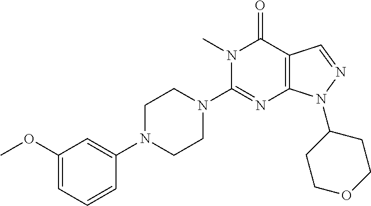 [SMILES: COc1cccc(N2CCN(c3nc4c(cnn4C4CCOCC4)c(=O)n3C)CC2)c1]

The title compound was prepared in a manner analogous to Example 2 using Intermediate 3 and the appropriate starting material substitutions. ¹H NMR (400 MHz, CDCl₃) δ 8.03 (s, 1H), 7.29-7.33 (m, 1H), 6.70-6.81 (m, 2H), 6.64 (d, J=8.07 Hz, 1H), 4.69-4.80 (m, 1H), 4.17 (dd, J=3.36, 11.55 Hz, 2H), 3.85 (s, 3H), 3.59-3.66 (m, 5H), 3.52-3.58 (m, 4H), 3.44-3.50 (m, 4H), 2.42 (dq, J=4.40, 12.31 Hz, 2H), 1.95 (dd, J=2.08, 12.59 Hz, 2H). [M+H]=425.28.

### Example 6. 5-Methyl-1-(tetrahydro-2H-pyran-3-yl)-6-(3-(2,2,2-trifluoroethoxy)phenoxy)-1H-pyrazolo[3,4-d]pyrimidin-4(5H)-one (or 5-methyl-1-(oxan-3-yl)-6-[3-(2,2,2-trifluoroethoxy)phenoxy]-1H,4H,5H-pyrazolo[3,4-d]pyrimidin-4-one)

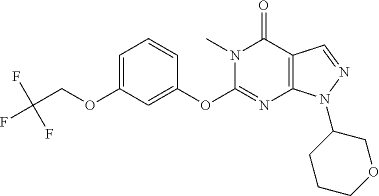 [SMILES: Cn1c(Oc2cccc(OCC(F)(F)F)c2)nc2c(cnn2C2CCCOC2)c1=O]

The title compound was prepared in a manner analogous to Example 3 using the appropriate starting material substitutions. ¹HNMR (400 MHz, CDCl₃) δ 8.01 (s, 1H), 7.44 (t, J=8.3 Hz, 1H), 6.96 (d, J=2.2 Hz, 1H), 6.94 (d, J=2.2 Hz, 1H), 6.91-6.88 (m, 1H), 4.47-4.39 (m, 3H), 3.94-3.86 (m, 2H), 3.70 (t, J=10.6 Hz, 1H), 3.66 (s, 3H), 3.42-3.33 (m, 1H), 2.21 (dd, J=5.4, 12.6 Hz, 1H), 2.13-1.98 (m, 2H), 1.81-1.69 (m, 2H). [M+H]=425.19.

### Example 7. (R)-6-([1,1′-Biphenyl]-4-ylamino)-5-methyl-1-(tetrahydro-2H-pyran-3-yl)-1,5-dihydro-4H-pyrazolo[3,4-d]pyrimidin-4-one (or 5-methyl-1-[(3R)-oxan-3-yl]-6-[(4-phenylphenyl)amino]-1H,4H,5H-pyrazolo[3,4-d]pyrimidin-4-one)

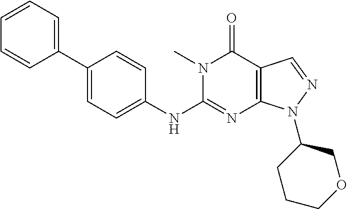 [SMILES: Cn1c(Nc2ccc(-c3ccccc3)cc2)nc2c(cnn2[C@@H]2CCCOC2)c1=O]

The title compound was prepared in a manner analogous Example 2, using the appropriate starting material substitutions. [M+H]=402.35.

### Example 8. (R)-6-(4-(1-(2-Fluoroethyl)-1H-pyrazol-4-yl)phenoxy)-5-methyl-1-(tetrahydro-2H-pyran-3-yl)-1H-pyrazolo[3,4-d]pyrimidin-4(5H)-one (6-{4-[1-(2-fluoroethyl)-1H-pyrazol-4-yl]phenoxy}-5-methyl-1-[(3R)-oxan-3-yl]-1H,4H,5H-pyrazolo[3,4-d]pyrimidin-4-one)

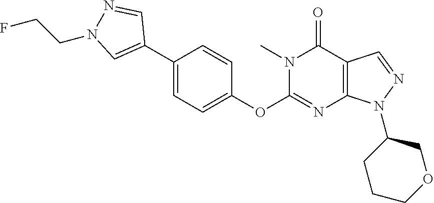 [SMILES: Cn1c(Oc2ccc(-c3cnn(CCF)c3)cc2)nc2c(cnn2[C@@H]2CCCOC2)c1=O]

The title compound was prepared in a manner analogous to Example 3 using the appropriate starting material substitutions. [M+H]=439.49.

### Example 9. 1-Isopropyl-5-methyl-6-((6-(2-oxoindolin-5-yl)pyridin-3-yl)oxy)-1,5-dihydro-4H-pyrazolo[3,4-d]pyrimidin-4-one (or 5-(5-{[5-methyl-4-oxo-1-(propan-2-yl)-1H,4H,5H-pyrazolo[3,4-d]pyrimidin-6-yl]oxy}pyridin-2-yl)-2,3-dihydro-1H-indol-2-one)

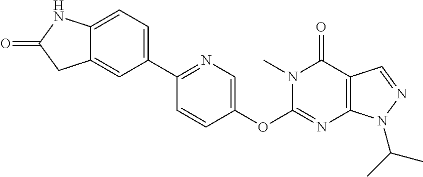 [SMILES: CC(C)n1ncc2c(=O)n(C)c(Oc3ccc(-c4ccc5c(c4)CC(=O)N5)nc3)nc21]

6-((6-Chloropyridin-3-yl)oxy)-1-isopropyl-5-methyl-1,5-dihydro-4H-pyrazolo[3,4-d]pyrimidin-4-one (26 mg, 0.081 mmol), and [1,1′-bis(diphenylphosphino)ferrocene]dichloropalladium(II) (3.0 mg, 0.004 mmol) were combined in a microwave vial. ACN (0.81 mL) and an aqueous solution of sodium bicarbonate (0.21 mL, 1.2 M, 0.24 mol) were added followed by 5-(4,4,5,5-tetramethyl-1,3,2-dioxaborolan-2-yl)indolin-2-one (25 mg, 0.096 mmol). The mixture was heated at 120° C. for 30 min, filtered, diluted with DMA and purified by preparative HPLC (elution with 20-70% ACN in water). Fractions containing product were lyophilized to afford the title compound (16 mg, 90%) as an off-white solid. ¹H NMR (400 MHz, CDCl₃) δ 8.87-8.83 (m, 1H), 8.64 (br s, 1H), 8.06 (s, 1H), 7.96-7.82 (m, 4H), 7.08 (d, J=8.2 Hz, 1H), 4.65 (td, J=6.7, 13.4 Hz, 1H), 3.77-3.62 (m, 5H), 1.41 (d, J=6.7 Hz, 6H). [M+H]=417.20.

### Example 10. 1-Isopropyl-6-(4-(1-isopropyl-1H-pyrazol-4-yl)phenoxy)-5-methyl-1,5-dihydro-4H-pyrazolo[3,4-d]pyrimidin-4-one (or 5-methyl-1-(propan-2-yl)-6-{4-[1-(propan-2-yl)-1H-pyrazol-4-yl]phenoxy}-1H,4H,5H-pyrazolo[3,4-d]pyrimidin-4-one)

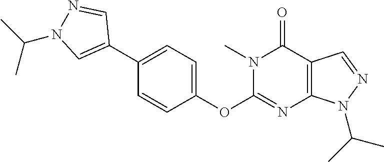 [SMILES: CC(C)n1cc(-c2ccc(Oc3nc4c(cnn4C(C)C)c(=O)n3C)cc2)cn1]

The title compound was prepared in a manner analogous to Example 9 using the appropriate starting material substitutions. ¹H NMR (400 MHz, CDCl₃) δ 7.76 (s, 1H), 7.66 (s, 1H), 7.37 (d, J=8.6 Hz, 2H), 6.86 (d, J=8.6 Hz, 2H), 5.69-4.97 (m, 1H), 4.85 (t, J=4.7 Hz, 1H), 4.74 (t, J=4.7 Hz, 1H), 4.48 (t, J=4.7 Hz, 1H), 4.42 (t, J=4.7 Hz, 1H). [M+H]=393.27.

### Example 11. 6-(4-(1-(2-Fluoroethyl)-1H-pyrazol-4-yl)phenoxy)-1-isopropyl-5-methyl-1,5-dihydro-4H-pyrazolo[3,4-d]pyrimidin-4-one (or 6-{4-[1-(2-fluoroethyl)-1H-pyrazol-4-yl]phenoxy}-5-methyl-1-(propan-2-yl)-1H,4H,5H-pyrazolo[3,4-d]pyrimidin-4-one)

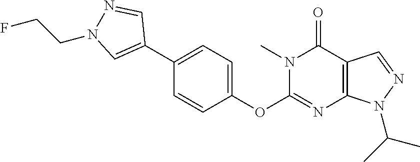 [SMILES: CC(C)n1ncc2c(=O)n(C)c(Oc3ccc(-c4cnn(CCF)c4)cc3)nc21]

The title compound was prepared in a manner analogous to Example 9 using the appropriate starting material substitutions. ¹H NMR (400 MHz, CDCl₃) δ 8.00 (s, 1H), 7.86 (s, 1H), 7.79 (s, 1H), 7.60-7.54 (m, 2H), 7.26-7.21 (m, 2H), 4.88 (t, J=4.6 Hz, 1H), 4.76 (t, J=4.6 Hz, 1H), 4.62 (td, J=6.7, 13.4 Hz, 1H), 4.52 (t, J=4.6 Hz, 1H), 4.45 (t, J=4.6 Hz, 1H), 3.66 (s, 3H), 1.39 (d, J=6.7 Hz, 6H). [M+H]=397.30.

### Example 12. 6-(4-(1-Isopropyl-1H-pyrazol-4-yl)phenoxy)-5-methyl-1-(tetrahydro-2H-pyran-4-yl)-1,5-dihydro-4H-pyrazolo[3,4-d]pyrimidin-4-one (or 5-methyl-1-(oxan-4-yl)-6-{4-[1-(propan-2-yl)-1H-pyrazol-4-yl]phenoxy}-1H,4H,5H-pyrazolo[3,4-d]pyrimidin-4-one)

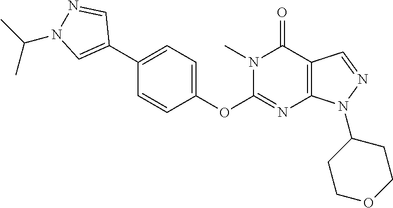 [SMILES: CC(C)n1cc(-c2ccc(Oc3nc4c(cnn4C4CCOCC4)c(=O)n3C)cc2)cn1]

The title compound was prepared in a manner analogous to Example 9 using the appropriate starting material substitutions. ¹H NMR (400 MHz, CDCl₃) δ 8.02 (s, 1H), 7.83 (s, 1H), 7.73 (s, 1H), 7.58 (d, J=8.4 Hz, 2H), 7.23 (d, J=8.4 Hz, 2H), 4.57 (td, J=6.6, 13.4 Hz, 1H), 4.50-4.36 (m, 1H), 4.02 (d, J=8.9 Hz, 2H), 3.67 (s, 3H), 3.45 (t, J=11.8 Hz, 2H), 2.23 (dq, J=4.5, 12.2 Hz, 2H), 1.85-1.75 (m, 2H), 1.59 (d, J=6.7 Hz, 6H). [M+H]=435.41.

### Example 13. 6-(4-(1-Cyclopropyl-1H-pyrazol-4-yl)phenoxy)-5-methyl-1-(tetrahydro-2H-pyran-4-yl)-1,5-dihydro-4H-pyrazolo[3,4-d]pyrimidin-4-one (or 6-[4-(1-cyclopropyl-1H-pyrazol-4-yl)phenoxy]-5-methyl-1-(oxan-4-yl)-1H,4H,5H-pyrazolo[3,4-d]pyrimidin-4-one)

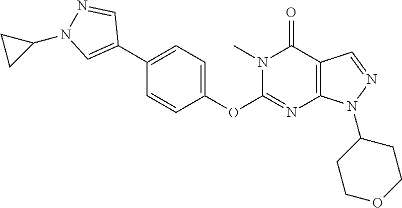 [SMILES: Cn1c(Oc2ccc(-c3cnn(C4CC4)c3)cc2)nc2c(cnn2C2CCOCC2)c1=O]

The title compound was prepared in a manner analogous to Example 9 using the appropriate starting material substitutions. ¹H NMR (400 MHz, CDCl₃) δ 8.01 (s, 1H), 7.79 (s, 1H), 7.76 (s, 1H), 7.58-7.53 (m, 2H), 7.25-7.20 (m, 2H), 4.42 (tt, J=4.2, 11.4 Hz, 1H), 4.08-3.94 (m, 2H), 3.72-3.60 (m, 4H), 3.50-3.37 (m, 2H), 2.23 (dq, J=4.5, 12.2 Hz, 2H), 1.79 (dd, J=2.2, 12.6 Hz, 2H), 1.24-1.02, (m, 4H). [M+H]=433.43.

### Example 14. 6-(2-Fluoro-4-(1-isopropyl-1H-pyrazol-4-yl)phenoxy)-1-isopropyl-5-methyl-1,5-dihydro-4H-pyrazolo[3,4-d]pyrimidin-4-one (or 6-{2-fluoro-4-[1-(propan-2-yl)-1H-pyrazol-4-yl]phenoxy}-5-methyl-1-(propan-2-yl)-1H,4H,5H-pyrazolo[3,4-d]pyrimidin-4-one)

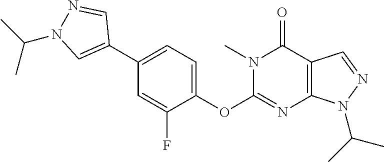 [SMILES: CC(C)n1cc(-c2ccc(Oc3nc4c(cnn4C(C)C)c(=O)n3C)c(F)c2)cn1]

The title compound was prepared in a manner analogous to Example 9 using the appropriate starting material substitutions. ¹H NMR (400 MHz, CDCl₃) δ 8.03 (s, 1H), 7.88 (s, 1H), 7.75 (s, 1H), 7.38-7.27 (m, 3H), 4.60 (sxtd, J=6.8, 14.0 Hz, 2H), 3.69 (s, 3H), 1.59 (d, J=6.6 Hz, 6H), 1.38 (d, J=6.7 Hz, 6H). [M+H]=411.37.

### Example 15. 6-(3-Fluoro-4-(1-isopropyl-1H-pyrazol-4-yl)phenoxy)-1-isopropyl-5-methyl-1,5-dihydro-4H-pyrazolo[3,4-d]pyrimidin-4-one (or 6-{3-fluoro-4-[1-(propan-2-yl)-1H-pyrazol-4-yl]phenoxy}-5-methyl-1-(propan-2-yl)-1H,4H,5H-pyrazolo[3,4-d]pyrimidin-4-one)

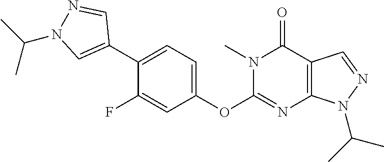 [SMILES: CC(C)n1cc(-c2ccc(Oc3nc4c(cnn4C(C)C)c(=O)n3C)cc2F)cn1]

The title compound was prepared in a manner analogous to Example 9, using the appropriate starting material substitutions. ¹H NMR (400 MHz, CDCl₃) δ 8.02 (s, 1H), 7.94 (s, 1H), 7.89 (d, J=2.3 Hz, 1H), 7.70-7.58 (m, 1H), 7.14-7.03 (m, 2H), 4.70-4.53 (m, 2H), 3.71-3.60 (m, 3H), 1.59 (d, J=6.7 Hz, 6H), 1.40 (d, J=6.7 Hz, 6H). [M+H]=411.37.

### Example 16. 6-(4-(1-Ethyl-1H-pyrazol-4-yl)-2,3-difluorophenoxy)-1-isopropyl-5-methyl-1,5-dihydro-4H-pyrazolo[3,4-d]pyrimidin-4-one (6-[4-(1-ethyl-1H-pyrazol-4-yl)-2,3-difluorophenoxy]-5-methyl-1-(propan-2-yl)-1H,4H,5H-pyrazolo[3,4-d]pyrimidin-4-one)

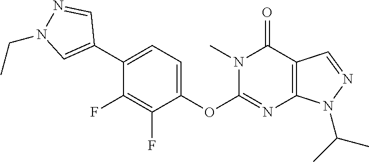 [SMILES: CCn1cc(-c2ccc(Oc3nc4c(cnn4C(C)C)c(=O)n3C)c(F)c2F)cn1]

The title compound was prepared in a manner analogous to Example 9, using the appropriate starting material substitutions. ¹H NMR (400 MHz, CDCl₃) δ 8.02 (s, 1H), 7.93 (s, 1H), 7.89 (d, J=2.1 Hz, 1H), 7.39 (dt, J=2.2, 8.1 Hz, 1H), 7.10 (ddd, J=1.9, 6.9, 8.8 Hz, 1H), 4.60 (spt, J=6.7 Hz, 1H), 4.29 (q, J=7.3 Hz, 2H), 3.68 (s, 3H), 1.58 (t, J=7.3 Hz, 3H), 1.39 (d, J=6.7 Hz, 6H). [M+H]=415.34.

### Example 17. 6-(4-(1,3-Dimethyl-1H-pyrazol-4-yl)-2,6-difluorophenoxy)-1-isopropyl-5-methyl-1,5-dihydro-4H-pyrazolo[3,4-d]pyrimidin-4-one (or 6-[4-(1,3-dimethyl-1H-pyrazol-4-yl)-2,6-difluorophenoxy]-5-methyl-1-(propan-2-yl)-1H,4H,5H-pyrazolo[3,4-d]pyrimidin-4-one)

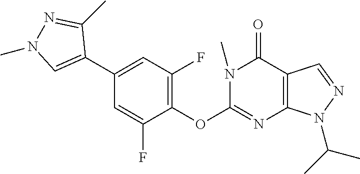 [SMILES: Cc1nn(C)cc1-c1cc(F)c(Oc2nc3c(cnn3C(C)C)c(=O)n2C)c(F)c1]

The title compound was prepared in a manner analogous to Example 9, using the appropriate starting material substitutions. ¹H NMR (400 MHz, CDCl₃) δ 8.04 (s, 1H), 7.56 (s, 1H), 7.10 (d, J=8.7 Hz, 2H), 4.61 (spt, J=6.7 Hz, 1H), 3.96 (s, 3H), 3.72 (s, 3H), 2.48 (s, 3H), 1.40 (d, J=6.8 Hz, 6H). [M+H]=415.33.

### Example 18. 6-(4-(1,3-Dimethyl-1H-pyrazol-4-yl)-2,5-difluorophenoxy)-1-isopropyl-5-methyl-1,5-dihydro-4H-pyrazolo[3,4-d]pyrimidin-4-one (or 6-[4-(1,3-dimethyl-1H-pyrazol-4-yl)-2,5-difluorophenoxy]-5-methyl-1-(propan-2-yl)-1H,4H,5H-pyrazolo[3,4-d]pyrimidin-4-one)

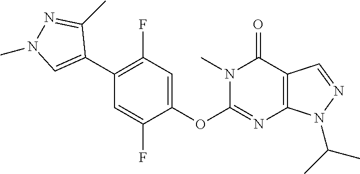 [SMILES: Cc1nn(C)cc1-c1cc(F)c(Oc2nc3c(cnn3C(C)C)c(=O)n2C)cc1F]

The title compound was prepared in a manner analogous to Example 9, using the appropriate starting material substitutions. ¹H NMR (400 MHz, CDCl₃) δ 8.04 (s, 1H), 7.65 (s, 1H), 7.19 (dd, J=6.5, 9.8 Hz, 1H), 4.63 (quin, J=6.7 Hz, 1H), 3.98 (s, 3H), 3.69 (s, 3H), 2.44 (s, 3H), 1.42 (d, J=6.7 Hz, 6H). [M+H]=415.36.

### Example 19. 6-(4-(1-Acetylpiperidin-4-yl)phenoxy)-1-isopropyl-5-methyl-1,5-dihydro-4H-pyrazolo[3,4-d]pyrimidin-4-one (or 6-[4-(1-acetylpiperidin-4-yl)phenoxy]-5-methyl-1-(propan-2-yl)-1H,4H,5H-pyrazolo[3,4-d]pyrimidin-4-one)

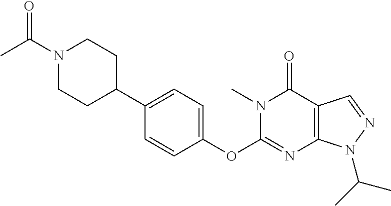 [SMILES: CC(=O)N1CCC(c2ccc(Oc3nc4c(cnn4C(C)C)c(=O)n3C)cc2)CC1]

To a solution of 1-isopropyl-5-methyl-6-(4-(piperidin-4-yl)phenoxy)-1,5-dihydro-4H-pyrazolo[3,4-d]pyrimidin-4-one (Intermediate 17, 48 mg, 0.12 mmol) in DMF (1.3 mL) was added DIEA (0.11 mL, 0.65 mmol) and acetyl chloride (0.019 mL, 0.26 mmol) and the reaction was stirred for 1 h. The mixture was filtered and purified by preparative HPLC (elution with 20-70% ACN in water). Fractions containing product were lyophilized to afford the title compound (56 mg, 99%) as a white powder. ¹H NMR (400 MHz, CDCl₃) δ 8.02 (s, 1H), 7.32-7.27 (m, 2H), 7.23-7.17 (m, 2H), 4.84 (d, J=12.2 Hz, 1H), 4.61 (td, J=6.7, 13.4 Hz, 1H), 4.00 (d, J=13.0 Hz, 1H), 3.65 (s, 3H), 3.26 (t, J=12.9 Hz, 1H), 2.86 (tt, J=3.5, 12.1 Hz, 1H), 2.73 (t, J=12.4 Hz, 1H), 2.22 (s, 3H), 2.01 (t, J=15.0 Hz, 2H), 1.81-1.62 (m, 2H), 1.39 (d, J=6.8 Hz, 6H). [M+H]=410.24.

### Example 20. 3-Isopropyl-7-methyl-6-(4-(tetrahydro-2H-pyran-4-yl)phenoxy)imidazo[1,5-a]pyrazin-8(7H)-one (or 7-methyl-6-[4-(oxan-4-yl)phenoxy]-3-(propan-2-yl)-7H,8H-imidazo[1,5-a]pyrazin-8-one)

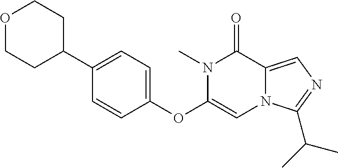 [SMILES: CC(C)c1ncc2c(=O)n(C)c(Oc3ccc(C4CCOCC4)cc3)cn12]

A mixture of 6-bromo-3-isopropyl-7-methylimidazo[1,5-a]pyrazin-8(7H)-one (220 mg, 0.81 mmol), 4-(tetrahydro-2H-pyran-4-yl)phenol (174 mg, 0.98 mmol), and Cs₂CO₃ (318 mg, 0.98 mmol) in DMF (8 mL) was heated in a Biotage microwave apparatus at 180° C. for 40 min. Then, the reaction was cooled, diluted with CH₂Cl₂, washed with water, dried (MgSO₄) and evaporated. The residue was purified (FCC, SiO₂, 10-75% acetone/heptanes) to provide the title compound (139 mg, 46%) as a white solid. ¹H NMR (400 MHz, CDCl₃) δ 7.93 (s, 1H), 7.28 (d, J=8.6 Hz, 2H), 7.06 (d, J=8.7 Hz, 2H), 6.59 (s, 1H), 4.12 (d, J=11.1 Hz, 2H), 3.61-3.51 (m, 2H), 3.43 (s, 3H), 3.03 (spt, J=6.8 Hz, 1H), 2.86-2.76 (m, 1H), 1.91-1.75 (m, 4H), 1.37 (d, J=6.8 Hz, 6H). [M+H]=368.4.

### Example 21. 3-Isopropyl-7-methyl-6-(((1R,4R)-4-(trifluoromethyl)cyclohexyl)methoxy)imidazo[1,5-a]pyrazin-8(7H)-one (or 7-methyl-3-(propan-2-yl)-6-{[(1R,4R)-4-(trifluoromethyl)cyclohexyl]methoxy}-7H,8H-imidazo[1,5-a]pyrazin-8-one)

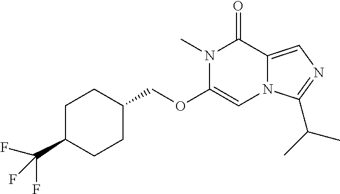 [SMILES: CC(C)c1ncc2c(=O)n(C)c(OC[C@H]3CC[C@H](C(F)(F)F)CC3)cn12]

To a solution of trans-4-(trifluoromethyl)cyclohexyl)methanol (209 mg, 1.15 mmol) in DMF (4 mL) was added sodium bis(trimethylsilyl)amide (1.15 mL, 1.0 M, 1.15 mmol) at rt and after stirring at rt for 5 min., 6-bromo-3-isopropyl-7-methylimidazo[1,5-a]pyrazin-8(7H)-one (238 mg, 0.88 mmol) was added as a solid. After stirring at rt for 2 h, the mixture was diluted with a 1 M solution of NH₄Cl, water and DCM, partitioned and the aqueous layer was extracted with DCM (2×15 mL). The combined organic extracts were dried (MgSO₄) and evaporated. The residue was purified (FCC, SiO₂, 10-100% EtOAc/heptanes) to provide the title compound (131 mg, 40%) as a white solid. ¹H NMR (400 MHz, CDCl₃) δ 7.86 (s, 1H), 6.34 (s, 1H), 3.81 (d, J=6.0 Hz, 2H), 3.42 (s, 3H), 3.14 (td, J=6.8, 13.7 Hz, 1H), 2.14-2.02 (m, 5H), 1.92 (dtt, J=3.2, 6.0, 12.0 Hz, 1H), 1.47-1.40 (m, 8H), 1.28-1.12 (m, 2H). [M+H]=372.3.

### Example 22 and 23. 6-((3-Isopropyl-7-methyl-8-oxo-7,8-dihydroimidazo[1,5-a]pyrazin-6-yl)oxy)-3,4-dihydroquinolin-2(1H)-one (or 6-{[7-methyl-8-oxo-3-(propan-2-yl)-7H,8H-imidazo[1,5-a]pyrazin-6-yl]oxy}-1,2,3,4-tetrahydroquinolin-2-one) and 6-((3-isopropyl-7-methyl-8-oxo-7,8-dihydroimidazo[1,5-a]pyrazin-6-yl)oxy)quinolin-2(1H)-one (or 6-{[7-methyl-8-oxo-3-(propan-2-yl)-7H,8H-imidazo[1,5-a]pyrazin-6-yl]oxy}-1,2-dihydroquinolin-2-one)

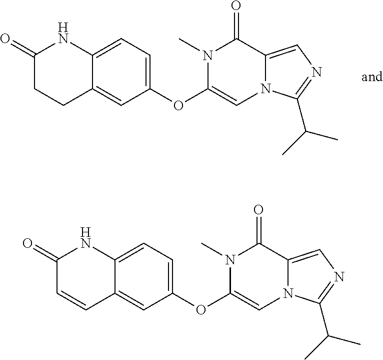 [SMILES: CC(C)c1ncc2c(=O)n(C)c(Oc3ccc4c(c3)CCC(=O)N4)cn12 ; CC(C)c1ncc2c(=O)n(C)c(Oc3ccc4[nH]c(=O)ccc4c3)cn12]

The title compound was prepared in a manner analogous to Example 20, using 6-hydroxy-3,4-dihydroquinolin-2(1H)-one (141 mg, 0.86 mmol) as the nucleophile and any appropriate reagent substitutions. The residue obtained was purified (FCC, SiO₂, 10-70% acetone/heptanes) to provide Example 22 (113 mg, 37%) as a white solid. ¹H NMR (400 MHz, DMSO-d₆) δ 10.10 (s, 1H), 7.71 (s, 1H), 7.31 (s, 1H), 7.10 (d, J=2.8 Hz, 1H), 7.02 (dd, J=2.8, 8.7 Hz, 1H), 6.88 (d, J=8.7 Hz, 1H), 3.31-3.26 (m, 1H), 3.24 (s, 3H), 2.88 (t, J=7.5 Hz, 2H), 2.48-2.40 (m, 2H), 1.23 (d, J=6.7 Hz, 6H). [M+H]=353.3. Continued elution provided Example 23 (38 mg, 13% yield) as a white solid. ¹H NMR (400 MHz, DMSO-d₆) δ 11.81 (s, 1H), 7.89 (d, J=9.7 Hz, 1H), 7.72 (s, 1H), 7.55 (d, J=2.8 Hz, 1H), 7.51-7.44 (m, 1H), 7.36 (d, J=8.9 Hz, 1H), 6.54 (dd, J=1.8, 9.5 Hz, 1H), 3.31-3.27 (m, 1H), 3.25 (s, 3H), 1.22 (d, J=6.7 Hz, 7H). [M+H]=351.3.

### Examples 24 and 25. 6-((3-Isopropyl-7-methyl-8-oxo-7,8-dihydroimidazo[1,5-a]pyrazin-6-yl)oxy)-1-(2-methoxyethyl)quinolin-2(1H)-one (or 1-(2-methoxyethyl)-6-{[7-methyl-8-oxo-3-(propan-2-yl)-7H,8H-imidazo[1,5-a]pyrazin-6-yl]oxy}-1,2-dihydroquinolin-2-one) and 3-isopropyl-6-((2-(2-methoxyethoxy)quinolin-6-yl)oxy)-7-methylimidazo[1,5-a]pyrazin-8(7H)-one (or 6-{[2-(2-methoxyethoxy)quinolin-6-yl]oxy}-7-methyl-3-(propan-2-yl)-7H,8H-imidazo[1,5-a]pyrazin-8-one)

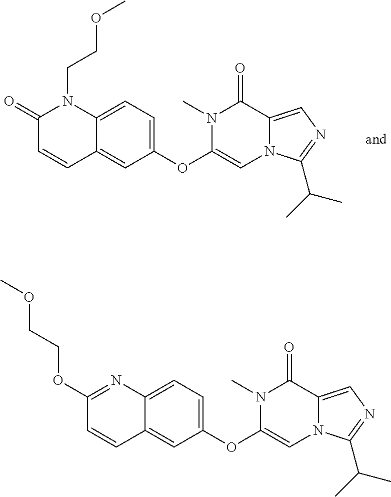

A mixture of 6-((3-isopropyl-7-methyl-8-oxo-7,8-dihydroimidazo[1,5-a]pyrazin-6-yl)oxy)quinolin-2(1H)-one (34 mg, 0.10 mmol) 2-bromoethyl methyl ether (18 μL, 0.19 mmol), K₂CO₃ (27 mg, 0.19 mmol) in DMF (1 mL) was stirred at rt for 16 h, then at 80° C. for 1 h. Then, 2-bromoethyl methyl ether (12 μL, 0.12 mmol) and Cs₂CO₃ (63 mg, 0.19 mmol) were added and the mixture stirred at 80° C. for 3 h. Then, the mixture was filtered and purified using preparative HPLC performed on a Waters Fractionlynx system equipped with a XBridge Prep C₁₈ OBD column (5 μm, 30×100 mm) and the solvent system: 15-40% acetonitrile in H₂O with 0.1% TFA gradient over a 9 min run time with a flow rate of 60 mL/min to provide fractions containing Example 24, which were diluted with CH₂Cl₂ and washed with aqueous K₂CO₃, dried (MgSO₄) and evaporated to provide the title compound (12 mg, 30%) as a white solid. ¹H NMR (400 MHz, CDCl₃) δ 7.96 (s, 1H), 7.68 (d, J=9.3 Hz, 1H), 7.65 (d, J=9.7 Hz, 1H), 7.35 (dd, J=2.8, 9.3 Hz, 1H), 7.27 (d, J=2.8 Hz, 1H), 6.80 (d, J=9.4 Hz, 1H), 6.54 (s, 1H), 4.52 (t, J=5.6 Hz, 2H), 3.80 (t, J=5.6 Hz, 2H), 3.48 (s, 3H), 3.37 (s, 3H), 3.03 (spt, J=6.9 Hz, 1H), 1.37 (d, J=6.8 Hz, 6H). [M+H]=409.4. Fractions containing Example 25 were treated similarly to provide the title compound (11 mg, 28%) as a white solid. ¹H NMR (400 MHz, CDCl₃) δ 7.96 (s, 1H), 7.95 (d, J=8.8 Hz, 1H), 7.91 (d, J=9.0 Hz, 1H), 7.45 (dd, J=2.8, 9.0 Hz, 1H), 7.37 (d, J=2.7 Hz, 1H), 7.06 (d, J=8.8 Hz, 1H), 6.54 (s, 1H), 4.72-4.65 (m, 2H), 3.87-3.81 (m, 2H), 3.49 (d, J=3.2 Hz, 6H), 3.01 (spt, J=6.9 Hz, 1H), 1.35 (d, J=7.0 Hz, 6H). [M+H]=409.4.

Example 26-Example 27 were prepared in a manner analogous to Example 1, with the appropriate starting material substitutions.

### Example 26. 6-{[4-(Cyclopropylmethoxy)phenyl]methyl}-5-methyl-1-(propan-2-yl)-1H,4H,5H-pyrazolo[3,4-d]pyrimidin-4-one

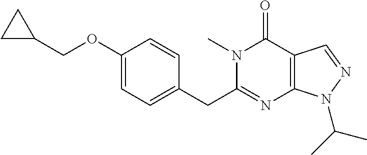 [SMILES: CC(C)n1ncc2c(=O)n(C)c(Cc3ccc(OCC4CC4)cc3)nc21]

¹H NMR (400 MHz, CDCl₃) δ 8.08 (s, 1H), 7.14 (d, J=8.7 Hz, 2H), 6.91-6.86 (m, 2H), 5.12-5.03 (m, 1H), 4.17 (s, 2H), 3.80 (d, J=6.9 Hz, 2H), 3.48 (s, 3H), 1.58 (d, J=6.8 Hz, 7H), 1.33-1.23 (m, 1H), 0.69-0.63 (m, 2H), 0.38-0.33 (m, 2H). [M+H]=353.0.

### Example 27. 5-Methyl-6-{[4-(oxan-4-yloxy)phenyl]methyl}-1-(propan-2-yl)-1H,4H,5H-pyrazolo[3,4-d]pyrimidin-4-one

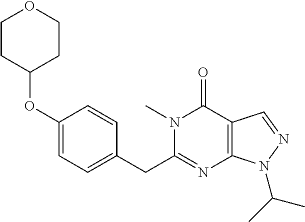 [SMILES: CC(C)n1ncc2c(=O)n(C)c(Cc3ccc(OC4CCOCC4)cc3)nc21]

¹H NMR (400 MHz, CDCl₃) δ 8.07 (s, 1H), 7.14 (d, J=8.5 Hz, 2H), 6.89 (d, J=8.8 Hz, 2H), 5.06 (td, J=6.7, 13.4 Hz, 1H), 4.47 (tt, J=3.8, 7.8 Hz, 1H), 4.16 (s, 2H), 4.03-3.93 (m, 2H), 3.57 (ddd, J=3.1, 8.3, 11.6 Hz, 2H), 3.48 (s, 3H), 2.06-1.96 (m, 2H), 1.85-1.73 (m, 2H), 1.57 (d, J=6.5 Hz, 6H). [M+H]=383.2.

Example 28-Example 34 were prepared in a manner analogous to Example 2, with the appropriate starting material substitutions.

### Example 28. 6-(7-Methoxy-1,2,3,4-tetrahydroisoquinolin-2-yl)-5-methyl-1-(propan-2-yl)-1H,4H,5H-pyrazolo[3,4-d]pyrimidin-4-one

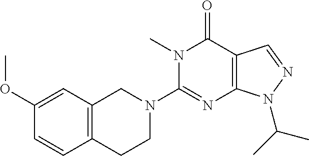 [SMILES: COc1ccc2c(c1)CN(c1nc3c(cnn3C(C)C)c(=O)n1C)CC2]

¹H NMR (400 MHz, CDCl₃) δ 8.04 (s, 1H), 7.12 (d, J=8.4 Hz, 1H), 6.81 (dd, J=2.4, 8.4 Hz, 1H), 6.71 (d, J=2.3 Hz, 1H), 4.98 (spt, J=6.8 Hz, 1H), 4.45 (s, 2H), 3.82 (s, 3H), 3.60 (s, 3H), 3.51 (t, J=5.9 Hz, 2H), 3.04 (t, J=5.7 Hz, 2H), 1.55 (d, J=6.8 Hz, 6H). [M+H]=354.22.

### Example 29. 6-[4-(2-Methoxyphenyl)piperazin-1-yl]-5-methyl-1-(propan-2-yl)-1H,4H,5H-pyrazolo[3,4-d]pyrimidin-4-one

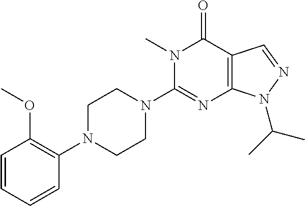 [SMILES: COc1ccccc1N1CCN(c2nc3c(cnn3C(C)C)c(=O)n2C)CC1]

¹H NMR (400 MHz, CDCl₃) δ 8.06 (s, 1H), 7.59 (d, J=7.82 Hz, 1H), 7.35-7.43 (m, 1H), 7.04-7.13 (m, 2H), 4.90-5.02 (m, 1H), 3.95 (s, 3H), 3.79 (d, J=6.11 Hz, 8H), 3.60 (s, 3H), 1.57 (d, J=6.72 Hz, 6H). [M+H]=383.27.

### Example 30. 6-[4-(3-Methoxyphenyl)piperazin-1-yl]-5-methyl-1-(propan-2-yl)-1H,4H,5H-pyrazolo[3,4-d]pyrimidin-4-one

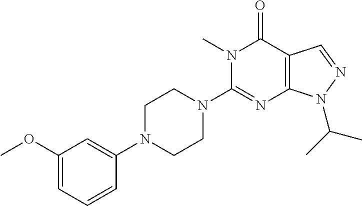 [SMILES: COc1cccc(N2CCN(c3nc4c(cnn4C(C)C)c(=O)n3C)CC2)c1]

¹H NMR (400 MHz, CDCl₃) δ 8.04 (s, 1H), 7.29-7.35 (m, 1H), 6.77-6.84 (m, 2H), 6.65-6.71 (m, 1H), 4.91-5.01 (m, 1H), 3.85 (s, 3H), 3.57-3.63 (m, 7H), 3.47-3.53 (m, 4H), 1.56 (d, J=6.72 Hz, 6H). [M+H]=383.27.

### Example 31. 5-Methyl-1-(propan-2-yl)-6-[4-(pyridin-3-yl)piperazin-1-yl]-1H,4H,5H-pyrazolo[3,4-d]pyrimidin-4-one

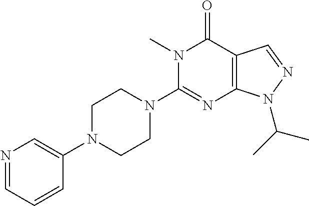 [SMILES: CC(C)n1ncc2c(=O)n(C)c(N3CCN(c4cccnc4)CC3)nc21]

¹H NMR (400 MHz, CDCl₃) δ 8.64-8.71 (m, 1H), 8.20 (d, J=4.77 Hz, 1H), 8.04 (s, 1H), 7.70-7.82 (m, 2H), 4.90-5.00 (m, 1H), 3.54-3.69 (m, 7H), 3.46-3.54 (m, 4H), 1.55 (d, J=6.72 Hz, 6H). [M+H]=354.27.

### Example 32. 5-Methyl-6-[4-(6-methylpyridin-2-yl)piperazin-1-yl]-1-(propan-2-yl)-1H,4H,5H-pyrazolo[3,4-d]pyrimidin-4-one

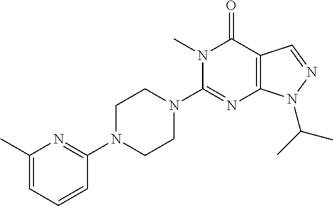 [SMILES: Cc1cccc(N2CCN(c3nc4c(cnn4C(C)C)c(=O)n3C)CC2)n1]

¹H NMR (400 MHz, CDCl₃) δ 8.03 (s, 1H), 7.84 (dd, J=7.46, 8.80 Hz, 1H), 6.86 (d, J=9.05 Hz, 1H), 6.76 (d, J=7.34 Hz, 1H), 4.88-4.98 (m, 1H), 3.93-4.01 (m, 4H), 3.58 (s, 3H), 3.47-3.56 (m, 4H), 2.71 (s, 3H), 1.54 (d, J=6.72 Hz, 6H). [M+H]=368.31.

### Example 33. 5-Methyl-6-[4-(4-methylpyridin-2-yl)piperazin-1-yl]-1-(propan-2-yl)-1H,4H,5H-pyrazolo[3,4-d]pyrimidin-4-one

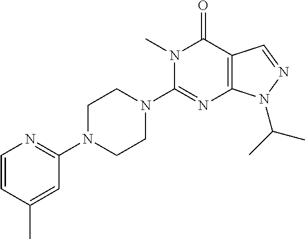 [SMILES: Cc1ccnc(N2CCN(c3nc4c(cnn4C(C)C)c(=O)n3C)CC2)c1]

¹H NMR (400 MHz, CDCl₃) δ 8.11 (d, J=6.36 Hz, 1H), 8.03 (s, 1H), 6.79-6.88 (m, 2H), 4.88-4.99 (m, 1H), 3.91-3.99 (m, 4H), 3.59 (s, 3H), 3.49-3.55 (m, 4H), 2.51 (s, 3H), 1.54 (d, J=6.72 Hz, 6H). [M+H]=368.29.

### Example 34. 6-[(7-Methoxyquinolin-3-yl)amino]-5-methyl-1-(propan-2-yl)-1H,4H,5H-pyrazolo[3,4-d]pyrimidin-4-one

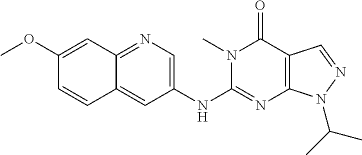 [SMILES: COc1ccc2cc(Nc3nc4c(cnn4C(C)C)c(=O)n3C)cnc2c1]

¹H NMR (400 MHz, CDCl₃) δ 9.65 (d, J=2.45 Hz, 1H) 9.13 (d, J=2.20 Hz, 1H) 9.12 (br s, 1H) 7.96 (s, 1H) 7.79 (d, J=9.17 Hz, 1H) 7.44 (d, J=2.20 Hz, 1H) 7.34 (dd, J=9.17, 2.32 Hz, 1H) 4.71 (spt, J=6.68 Hz, 1H) 4.04 (s, 3H) 3.70 (s, 3H) 1.52 (d, J=6.72 Hz, 6H). [M+H]=365.31.

Example 35-Example 77 were prepared in a manner analogous to Example 3, with the appropriate starting material substitutions.

### Example 35. 1-(2-Methoxyethyl)-6-{[5-methyl-4-oxo-1-(propan-2-yl)-1H,4H,5H-pyrazolo[3,4-d]pyrimidin-6-yl]oxy}-1,2,3,4-tetrahydroquinolin-2-one

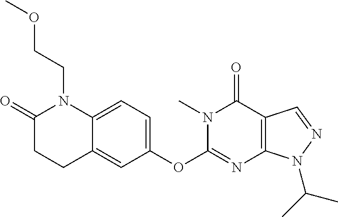 [SMILES: COCCN1C(=O)CCc2cc(Oc3nc4c(cnn4C(C)C)c(=O)n3C)ccc21]

¹H NMR (400 MHz, CDCl₃) δ 8.05 (s, 1H) 7.34 (d, J=8.80 Hz, 1H) 7.13 (dd, J=8.86, 2.63 Hz, 1H) 7.00-7.08 (m, 1H) 4.65 (spt, J=6.74 Hz, 1H) 4.17 (t, J=5.69 Hz, 2H) 3.72 (t, J=5.69 Hz, 2H) 3.65 (s, 3H) 3.40 (s, 3H) 2.93-2.99 (m, 2H) 2.70-2.80 (m, 2H) 1.41 (d, J=6.72 Hz, 6H). [M+H]=412.25.

### Example 36. 6-[4-(2,4-Dimethoxyphenyl)phenoxy]-5-methyl-1-(propan-2-yl)-1H,4H,5H-pyrazolo[3,4-d]pyrimidin-4-one

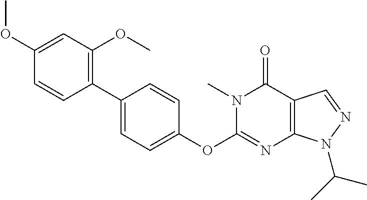 [SMILES: COc1ccc(-c2ccc(Oc3nc4c(cnn4C(C)C)c(=O)n3C)cc2)c(OC)c1]

¹H NMR (400 MHz, CDCl₃) δ 8.02 (s, 1H), 7.60 (d, J=8.7 Hz, 2H), 7.31 (d, J=8.8 Hz, 1H), 7.29-7.23 (m, 2H), 6.66-6.55 (m, 2H), 4.68 (spt, J=6.7 Hz, 1H), 3.86 (d, J=13.6 Hz, 6H), 3.67 (s, 3H), 1.41 (d, J=6.7 Hz, 6H). [M+H]=421.22.

### Example 37. 5-Methyl-1-(propan-2-yl)-6-({6-[1-(propan-2-yl)-1H-pyrazol-4-yl]pyridin-3-yl}oxy)-1H,4H,5H-pyrazolo[3,4-d]pyrimidin-4-one

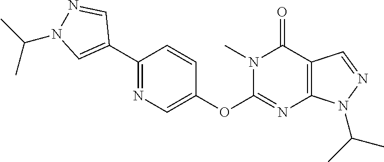 [SMILES: CC(C)n1cc(-c2ccc(Oc3nc4c(cnn4C(C)C)c(=O)n3C)cn2)cn1]

¹H NMR (400 MHz, CDCl₃) δ 8.65-8.61 (m, 1H), 8.17 (s, 1H), 8.02 (s, 2H), 7.69-7.63 (m, 2H), 4.61 (quind, J=6.7, 10.0 Hz, 2H), 3.71 (s, 3H), 1.59 (d, J=6.7 Hz, 6H), 1.40 (d, J=6.7 Hz, 6H). [M+H]=394.35.

### Example 38. 6-{[6-(1-Cyclopropyl-1H-pyrazol-4-yl)pyridin-3-yl]oxy}-5-methyl-1-(propan-2-yl)-1H,4H,5H-pyrazolo[3,4-d]pyrimidin-4-one

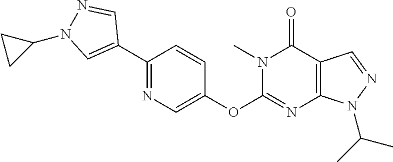 [SMILES: CC(C)n1ncc2c(=O)n(C)c(Oc3ccc(-c4cnn(C5CC5)c4)nc3)nc21]

¹H NMR (400 MHz, CDCl₃) δ 8.79 (d, J=2.2 Hz, 1H), 8.30 (s, 1H), 8.03 (s, 2H), 7.87-7.77 (m, 1H), 7.77-7.68 (m, 1H), 4.63 (td, J=6.6, 13.4 Hz, 1H), 3.78-3.62 (m, 4H), 1.40 (d, J=6.7 Hz, 6H), 1.28-1.06 (m, 4H). [M+H]=392.26.

### Example 39. 5-Methyl-1-(propan-2-yl)-6-[4-(pyridin-3-yl)phenoxy]-1H,4H,5H-pyrazolo[3,4-d]pyrimidin-4-one

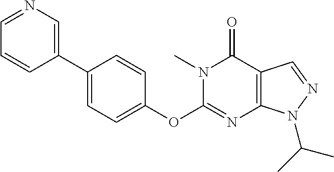 [SMILES: CC(C)n1ncc2c(=O)n(C)c(Oc3ccc(-c4cccnc4)cc3)nc21]

¹H NMR (400 MHz, CDCl₃) δ 9.18 (s, 1H), 8.76 (d, J=4.9 Hz, 1H), 8.42 (d, J=7.9 Hz, 1H), 8.03 (s, 1H), 7.85 (dd, J=5.4, 7.9 Hz, 1H), 7.75 (d, J=8.6 Hz, 2H), 7.46 (d, J=8.6 Hz, 2H), 4.63 (td, J=6.7, 13.4 Hz, 1H), 3.69 (s, 3H), 1.41 (d, J=6.7 Hz, 6H). [M+H]=362.21.

### Example 40. 6-[4-(1-Cyclopropyl-1H-pyrazol-4-yl)phenoxy]-5-methyl-1-(propan-2-yl)-1H,4H,5H-pyrazolo[3,4-d]pyrimidin-4-one

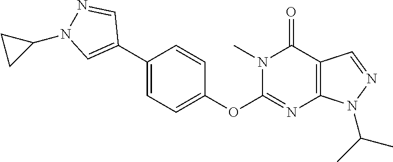 [SMILES: CC(C)n1ncc2c(=O)n(C)c(Oc3ccc(-c4cnn(C5CC5)c4)cc3)nc21]

¹H NMR (400 MHz, CDCl₃) δ 8.00 (s, 1H), 7.77 (d, J=14.3 Hz, 2H), 7.55 (d, J=8.7 Hz, 2H), 7.23 (d, J=8.7 Hz, 2H), 4.68-4.54 (m, 1H), 3.70-3.61 (m, 4H), 1.39 (d, J=6.8 Hz, 6H), 1.22-1.16 (m, 2H), 1.12-1.05 (m, 2H). [M+H]=391.37.

### Example 41. 3-[4-(4-{[5-Methyl-4-oxo-1-(propan-2-yl)-1H,4H,5H-pyrazolo[3,4-d]pyrimidin-6-yl]oxy}phenyl)-1H-pyrazol-1-yl]propanenitrile

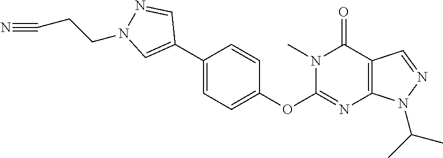 [SMILES: CC(C)n1ncc2c(=O)n(C)c(Oc3ccc(-c4cnn(CCC#N)c4)cc3)nc21]

¹H NMR (400 MHz, CDCl₃) δ 8.04 (s, 1H), 7.91 (s, 1H), 7.83 (s, 1H), 7.57 (d, J=8.6 Hz, 2H), 7.25 (d, J=8.8 Hz, 2H), 4.64 (td, J=6.8, 13.4 Hz, 1H), 4.49 (t, J=6.4 Hz, 2H), 3.67 (s, 3H), 3.02 (t, J=6.3 Hz, 2H), 1.40 (d, J=6.7 Hz, 6H). [M+H]=404.22.

### Example 42. 5-Methyl-1-(propan-2-yl)-6-[4-(pyridin-2-yl)phenoxy]-1H,4H,5H-pyrazolo[3,4-d]pyrimidin-4-one

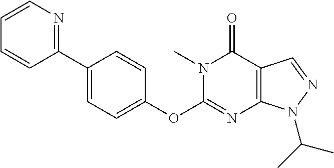 [SMILES: CC(C)n1ncc2c(=O)n(C)c(Oc3ccc(-c4ccccn4)cc3)nc21]

¹H NMR (400 MHz, CDCl₃) δ 9.04-8.95 (m, 1H), 8.18 (t, J=7.7 Hz, 1H), 8.08-8.01 (m, 3H), 7.96 (d, J=7.9 Hz, 1H), 7.68-7.60 (m, 1H), 7.47 (d, J=8.4 Hz, 2H), 4.63 (td, J=6.7, 13.4 Hz, 1H), 3.69 (s, 3H), 1.40 (d, J=6.7 Hz, 6H). [M+H]=362.19.

### Example 43. 6-[4-(1-Ethyl-3-methyl-1H-pyrazol-4-yl)phenoxy]-5-methyl-1-(propan-2-yl)-1H,4H,5H-pyrazolo[3,4-d]pyrimidin-4-one

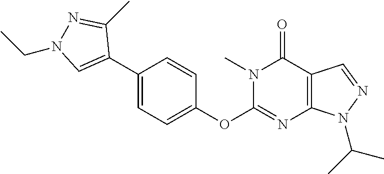 [SMILES: CCn1cc(-c2ccc(Oc3nc4c(cnn4C(C)C)c(=O)n3C)cc2)c(C)n1]

¹H NMR (400 MHz, CDCl₃) δ 8.02 (s, 1H), 7.57 (s, 1H), 7.47 (d, J=8.6 Hz, 2H), 7.29 (s, 2H), 4.63 (td, J=6.6, 13.4 Hz, 1H), 4.26 (q, J=7.3 Hz, 2H), 3.67 (s, 3H), 2.54-2.42 (m, 3H), 1.55 (t, J=7.3 Hz, 3H), 1.40 (d, J=6.7 Hz, 6H). [M+H]=393.23.

### Example 44. 5-Methyl-6-{4-[6-(methylamino)pyridin-3-yl]phenoxy}-1-(propan-2-yl)-1H,4H,5H-pyrazolo[3,4-d]pyrimidin-4-one

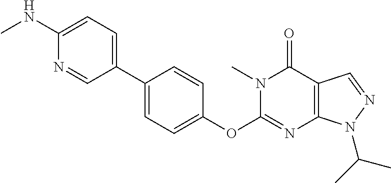 [SMILES: CNc1ccc(-c2ccc(Oc3nc4c(cnn4C(C)C)c(=O)n3C)cc2)cn1]

¹H NMR (400 MHz, CDCl₃) δ 9.99 (br s, 1H), 8.13 (d, J=9.3 Hz, 1H), 8.07 (s, 1H), 8.02 (s, 1H), 7.61-7.50 (m, J=8.6 Hz, 2H), 7.37 (d, J=8.6 Hz, 2H), 6.90 (d, J=9.3 Hz, 1H), 4.61 (td, J=6.7, 13.4 Hz, 1H), 3.68 (s, 3H), 3.07 (s, 3H), 1.40 (d, J=6.7 Hz, 6H). [M+H]=391.17.

### Example 45. 5-Methyl-6-[4-(1-methyl-1H-imidazol-4-yl)phenoxy]-1-(propan-2-yl)-1H,4H,5H-pyrazolo[3,4-d]pyrimidin-4-one

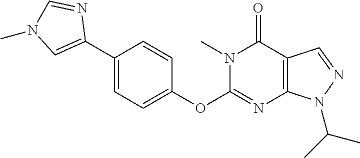 [SMILES: CC(C)n1ncc2c(=O)n(C)c(Oc3ccc(-c4cn(C)cn4)cc3)nc21]

¹H NMR (400 MHz, CDCl₃) δ 8.83 (br s, 1H), 8.02 (s, 1H), 7.83 (d, J=8.6 Hz, 2H), 7.38 (d, J=8.6 Hz, 2H), 7.32 (s, 1H), 4.61 (td, J=6.8, 13.4 Hz, 1H), 3.98 (s, 3H), 3.67 (s, 3H), 1.39 (d, J=6.7 Hz, 6H). [M+H]=365.17.

### Example 46. 5-Methyl-6-[4-(4-methyl-1,3-thiazol-2-yl)phenoxy]-1-(propan-2-yl)-1H,4H,5H-pyrazolo[3,4-d]pyrimidin-4-one

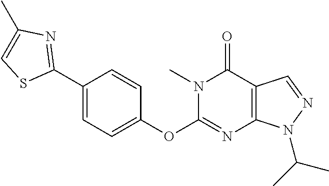 [SMILES: Cc1csc(-c2ccc(Oc3nc4c(cnn4C(C)C)c(=O)n3C)cc2)n1]

¹H NMR (400 MHz, CDCl₃) δ 8.11-7.96 (m, 3H), 7.33 (d, J=8.7 Hz, 2H), 6.97 (s, 1H), 4.61 (td, J=6.6, 13.4 Hz, 1H), 3.67 (s, 3H), 2.56 (s, 3H), 1.39 (d, J=6.7 Hz, 6H). [M+H]=382.15.

### Example 47. 6-[4-(1,2-Dimethyl-1H-imidazol-4-yl)phenoxy]-5-methyl-1-(propan-2-yl)-1H,4H,5H-pyrazolo[3,4-d]pyrimidin-4-one

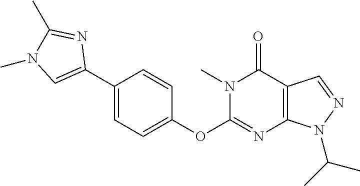 [SMILES: Cc1nc(-c2ccc(Oc3nc4c(cnn4C(C)C)c(=O)n3C)cc2)cn1C]

¹H NMR (400 MHz, CDCl₃) δ 7.99 (s, 1H), 7.88-7.77 (m, 2H), 7.25 (d, J=8.8 Hz, 2H), 7.15 (s, 1H), 4.60 (spt, J=6.8 Hz, 1H), 3.69 (s, 3H), 3.65 (s, 3H), 2.56 (s, 3H), 1.37 (d, J=6.7 Hz, 6H). [M+H]=379.34.

### Example 48. 5-Methyl-6-[4-(1-methyl-1H-pyrazol-3-yl)phenoxy]-1-(propan-2-yl)-1H,4H,5H-pyrazolo[3,4-d]pyrimidin-4-one

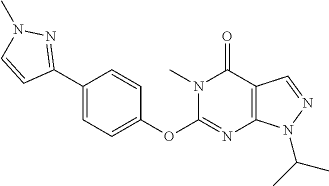 [SMILES: CC(C)n1ncc2c(=O)n(C)c(Oc3ccc(-c4ccn(C)n4)cc3)nc21]

¹H NMR (400 MHz, CDCl₃) δ 8.02 (s, 1H), 7.88 (d, J=8.7 Hz, 2H), 7.44 (d, J=2.1 Hz, 1H), 7.27-7.24 (m, 2H), 6.63-6.56 (m, 1H), 4.61 (td, J=6.6, 13.4 Hz, 1H), 4.00 (s, 3H), 3.67 (s, 3H), 1.38 (d, J=6.7 Hz, 6H). [M+H]=365.18.

### Example 49. 6-[4-(Dimethyl-1,3-oxazol-5-yl)phenoxy]-5-methyl-1-(propan-2-yl)-1H,4H,5H-pyrazolo[3,4-d]pyrimidin-4-one

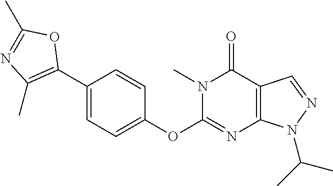 [SMILES: Cc1nc(C)c(-c2ccc(Oc3nc4c(cnn4C(C)C)c(=O)n3C)cc2)o1]

¹H NMR (400 MHz, CDCl₃) δ 8.02 (s, 1H), 7.68 (d, J=8.8 Hz, 2H), 7.33 (d, J=8.8 Hz, 2H), 4.62 (td, J=6.7, 13.4 Hz, 1H), 3.67 (s, 3H), 2.57 (s, 3H), 2.46 (s, 3H), 1.39 (d, J=6.7 Hz, 6H). [M+H]=380.31.

### Example 50. 5-Methyl-6-[4-(2-methyl-1,3-thiazol-5-yl)phenoxy]-1-(propan-2-yl)-1H,4H,5H-pyrazolo[3,4-d]pyrimidin-4-one

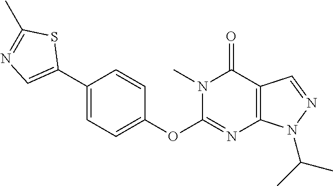 [SMILES: Cc1ncc(-c2ccc(Oc3nc4c(cnn4C(C)C)c(=O)n3C)cc2)s1]

¹H NMR (400 MHz, CDCl₃) δ 7.94 (s, 1H), 7.85 (s, 1H), 7.54 (d, J=8.6 Hz, 2H), 7.22 (d, J=8.6 Hz, 2H), 4.62-4.47 (m, 1H), 3.58 (s, 3H), 2.75 (s, 3H), 1.31 (d, J=6.7 Hz, 6H). [M+H]=382.13.

### Example 51. 5-Methyl-6-{4-[3-methyl-1-(propan-2-yl)-1H-pyrazol-4-yl]phenoxy}-1-(propan-2-yl)-1H,4H,5H-pyrazolo[3,4-d]pyrimidin-4-one

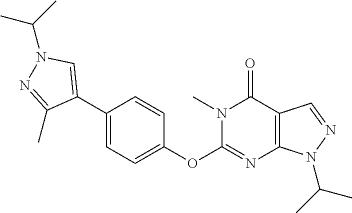 [SMILES: Cc1nn(C(C)C)cc1-c1ccc(Oc2nc3c(cnn3C(C)C)c(=O)n2C)cc1]

¹H NMR (400 MHz, CDCl₃) δ 8.05-8.01 (m, 1H), 7.61 (s, 1H), 7.48 (d, J=8.6 Hz, 2H), 7.29 (d, J=8.7 Hz, 2H), 4.76-4.53 (m, 2H), 3.71-3.62 (m, 3H), 2.50 (s, 3H), 1.59 (d, J=6.7 Hz, 6H), 1.42-1.34 (m, 6H). [M+H]=408.45.

### Example 52. 6-[4-(1-Ethyl-5-methyl-1H-pyrazol-4-yl)phenoxy]-5-methyl-1-(propan-2-yl)-1H,4H,5H-pyrazolo[3,4-d]pyrimidin-4-one

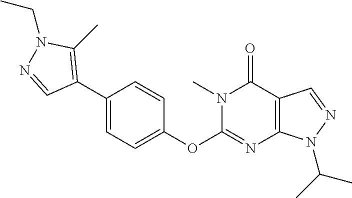 [SMILES: CCn1ncc(-c2ccc(Oc3nc4c(cnn4C(C)C)c(=O)n3C)cc2)c1C]

¹H NMR (400 MHz, CDCl₃) δ 8.02 (s, 1H), 7.75 (s, 1H), 7.47-7.42 (m, 2H), 7.31-7.27 (m, 2H), 4.64 (spt, J=6.8 Hz, 1H), 4.26 (q, J=7.3 Hz, 2H), 3.68 (s, 3H), 2.46 (s, 3H), 1.50 (t, J=7.3 Hz, 3H), 1.40 (d, J=6.7 Hz, 6H). [M+H]=394.21.

### Example 53. 6-{4-[1-(Difluoromethyl)-3-methyl-1H-pyrazol-4-yl]phenoxy}-5-methyl-1-(propan-2-yl)-1H,4H,5H-pyrazolo[3,4-d]pyrimidin-4-one

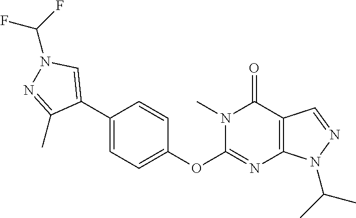 [SMILES: Cc1nn(C(F)F)cc1-c1ccc(Oc2nc3c(cnn3C(C)C)c(=O)n2C)cc1]

¹H NMR (400 MHz, CDCl₃) δ 8.05 (s, 1H), 7.93 (s, 1H), 7.54-7.48 (m, 2H), 7.35 (s, 1H), 7.33-7.28 (m, 2H), 7.26-7.03 (m, 1H), 4.64 (spt, J=6.7 Hz, 1H), 3.69 (s, 3H), 2.47 (s, 3H), 1.40 (d, J=6.7 Hz, 6H). [M+H]=414.87.

### Example 54. 6-[4-(1,3-Dimethyl-1H-pyrazol-4-yl)phenoxy]-5-methyl-1-(propan-2-yl)-1H,4H,5H-pyrazolo[3,4-d]pyrimidin-4-one

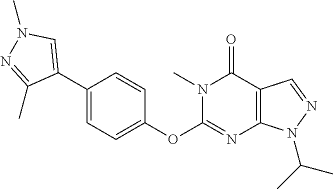 [SMILES: Cc1nn(C)cc1-c1ccc(Oc2nc3c(cnn3C(C)C)c(=O)n2C)cc1]

¹H NMR (400 MHz, CDCl₃) δ 8.02 (s, 1H), 7.52 (s, 1H), 7.46 (d, J=8.7 Hz, 2H), 7.30-7.27 (m, 2H), 4.70-4.55 (m, 1H), 3.97 (s, 3H), 3.67 (s, 3H), 2.47 (s, 3H), 1.40 (d, J=6.7 Hz, 6H). [M+H]=379.37.

### Example 55. 6-[4-(1,5-Dimethyl-1H-pyrazol-4-yl)phenoxy]-5-methyl-1-(propan-2-yl)-1H,4H,5H-pyrazolo[3,4-d]pyrimidin-4-one

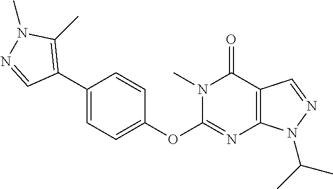 [SMILES: Cc1c(-c2ccc(Oc3nc4c(cnn4C(C)C)c(=O)n3C)cc2)cnn1C]

¹H NMR (400 MHz, CDCl₃) δ 8.02 (s, 1H), 7.69 (s, 1H), 7.44 (d, J=8.7 Hz, 2H), 7.29 (d, J=8.9 Hz, 2H), 4.64 (spt, J=6.8 Hz, 1H), 3.92 (s, 3H), 3.67 (s, 3H), 2.45 (s, 3H), 1.40 (d, J=6.7 Hz, 6H). [M+H]=379.34.

### Example 56. 6-[4-(1-tert-Butyl-1H-pyrazol-4-yl)phenoxy]-5-methyl-1-(propan-2-yl)-1H,4H,5H-pyrazolo[3,4-d]pyrimidin-4-one

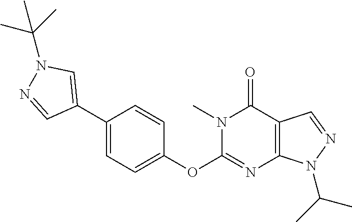 [SMILES: CC(C)n1ncc2c(=O)n(C)c(Oc3ccc(-c4cnn(C(C)(C)C)c4)cc3)nc21]

¹H NMR (400 MHz, CDCl₃) δ 8.00 (s, 1H), 7.86 (s, 1H), 7.82 (s, 1H), 7.58 (d, J=8.6 Hz, 2H), 7.23 (d, J=8.7 Hz, 2H), 4.62 (spt, J=6.6 Hz, 1H), 3.66 (s, 3H), 1.66 (s, 9H), 1.39 (d, J=6.7 Hz, 6H). [M+H]=407.39.

### Example 57. 6-[4-(1-tert-Butyl-3-methyl-1H-pyrazol-4-yl)phenoxy]-5-methyl-1-(propan-2-yl)-1H,4H,5H-pyrazolo[3,4-d]pyrimidin-4-one

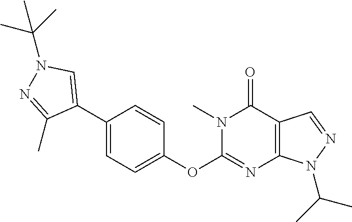 [SMILES: Cc1nn(C(C)(C)C)cc1-c1ccc(Oc2nc3c(cnn3C(C)C)c(=O)n2C)cc1]

¹H NMR (400 MHz, CDCl₃) δ 8.03 (s, 1H), 7.67 (s, 1H), 7.48 (d, J=8.7 Hz, 2H), 7.30-7.27 (m, 2H), 4.63 (td, J=6.8, 13.5 Hz, 1H), 3.67 (s, 3H), 2.49 (s, 3H), 1.67 (s, 9H), 1.40 (d, J=6.7 Hz, 6H). [M+H]=421.40.

### Example 58. 6-{[6-(1,3-Dimethyl-1H-pyrazol-4-yl)pyridin-3-yl]oxy}-5-methyl-1-(propan-2-yl)-1H,4H,5H-pyrazolo[3,4-d]pyrimidin-4-one

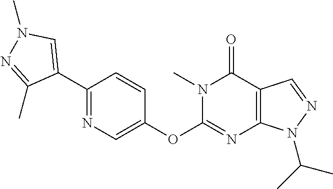 [SMILES: Cc1nn(C)cc1-c1ccc(Oc2nc3c(cnn3C(C)C)c(=O)n2C)cn1]

¹H NMR (400 MHz, CDCl₃) δ 8.55 (s, 1H), 8.02 (s, 1H), 7.87 (s, 1H), 7.64-7.49 (m, 2H), 4.62 (spt, J=6.7 Hz, 1H), 3.95-3.88 (m, 3H), 3.68 (s, 3H), 2.57 (s, 3H), 1.39 (d, J=6.7 Hz, 6H). [M+H]=380.33.

### Example 59. 6-{[6-(1,5-Dimethyl-1H-pyrazol-4-yl)pyridin-3-yl]oxy}-5-methyl-1-(propan-2-yl)-1H,4H,5H-pyrazolo[3,4-d]pyrimidin-4-one

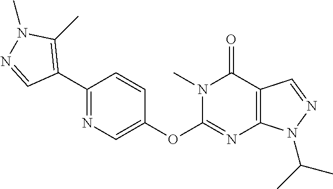 [SMILES: Cc1c(-c2ccc(Oc3nc4c(cnn4C(C)C)c(=O)n3C)cn2)cnn1C]

¹H NMR (400 MHz, CDCl₃) δ 8.77 (d, J=2.4 Hz, 1H), 8.05 (s, 1H), 7.95 (s, 1H), 7.77 (dd, J=2.6, 8.7 Hz, 1H), 7.62 (d, J=8.7 Hz, 1H), 4.64 (spt, J=6.7 Hz, 1H), 3.94 (s, 3H), 3.69 (s, 3H), 2.64 (s, 3H), 1.41 (d, J=6.7 Hz, 6H). [M+H]=380.34.

### Example 60. 5-Methyl-6-{4-[1-methyl-3-(trifluoromethyl)-1H-pyrazol-4-yl]phenoxy}-1-(propan-2-yl)-1H,4H,5H-pyrazolo[3,4-d]pyrimidin-4-one

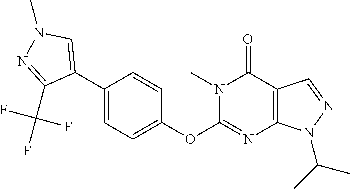 [SMILES: CC(C)n1ncc2c(=O)n(C)c(Oc3ccc(-c4cn(C)nc4C(F)(F)F)cc3)nc21]

¹H NMR (400 MHz, CDCl₃) δ 8.63 (s, 1H), 8.03 (s, 2H), 7.79-7.63 (m, 2H), 4.62 (td, J=6.8, 13.4 Hz, 1H), 4.04 (s, 3H), 3.69 (s, 3H), 1.40 (d, J=6.7 Hz, 6H). [M+H]=433.36.

### Example 61. 5-Methyl-6-({6-[1-methyl-3-(trifluoromethyl)-1H-pyrazol-4-yl]pyridin-3-yl}oxy)-1-(propan-2-yl)-1H,4H,5H-pyrazolo[3,4-d]pyrimidin-4-one

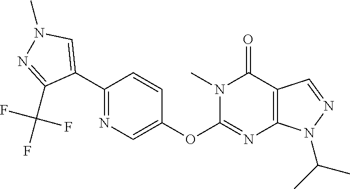 [SMILES: CC(C)n1ncc2c(=O)n(C)c(Oc3ccc(-c4cn(C)nc4C(F)(F)F)nc3)nc21]

¹H NMR (400 MHz, CDCl₃) δ 8.63 (s, 1H), 8.03 (s, 2H), 7.79-7.63 (m, 2H), 4.62 (td, J=6.8, 13.4 Hz, 1H), 4.04 (s, 3H), 3.69 (s, 3H), 1.40 (d, J=6.7 Hz, 6H). [M+H]=434.34.

### Example 62. 5-Methyl-1-(propan-2-yl)-6-{4-[4-(propan-2-yl)pyrimidin-2-yl]phenoxy}-1H,4H,5H-pyrazolo[3,4-d]pyrimidin-4-one

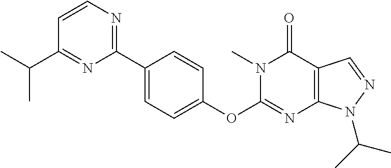 [SMILES: CC(C)c1ccnc(-c2ccc(Oc3nc4c(cnn4C(C)C)c(=O)n3C)cc2)n1]

¹H NMR (400 MHz, CDCl₃) δ 8.76 (d, J=5.3 Hz, 1H), 8.58 (d, J=8.8 Hz, 2H), 8.03 (s, 1H), 7.37 (d, J=8.8 Hz, 2H), 7.16 (d, J=5.3 Hz, 1H), 4.62 (spt, J=6.8 Hz, 1H), 3.76-3.63 (m, 3H), 3.12 (quin, J=6.9 Hz, 1H), 1.39 (t, J=7.4 Hz, 12H). [M+H]=405.36.

### Example 63. 6-[4-(4-Methoxypyrimidin-2-yl)phenoxy]-5-methyl-1-(propan-2-yl)-1H,4H,5H-pyrazolo[3,4-d]pyrimidin-4-one

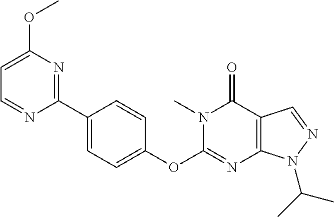 [SMILES: COc1ccnc(-c2ccc(Oc3nc4c(cnn4C(C)C)c(=O)n3C)cc2)n1]

¹H NMR (400 MHz, CDCl₃) δ 8.63 (d, J=5.9 Hz, 1H), 8.55 (d, J=8.8 Hz, 2H), 8.03 (s, 1H), 7.38 (d, J=8.8 Hz, 2H), 6.75 (d, J=5.9 Hz, 1H), 4.62 (spt, J=6.7 Hz, 1H), 4.16 (s, 3H), 3.69 (s, 3H), 1.38 (d, J=6.7 Hz, 6H). [M+H]=393.35.

### Example 64. 6-(4-{Furo[2,3-d]pyrimidin-2-yl}phenoxy)-5-methyl-1-(propan-2-yl)-1H,4H,5H-pyrazolo[3,4-d]pyrimidin-4-one

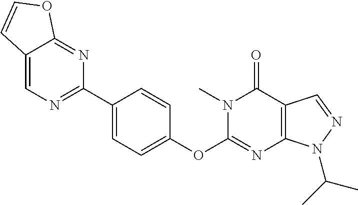 [SMILES: CC(C)n1ncc2c(=O)n(C)c(Oc3ccc(-c4ncc5ccoc5n4)cc3)nc21]

¹H NMR (400 MHz, CDCl₃) δ 9.14 (s, 1H), 8.64 (d, J=8.8 Hz, 2H), 8.02 (s, 1H), 7.77 (d, J=2.4 Hz, 1H), 7.39 (d, J=8.8 Hz, 2H), 6.92 (d, J=2.6 Hz, 1H), 4.62 (spt, J=6.7 Hz, 1H), 3.69 (s, 3H), 1.38 (d, J=6.7 Hz, 6H). [M+H]=403.32.

### Example 65. 6-[4-(3-Ethyl-1-methyl-1H-pyrazol-4-yl)phenoxy]-5-methyl-1-(propan-2-yl)-1H,4H,5H-pyrazolo[3,4-d]pyrimidin-4-one

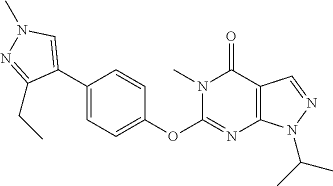 [SMILES: CCc1nn(C)cc1-c1ccc(Oc2nc3c(cnn3C(C)C)c(=O)n2C)cc1]

¹H NMR (400 MHz, CDCl₃) δ 8.00 (s, 1H), 7.48-7.42 (m, 3H), 7.26-7.22 (m, 2H), 4.68-4.55 (m, 1H), 3.92 (s, 3H), 3.71-3.63 (m, 3H), 2.83 (q, J=7.5 Hz, 2H), 1.39 (d, J=6.8 Hz, 6H), 1.28 (t, J=7.6 Hz, 3H). [M+H]=393.3.

### Example 66. 6-[4-(4-Ethylpyrimidin-2-yl)phenoxy]-5-methyl-1-(propan-2-yl)-1H,4H,5H-pyrazolo[3,4-d]pyrimidin-4-one

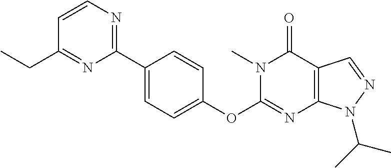 [SMILES: CCc1ccnc(-c2ccc(Oc3nc4c(cnn4C(C)C)c(=O)n3C)cc2)n1]

¹H NMR (400 MHz, CDCl₃) δ 8.75 (d, J=5.1 Hz, 1H), 8.57 (d, J=8.0 Hz, 2H), 8.04-8.04 (m, 1H), 7.37 (d, J=7.7 Hz, 2H), 7.15 (d, J=5.1 Hz, 1H), 4.69-4.53 (m, 1H), 3.69 (s, 3H), 2.91 (q, J=7.7 Hz, 2H), 1.47-1.32 (m, 9H). [M+H]=391.36.

### Example 67. 6-[4-(3-Cyclopropyl-1-methyl-1H-pyrazol-4-yl)phenoxy]-5-methyl-1-(propan-2-yl)-1H,4H,5H-pyrazolo[3,4-d]pyrimidin-4-one

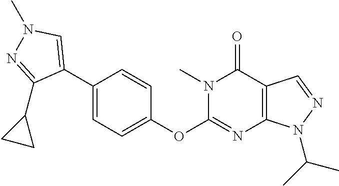 [SMILES: CC(C)n1ncc2c(=O)n(C)c(Oc3ccc(-c4cn(C)nc4C4CC4)cc3)nc21]

¹H NMR (400 MHz, CDCl₃) δ 8.00 (s, 1H), 7.66-7.60 (m, 2H), 7.47 (s, 1H), 7.27-7.22 (m, 2H), 4.63 (quin, J=6.7 Hz, 1H), 3.90-3.83 (m, 3H), 3.67 (s, 3H), 2.03-1.92 (m, 1H), 1.43-1.35 (m, 6H), 1.01-0.92 (m, 4H). [M+H]=405.38.

### Example 68. 6-[2-Fluoro-4-(1-methyl-1H-pyrazol-3-yl)phenoxy]-5-methyl-1-(propan-2-yl)-1H,4H,5H-pyrazolo[3,4-d]pyrimidin-4-one

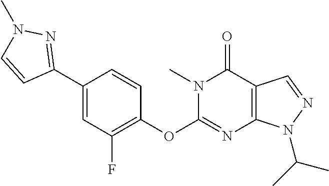 [SMILES: CC(C)n1ncc2c(=O)n(C)c(Oc3ccc(-c4ccn(C)n4)cc3F)nc21]

¹H NMR (400 MHz, CDCl₃) δ 8.03 (s, 1H), 7.71-7.61 (m, 2H), 7.45 (d, J=2.2 Hz, 1H), 7.31 (t, J=8.1 Hz, 1H), 6.58 (d, J=2.2 Hz, 1H), 4.59 (td, J=6.7, 13.5 Hz, 1H), 4.00 (s, 3H), 3.69 (s, 3H), 1.37 (d, J=6.7 Hz, 6H). [M+H]=383.34.

### Example 69. 6-[4-(1,2-Dimethyl-1H-imidazol-4-yl)-3-fluorophenoxy]-5-methyl-1-(propan-2-yl)-1H,4H,5H-pyrazolo[3,4-d]pyrimidin-4-one

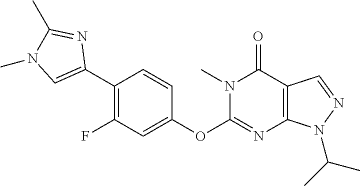 [SMILES: Cc1nc(-c2ccc(Oc3nc4c(cnn4C(C)C)c(=O)n3C)cc2F)cn1C]

¹H NMR (400 MHz, CDCl₃) δ 8.23 (t, J=8.6 Hz, 1H), 8.02 (s, 1H), 7.43 (d, J=2.8 Hz, 1H), 7.28 (d, J=1.8 Hz, 1H), 7.16 (dd, J=2.2, 11.6 Hz, 1H), 4.63 (spt, J=6.6 Hz, 1H), 3.84 (s, 3H), 3.65 (s, 3H), 2.81 (s, 3H), 1.41 (d, J=6.7 Hz, 6H). [M+H]=397.34.

### Example 70. 6-[4-(1,2-Dimethyl-1H-imidazol-4-yl)-2-fluorophenoxy]-5-methyl-1-(propan-2-yl)-1H,4H,5H-pyrazolo[3,4-d]pyrimidin-4-one

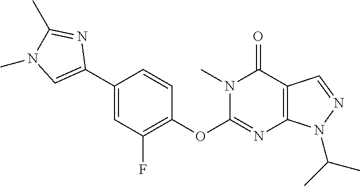 [SMILES: Cc1nc(-c2ccc(Oc3nc4c(cnn4C(C)C)c(=O)n3C)c(F)c2)cn1C]

¹H NMR (400 MHz, CDCl₃) δ 8.01 (s, 1H), 7.67 (d, J=8.7 Hz, 1H), 7.60 (dd, J=1.6, 10.6 Hz, 1H), 7.42 (t, J=8.1 Hz, 1H), 7.25 (s, 1H), 4.57 (spt, J=6.7 Hz, 1H), 3.83 (s, 3H), 3.67 (s, 3H), 2.79 (s, 3H), 1.37 (d, J=6.7 Hz, 6H). [M+H]=397.32.

### Example 71. 6-[4-(1,5-Dimethyl-1H-pyrazol-3-yl)phenoxy]-5-methyl-1-(propan-2-yl)-1H,4H,5H-pyrazolo[3,4-d]pyrimidin-4-one

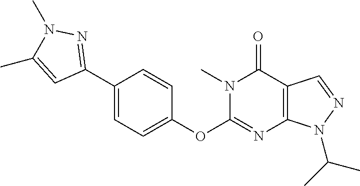 [SMILES: Cc1cc(-c2ccc(Oc3nc4c(cnn4C(C)C)c(=O)n3C)cc2)nn1C]

¹H NMR (400 MHz, CDCl₃) δ 8.01 (s, 1H), 7.89-7.80 (m, 2H), 7.27-7.23 (m, 2H), 6.38 (s, 1H), 4.61 (spt, J=6.7 Hz, 1H), 3.92-3.83 (m, 3H), 3.66 (s, 3H), 2.35 (s, 3H), 1.37 (d, J=6.7 Hz, 6H). [M+H]=379.36.

### Example 72. 6-{2,3-Difluoro-4-[1-(propan-2-yl)-1H-pyrazol-4-yl]phenoxy}-5-methyl-1-(propan-2-yl)-1H,4H,5H-pyrazolo[3,4-d]pyrimidin-4-one

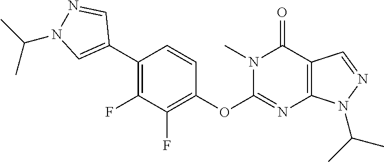 [SMILES: CC(C)n1cc(-c2ccc(Oc3nc4c(cnn4C(C)C)c(=O)n3C)c(F)c2F)cn1]

¹H NMR (400 MHz, CDCl₃) δ 8.04 (s, 1H), 7.97 (s, 1H), 7.92 (d, J=2.2 Hz, 1H), 7.41 (dt, J=2.3, 8.1 Hz, 1H), 7.12 (ddd, J=1.9, 6.9, 8.8 Hz, 1H), 4.62 (sxtd, J=6.8, 13.9 Hz, 2H), 3.70 (s, 3H), 1.61 (d, J=6.7 Hz, 6H), 1.40 (d, J=6.7 Hz, 6H). [M+H]=429.38.

### Example 73. 6-[2,3-Difluoro-4-(1-methyl-1H-pyrazol-4-yl)phenoxy]-5-methyl-1-(propan-2-yl)-1H,4H,5H-pyrazolo[3,4-d]pyrimidin-4-one

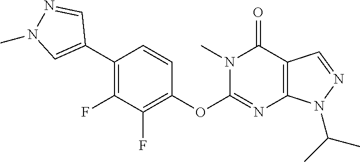 [SMILES: CC(C)n1ncc2c(=O)n(C)c(Oc3ccc(-c4cnn(C)c4)c(F)c3F)nc21]

¹H NMR (400 MHz, CDCl₃) δ 8.04 (s, 1H), 7.94 (s, 1H), 7.87 (d, J=2.1 Hz, 1H), 7.45-7.35 (m, 1H), 7.12 (ddd, J=2.0, 6.9, 8.7 Hz, 1H), 4.62 (quind, J=6.7, 13.4 Hz, 1H), 4.04 (s, 3H), 3.70 (s, 3H), 1.41 (d, J=6.8 Hz, 6H). [M+H]=401.34.

### Example 74. 6-[4-(1,3-Dimethyl-1H-pyrazol-4-yl)-2,3-difluorophenoxy]-5-methyl-1-(propan-2-yl)-1H,4H,5H-pyrazolo[3,4-d]pyrimidin-4-one

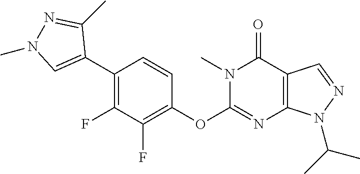 [SMILES: Cc1nn(C)cc1-c1ccc(Oc2nc3c(cnn3C(C)C)c(=O)n2C)c(F)c1F]

¹H NMR (400 MHz, CDCl₃) δ 8.04 (s, 1H), 7.64 (d, J=1.7 Hz, 1H), 7.27-7.21 (m, 1H), 7.18-7.11 (m, 1H), 4.62 (spt, J=6.7 Hz, 1H), 3.98 (s, 3H), 3.71 (s, 3H), 2.43 (s, 3H), 1.42 (d, J=6.7 Hz, 6H). [M+H]=415.37.

### Example 75. 6-[4-(1-Ethyl-1H-pyrazol-4-yl)-2,5-difluorophenoxy]-5-methyl-1-(propan-2-yl)-1H,4H,5H-pyrazolo[3,4-d]pyrimidin-4-one

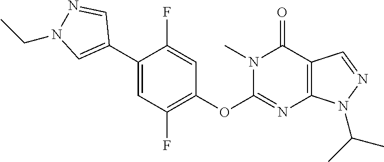 [SMILES: CCn1cc(-c2cc(F)c(Oc3nc4c(cnn4C(C)C)c(=O)n3C)cc2F)cn1]

¹H NMR (400 MHz, CDCl₃) δ 8.03 (s, 1H), 7.92 (s, 1H), 7.89 (d, J=2.3 Hz, 1H), 7.43 (dd, J=6.8, 10.8 Hz, 1H), 7.16 (dd, J=6.4, 10.3 Hz, 1H), 4.63 (quin, J=6.7 Hz, 1H), 4.29 (q, J=7.3 Hz, 2H), 3.69 (s, 3H), 1.59 (t, J=7.3 Hz, 3H), 1.41 (d, J=6.7 Hz, 6H). [M+H]=415.33.

### Example 76. 6-[4-(1-Ethyl-5-methyl-1H-pyrazol-3-yl)phenoxy]-5-methyl-1-(propan-2-yl)-1H,4H,5H-pyrazolo[3,4-d]pyrimidin-4-one

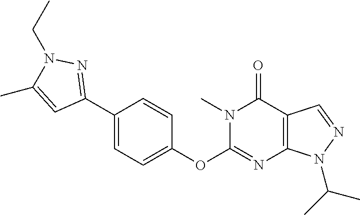 [SMILES: CCn1nc(-c2ccc(Oc3nc4c(cnn4C(C)C)c(=O)n3C)cc2)cc1C]

¹H NMR (400 MHz, CDCl₃) δ 8.03 (s, 1H), 7.86 (d, J=8.7 Hz, 2H), 7.30-7.26 (m, 2H), 6.40 (s, 1H), 4.63 (td, J=6.7, 13.4 Hz, 1H), 4.25 (q, J=7.3 Hz, 2H), 3.68 (s, 3H), 2.39 (s, 3H), 1.51 (t, J=7.3 Hz, 3H), 1.39 (d, J=6.7 Hz, 6H). [M+H]=393.44.

### Example 77. 6-{4-[4-Fluoro-1-(propan-2-yl)-1H-pyrazol-3-yl]phenoxy}-5-methyl-1-(propan-2-yl)-1H,4H,5H-pyrazolo[3,4-d]pyrimidin-4-one

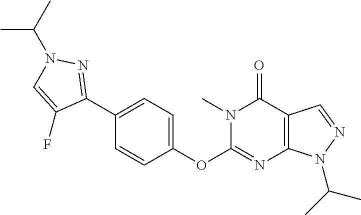 [SMILES: CC(C)n1cc(F)c(-c2ccc(Oc3nc4c(cnn4C(C)C)c(=O)n3C)cc2)n1]

¹H NMR (400 MHz, CDCl₃) δ 8.03 (s, 1H), 8.00-7.94 (m, 2H), 7.42 (d, J=4.8 Hz, 1H), 7.32-7.28 (m, 2H), 4.63 (quin, J=6.7 Hz, 1H), 4.48 (td, J=6.7, 13.3 Hz, 1H), 3.68 (s, 3H), 1.56 (d, J=6.7 Hz, 6H), 1.43-1.36 (m, 6H). [M+H]=411.06.

Example 78 was prepared in a manner analogous to Example 21, with the appropriate starting material substitutions.

### Example 78. 5-Methyl-1-(propan-2-yl)-6-{[(1r4r)-4-(trifluoromethyl)cyclohexyl]methoxy}-1H4H5H-pyrazolo[3,4-d]pyrimidin-4-one

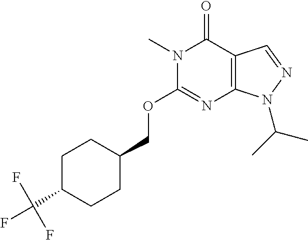 [SMILES: CC(C)n1ncc2c(=O)n(C)c(OC[C@H]3CC[C@H](C(F)(F)F)CC3)nc21]

¹H NMR (400 MHz, CDCl₃) δ 7.99 (s, 1H), 4.89 (spt, J=6.7 Hz, 1H), 4.31 (d, J=6.2 Hz, 2H), 3.47 (s, 3H), 2.11-2.02 (m, 5H), 1.90 (dtt, J=3.4, 6.2, 12.0 Hz, 1H), 1.54 (d, J=6.7 Hz, 6H), 1.49-1.34 (m, 2H), 1.25-1.13 (m, 2H). [M+H]=373.4.

Example 79 was prepared in a manner analogous to Example 3, with the appropriate starting material substitutions.

### Example 79. 6-[4-(4-Acetylpiperazin-1-yl)-2-fluorophenoxy]-5-methyl-1-(propan-2-yl)-1H,4H,5H-pyrazolo[3,4-d]pyrimidin-4-one

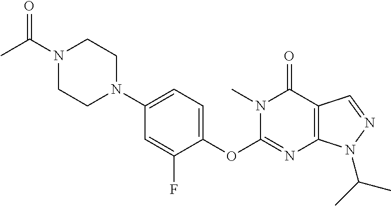 [SMILES: CC(=O)N1CCN(c2ccc(Oc3nc4c(cnn4C(C)C)c(=O)n3C)c(F)c2)CC1]

¹H NMR (400 MHz, CDCl₃) δ 8.03 (s, 1H), 7.20 (t, J=8.7 Hz, 1H), 6.82-6.74 (m, 2H), 4.68-4.55 (m, 1H), 3.88-3.79 (m, 2H), 3.74-3.68 (m, 2H), 3.67 (s, 3H), 3.27 (td, J=5.1, 13.9 Hz, 4H), 2.21 (s, 3H), 1.40 (d, J=6.8 Hz, 6H). [M+H]=429.4.

Example 80-Example 88 were prepared in a manner analogous to Example 4, with the appropriate starting material substitutions.

### Example 80. 5-Methyl-6-{4-[3-methyl-1-(propan-2-yl)-1H-pyrazol-4-yl]phenoxy}-1-(oxan-4-yl)-1H,4H,5H-pyrazolo[3,4-d]pyrimidin-4-one

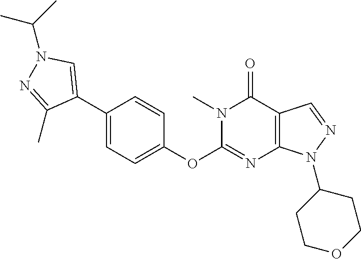 [SMILES: Cc1nn(C(C)C)cc1-c1ccc(Oc2nc3c(cnn3C3CCOCC3)c(=O)n2C)cc1]

¹H NMR (400 MHz, CDCl₃) δ 8.03 (s, 1H), 7.62 (s, 1H), 7.48 (d, J=8.7 Hz, 2H), 7.29 (d, J=8.7 Hz, 2H), 4.70 (td, J=6.7, 13.4 Hz, 1H), 4.43 (tt, J=4.2, 11.4 Hz, 1H), 4.03 (dd, J=2.4, 11.8 Hz, 2H), 3.68 (s, 3H), 3.43 (t, J=11.1 Hz, 2H), 2.50 (s, 3H), 2.23 (dq, J=4.4, 12.2 Hz, 2H), 1.85-1.76 (m, 2H), 1.59 (d, J=6.7 Hz, 6H). [M+H]=450.43.

### Example 81. 6-{4-[1-(Difluoromethyl)-5-methyl-1H-pyrazol-4-yl]phenoxy}-5-methyl-1-(oxan-4-yl)-1H,4H,5H-pyrazolo[3,4-d]pyrimidin-4-one

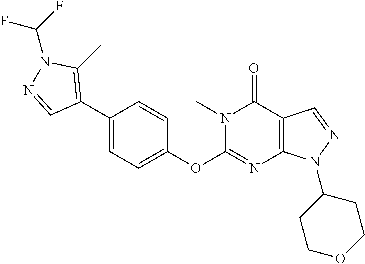 [SMILES: Cc1c(-c2ccc(Oc3nc4c(cnn4C4CCOCC4)c(=O)n3C)cc2)cnn1C(F)F]

¹H NMR (400 MHz, CDCl₃) δ 8.04 (s, 1H), 7.73 (s, 1H), 7.49-7.44 (m, 2H), 7.35-7.29 (m, 2H), 7.16 (s, 1H), 4.44 (tt, J=4.2, 11.5 Hz, 1H), 4.09-3.96 (m, 2H), 3.69 (s, 3H), 3.50-3.37 (m, 2H), 2.61 (s, 3H), 2.25 (br s, 2H), 1.82 (dd, J=2.0, 12.5 Hz, 2H). [M+H]=457.37.

### Example 82. 6-[4-(1-Ethyl-5-methyl-1H-pyrazol-4-yl)phenoxy]-5-methyl-1-(oxan-4-yl)-1H,4H,5H-pyrazolo[3,4-d]pyrimidin-4-one

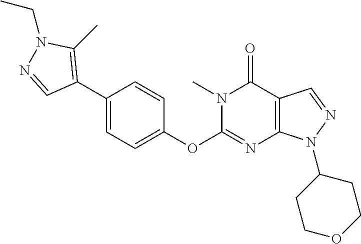 [SMILES: CCn1ncc(-c2ccc(Oc3nc4c(cnn4C4CCOCC4)c(=O)n3C)cc2)c1C]

¹H NMR (400 MHz, CDCl₃) δ 8.02 (s, 1H), 7.74 (s, 1H), 7.47-7.42 (m, 2H), 7.29 (d, J=8.7 Hz, 2H), 4.49-4.37 (m, 1H), 4.26 (q, J=7.2 Hz, 2H), 4.02 (dd, J=2.4, 11.4 Hz, 2H), 3.71-3.64 (m, 3H), 3.43 (t, J=11.4 Hz, 2H), 2.46 (s, 3H), 2.27-2.16 (m, 2H), 1.81 (d, J=10.6 Hz, 2H), 1.50 (t, J=7.2 Hz, 3H). [M+H]=436.16.

### Example 83. 6-{4-[1-(Difluoromethyl)-3-methyl-1H-pyrazol-4-yl]phenoxy}-5-methyl-1-(oxan-4-yl)-1H,4H,5H-pyrazolo[3,4-d]pyrimidin-4-one

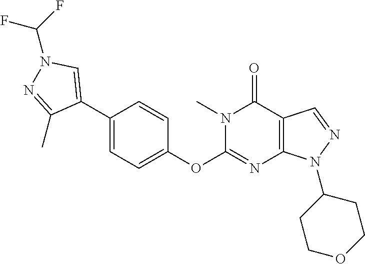 [SMILES: Cc1nn(C(F)F)cc1-c1ccc(Oc2nc3c(cnn3C3CCOCC3)c(=O)n2C)cc1]

¹H NMR (400 MHz, CDCl₃) δ 8.04 (s, 1H), 7.92 (s, 1H), 7.53-7.49 (m, 2H), 7.35 (s, 1H), 7.32-7.28 (m, 2H), 7.21-7.04 (m, 1H), 4.44 (tt, J=4.1, 11.5 Hz, 1H), 4.04 (dd, J=2.5, 12.0 Hz, 2H), 3.69 (s, 3H), 3.49-3.39 (m, 2H), 2.46 (s, 3H), 2.28-2.17 (m, 2H), 1.82 (dd, J=2.2, 12.7 Hz, 2H). [M+H]=457.39.

### Example 84. 6-[4-(1-tert-Butyl-3-methyl-1H-pyrazol-4-yl)phenoxy]-5-methyl-1-(oxan-4-yl)-1H,4H,5H-pyrazolo[3,4-d]pyrimidin-4-one

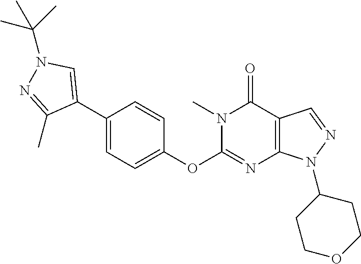 [SMILES: Cc1nn(C(C)(C)C)cc1-c1ccc(Oc2nc3c(cnn3C3CCOCC3)c(=O)n2C)cc1]

¹H NMR (400 MHz, CDCl₃) δ 8.01 (s, 1H), 7.64 (s, 1H), 7.49 (d, J=8.7 Hz, 2H), 7.24 (d, J=8.7 Hz, 2H), 4.43 (tt, J=4.2, 11.4 Hz, 1H), 4.03 (dd, J=2.6, 11.8 Hz, 2H), 3.67 (s, 3H), 3.44 (dt, J=1.6, 11.9 Hz, 2H), 2.46 (s, 3H), 2.31-2.15 (m, 2H), 1.86-1.76 (m, 2H), 1.64 (s, 9H). [M+H]=463.44.

### Example 85. 6-[4-(1-tert-Butyl-1H-pyrazol-4-yl)phenoxy]-5-methyl-1-(oxan-4-yl)-1H,4H,5H-pyrazolo[3,4-d]pyrimidin-4-one

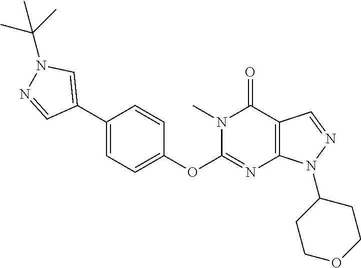 [SMILES: Cn1c(Oc2ccc(-c3cnn(C(C)(C)C)c3)cc2)nc2c(cnn2C2CCOCC2)c1=O]

¹H NMR (400 MHz, CDCl₃) δ 8.02 (s, 1H), 7.88 (s, 1H), 7.84-7.84 (m, 1H), 7.59 (d, J=8.6 Hz, 2H), 7.23 (d, J=8.7 Hz, 2H), 4.43 (tt, J=4.1, 11.4 Hz, 1H), 4.03 (dd, J=2.5, 11.8 Hz, 2H), 3.67 (s, 3H), 3.52-3.39 (m, 2H), 2.27-2.17 (m, 2H), 1.80 (dd, J=2.0, 12.5 Hz, 2H), 1.67 (s, 9H). [M+H]=449.42.

### Example 86. 6-[4-(1,3-Dimethyl-1H-pyrazol-4-yl)phenoxy]-5-methyl-1-(oxan-4-yl)-1H,4H,5H-pyrazolo[3,4-d]pyrimidin-4-one

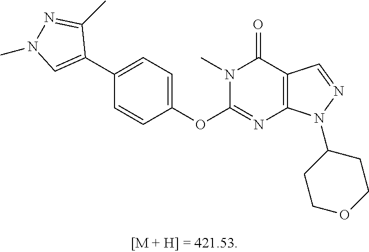 [SMILES: Cc1nn(C)cc1-c1ccc(Oc2nc3c(cnn3C3CCOCC3)c(=O)n2C)cc1]

### Example 87. 6-[4-(Dimethyl-1,3-oxazol-5-yl)phenoxy]-5-methyl-1-(oxan-4-yl)-1H,4H,5H-pyrazolo[3,4-d]pyrimidin-4-one

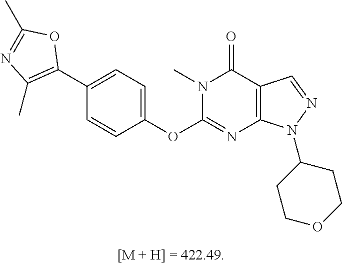 [SMILES: Cc1nc(C)c(-c2ccc(Oc3nc4c(cnn4C4CCOCC4)c(=O)n3C)cc2)o1]

### Example 88. 6-{[6-(1,3-Dimethyl-1H-pyrazol-4-yl)pyridin-3-yl]oxy}-5-methyl-1-(oxan-4-yl)-1H,4H,5H-pyrazolo[3,4-d]pyrimidin-4-one

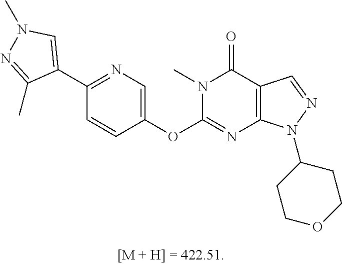 [SMILES: Cc1nn(C)cc1-c1ccc(Oc2nc3c(cnn3C3CCOCC3)c(=O)n2C)cn1]

Example 89 was prepared in a manner analogous to Example 21, with the appropriate starting material substitutions.

### Example 89. 5-Methyl-1-(oxan-4-yl)-6-{[(1R,4R)-4-(trifluoromethyl)cyclohexyl]methoxy}-1H,4H,5H-pyrazolo[3,4-d]pyrimidin-4-one

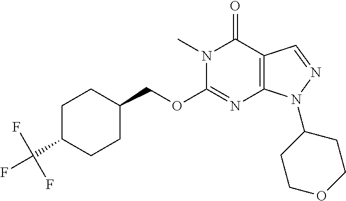 [SMILES: Cn1c(OC[C@H]2CC[C@H](C(F)(F)F)CC2)nc2c(cnn2C2CCOCC2)c1=O]

¹H NMR (400 MHz, CDCl₃) δ 7.98 (s, 1H), 4.66 (tt, J=4.2, 11.6 Hz, 1H), 4.30 (d, J=6.1 Hz, 2H), 4.15 (dd, J=4.0, 11.1 Hz, 2H), 3.59 (dt, J=1.8, 12.0 Hz, 2H), 3.46 (s, 3H), 2.39 (dq, J=4.6, 12.3 Hz, 2H), 2.10-2.00 (m, 5H), 1.95-1.81 (m, 3H), 1.48-1.35 (m, 2H), 1.28-1.13 (m, 2H). [M+H]=415.3.

Example 90-Example 93 were prepared in a manner analogous to Example 8, with the appropriate starting material substitutions.

### Example 90. 5-Methyl-1-[(3R)-oxan-3-yl]-6-{4-[1-(propan-2-yl)-1H-pyrazol-4-yl]phenoxy}-1H,4H,5H-pyrazolo[3,4-d]pyrimidin-4-one

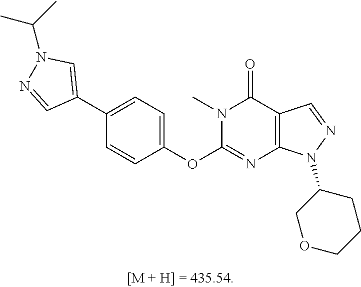 [SMILES: CC(C)n1cc(-c2ccc(Oc3nc4c(cnn4[C@@H]4CCCOC4)c(=O)n3C)cc2)cn1]

### Example 91. 6-[4-(Dimethyl-1,3-oxazol-5-yl)phenoxy]-5-methyl-1-[(3R)-oxan-3-yl]-1H,4H,5H-pyrazolo[3,4-d]pyrimidin-4-one

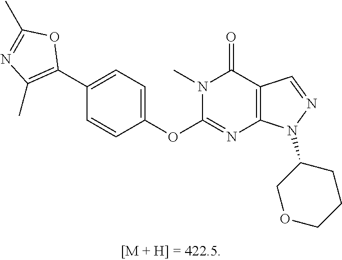 [SMILES: Cc1nc(C)c(-c2ccc(Oc3nc4c(cnn4[C@@H]4CCCOC4)c(=O)n3C)cc2)o1]

### Example 92. 6-[4-(1-Ethyl-1H-pyrazol-4-yl)phenoxy]-5-methyl-1-[(3R)-oxan-3-yl]-1H,4H,5H-pyrazolo[3,4-d]pyrimidin-4-one

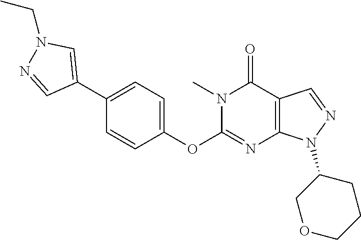 [SMILES: CCn1cc(-c2ccc(Oc3nc4c(cnn4[C@@H]4CCCOC4)c(=O)n3C)cc2)cn1]

¹H NMR (400 MHz, CDCl₃) δ=8.01 (s, 1H), 7.83 (s, 1H), 7.72 (s, 1H), 7.58 (d, J=8.7 Hz, 2H), 7.23 (d, J=8.6 Hz, 2H), 4.48-4.39 (m, 1H), 4.27 (q, J=7.3 Hz, 2H), 3.93-3.85 (m, 2H), 3.73-3.66 (m, 4H), 3.41-3.32 (m, 1H), 2.21 (dd, J=5.7, 12.2 Hz, 1H), 2.03 (d, J=12.7 Hz, 1H), 1.78-1.68 (m, 2H), 1.58 (t, J=7.3 Hz, 3H). [M+H]=421.4.

### Example 93. 6-[2-Fluoro-4-(morpholin-4-yl)phenoxy]-5-methyl-1-[(3R)-oxan-3-yl]-1H,4H,5H-pyrazolo[3,4-d]pyrimidin-4-one

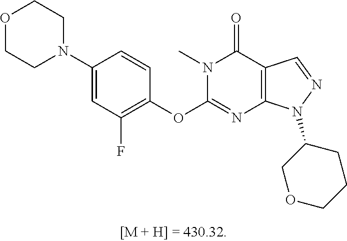 [SMILES: Cn1c(Oc2ccc(N3CCOCC3)cc2F)nc2c(cnn2[C@@H]2CCCOC2)c1=O]

Example 94 was prepared in a manner analogous to Example 10, with the appropriate starting material substitutions.

### Example 94. 6-[4-(1-Ethyl-1H-pyrazol-4-yl)phenoxy]-5-methyl-1-(propan-2-yl)-1H,4H,5H-pyrazolo[3,4-d]pyrimidin-4-one

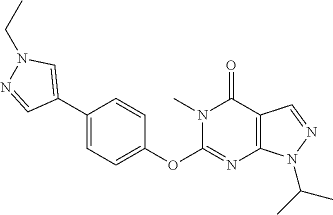 [SMILES: CCn1cc(-c2ccc(Oc3nc4c(cnn4C(C)C)c(=O)n3C)cc2)cn1]

¹H NMR (400 MHz, CDCl₃) δ 8.03 (s, 1H), 7.90 (s, 1H), 7.73 (s, 1H), 7.57 (d, J=8.7 Hz, 2H), 7.25 (d, J=8.6 Hz, 2H), 4.62 (td, J=6.7, 13.4 Hz, 1H), 4.30 (q, J=7.3 Hz, 2H), 3.67 (s, 3H), 1.57 (t, J=7.3 Hz, 3H), 1.39 (d, J=6.7 Hz, 6H). [M+H]=379.20.

Example 95-Example 201 were prepared in a manner analogous to Example 20, with the appropriate starting material substitutions.

### Example 95. 6-[4-(1-Acetylpiperidin-4-yl)phenoxy]-7-methyl-3-(propan-2-yl)-7H,8H-imidazo[1,5-a]pyrazin-8-one

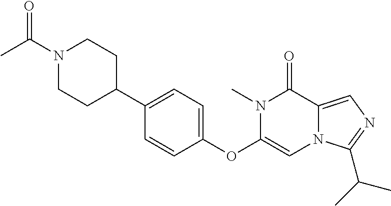 [SMILES: CC(=O)N1CCC(c2ccc(Oc3cn4c(C(C)C)ncc4c(=O)n3C)cc2)CC1]

¹H NMR (400 MHz, CDCl₃) δ 7.92 (s, 1H), 7.25 (d, J=8.7 Hz, 2H), 7.05 (d, J=8.7 Hz, 2H), 6.60 (s, 1H), 4.93-4.76 (m, 1H), 4.06-3.92 (m, 1H), 3.41 (s, 3H), 3.20 (dt, J=2.2, 13.1 Hz, 1H), 3.02 (spt, J=6.9 Hz, 1H), 2.79 (tt, J=3.5, 12.2 Hz, 1H), 2.70-2.58 (m, 1H), 2.16 (s, 3H), 2.00-1.89 (m, 2H), 1.73-1.56 (m, 2H), 1.36 (d, J=6.8 Hz, 6H). [M+H]=409.5.

### Example 96. 6-[4-(4-Acetylpiperazin-1-yl)phenoxy]-7-methyl-3-(propan-2-yl)-7H,8H-imidazo[1,5-a]pyrazin-8-one

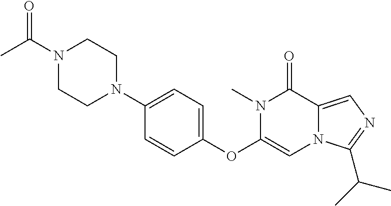 [SMILES: CC(=O)N1CCN(c2ccc(Oc3cn4c(C(C)C)ncc4c(=O)n3C)cc2)CC1]

¹H NMR (400 MHz, CDCl₃) δ 7.91 (s, 1H), 7.09-7.04 (m, 2H), 7.01-6.95 (m, 2H), 6.42 (s, 1H), 3.87-3.79 (m, 2H), 3.70-3.64 (m, 2H), 3.46 (s, 3H), 3.19 (td, J=5.2, 12.5 Hz, 4H), 2.98 (td, J=6.9, 13.7 Hz, 1H), 2.17 (s, 3H), 1.34 (d, J=6.8 Hz, 6H). [M+H]=410.4.

### Example 97. 6-{[6-(1,3-Dimethyl-1H-pyrazol-4-yl)pyridin-3-yl]oxy}-7-methyl-3-(propan-2-yl)-7H,8H-imidazo[1,5-a]pyrazin-8-one

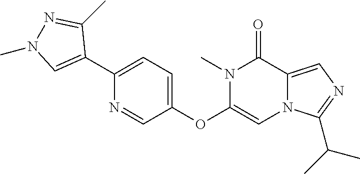 [SMILES: Cc1nn(C)cc1-c1ccc(Oc2cn3c(C(C)C)ncc3c(=O)n2C)cn1]

¹H NMR (400 MHz, CDCl₃) δ 8.50 (d, J=2.8 Hz, 1H), 7.94 (s, 1H), 7.82 (s, 1H), 7.53-7.48 (m, 1H), 7.45-7.41 (m, 1H), 6.55 (s, 1H), 3.92 (s, 3H), 3.49 (s, 3H), 3.02 (spt, J=6.9 Hz, 1H), 2.55 (s, 3H), 1.36 (d, J=6.8 Hz, 6H). [M+H]=379.4.

### Example 98. 7-Methyl-3-(propan-2-yl)-6-{[6-(pyridin-3-yl)pyridin-3-yl]oxy}-7H,8H-imidazo[1,5-a]pyrazin-8-one

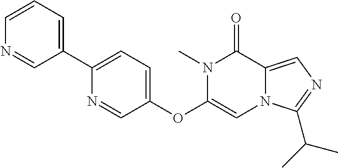 [SMILES: CC(C)c1ncc2c(=O)n(C)c(Oc3ccc(-c4cccnc4)nc3)cn12]

¹H NMR (400 MHz, CDCl₃) δ 9.22 (s, 1H), 8.70 (d, J=4.0 Hz, 1H), 8.64 (d, J=2.9 Hz, 1H), 8.34 (d, J=7.9 Hz, 1H), 7.96 (s, 1H), 7.85 (d, J=8.7 Hz, 1H), 7.55 (dd, J=2.9, 8.7 Hz, 1H), 7.46 (dd, J=4.8, 7.8 Hz, 1H), 6.67 (s, 1H), 3.48 (s, 3H), 3.05 (spt, J=6.9 Hz, 1H), 1.38 (d, J=7.0 Hz, 6H). [M+H]=362.3.

### Example 99. 6-[4-(1-Hydroxycyclobutyl)phenoxy]-7-methyl-3-(propan-2-yl)-7H,8H-imidazo[1,5-a]pyrazin-8-one

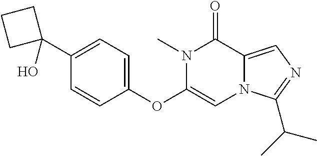 [SMILES: CC(C)c1ncc2c(=O)n(C)c(Oc3ccc(C4(O)CCC4)cc3)cn12]

¹H NMR (400 MHz, CDCl₃) δ 7.93 (s, 1H), 7.58 (d, J=8.8 Hz, 2H), 7.11 (d, J=8.8 Hz, 2H), 6.61 (s, 1H), 3.43 (s, 3H), 3.03 (spt, J=6.9 Hz, 1H), 2.68-2.53 (m, 2H), 2.42 (ddd, J=7.5, 9.5, 12.6 Hz, 2H), 2.15-2.00 (m, 2H), 1.84-1.68 (m, 1H), 1.37 (d, J=6.8 Hz, 6H). [M+H]=354.4.

### Example 100. 6-(4-Cyclobutoxyphenoxy)-7-methyl-3-(propan-2-yl)-7H,8H-imidazo[1,5-a]pyrazin-8-one

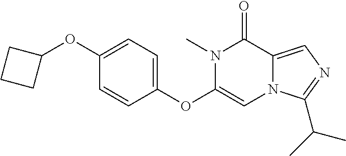 [SMILES: CC(C)c1ncc2c(=O)n(C)c(Oc3ccc(OC4CCC4)cc3)cn12]

¹H NMR (400 MHz, CDCl₃) δ 7.90 (s, 1H), 7.04 (d, J=9.0 Hz, 2H), 6.87 (d, J=9.0 Hz, 2H), 6.37 (s, 1H), 4.65 (quin, J=7.1 Hz, 1H), 3.47 (s, 3H), 2.97 (spt, J=6.9 Hz, 1H), 2.55-2.43 (m, 2H), 2.32-2.15 (m, 2H), 1.98-1.85 (m, 1H), 1.81-1.65 (m, 1H), 1.33 (d, J=6.8 Hz, 6H). [M+H]=354.4.

### Example 101. 6-[4-(Cyclobutylmethoxy)phenoxy]-7-methyl-3-(propan-2-yl)-7H,8H-imidazo[1,5-a]pyrazin-8-one

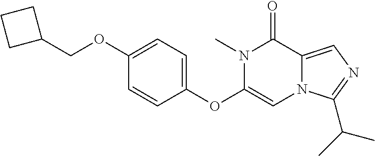 [SMILES: CC(C)c1ncc2c(=O)n(C)c(Oc3ccc(OCC4CCC4)cc3)cn12]

¹H NMR (400 MHz, CDCl₃) δ 7.90 (s, 1H), 7.10-7.03 (m, 2H), 6.98-6.93 (m, 2H), 6.35 (s, 1H), 3.96 (d, J=6.6 Hz, 2H), 3.48 (s, 3H), 2.97 (spt, J=6.9 Hz, 1H), 2.81 (quind, J=7.3, 14.6 Hz, 1H), 2.26-2.13 (m, 2H), 2.07-1.85 (m, 4H), 1.33 (d, J=6.8 Hz, 6H). [M+H]=368.4.

### Example 102. 7-Methyl-6-[4-(oxetan-2-ylmethoxy)phenoxy]-3-(propan-2-yl)-7H,8H-imidazo[1,5-a]pyrazin-8-one

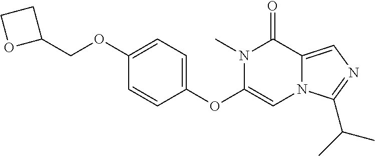 [SMILES: CC(C)c1ncc2c(=O)n(C)c(Oc3ccc(OCC4CCO4)cc3)cn12]

¹H NMR (400 MHz, CDCl₃) δ 7.90 (s, 1H), 7.12-6.99 (m, 4H), 6.35 (s, 1H), 5.22-5.14 (m, 1H), 4.77 (ddd, J=5.9, 7.2, 8.4 Hz, 1H), 4.69 (td, J=6.1, 9.0 Hz, 1H), 4.17 (dd, J=1.6, 4.2 Hz, 2H), 3.48 (s, 3H), 2.97 (spt, J=6.9 Hz, 1H), 2.88-2.79 (m, 1H), 2.73 (tdd, J=7.0, 8.9, 11.1 Hz, 1H), 1.33 (d, J=6.8 Hz, 6H). [M+H]=370.4.

### Example 103. 7-Methyl-6-[4-(oxan-4-ylmethyl)phenoxy]-3-(propan-2-yl)-7H,8H-imidazo[1,5-a]pyrazin-8-one

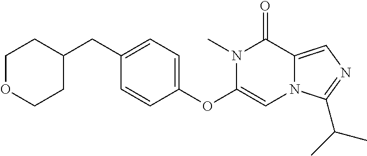 [SMILES: CC(C)c1ncc2c(=O)n(C)c(Oc3ccc(CC4CCOCC4)cc3)cn12]

¹H NMR (400 MHz, CDCl₃) δ 7.92 (s, 1H), 7.20 (d, J=8.6 Hz, 2H), 7.02 (d, J=8.6 Hz, 2H), 6.57 (s, 1H), 3.98 (dd, J=3.7, 11.1 Hz, 2H), 3.42 (s, 3H), 3.37 (dt, J=1.8, 11.7 Hz, 2H), 3.02 (spt, J=6.8 Hz, 1H), 2.59 (d, J=7.2 Hz, 2H), 1.84-1.71 (m, 1H), 1.57 (d, J=13.0 Hz, 2H), 1.43-1.38 (m, 1H), 1.36 (d, J=6.8 Hz, 6H), 1.32 (d, J=4.4 Hz, 1H). [M+H]=382.4.

### Example 104. 7-Methyl-6-[4-(oxetan-3-yloxy)phenoxy]-3-(propan-2-yl)-7H,8H-imidazo[1,5-a]pyrazin-8-one

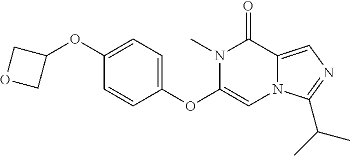 [SMILES: CC(C)c1ncc2c(=O)n(C)c(Oc3ccc(OC4COC4)cc3)cn12]

¹H NMR (400 MHz, CDCl₃) δ 7.91 (s, 1H), 7.06 (d, J=9.0 Hz, 2H), 6.76 (d, J=9.2 Hz, 2H), 6.41 (s, 1H), 5.23 (quin, J=5.6 Hz, 1H), 5.00 (t, J=6.8 Hz, 2H), 4.85-4.74 (m, 2H), 3.46 (s, 3H), 2.98 (spt, J=6.9 Hz, 1H), 1.34 (d, J=6.8 Hz, 6H). [M+H]=356.3.

### Example 105. 7-Methyl-6-[4-(oxolan-3-yloxy)phenoxy]-3-(propan-2-yl)-7H,8H-imidazo[1,5-a]pyrazin-8-one

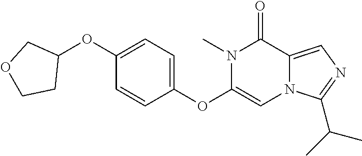 [SMILES: CC(C)c1ncc2c(=O)n(C)c(Oc3ccc(OC4CCOC4)cc3)cn12]

¹H NMR (400 MHz, CDCl₃) δ 7.90 (s, 1H), 7.07 (d, J=9.0 Hz, 2H), 6.92 (d, J=9.2 Hz, 2H), 6.39 (s, 1H), 4.94 (d, J=2.6 Hz, 1H), 4.11-4.00 (m, 3H), 3.95 (dt, J=4.4, 8.3 Hz, 1H), 3.47 (s, 3H), 2.98 (spt, J=6.8 Hz, 1H), 2.36-2.12 (m, 2H), 1.34 (d, J=6.8 Hz, 6H). [M+H]=370.4.

### Example 106. 7-Methyl-6-[4-(oxetan-3-ylmethoxy)phenoxy]-3-(propan-2-yl)-7H,8H-imidazo[1,5-a]pyrazin-8-one

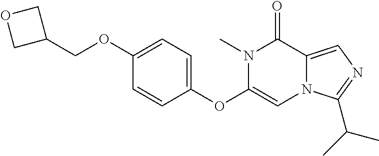 [SMILES: CC(C)c1ncc2c(=O)n(C)c(Oc3ccc(OCC4COC4)cc3)cn12]

¹H NMR (400 MHz, CDCl₃) δ 7.91 (s, 1H), 7.12-7.06 (m, 2H), 7.01-6.94 (m, 2H), 6.39 (s, 1H), 4.93 (dd, J=6.3, 7.8 Hz, 2H), 4.61 (t, J=6.1 Hz, 2H), 4.24 (d, J=6.7 Hz, 2H), 3.56-3.48 (m, 1H), 3.47 (s, 3H), 2.98 (spt, J=6.9 Hz, 1H), 1.34 (d, J=6.8 Hz, 6H). [M+H]=370.4.

### Example 107. 7-Methyl-6-[4-(oxolan-3-yl)phenoxy]-3-(propan-2-yl)-7H,8H-imidazo[1,5-a]pyrazin-8-one

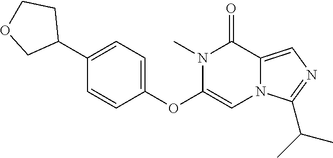 [SMILES: CC(C)c1ncc2c(=O)n(C)c(Oc3ccc(C4CCOC4)cc3)cn12]

¹H NMR (400 MHz, CDCl₃) δ 7.92 (s, 1H), 7.31 (d, J=8.6 Hz, 2H), 7.05 (d, J=8.7 Hz, 2H), 6.58 (s, 1H), 4.20-4.14 (m, 1H), 4.11 (dt, J=4.6, 8.4 Hz, 1H), 3.95 (q, J=7.9 Hz, 1H), 3.75 (dd, J=7.2, 8.4 Hz, 1H), 3.55-3.44 (m, 1H), 3.42 (s, 3H), 3.02 (spt, J=6.9 Hz, 1H), 2.42 (dtd, J=4.5, 7.8, 12.3 Hz, 1H), 2.02 (qd, J=8.0, 12.4 Hz, 1H), 1.36 (d, J=6.8 Hz, 6H). [M+H]=354.4.

### Example 108. 6-[4-(4-Hydroxyoxan-4-yl)phenoxy]-7-methyl-3-(propan-2-yl)-7H,8H-imidazo[1,5-a]pyrazin-8-one

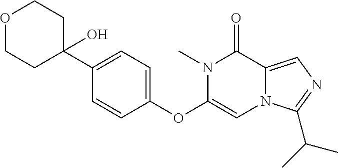 [SMILES: CC(C)c1ncc2c(=O)n(C)c(Oc3ccc(C4(O)CCOCC4)cc3)cn12]

¹H NMR (400 MHz, CDCl₃) δ 7.92 (s, 1H), 7.57 (d, J=8.8 Hz, 2H), 7.10 (d, J=8.9 Hz, 2H), 6.64 (s, 1H), 4.03-3.89 (m, 4H), 3.41 (s, 3H), 3.04 (spt, J=6.9 Hz, 1H), 2.26-2.15 (m, 2H), 1.77-1.70 (m, 3H), 1.37 (d, J=7.0 Hz, 6H). [M+H]=384.4.

### Example 109. 7-Methyl-6-{4-[(2R)-oxetan-2-ylmethoxy]phenoxy}-3-(propan-2-yl)-7H,8H-imidazo[1,5-a]pyrazin-8-one

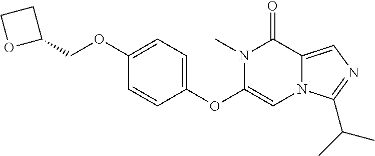 [SMILES: CC(C)c1ncc2c(=O)n(C)c(Oc3ccc(OC[C@H]4CCO4)cc3)cn12]

¹H NMR (400 MHz, CDCl₃) δ 7.90 (s, 1H), 7.12-7.01 (m, 4H), 6.34 (s, 1H), 5.22-5.13 (m, 1H), 4.77 (ddd, J=5.9, 7.2, 8.4 Hz, 1H), 4.69 (td, J=6.1, 9.0 Hz, 1H), 4.21-4.12 (m, 2H), 3.48 (s, 3H), 2.97 (spt, J=6.9 Hz, 1H), 2.88-2.80 (m, 1H), 2.79-2.69 (m, 1H), 1.33 (d, J=6.8 Hz, 6H). [M+H]=370.4.

### Example 110. 7-Methyl-6-{4-[(2S)-oxetan-2-ylmethoxy]phenoxy}-3-(propan-2-yl)-7H,8H-imidazo[1,5-a]pyrazin-8-one

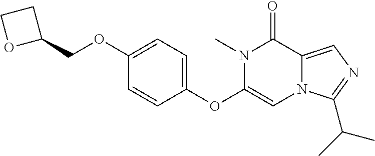 [SMILES: CC(C)c1ncc2c(=O)n(C)c(Oc3ccc(OC[C@@H]4CCO4)cc3)cn12]

¹H NMR (400 MHz, CDCl₃) δ 7.90 (s, 1H), 7.12-7.00 (m, 4H), 6.34 (s, 1H), 5.23-5.14 (m, 1H), 4.77 (ddd, J=5.9, 7.2, 8.4 Hz, 1H), 4.69 (td, J=6.1, 9.0 Hz, 1H), 4.27-4.13 (m, 2H), 3.48 (s, 3H), 2.97 (spt, J=6.9 Hz, 1H), 2.89-2.79 (m, 1H), 2.79-2.66 (m, 1H), 1.33 (d, J=6.8 Hz, 6H). [M+H]=370.4.

### Example 111. 6-(4-Cyclopropoxyphenoxy)-7-methyl-3-(propan-2-yl)-7H,8H-imidazo[1,5-a]pyrazin-8-one

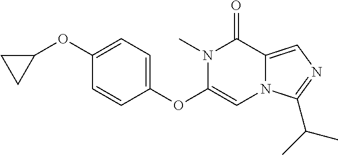 [SMILES: CC(C)c1ncc2c(=O)n(C)c(Oc3ccc(OC4CC4)cc3)cn12]

¹H NMR (400 MHz, CDCl₃) δ 7.91 (s, 1H), 7.14-7.09 (m, 2H), 7.08-7.03 (m, 2H), 6.38 (s, 1H), 3.82-3.72 (m, 1H), 3.48 (s, 3H), 2.99 (spt, J=6.9 Hz, 1H), 1.34 (d, J=6.8 Hz, 6H), 0.86-0.79 (m, 4H). [M+H]=340.3.

### Example 112. 7-Methyl-6-[4-(oxan-4-yloxy)phenoxy]-3-(propan-2-yl)-7H,8H-imidazo[1,5-a]pyrazin-8-one

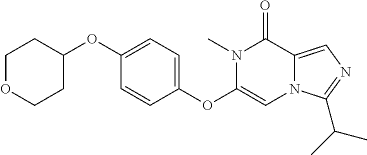 [SMILES: CC(C)c1ncc2c(=O)n(C)c(Oc3ccc(OC4CCOCC4)cc3)cn12]

¹H NMR (400 MHz, CDCl₃) δ 7.91 (s, 1H), 7.11-7.04 (m, 2H), 7.00-6.96 (m, 2H), 6.40 (s, 1H), 4.49 (tt, J=3.9, 7.9 Hz, 1H), 4.08-3.98 (m, 2H), 3.61 (ddd, J=3.1, 8.4, 11.7 Hz, 2H), 3.47 (s, 3H), 2.99 (spt, J=6.8 Hz, 1H), 2.11-2.01 (m, 2H), 1.89-1.76 (m, 2H), 1.35 (d, J=6.8 Hz, 6H). [M+H]=384.4.

### Example 113. 7-Methyl-6-{4-[(3R)-oxolan-3-yloxy]phenoxy}-3-(propan-2-yl)-7H,8H-imidazo[1,5-a]pyrazin-8-one

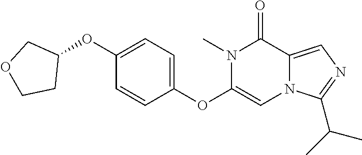 [SMILES: CC(C)c1ncc2c(=O)n(C)c(Oc3ccc(O[C@@H]4CCOC4)cc3)cn12]

¹H NMR (400 MHz, CDCl₃) δ 7.92 (s, 1H), 7.07 (d, J=9.2 Hz, 2H), 6.93 (d, J=9.2 Hz, 2H), 6.38 (s, 1H), 4.94 (dt, J=3.2, 5.5 Hz, 1H), 4.11-4.00 (m, 3H), 3.98-3.88 (m, 1H), 3.48 (s, 3H), 2.99 (spt, J=6.8 Hz, 1H), 2.33-2.14 (m, 2H), 1.35 (d, J=6.8 Hz, 6H). [M+H]=370.4.

### Example 114. 7-Methyl-6-{4-[(3S)-oxolan-3-yloxy]phenoxy}-3-(propan-2-yl)-7H,8H-imidazo[1,5-a]pyrazin-8-one

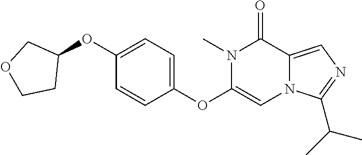 [SMILES: CC(C)c1ncc2c(=O)n(C)c(Oc3ccc(O[C@H]4CCOC4)cc3)cn12]

¹H NMR (400 MHz, CDCl₃) δ 7.92 (s, 1H), 7.07 (d, J=9.2 Hz, 2H), 6.93 (d, J=9.0 Hz, 2H), 6.38 (s, 1H), 5.00-4.91 (m, 1H), 4.10-4.01 (m, 3H), 3.99-3.88 (m, 1H), 3.50-3.47 (m, 3H), 2.99 (spt, J=6.8 Hz, 1H), 2.34-2.15 (m, 2H), 1.35 (d, J=6.8 Hz, 6H). [M+H]=370.3.

### Example 115. 7-Methyl-6-{4-[(2R)-oxolan-2-ylmethoxy]phenoxy}-3-(propan-2-yl)-7H,8H-imidazo[1,5-a]pyrazin-8-one

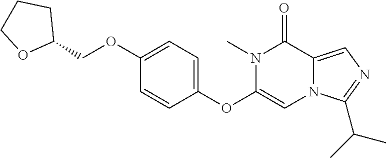 [SMILES: CC(C)c1ncc2c(=O)n(C)c(Oc3ccc(OC[C@H]4CCCO4)cc3)cn12]

¹H NMR (400 MHz, CDCl₃) δ 7.92 (s, 1H), 7.10-7.05 (m, 2H), 7.02-6.96 (m, 2H), 6.30 (s, 1H), 4.37-4.27 (m, 1H), 4.07-3.94 (m, 3H), 3.92-3.82 (m, 1H), 3.49 (s, 3H), 2.99 (spt, J=6.9 Hz, 1H), 2.19-2.07 (m, 1H), 2.07-1.91 (m, 2H), 1.85-1.72 (m, 1H), 1.35 (d, J=7.0 Hz, 6H). [M+H]=384.3.

### Example 116. 7-Methyl-6-{4-[(2S)-oxolan-2-ylmethoxy]phenoxy}-3-(propan-2-yl)-7H,8H-imidazo[1,5-a]pyrazin-8-one

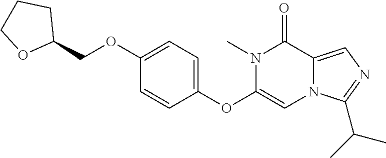 [SMILES: CC(C)c1ncc2c(=O)n(C)c(Oc3ccc(OC[C@@H]4CCCO4)cc3)cn12]

¹H NMR (400 MHz, CDCl₃) δ 7.91 (s, 1H), 7.10-7.05 (m, 2H), 7.02-6.97 (m, 2H), 6.31 (s, 1H), 4.36-4.28 (m, 1H), 4.07-4.00 (m, 2H), 3.99-3.95 (m, 1H), 3.91-3.84 (m, 1H), 3.49 (s, 3H), 3.08-2.90 (m, 1H), 2.19-2.09 (m, 1H), 2.05-1.92 (m, 2H), 1.85-1.75 (m, 1H), 1.34 (d, J=6.8 Hz, 6H). [M+H]=384.4.

### Example 117. 7-Methyl-6-{4-[(3R)-oxolan-3-yl]phenoxy}-3-(propan-2-yl)-7H,8H-imidazo[1,5-a]pyrazin-8-one

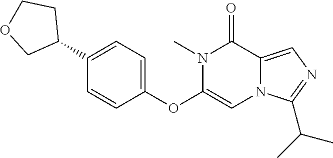 [SMILES: CC(C)c1ncc2c(=O)n(C)c(Oc3ccc([C@H]4CCOC4)cc3)cn12]

¹H NMR (400 MHz, CDCl₃) δ=7.94 (s, 1H), 7.32 (d, J=8.7 Hz, 2H), 7.06 (d, J=8.7 Hz, 2H), 6.57 (s, 1H), 4.19-4.14 (m, 1H), 4.11 (dt, J=4.6, 8.4 Hz, 1H), 4.00-3.90 (m, 1H), 3.75 (dd, J=7.2, 8.4 Hz, 1H), 3.52-3.45 (m, 1H), 3.43 (s, 3H), 3.04 (spt, J=6.9 Hz, 1H), 2.42 (dtd, J=4.6, 7.8, 12.4 Hz, 1H), 2.02 (qd, J=8.0, 12.4 Hz, 1H), 1.37 (d, J=6.8 Hz, 6H). [M+H]=354.4.

### Example 118. 7-Methyl-6-{4-[(3S)-oxolan-3-yl]phenoxy}-3-(propan-2-yl)-7H,8H-imidazo[1,5-a]pyrazin-8-one

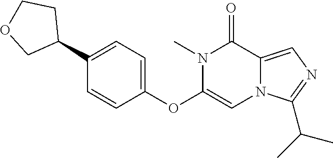 [SMILES: CC(C)c1ncc2c(=O)n(C)c(Oc3ccc([C@@H]4CCOC4)cc3)cn12]

¹H NMR (400 MHz, CDCl₃) δ 7.93 (s, 1H), 7.32 (d, J=8.6 Hz, 2H), 7.06 (d, J=8.7 Hz, 2H), 6.57 (s, 1H), 4.19-4.14 (m, 1H), 4.11 (dt, J=4.6, 8.4 Hz, 1H), 3.99-3.89 (m, 1H), 3.75 (dd, J=7.2, 8.4 Hz, 1H), 3.53-3.44 (m, 1H), 3.43 (s, 3H), 3.03 (spt, J=6.9 Hz, 1H), 2.42 (dtd, J=4.5, 7.8, 12.3 Hz, 1H), 2.02 (qd, J=8.0, 12.4 Hz, 1H), 1.37 (d, J=6.8 Hz, 6H). [M+H]=354.4.

### Example 119. 7-Methyl-3-(propan-2-yl)-6-({6-[1-(propan-2-yl)-1H-pyrazol-4-yl]pyridin-3-yl}oxy)-7H,8H-imidazo[1,5-a]pyrazin-8-one

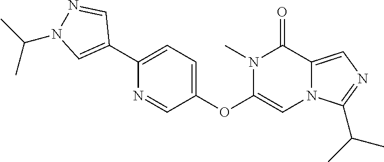 [SMILES: CC(C)c1ncc2c(=O)n(C)c(Oc3ccc(-c4cnn(C(C)C)c4)nc3)cn12]

¹H NMR (400 MHz, CDCl₃) δ 8.47 (d, J=2.9 Hz, 1H), 8.03 (s, 1H), 7.97 (s, 1H), 7.94 (s, 1H), 7.56 (d, J=8.7 Hz, 1H), 7.45 (dd, J=2.9, 8.7 Hz, 1H), 6.48 (s, 1H), 4.59 (spt, J=6.7 Hz, 1H), 3.50 (s, 3H), 3.01 (spt, J=6.8 Hz, 1H), 1.59 (d, J=6.7 Hz, 6H), 1.36 (d, J=6.8 Hz, 6H). [M+H]=393.4.

### Example 120. 7-Methyl-3-(propan-2-yl)-6-{4-[1-(propan-2-yl)-1H-pyrazol-4-yl]phenoxy}-7H,8H-imidazo[1,5-a]pyrazin-8-one

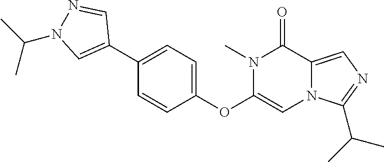 [SMILES: CC(C)c1ncc2c(=O)n(C)c(Oc3ccc(-c4cnn(C(C)C)c4)cc3)cn12]

¹H NMR (400 MHz, CDCl₃) δ 7.94 (s, 1H), 7.79 (s, 1H), 7.70 (s, 1H), 7.55 (d, J=8.8 Hz, 2H), 7.12 (d, J=8.8 Hz, 2H), 6.52 (s, 1H), 4.57 (spt, J=6.7 Hz, 1H), 3.47 (s, 3H), 3.03 (spt, J=6.9 Hz, 1H), 1.58 (d, J=6.7 Hz, 6H), 1.37 (d, J=7.0 Hz, 6H). [M+H]=392.4.

### Example 121. 7-Methyl-6-[4-(1-methyl-1H-pyrazol-4-yl)phenoxy]-3-(propan-2-yl)-7H,8H-imidazo[1,5-a]pyrazin-8-one

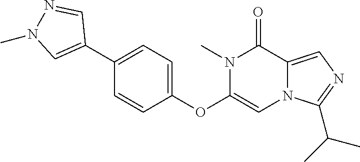 [SMILES: CC(C)c1ncc2c(=O)n(C)c(Oc3ccc(-c4cnn(C)c4)cc3)cn12]

¹H NMR (400 MHz, CDCl₃) δ 7.94 (s, 1H), 7.87 (d, J=8.8 Hz, 2H), 7.43 (d, J=2.3 Hz, 1H), 7.15 (d, J=8.8 Hz, 2H), 6.55 (d, J=2.2 Hz, 1H), 6.52 (s, 1H), 3.99 (s, 3H), 3.48 (s, 3H), 3.02 (spt, J=6.9 Hz, 1H), 1.36 (d, J=7.0 Hz, 6H). [M+H]=364.4.

### Example 122. 1-(2-Methoxyethyl)-6-{[7-methyl-8-oxo-3-(propan-2-yl)-7H,8H-imidazo[1,5-a]pyrazin-6-yl]oxy}-1,2,3,4-tetrahydroquinolin-2-one

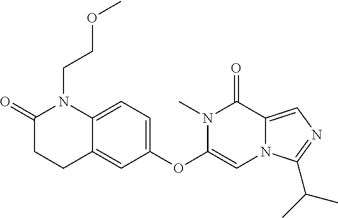 [SMILES: COCCN1C(=O)CCc2cc(Oc3cn4c(C(C)C)ncc4c(=O)n3C)ccc21]

¹H NMR (400 MHz, CDCl₃) δ 7.94 (s, 1H), 7.33 (d, J=8.8 Hz, 1H), 6.98 (dd, J=2.9, 8.9 Hz, 1H), 6.92 (d, J=2.9 Hz, 1H), 6.55 (s, 1H), 4.12 (t, J=5.6 Hz, 2H), 3.69 (t, J=5.6 Hz, 2H), 3.44 (s, 3H), 3.39 (s, 3H), 3.05 (spt, J=6.8 Hz, 1H), 2.96-2.88 (m, 2H), 2.74-2.66 (m, 2H), 1.38 (d, J=7.0 Hz, 6H). [M+H]=411.4.

### Example 123. 1-Methyl-6-{[7-methyl-8-oxo-3-(propan-2-yl)-7H,8H-imidazo[1,5-a]pyrazin-6-yl]oxy}-1,2,3,4-tetrahydroquinolin-2-one

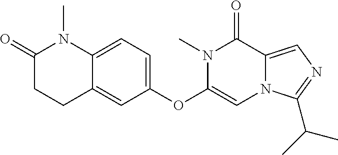 [SMILES: CC(C)c1ncc2c(=O)n(C)c(Oc3ccc4c(c3)CCC(=O)N4C)cn12]

¹H NMR (400 MHz, CDCl₃) δ 7.94 (s, 1H), 7.02 (d, J=1.2 Hz, 2H), 6.95 (s, 1H), 6.53 (s, 1H), 3.45 (s, 3H), 3.39 (s, 3H), 3.04 (spt, J=6.9 Hz, 1H), 2.97-2.90 (m, 2H), 2.73-2.65 (m, 2H), 1.37 (d, J=6.8 Hz, 6H). [M+H]=367.4.

### Example 124. 6-{4-[(3S)-1-Acetylpiperidin-3-yl]phenoxy}-7-methyl-3-(propan-2-yl)-7H,8H-imidazo[1,5-a]pyrazin-8-one

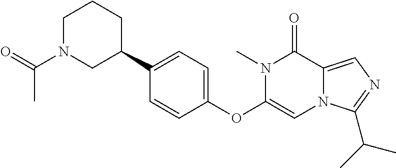 [SMILES: CC(=O)N1CCC[C@@H](c2ccc(Oc3cn4c(C(C)C)ncc4c(=O)n3C)cc2)C1]

¹H NMR (400 MHz, CDCl₃) δ 7.94 (s, 1H), 7.31 (d, J=2.9 Hz, 1H), 7.15-7.04 (m, 2H), 6.52 (s, 1H), 4.87-4.67 (m, 1H), 3.90 (d, J=13.6 Hz, 1H), 3.44 (s, 3H), 3.18-2.98 (m, 2H), 2.73 (tdd, J=3.8, 7.7, 15.4 Hz, 1H), 2.64-2.53 (m, 1H), 2.17 (s, 3H), 2.10 (d, J=8.6 Hz, 1H), 1.95-1.81 (m, 1H), 1.79-1.54 (m, 3H), 1.41-1.35 (m, 6H). [M+H]=409.5.

### Example 125. 6-{4-[(3R)-1-Acetylpiperidin-3-yl]phenoxy}-7-methyl-3-(propan-2-yl)-7H,8H-imidazo[1,5-a]pyrazin-8-one

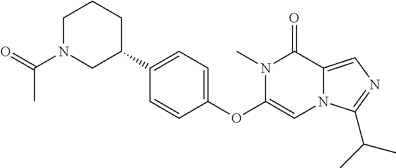 [SMILES: CC(=O)N1CCC[C@H](c2ccc(Oc3cn4c(C(C)C)ncc4c(=O)n3C)cc2)C1]

¹H NMR (400 MHz, CDCl₃) δ 7.94 (s, 1H), 7.31 (d, J=3.1 Hz, 1H), 7.12-7.03 (m, 2H), 6.51 (s, 1H), 4.85-4.68 (m, 1H), 3.90 (d, J=13.6 Hz, 1H), 3.45 (s, 3H), 3.17-2.99 (m, 2H), 2.73 (tdd, J=3.9, 7.7, 15.4 Hz, 1H), 2.65-2.51 (m, 1H), 2.17 (s, 3H), 2.10 (d, J=8.6 Hz, 1H), 1.95-1.81 (m, 1H), 1.79-1.54 (m, 3H), 1.38 (d, J=6.8 Hz, 6H). [M+H]=409.5.

### Example 126. 7-Methyl-6-phenoxy-3-(propan-2-yl)-7H,8H-imidazo[1,5-a]pyrazin-8-one

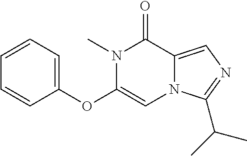 [SMILES: CC(C)c1ncc2c(=O)n(C)c(Oc3ccccc3)cn12]

¹H NMR (400 MHz, CDCl₃) δ 7.94 (s, 1H), 7.48-7.40 (m, 2H), 7.26 (t, J=7.3 Hz, 1H), 7.15-7.08 (m, 2H), 6.58 (s, 1H), 3.44 (s, 3H), 3.03 (spt, J=6.9 Hz, 1H), 1.37 (d, J=6.8 Hz, 6H). [M+H]=284.3.

### Example 127. 7-Methyl-3-(propan-2-yl)-6-{4-[4-(trifluoromethyl)pyrimidin-2-yl]phenoxy}-7H,8H-imidazo[1,5-a]pyrazin-8-one

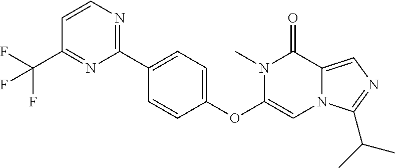 [SMILES: CC(C)c1ncc2c(=O)n(C)c(Oc3ccc(-c4nccc(C(F)(F)F)n4)cc3)cn12]

¹H NMR (400 MHz, CDCl₃) δ 9.07 (d, J=4.9 Hz, 1H), 8.62 (d, J=8.8 Hz, 2H), 7.97 (s, 1H), 7.55 (d, J=4.9 Hz, 1H), 7.24 (d, J=8.8 Hz, 2H), 6.71 (s, 1H), 3.45 (s, 3H), 3.16-3.01 (m, 1H), 1.40 (d, J=7.0 Hz, 6H). [M+H]=430.3.

### Example 128. 6-[4-(4-Methoxypyrimidin-2-yl)phenoxy]-7-methyl-3-(propan-2-yl)-7H,8H-imidazo[1,5-a]pyrazin-8-one

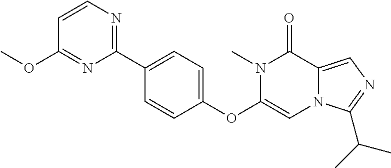 [SMILES: COc1ccnc(-c2ccc(Oc3cn4c(C(C)C)ncc4c(=O)n3C)cc2)n1]

¹H NMR (400 MHz, CDCl₃) δ 8.57-8.50 (m, 3H), 7.96 (s, 1H), 7.21 (d, J=8.9 Hz, 2H), 6.70-6.65 (m, 2H), 4.11 (s, 3H), 3.45 (s, 3H), 3.06 (spt, J=6.9 Hz, 1H), 1.38 (d, J=6.8 Hz, 6H). [M+H]=392.3.

### Example 129. 7-Methyl-6-{4-[4-(methylsulfanyl)pyrimidin-2-yl]phenoxy}-3-(propan-2-yl)-7H,8H-imidazo[1,5-a]pyrazin-8-one

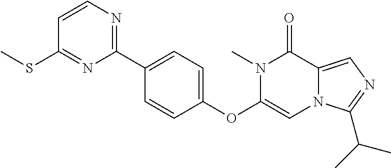 [SMILES: CSc1ccnc(-c2ccc(Oc3cn4c(C(C)C)ncc4c(=O)n3C)cc2)n1]

¹H NMR (400 MHz, CDCl₃) δ 8.56 (d, J=8.9 Hz, 2H), 8.44 (d, J=5.5 Hz, 1H), 7.96 (s, 1H), 7.21 (d, J=8.9 Hz, 2H), 7.11 (d, J=5.4 Hz, 1H), 6.67 (s, 1H), 3.45 (s, 3H), 3.12-3.00 (m, 1H), 2.69 (s, 3H), 1.39 (d, J=6.8 Hz, 6H). [M+H]=408.3.

### Example 130. 6-[4-(Dimethyl-1,3-oxazol-5-yl)phenoxy]-7-methyl-3-(propan-2-yl)-7H,8H-imidazo[1,5-a]pyrazin-8-one

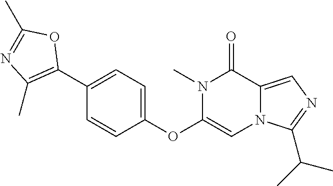 [SMILES: Cc1nc(C)c(-c2ccc(Oc3cn4c(C(C)C)ncc4c(=O)n3C)cc2)o1]

87% HPLC pure but 80% pure by NMR: ¹H NMR (400 MHz, CDCl₃) δ 7.97 (s, 1H), 7.65 (d, J=8.8 Hz, 2H), 7.18 (d, J=8.8 Hz, 2H), 6.59 (s, 1H), 3.46 (s, 3H), 3.08 (quind, J=6.9, 13.8 Hz, 1H), 2.51 (s, 3H), 2.41 (s, 3H), 1.40 (d, J=6.8 Hz, 6H). [M+H]=379.3.

### Example 131. 7-Methyl-6-[4-(4-methylpyrimidin-2-yl)phenoxy]-3-(propan-2-yl)-7H,8H-imidazo[1,5-a]pyrazin-8-one

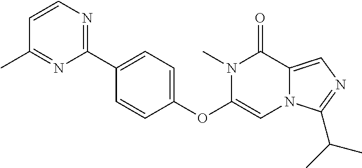 [SMILES: Cc1ccnc(-c2ccc(Oc3cn4c(C(C)C)ncc4c(=O)n3C)cc2)n1]

¹H NMR (400 MHz, CDCl₃) δ 8.67 (d, J=5.01 Hz, 1H), 8.54 (d, J=8.68 Hz, 2H), 7.94 (s, 1H), 7.21 (d, J=8.68 Hz, 2H), 7.10 (d, J=5.01 Hz, 1H), 6.66 (s, 1H), 3.45 (s, 3H), 3.04 (spt, J=6.77 Hz, 1H), 2.62 (s, 3H), 1.37 (d, J=6.97 Hz, 6H). [M+H]=376.3.

### Example 132. 7-Methyl-3-(propan-2-yl)-6-{4-[6-(trifluoromethyl)pyridin-2-yl]phenoxy}-7H,8H-imidazo[1,5-a]pyrazin-8-one

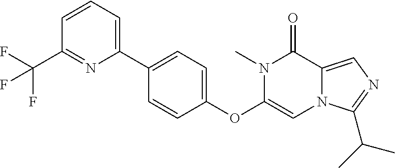 [SMILES: CC(C)c1ncc2c(=O)n(C)c(Oc3ccc(-c4cccc(C(F)(F)F)n4)cc3)cn12]

¹H NMR (400 MHz, CDCl₃) δ 8.17 (d, J=8.7 Hz, 2H), 8.02-7.96 (m, 2H), 7.95-7.91 (m, 1H), 7.67 (d, J=7.5 Hz, 1H), 7.25 (d, J=8.7 Hz, 2H), 6.63 (s, 1H), 3.47 (s, 3H), 3.14-2.99 (m, 1H), 1.40 (d, J=6.8 Hz, 6H). [M+H]=429.3.

### Example 133. 6-[4-(3-Cyclopropyl-1-methyl-1H-pyrazol-4-yl)phenoxy]-7-methyl-3-(propan-2-yl)-7H,8H-imidazo[1,5-a]pyrazin-8-one

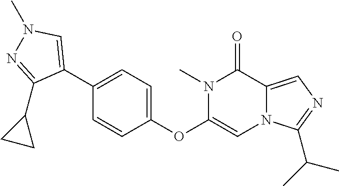 [SMILES: CC(C)c1ncc2c(=O)n(C)c(Oc3ccc(-c4cn(C)nc4C4CC4)cc3)cn12]

¹H NMR (400 MHz, CDCl₃) δ 8.00 (s, 1H), 7.63 (d, J=8.6 Hz, 2H), 7.45 (s, 1H), 7.15 (d, J=8.6 Hz, 2H), 6.53 (s, 1H), 3.88 (s, 3H), 3.51 (s, 3H), 3.18-3.03 (m, 1H), 1.99-1.86 (m, 1H), 1.42 (d, J=6.8 Hz, 6H), 1.01-0.94 (m, 4H). [M+H]=404.4.

### Example 134. 7-Methyl-3-(propan-2-yl)-6-{4-[4-(propan-2-yl)pyrimidin-2-yl]phenoxy}-7H,8H-imidazo[1,5-a]pyrazin-8-one

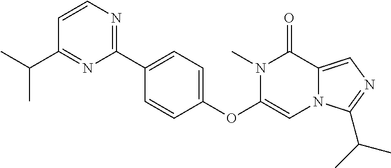 [SMILES: CC(C)c1ccnc(-c2ccc(Oc3cn4c(C(C)C)ncc4c(=O)n3C)cc2)n1]

¹H NMR (400 MHz, CDCl₃) δ 8.79 (d, J=6.1 Hz, 1H), 8.63 (d, J=8.7 Hz, 2H), 7.95 (s, 1H), 7.38 (d, J=6.0 Hz, 1H), 7.27 (br s, 2H), 6.54 (s, 1H), 3.39 (s, 3H), 3.34-3.25 (m, 1H), 3.17 (td, J=6.8, 13.6 Hz, 1H), 1.50-1.42 (m, 12H). [M+H]=404.3.

### Example 135. 7-Methyl-3-(propan-2-yl)-6-{4-[4-(trifluoromethyl)pyridin-2-yl]phenoxy}-7H,8H-imidazo[1,5-a]pyrazin-8-one

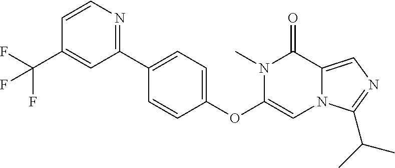 [SMILES: CC(C)c1ncc2c(=O)n(C)c(Oc3ccc(-c4cc(C(F)(F)F)ccn4)cc3)cn12]

¹H NMR (400 MHz, CDCl₃) δ 8.90 (d, J=5.01 Hz, 1H), 8.13 (d, J=8.93 Hz, 2H), 7.95 (s, 1H), 7.93 (s, 1H), 7.50 (d, J=5.01 Hz, 1H), 7.25 (d, J=8.93 Hz, 2H), 6.69 (s, 1H), 3.45 (s, 3H), 3.05 (spt, J=6.91 Hz, 1H), 1.38 (d, J=6.85 Hz, 6H). [M+H]=429.3.

### Example 136. 7-Methyl-3-(propan-2-yl)-6-{4-[5-(trifluoromethyl)pyridin-3-yl]phenoxy}-7H,8H-imidazo[1,5-a]pyrazin-8-one

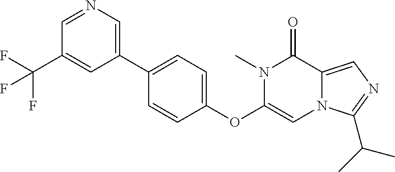 [SMILES: CC(C)c1ncc2c(=O)n(C)c(Oc3ccc(-c4cncc(C(F)(F)F)c4)cc3)cn12]

¹H NMR (400 MHz, CDCl₃) δ 9.04 (d, J=1.47 Hz, 1H), 8.91 (s, 1H), 8.10 (s, 1H), 7.96 (s, 1H), 7.68 (d, J=8.68 Hz, 2H), 7.26 (d, J=8.68 Hz, 2H), 6.72 (s, 1H), 3.44 (s, 3H), 3.07 (spt, J=6.87 Hz, 1H), 1.39 (d, J=6.85 Hz, 6H). [M+H]=429.3.

### Example 137. 7-Methyl-3-(propan-2-yl)-6-{4-[2-(trifluoromethyl)pyridin-4-yl]phenoxy}-7H,8H-imidazo[1,5-a]pyrazin-8-one

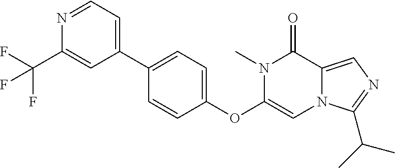 [SMILES: CC(C)c1ncc2c(=O)n(C)c(Oc3ccc(-c4ccnc(C(F)(F)F)c4)cc3)cn12]

¹H NMR (400 MHz, CDCl₃) δ 8.82 (d, J=5.01 Hz, 1H), 7.96 (s, 1H), 7.89 (s, 1H), 7.74 (d, J=8.80 Hz, 2H), 7.70 (d, J=5.01 Hz, 1H), 7.26 (d, J=8.68 Hz, 2H), 6.75 (s, 1H), 3.43 (s, 3H), 3.07 (spt, J=6.79 Hz, 1H), 1.39 (d, J=6.85 Hz, 6H). [M+H]=429.3.

### Example 138. 7-Methyl-6-{4-[1-methyl-3-(trifluoromethyl)-1H-pyrazol-4-yl]phenoxy}-3-(propan-2-yl)-7H,8H-imidazo[1,5-a]pyrazin-8-one

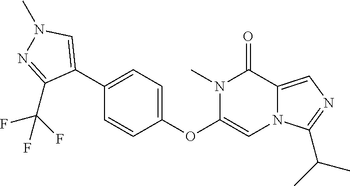 [SMILES: CC(C)c1ncc2c(=O)n(C)c(Oc3ccc(-c4cn(C)nc4C(F)(F)F)cc3)cn12]

¹H NMR (400 MHz, CDCl₃) δ 7.94 (s, 1H), 7.52 (s, 1H), 7.45 (d, J=8.56 Hz, 2H), 7.13 (d, J=8.80 Hz, 2H), 6.68 (s, 1H), 4.03 (s, 3H), 3.43 (s, 3H), 3.06 (spt, J=6.91 Hz, 1H), 1.38 (d, J=6.85 Hz, 6H). [M+H]=432.3.

### Example 139. 7-Methyl-6-({6-[1-methyl-3-(trifluoromethyl)-1H-pyrazol-4-y]pyridin-3-yl}oxy)-3-(propan-2-yl)-7H,8H-imidazo[1,5-a]pyrazin-8-one

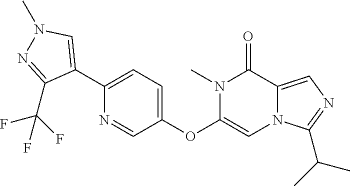 [SMILES: CC(C)c1ncc2c(=O)n(C)c(Oc3ccc(-c4cn(C)nc4C(F)(F)F)nc3)cn12]

¹H NMR (400 MHz, CDCl₃) δ 8.51 (d, J=2.93 Hz, 1H), 7.96 (d, J=4.40 Hz, 2H), 7.64 (d, J=8.68 Hz, 1H), 7.48 (dd, J=2.93, 8.80 Hz, 1H), 6.64 (s, 1H), 4.03 (s, 3H), 3.47 (s, 3H), 2.98-3.15 (m, 1H), 1.38 (d, J=6.85 Hz, 6H). [M+H]=433.3.

### Example 140. 7-Methyl-6-{4-[1-methyl-3-(trifluoromethyl)-1H-pyrazol-4-yl]phenoxy}-3-(oxan-4-yl)-7H,8H-imidazo[1,5-a]pyrazin-8-one

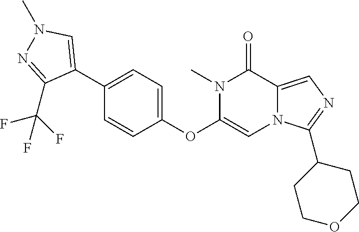 [SMILES: Cn1cc(-c2ccc(Oc3cn4c(C5CCOCC5)ncc4c(=O)n3C)cc2)c(C(F)(F)F)n1]

¹H NMR (400 MHz, CDCl₃) δ 7.97 (s, 1H), 7.52 (s, 1H), 7.46 (d, J=8.44 Hz, 2H), 7.13 (d, J=8.56 Hz, 2H), 6.68 (s, 1H), 4.10 (d, J=11.62 Hz, 2H), 4.03 (s, 3H), 3.53 (t, J=11.13 Hz, 2H), 3.45 (s, 3H), 2.97-3.10 (m, 1H), 2.04-2.17 (m, 2H), 1.84 (d, J=12.72 Hz, 2H). [M+H]=474.3.

### Example 141. 7-Methyl-6-[4-(1-methyl-1H-pyrazol-5-yl)phenoxy]-3-(oxan-4-yl)-7H,8H-imidazo[1,5-a]pyrazin-8-one

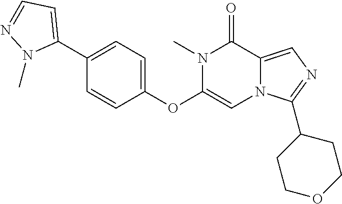 [SMILES: Cn1nccc1-c1ccc(Oc2cn3c(C4CCOCC4)ncc3c(=O)n2C)cc1]

¹H NMR (400 MHz, CDCl₃) δ 7.98 (s, 1H), 7.55 (d, J=1.71 Hz, 1H), 7.49 (d, J=8.56 Hz, 2H), 7.28 (s, 2H), 7.18 (d, J=8.56 Hz, 2H), 6.79 (s, 1H), 6.34 (d, J=1.71 Hz, 1H), 4.11 (d, J=11.49 Hz, 2H), 3.92 (s, 3H), 3.54 (t, J=10.94 Hz, 2H), 3.43 (s, 3H), 2.97-3.10 (m, 1H), 2.03-2.17 (m, 2H). [M+H]=406.2.

### Example 142. 7-Methyl-6-[4-(1-methyl-1H-pyrazol-5-yl)phenoxy]-3-(propan-2-yl)-7H,8H-imidazo[1,5-a]pyrazin-8-one

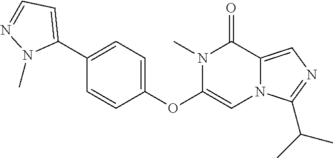 [SMILES: CC(C)c1ncc2c(=O)n(C)c(Oc3ccc(-c4ccnn4C)cc3)cn12]

¹H NMR (400 MHz, CDCl₃) δ 7.96 (s, 1H), 7.55 (s, 1H), 7.49 (d, J=8.68 Hz, 2H), 7.20 (d, J=8.68 Hz, 2H), 6.73 (s, 1H), 6.34 (s, 1H), 3.93 (s, 3H), 3.44 (s, 3H), 3.09 (spt, J=6.77 Hz, 1H), 1.40 (d, J=6.85 Hz, 6H). [M+H]=364.3.

### Example 143. 7-Methyl-6-({5-[1-methyl-3-(trifluoromethyl)-1H-pyrazol-4-yl]pyridin-2-yl}oxy)-3-(propan-2-yl)-7H,8H-imidazo[1,5-a]pyrazin-8-one

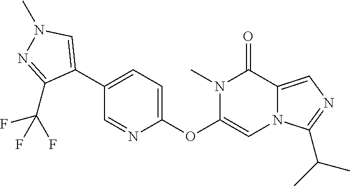 [SMILES: CC(C)c1ncc2c(=O)n(C)c(Oc3ccc(-c4cn(C)nc4C(F)(F)F)cn3)cn12]

¹H NMR (400 MHz, CDCl₃) δ 8.19 (d, J=1.83 Hz, 1H), 7.99 (s, 1H), 7.87 (d, J=8.56 Hz, 1H), 7.55 (s, 1H), 7.13 (d, J=8.56 Hz, 1H), 7.01 (s, 1H), 4.04 (s, 3H), 3.35 (s, 3H), 3.14-3.25 (m, 1H), 1.46 (d, J=6.85 Hz, 6H). [M+H]=433.1.

### Example 144. 6-[4-(3-Cyclopropyl-1-methyl-1H-pyrazol-4-yl)phenoxy]-7-methyl-3-(oxan-4-yl)-7H,8H-imidazo[1,5-a]pyrazin-8-one

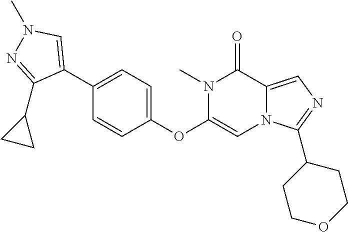 [SMILES: Cn1cc(-c2ccc(Oc3cn4c(C5CCOCC5)ncc4c(=O)n3C)cc2)c(C2CC2)n1]

¹H NMR (400 MHz, CDCl₃) δ 7.96 (s, 1H), 7.60 (d, J=8.68 Hz, 2H), 7.43 (s, 1H), 7.12 (d, J=8.68 Hz, 2H), 6.67 (s, 1H), 4.07-4.13 (m, 2H), 3.88 (s, 3H), 3.53 (dt, J=1.83, 11.68 Hz, 2H), 3.46 (s, 3H), 2.94-3.06 (m, 1H), 2.06-2.23 (m, 2H), 1.89-1.99 (m, 1H), 1.78-1.87 (m, 2H), 0.92-0.98 (m, 4H). [M+H]=446.3.

### Example 145. 1-Methyl-5-(4-{[7-methyl-8-oxo-3-(propan-2-yl)-7H,8H-imidazo[1,5-a]pyrazin-6-yl]oxy}phenyl)-1H-pyrazole-4-carbonitrile

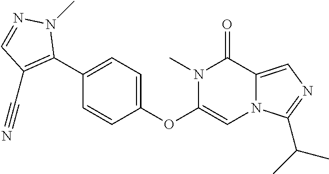 [SMILES: CC(C)c1ncc2c(=O)n(C)c(Oc3ccc(-c4c(C#N)cnn4C)cc3)cn12]

¹H NMR (400 MHz, CDCl₃) δ 7.98 (s, 1H), 7.87 (s, 1H), 7.57 (d, J=8.68 Hz, 2H), 7.28 (d, J=8.70 Hz, 2H), 6.82 (s, 1H), 3.93 (s, 3H), 3.43 (s, 3H), 3.13 (quind, J=6.80, 13.65 Hz, 1H), 1.42 (d, J=6.97 Hz, 6H). [M+H]=389.2.

### Example 146. 6-[4-(1-Ethyl-1H-pyrazol-5-yl)phenoxy]-7-methyl-3-(propan-2-yl)-7H,8H-imidazo[1,5-a]pyrazin-8-one

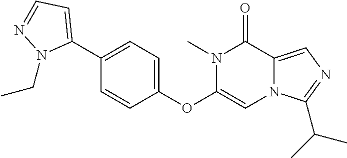 [SMILES: CCn1nccc1-c1ccc(Oc2cn3c(C(C)C)ncc3c(=O)n2C)cc1]

¹H NMR (400 MHz, CDCl₃) δ 7.97 (s, 1H), 7.58 (d, J=1.83 Hz, 1H), 7.47 (d, J=8.68 Hz, 2H), 7.20 (d, J=8.80 Hz, 2H), 6.72 (s, 1H), 6.31 (d, J=1.83 Hz, 1H), 4.19 (q, J=7.21 Hz, 2H), 3.45 (s, 3H), 3.10 (spt, J=6.79 Hz, 1H), 1.46 (t, J=7.21 Hz, 3H), 1.41 (d, J=6.85 Hz, 6H). [M+H]=378.2.

### Example 147. 6-[4-(1,3-Dimethyl-1H-pyrazol-4-yl)phenoxy]-7-methyl-3-(propan-2-yl)-7H,8H-imidazo[1,5-a]pyrazin-8-one

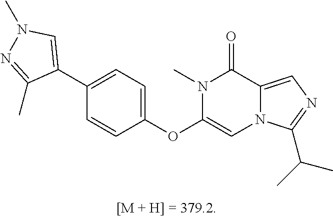 [SMILES: Cc1nn(C)cc1-c1ccc(Oc2cn3c(C(C)C)ncc3c(=O)n2C)cc1]

### Example 148. 6-[4-(1,2-Dimethyl-1H-imidazol-4-yl)phenoxy]-7-methyl-3-(propan-2-yl)-7H,8H-imidazo[1,5-a]pyrazin-8-one

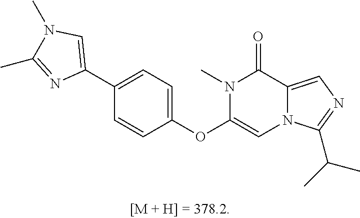 [SMILES: Cc1nc(-c2ccc(Oc3cn4c(C(C)C)ncc4c(=O)n3C)cc2)cn1C]

### Example 149. 7-Methyl-3-(propan-2-yl)-6-[4-(1H-pyrazol-1-yl)phenoxy]-7H,8H-imidazo[1,5-a]pyrazin-8-one

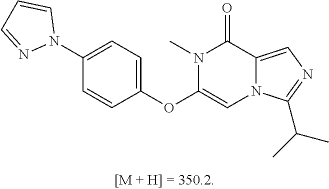 [SMILES: CC(C)c1ncc2c(=O)n(C)c(Oc3ccc(-n4cccn4)cc3)cn12]

### Example 150. 7-Methyl-6-{[6-(1-methyl-1H-pyrazol-4-yl)pyridin-3-yl]oxy}-3-(propan-2-yl)-7H,8H-imidazo[1,5-a]pyrazin-8-one

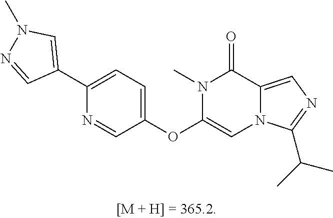 [SMILES: CC(C)c1ncc2c(=O)n(C)c(Oc3ccc(-c4cnn(C)c4)nc3)cn12]

### Example 151. 7-Methyl-3-(propan-2-yl)-6-[4-(1H-pyrazol-1-ylmethyl)phenoxy]-7H,8H-imidazo[1,5-a]pyrazin-8-one

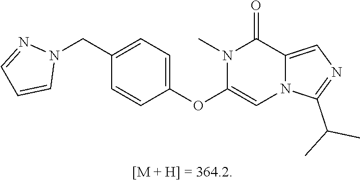 [SMILES: CC(C)c1ncc2c(=O)n(C)c(Oc3ccc(Cn4cccn4)cc3)cn12]

### Example 152. 7-Methyl-3-(propan-2-yl)-6-{4-[1-(propan-2-yl)-1H-pyrazol-5-yl]phenoxy}-7H,8H-imidazo[1,5-a]pyrazin-8-one

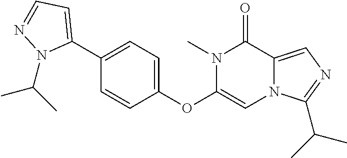 [SMILES: CC(C)c1ncc2c(=O)n(C)c(Oc3ccc(-c4ccnn4C(C)C)cc3)cn12]

¹H NMR (400 MHz, CDCl₃) δ 7.98 (s, 1H), 7.61 (d, J=1.83 Hz, 1H), 7.44 (d, J=8.68 Hz, 2H), 7.20 (d, J=8.68 Hz, 2H), 6.73 (s, 1H), 6.27 (d, J=1.71 Hz, 1H), 4.53 (spt, J=6.62 Hz, 1H), 3.45 (s, 3H), 3.03-3.17 (m, 1H), 1.51 (d, J=6.60 Hz, 6H), 1.42 (d, J=6.85 Hz, 6H). [M+H]=392.2.

### Example 153. 6-[4-(5-Cyclopropyl-3-methyl-1H-pyrazol-1-yl)phenoxy]-7-methyl-3-(propan-2-yl)-7H,8H-imidazo[1,5-a]pyrazin-8-one

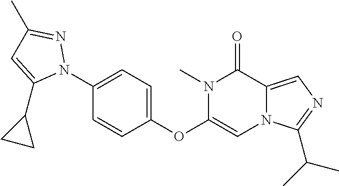 [SMILES: Cc1cc(C2CC2)n(-c2ccc(Oc3cn4c(C(C)C)ncc4c(=O)n3C)cc2)n1]

¹H NMR (400 MHz, CDCl₃) δ 7.95 (s, 1H), 7.70 (d, J=9.05 Hz, 2H), 7.22 (d, J=9.05 Hz, 2H), 6.58 (s, 1H), 5.82 (s, 1H), 3.47 (s, 3H), 3.05 (spt, J=6.91 Hz, 1H), 2.30 (s, 3H), 1.78 (tt, J=5.10, 8.34 Hz, 1H), 1.38 (d, J=6.85 Hz, 6H), 0.97-1.06 (m, 2H), 0.74-0.82 (m, 2H). [M+H]=404.2.

### Example 154. 6-[4-(5-Cyclopropyl-3-methyl-1H-pyrazol-1-yl)phenoxy]-7-methyl-3-(oxan-4-yl)-7H,8H-imidazo[1,5-a]pyrazin-8-one

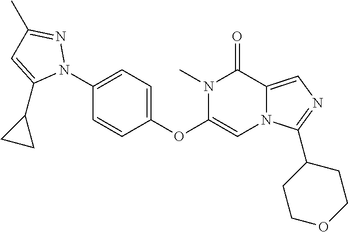 [SMILES: Cc1cc(C2CC2)n(-c2ccc(Oc3cn4c(C5CCOCC5)ncc4c(=O)n3C)cc2)n1]

¹H NMR (400 MHz, CDCl₃) δ 7.97 (s, 1H), 7.70 (d, J=8.93 Hz, 2H), 7.21 (d, J=9.05 Hz, 2H), 6.63 (s, 1H), 5.82 (s, 1H), 4.05-4.15 (m, 2H), 3.56 (dt, J=2.08, 11.74 Hz, 2H), 3.47 (s, 3H), 3.00 (tt, J=3.76, 11.34 Hz, 1H), 2.31 (s, 3H), 2.05-2.20 (m, 2H), 1.72-1.87 (m, 3H), 0.95-1.07 (m, 2H), 0.74-0.82 (m, 2H). [M+H]=446.3.

### Example 155. 7-Methyl-6-[4-(1-methyl-1H-pyrazol-3-yl)phenoxy]-3-(propan-2-yl)-7H,8H-imidazo[1,5-a]pyrazin-8-one

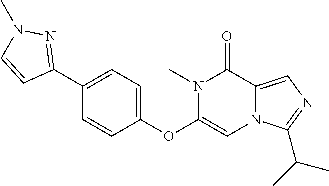 [SMILES: CC(C)c1ncc2c(=O)n(C)c(Oc3ccc(-c4ccn(C)n4)cc3)cn12]

¹H NMR (400 MHz, CDCl₃) δ 7.93 (s, 1H), 7.87 (d, J=8.80 Hz, 2H), 7.43 (d, J=2.20 Hz, 1H), 7.15 (d, J=8.93 Hz, 2H), 6.55 (d, J=2.32 Hz, 1H), 6.54 (s, 1H), 3.99 (s, 3H), 3.47 (s, 3H), 3.01 (spt, J=6.85 Hz, 1H), 1.35 (d, J=6.85 Hz, 6H). [M+H]=364.1.

### Example 156. 7-Methyl-6-[4-(1-methyl-1H-pyrazol-3-yl)phenoxy]-3-(oxan-4-yl)-7H,8H-imidazo[1,5-a]pyrazin-8-one

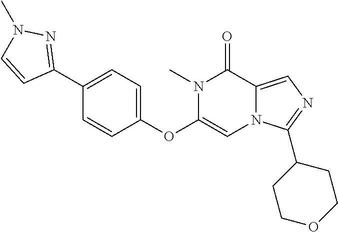 [SMILES: Cn1ccc(-c2ccc(Oc3cn4c(C5CCOCC5)ncc4c(=O)n3C)cc2)n1]

¹H NMR (400 MHz, CDCl₃) δ 7.95 (s, 1H), 7.86 (d, J=8.80 Hz, 2H), 7.43 (d, J=2.32 Hz, 1H), 7.14 (d, J=8.93 Hz, 2H), 6.59 (s, 1H), 6.55 (d, J=2.32 Hz, 1H), 4.03-4.13 (m, 2H), 3.99 (s, 3H), 3.53 (dt, J=2.14, 11.71 Hz, 2H), 3.46 (s, 3H), 2.95 (tt, J=3.93, 11.29 Hz, 1H), 2.01-2.17 (m, 2H), 1.81 (dd, J=1.90, 13.27 Hz, 2H). [M+H]=406.2.

### Example 157. 6-[4-(4-Cyclopropyl-1-methyl-1H-pyrazol-3-yl)phenoxy]-7-methyl-3-(propan-2-yl)-7H,8H-imidazo[1,5-a]pyrazin-8-one

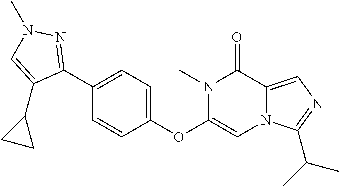 [SMILES: CC(C)c1ncc2c(=O)n(C)c(Oc3ccc(-c4nn(C)cc4C4CC4)cc3)cn12]

¹H NMR (400 MHz, CDCl₃) δ 7.99 (d, J=8.80 Hz, 2H), 7.95 (s, 1H), 7.18 (d, J=8.80 Hz, 2H), 7.11 (s, 1H), 6.55 (s, 1H), 3.91 (s, 3H), 3.49 (s, 3H), 2.97-3.11 (m, 1H), 1.73-1.87 (m, 1H), 1.37 (d, J=6.85 Hz, 6H), 0.90-1.01 (m, 2H), 0.51-0.62 (m, 2H). [M+H]=404.2.

### Example 158. 6-[4-(4-Cyclopropyl-1-methyl-1H-pyrazol-3-yl)phenoxy]-7-methyl-3-(oxan-4-yl)-7H,8H-imidazo[1,5-a]pyrazin-8-one

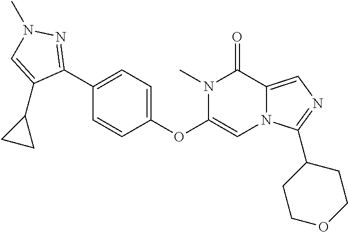 [SMILES: Cn1cc(C2CC2)c(-c2ccc(Oc3cn4c(C5CCOCC5)ncc4c(=O)n3C)cc2)n1]

¹H NMR (400 MHz, CDCl₃) δ 7.95-8.03 (m, 3H), 7.16 (d, J=8.80 Hz, 2H), 7.12 (s, 1H), 6.60 (s, 1H), 4.08 (td, J=1.85, 9.75 Hz, 2H), 3.91 (s, 3H), 3.54 (dt, J=1.96, 11.68 Hz, 2H), 3.48 (s, 3H), 2.93-3.03 (m, 1H), 2.05-2.18 (m, 2H), 1.83 (br s, 2H), 1.72-1.78 (m, 1H), 0.91-0.97 (m, 2H), 0.55-0.60 (m, 2H). [M+H]=446.2.

### Example 159. 6-[4-(4-Bromo-1-methyl-1H-pyrazol-3-yl)phenoxy]-7-methyl-3-(propan-2-yl)-7H,8H-imidazo[1,5-a]pyrazin-8-one

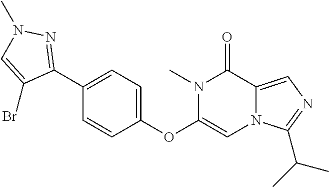 [SMILES: CC(C)c1ncc2c(=O)n(C)c(Oc3ccc(-c4nn(C)cc4Br)cc3)cn12]

¹H NMR (400 MHz, CDCl₃) δ 7.97 (d, J=8.93 Hz, 2H), 7.94 (s, 1H), 7.51 (s, 1H), 7.18 (d, J=8.93 Hz, 2H), 6.61 (s, 1H), 3.97 (s, 3H), 3.46 (s, 3H), 3.04 (spt, J=6.89 Hz, 1H), 1.37 (d, J=6.85 Hz, 6H). [M+H]=442.0.

### Example 160. 6-[4-(4-Chloro-1-methyl-1H-pyrazol-3-yl)phenoxy]-7-methyl-3-(propan-2-yl)-7H,8H-imidazo[1,5-a]pyrazin-8-one

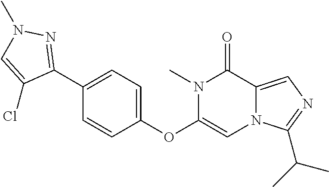 [SMILES: CC(C)c1ncc2c(=O)n(C)c(Oc3ccc(-c4nn(C)cc4Cl)cc3)cn12]

¹H NMR (400 MHz, CDCl₃) δ 7.98 (d, J=8.93 Hz, 2H), 7.94 (s, 1H), 7.48 (s, 1H), 7.18 (d, J=8.93 Hz, 2H), 6.60 (s, 1H), 3.95 (s, 3H), 3.46 (s, 3H), 3.04 (spt, J=6.87 Hz, 1H), 1.37 (d, J=6.85 Hz, 6H). [M+H]=398.1.

### Example 161. 6-[4-(4-Chloro-1-methyl-1H-pyrazol-3-yl)phenoxy]-7-methyl-3-(oxan-4-yl)-7H,8H-imidazo[1,5-a]pyrazin-8-one

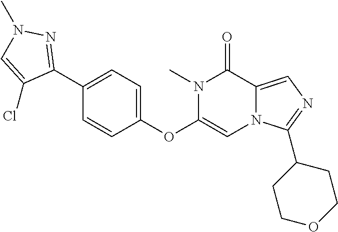 [SMILES: Cn1cc(Cl)c(-c2ccc(Oc3cn4c(C5CCOCC5)ncc4c(=O)n3C)cc2)n1]

¹H NMR (400 MHz, CDCl₃) δ 7.94-8.00 (m, 3H), 7.49 (s, 1H), 7.17 (d, J=8.80 Hz, 2H), 6.64 (s, 1H), 4.03-4.12 (m, 2H), 3.95 (s, 3H), 3.54 (dt, J=1.96, 11.68 Hz, 2H), 3.46 (s, 3H), 2.90-3.10 (m, 1H), 2.07-2.20 (m, 2H), 1.83 (d, J=11.74 Hz, 2H). [M+H]=440.1.

### Example 162. 6-[4-(3-Methoxy-1-methyl-1H-pyrazol-4-yl)phenoxy]-7-methyl-3-(propan-2-yl)-7H,8H-imidazo[1,5-a]pyrazin-8-one

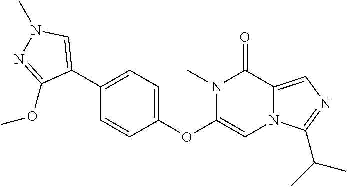 [SMILES: COc1nn(C)cc1-c1ccc(Oc2cn3c(C(C)C)ncc3c(=O)n2C)cc1]

¹H NMR (400 MHz, CDCl₃) δ 7.93 (s, 1H), 7.67 (d, J=8.80 Hz, 2H), 7.45 (s, 1H), 7.09 (d, J=8.80 Hz, 2H), 6.53 (s, 1H), 4.04 (s, 3H), 3.82 (s, 3H), 3.46 (s, 3H), 2.95-3.08 (m, 1H), 1.36 (d, J=6.85 Hz, 6H). [M+H]=394.2.

### Example 163. 6-[4-(4-Ethyl-1-methyl-1H-pyrazol-3-yl)phenoxy]-7-methyl-3-(propan-2-yl)-7H,8H-imidazo[1,5-a]pyrazin-8-one

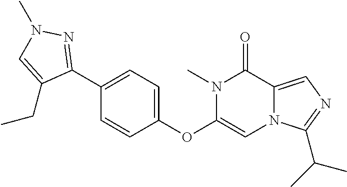 [SMILES: CCc1cn(C)nc1-c1ccc(Oc2cn3c(C(C)C)ncc3c(=O)n2C)cc1]

¹H NMR (400 MHz, CDCl₃) δ 7.94 (s, 1H), 7.72 (d, J=8.80 Hz, 2H), 7.27 (s, 1H), 7.16 (d, J=8.93 Hz, 2H), 6.55 (s, 1H), 3.94 (s, 3H), 3.48 (s, 3H), 3.03 (spt, J=6.87 Hz, 1H), 2.67 (q, J=7.90 Hz, 2H), 1.37 (d, J=6.85 Hz, 6H), 1.25 (t, J=7.46 Hz, 3H). [M+H]=392.2.

### Example 164. 6-[4-(4-Ethyl-1-methyl-1H-pyrazol-3-yl)phenoxy]-7-methyl-3-(oxan-4-yl)-7H,8H-imidazo[1,5-a]pyrazin-8-one

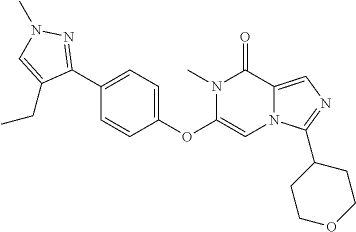 [SMILES: CCc1cn(C)nc1-c1ccc(Oc2cn3c(C4CCOCC4)ncc3c(=O)n2C)cc1]

¹H NMR (400 MHz, CDCl₃) δ 7.96 (s, 1H), 7.72 (d, J=8.93 Hz, 2H), 7.27 (s, 1H), 7.15 (d, J=8.93 Hz, 2H), 6.59 (s, 1H), 4.04-4.12 (m, 2H), 3.94 (s, 3H), 3.54 (dt, J=2.08, 11.74 Hz, 2H), 3.47 (s, 3H), 2.97 (tt, J=3.91, 11.31 Hz, 1H), 2.66 (q, J=7.38 Hz, 2H), 2.04-2.17 (m, 2H), 1.81 (dd, J=1.90, 13.27 Hz, 2H), 1.25 (t, J=7.46 Hz, 3H). [M+H]=434.3.

### Example 165. 6-[4-(3-Methoxy-1-methyl-1H-pyrazol-4-yl)phenoxy]-7-methyl-3-(oxan-4-yl)-7H,8H-imidazo[1,5-a]pyrazin-8-one

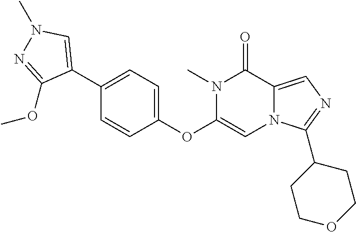 [SMILES: COc1nn(C)cc1-c1ccc(Oc2cn3c(C4CCOCC4)ncc3c(=O)n2C)cc1]

¹H NMR (400 MHz, CDCl₃) δ 7.96 (s, 1H), 7.67 (d, J=8.93 Hz, 2H), 7.45 (s, 1H), 7.08 (d, J=8.80 Hz, 2H), 6.58 (s, 1H), 4.05-4.14 (m, 2H), 4.04 (s, 3H), 3.82 (s, 3H), 3.53 (dt, J=2.08, 11.68 Hz, 2H), 3.46 (s, 3H), 2.97 (tt, J=3.70, 11.28 Hz, 1H), 2.04-2.18 (m, 2H), 1.81 (dd, J=1.77, 13.27 Hz, 2H). [M+H]=436.2.

### Example 166. 6-[4-(4-Methoxy-1-methyl-1H-pyrazol-3-yl)phenoxy]-7-methyl-3-(propan-2-yl)-7H,8H-imidazo[1,5-a]pyrazin-8-one

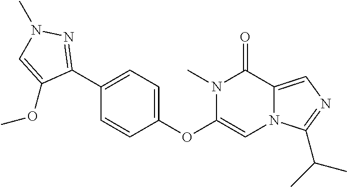 [SMILES: COc1cn(C)nc1-c1ccc(Oc2cn3c(C(C)C)ncc3c(=O)n2C)cc1]

¹H NMR (400 MHz, CDCl₃) δ 8.04 (d, J=8.80 Hz, 2H), 7.93 (s, 1H), 7.11-7.17 (m, 3H), 6.51 (s, 1H), 3.91 (s, 3H), 3.85 (s, 3H), 3.48 (s, 3H), 3.01 (spt, J=6.83 Hz, 1H), 1.35 (d, J=6.85 Hz, 6H). [M+H]=394.2.

### Example 167. 6-[4-(4-Methoxy-1-methyl-1H-pyrazol-3-yl)phenoxy]-7-methyl-3-(oxan-4-yl)-7H,8H-imidazo[1,5-a]pyrazin-8-one

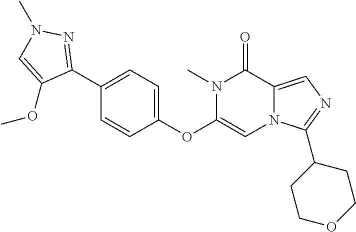 [SMILES: COc1cn(C)nc1-c1ccc(Oc2cn3c(C4CCOCC4)ncc3c(=O)n2C)cc1]

¹H NMR (400 MHz, CDCl₃) δ 8.03 (d, J=8.68 Hz, 2H), 7.95 (s, 1H), 7.09-7.16 (m, 3H), 6.56 (s, 1H), 4.04-4.12 (m, 2H), 3.91 (s, 3H), 3.85 (s, 3H), 3.53 (dt, J=1.83, 11.68 Hz, 2H), 3.47 (s, 3H), 2.94 (tt, J=3.79, 11.31 Hz, 1H), 2.01-2.16 (m, 2H), 1.77-1.84 (m, 2H). [M+H]=436.2.

### Example 168. 6-[4-(1-Ethyl-1H-pyrazol-3-yl)phenoxy]-7-methyl-3-(propan-2-yl)-7H,8H-imidazo[1,5-a]pyrazin-8-one

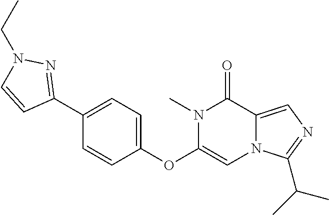 [SMILES: CCn1ccc(-c2ccc(Oc3cn4c(C(C)C)ncc4c(=O)n3C)cc2)n1]

¹H NMR (400 MHz, CDCl₃) δ 7.93 (s, 1H), 7.88 (d, J=8.80 Hz, 2H), 7.47 (d, J=2.32 Hz, 1H), 7.15 (d, J=8.80 Hz, 2H), 6.55 (d, J=2.32 Hz, 1H), 6.52 (s, 1H), 4.25 (q, J=7.30 Hz, 2H), 3.47 (s, 3H), 2.94-3.10 (m, 1H), 1.57 (t, J=7.27 Hz, 3H), 1.35 (d, J=6.85 Hz, 6H). [M+H]=378.1.

### Example 169. 6-[4-(4-Chloro-1-ethyl-1H-pyrazol-3-yl)phenoxy]-7-methyl-3-(propan-2-yl)-7H,8H-imidazo[1,5-a]pyrazin-8-one

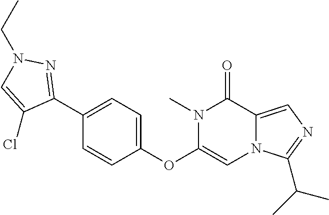 [SMILES: CCn1cc(Cl)c(-c2ccc(Oc3cn4c(C(C)C)ncc4c(=O)n3C)cc2)n1]

¹H NMR (400 MHz, CDCl₃) δ 7.99 (d, J=8.93 Hz, 2H), 7.94 (s, 1H), 7.51 (s, 1H), 7.18 (d, J=8.93 Hz, 2H), 6.59 (s, 1H), 4.21 (q, J=7.34 Hz, 2H), 3.47 (s, 3H), 2.99-3.16 (m, 1H), 1.55 (t, J=7.34 Hz, 3H), 1.37 (d, J=6.97 Hz, 6H). [M+H]=412.1.

### Example 170. 6-[4-(1-Ethyl-1H-pyrazol-3-yl)phenoxy]-7-methyl-3-(oxan-4-yl)-7H,8H-imidazo[1,5-a]pyrazin-8-one

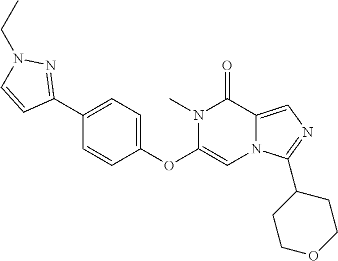 [SMILES: CCn1ccc(-c2ccc(Oc3cn4c(C5CCOCC5)ncc4c(=O)n3C)cc2)n1]

¹H NMR (400 MHz, CDCl₃) δ 7.95 (s, 1H), 7.88 (d, J=8.80 Hz, 2H), 7.47 (d, J=2.20 Hz, 1H), 7.14 (d, J=8.80 Hz, 2H), 6.57 (s, 1H), 6.55 (d, J=2.20 Hz, 1H), 4.25 (q, J=7.25 Hz, 2H), 4.05-4.12 (m, 2H), 3.53 (dt, J=2.08, 11.68 Hz, 2H), 3.47 (s, 3H), 2.95 (tt, J=3.77, 11.32 Hz, 1H), 2.04-2.16 (m, 2H), 1.81 (dd, J=1.83, 13.33 Hz, 2H), 1.57 (t, J=7.34 Hz, 3H). [M+H]=420.2.

### Example 171. 6-[4-(4-Chloro-1-ethyl-1H-pyrazol-3-yl)phenoxy]-7-methyl-3-(oxan-4-yl)-7H,8H-imidazo[1,5-a]pyrazin-8-one

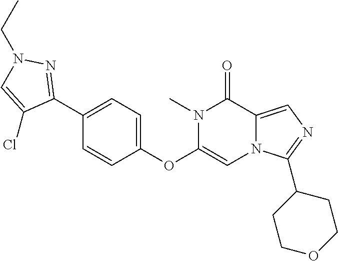 [SMILES: CCn1cc(Cl)c(-c2ccc(Oc3cn4c(C5CCOCC5)ncc4c(=O)n3C)cc2)n1]

¹H NMR (400 MHz, CDCl₃) δ 7.99 (d, J=8.80 Hz, 2H), 7.96 (s, 1H), 7.52 (s, 1H), 7.17 (d, J=8.80 Hz, 2H), 6.64 (s, 1H), 4.21 (q, J=7.34 Hz, 2H), 4.04-4.12 (m, 2H), 3.54 (dt, J=2.02, 11.71 Hz, 2H), 3.46 (s, 3H), 2.98 (tt, J=3.90, 11.32 Hz, 1H), 2.03-2.18 (m, 2H), 1.82 (dd, J=1.71, 13.33 Hz, 2H), 1.55 (t, J=7.34 Hz, 3H). [M+H]=454.2.

### Example 172. 1-Methyl-3-(4-{[7-methyl-8-oxo-3-(propan-2-yl)-7H, 8H-imidazo[1,5-a]pyrazin-6-yl]oxy}phenyl)-1H-pyrazole-4-carbonitrile

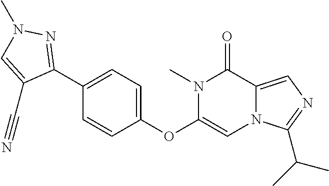 [SMILES: CC(C)c1ncc2c(=O)n(C)c(Oc3ccc(-c4nn(C)cc4C#N)cc3)cn12]

¹H NMR (400 MHz, CDCl₃) δ 8.04 (d, J=8.93 Hz, 2H), 7.95 (s, 1H), 7.89 (s, 1H), 7.21 (d, J=8.80 Hz, 2H), 6.65 (s, 1H), 4.02 (s, 3H), 3.45 (s, 3H), 3.06 (spt, J=6.87 Hz, 1H), 1.38 (d, J=6.85 Hz, 6H). [M+H]=389.2.

### Example 173. 1-Methyl-3-(4-{[7-methyl-3-(oxan-4-yl)-8-oxo-7H,8H-imidazo[1,5-a]pyrazin-6-yl]oxy}phenyl)-1H-pyrazole-4-carbonitrile

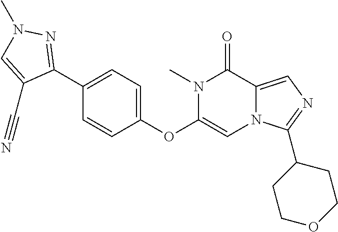 [SMILES: Cn1cc(C#N)c(-c2ccc(Oc3cn4c(C5CCOCC5)ncc4c(=O)n3C)cc2)n1]

¹H NMR (400 MHz, CDCl₃) δ 8.04 (d, J=8.80 Hz, 2H), 7.97 (s, 1H), 7.89 (s, 1H), 7.19 (d, J=8.80 Hz, 2H), 6.70 (s, 1H), 4.06-4.14 (m, 2H), 4.02 (s, 3H), 3.55 (dt, J=2.08, 11.68 Hz, 2H), 3.44 (s, 3H), 3.00 (tt, J=3.84, 11.32 Hz, 1H), 2.04-2.21 (m, 2H), 1.84 (dd, J=1.83, 13.33 Hz, 2H). [M+H]=431.2.

### Example 174. 7-Methyl-3-(propan-2-yl)-6-[4-(1-propyl-1H-pyrazol-5-yl)phenoxy]-7H,8H-imidazo[1,5-a]pyrazin-8-one

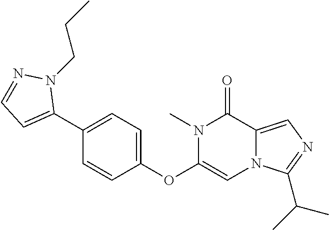 [SMILES: CCCn1nccc1-c1ccc(Oc2cn3c(C(C)C)ncc3c(=O)n2C)cc1]

¹H NMR (400 MHz, CDCl₃) δ 7.96 (s, 1H), 7.57 (d, J=1.83 Hz, 1H), 7.46 (d, J=8.80 Hz, 2H), 7.19 (d, J=8.80 Hz, 2H), 6.72 (s, 1H), 6.30 (d, J=1.83 Hz, 1H), 4.06-4.15 (m, 2H), 3.44 (s, 3H), 3.09 (quin, J=6.88 Hz, 1H), 1.87 (sxt, J=7.38 Hz, 2H), 1.40 (d, J=6.85 Hz, 6H), 0.86 (t, J=7.46 Hz, 3H). [M+H]=392.2.

### Example 175. 7-Methyl-3-(propan-2-yl)-6-[4-(1-propyl-1H-pyrazol-3-yl)phenoxy]-7H,8H-imidazo[1,5-a]pyrazin-8-one

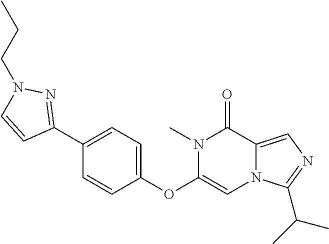 [SMILES: CCCn1ccc(-c2ccc(Oc3cn4c(C(C)C)ncc4c(=O)n3C)cc2)n1]

¹H NMR (400 MHz, CDCl₃) δ 7.94 (s, 1H), 7.88 (d, J=8.80 Hz, 2H), 7.45 (d, J=2.32 Hz, 1H), 7.15 (d, J=8.80 Hz, 2H), 6.55 (d, J=2.32 Hz, 1H), 6.52 (s, 1H), 4.15 (t, J=7.09 Hz, 2H), 3.48 (s, 3H), 3.02 (spt, J=6.85 Hz, 1H), 1.91-2.04 (m, 2H), 1.36 (d, J=6.85 Hz, 6H), 0.99 (t, J=7.46 Hz, 3H). [M+H]=392.2.

### Example 176. 7-Methyl-3-(oxan-4-yl)-6-[4-(1-propyl-1H-pyrazol-3-yl)phenoxy]-7H,8H-imidazo[1,5-a]pyrazin-8-one

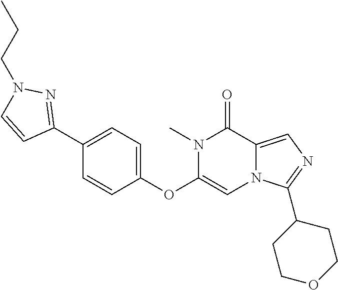 [SMILES: CCCn1ccc(-c2ccc(Oc3cn4c(C5CCOCC5)ncc4c(=O)n3C)cc2)n1]

¹H NMR (400 MHz, CDCl₃) δ 7.95 (s, 1H), 7.88 (d, J=8.80 Hz, 2H), 7.45 (d, J=2.20 Hz, 1H), 7.14 (d, J=8.80 Hz, 2H), 6.57 (s, 1H), 6.55 (d, J=2.20 Hz, 1H), 4.15 (t, J=7.15 Hz, 2H), 4.04-4.11 (m, 2H), 3.53 (dt, J=2.08, 11.74 Hz, 2H), 3.47 (s, 3H), 2.95 (tt, J=3.91, 11.31 Hz, 1H), 2.06-2.18 (m, 2H), 1.92-2.01 (m, 2H), 1.81 (dd, J=1.90, 13.27 Hz, 2H), 0.99 (t, J=7.40 Hz, 3H). [M+H]=434.3.

### Example 177. 6-[4-(4-Chloro-1-propyl-1H-pyrazol-3-yl)phenoxy]-7-methyl-3-(propan-2-yl)-7H,8H-imidazo[1,5-a]pyrazin-8-one

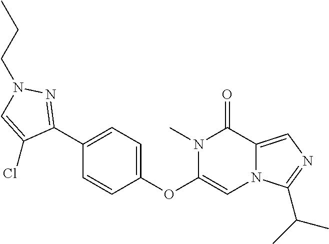 [SMILES: CCCn1cc(Cl)c(-c2ccc(Oc3cn4c(C(C)C)ncc4c(=O)n3C)cc2)n1]

¹H NMR (400 MHz, CDCl₃) δ 7.99 (d, J=8.93 Hz, 2H), 7.95 (s, 1H), 7.50 (s, 1H), 7.18 (d, J=8.93 Hz, 2H), 6.58 (s, 1H), 4.10 (t, J=7.09 Hz, 2H), 3.47 (s, 3H), 3.04 (spt, J=6.93 Hz, 1H), 1.95 (sxt, J=7.26 Hz, 2H), 1.38 (d, J=6.85 Hz, 6H), 0.99 (t, J=7.40 Hz, 3H). [M+H]=426.2.

### Example 178. 6-[4-(4-Chloro-1-propyl-1H-pyrazol-3-yl)phenoxy]-7-methyl-3-(oxan-4-yl)-7H,8H-imidazo[1,5-a]pyrazin-8-one

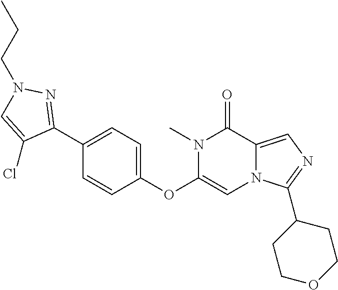 [SMILES: CCCn1cc(Cl)c(-c2ccc(Oc3cn4c(C5CCOCC5)ncc4c(=O)n3C)cc2)n1]

¹H NMR (400 MHz, CDCl₃) δ 7.99 (d, J=8.93 Hz, 2H), 7.96 (s, 1H), 7.50 (s, 1H), 7.16 (d, J=8.93 Hz, 2H), 6.63 (s, 1H), 4.05-4.13 (m, 4H), 3.54 (dt, J=2.08, 11.74 Hz, 2H), 3.46 (s, 3H), 2.98 (tt, J=3.85, 11.31 Hz, 1H), 2.06-2.20 (m, 2H), 1.91-1.98 (m, 2H), 1.82 (dd, J=1.77, 13.27 Hz, 2H), 0.99 (t, J=7.40 Hz, 3H). [M+H]=468.2.

### Example 179. 7-Methyl-3-(propan-2-yl)-6-{4-[1-(propan-2-yl)-1H-pyrazol-3-yl]phenoxy}-7H,8H-imidazo[1,5-a]pyrazin-8-one

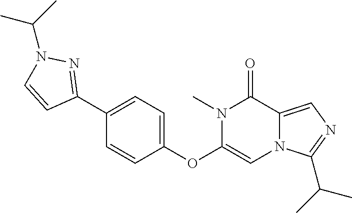 [SMILES: CC(C)c1ncc2c(=O)n(C)c(Oc3ccc(-c4ccn(C(C)C)n4)cc3)cn12]

¹H NMR (400 MHz, CDCl₃) δ 7.93 (s, 1H), 7.89 (d, J=8.80 Hz, 2H), 7.49 (d, J=2.32 Hz, 1H), 7.15 (d, J=8.80 Hz, 2H), 6.55 (d, J=2.32 Hz, 1H), 6.51 (s, 1H), 4.58 (spt, J=6.68 Hz, 1H), 3.48 (s, 3H), 3.00 (spt, J=6.87 Hz, 1H), 1.58 (d, J=6.60 Hz, 6H), 1.35 (d, J=6.85 Hz, 6H). [M+H]=392.2.

### Example 180. 7-Methyl-3-(oxan-4-yl)-6-{4-[1-(propan-2-yl)-1H-pyrazol-3-yl]phenoxy}-7H,8H-imidazo[1,5-a]pyrazin-8-one

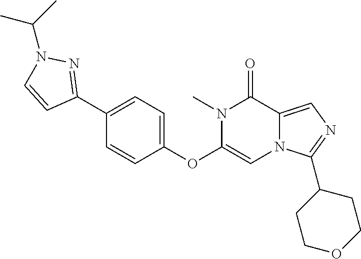 [SMILES: CC(C)n1ccc(-c2ccc(Oc3cn4c(C5CCOCC5)ncc4c(=O)n3C)cc2)n1]

¹H NMR (400 MHz, CDCl₃) δ 7.95 (s, 1H), 7.88 (d, J=8.80 Hz, 2H), 7.50 (d, J=2.32 Hz, 1H), 7.14 (d, J=8.80 Hz, 2H), 6.56 (s, 1H), 6.55 (d, J=2.32 Hz, 1H), 4.52-4.70 (m, 1H), 4.00-4.10 (m, 2H), 3.53 (dt, J=2.08, 11.74 Hz, 2H), 3.47 (s, 3H), 2.86-3.00 (m, 1H), 2.01-2.18 (m, 2H), 1.81 (dd, J=1.90, 13.27 Hz, 2H), 1.58 (d, J=6.72 Hz, 6H). [M+H]=434.3.

### Example 181. 6-{4-[4-Chloro-1-(propan-2-yl)-1H-pyrazol-3-yl]phenoxy}-7-methyl-3-(propan-2-yl)-7H,8H-imidazo[1,5-a]pyrazin-8-one

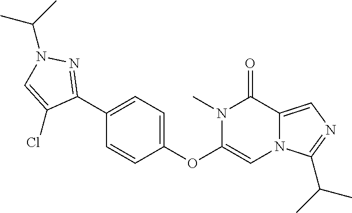 [SMILES: CC(C)c1ncc2c(=O)n(C)c(Oc3ccc(-c4nn(C(C)C)cc4Cl)cc3)cn12]

¹H NMR (400 MHz, CDCl₃) δ 8.00 (d, J=8.93 Hz, 2H), 7.94 (s, 1H), 7.53 (s, 1H), 7.17 (d, J=8.93 Hz, 2H), 6.59 (s, 1H), 4.52 (spt, J=6.70 Hz, 1H), 3.47 (s, 3H), 3.04 (spt, J=6.85 Hz, 1H), 1.56 (d, J=6.72 Hz, 6H), 1.37 (d, J=6.85 Hz, 6H). [M+H]=426.2.

### Example 182. 6-{4-[4-Chloro-1-(propan-2-yl)-1H-pyrazol-3-yl]phenoxy}-7-methyl-3-(oxan-4-yl)-7H,8H-imidazo[1,5-a]pyrazin-8-one

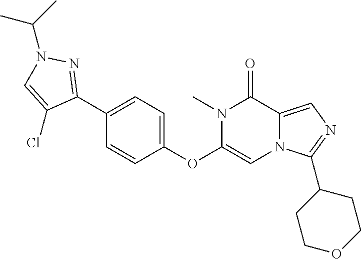 [SMILES: CC(C)n1cc(Cl)c(-c2ccc(Oc3cn4c(C5CCOCC5)ncc4c(=O)n3C)cc2)n1]

¹H NMR (400 MHz, CDCl₃) δ 8.00 (d, J=8.68 Hz, 2H), 7.97 (s, 1H), 7.53 (s, 1H), 7.17 (d, J=8.80 Hz, 2H), 6.62 (s, 1H), 4.52 (spt, J=6.68 Hz, 1H), 4.09 (td, J=1.82, 9.81 Hz, 2H), 3.54 (dt, J=1.83, 11.68 Hz, 2H), 3.47 (s, 3H), 2.90-3.03 (m, 1H), 2.04-2.18 (m, 2H), 1.82 (d, J=11.86 Hz, 2H), 1.57 (d, J=6.72 Hz, 6H). [M+H]=468.0.

### Example 183. 6-[4-(4-Iodo-1-methyl-1H-pyrazol-3-yl)phenoxy]-7-methyl-3-(propan-2-yl)-7H,8H-imidazo[1,5-a]pyrazin-8-one

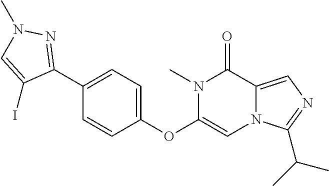 [SMILES: CC(C)c1ncc2c(=O)n(C)c(Oc3ccc(-c4nn(C)cc4I)cc3)cn12]

¹H NMR (400 MHz, CDCl₃) δ 7.87-7.97 (m, 3H), 7.53 (s, 1H), 7.18 (d, J=8.93 Hz, 2H), 6.62 (s, 1H), 3.99 (s, 3H), 3.46 (s, 3H), 3.05 (spt, J=6.91 Hz, 1H), 1.37 (d, J=6.85 Hz, 6H). [M+H]=490.1.

### Example 184. 6-[4-(4-Iodo-1-methyl-1H-pyrazol-3-yl)phenoxy]-7-methyl-3-(oxan-4-yl)-7H,8H-imidazo[1,5-a]pyrazin-8-one

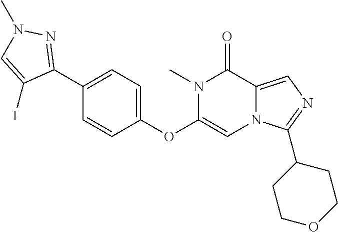 [SMILES: Cn1cc(I)c(-c2ccc(Oc3cn4c(C5CCOCC5)ncc4c(=O)n3C)cc2)n1]

¹H NMR (400 MHz, CDCl₃) δ 7.96 (s, 1H), 7.92 (d, J=8.68 Hz, 2H), 7.53 (s, 1H), 7.17 (d, J=8.80 Hz, 2H), 6.65 (s, 1H), 4.05-4.12 (m, 2H), 3.99 (s, 3H), 3.55 (dt, J=2.02, 11.71 Hz, 2H), 3.46 (s, 3H), 2.98 (tt, J=3.90, 11.32 Hz, 1H), 2.03-2.16 (m, 2H), 1.83 (dd, J=1.71, 13.33 Hz, 2H). [M+H]=532.2.

### Example 185. 6-[4-(1-Ethyl-4-methoxy-1H-pyrazol-3-yl)phenoxy]-7-methyl-3-(propan-2-yl)-7H,8H-imidazo[1,5-a]pyrazin-8-one

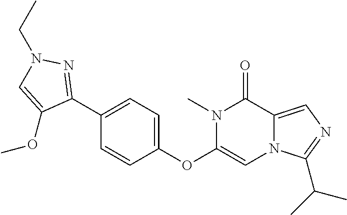 [SMILES: CCn1cc(OC)c(-c2ccc(Oc3cn4c(C(C)C)ncc4c(=O)n3C)cc2)n1]

¹H NMR (400 MHz, CDCl₃) δ 8.05 (d, J=8.93 Hz, 2H), 7.93 (s, 1H), 7.17 (s, 1H), 7.14 (d, J=8.93 Hz, 2H), 6.50 (s, 1H), 4.16 (q, J=7.25 Hz, 2H), 3.86 (s, 3H), 3.48 (s, 3H), 2.99-3.07 (m, 1H), 1.53 (t, J=7.34 Hz, 3H), 1.35 (d, J=6.85 Hz, 6H). [M+H]=408.2.

### Example 186. 6-[4-(1-Ethyl-4-methoxy-1H-pyrazol-5-yl)phenoxy]-7-methyl-3-(propan-2-yl)-7H,8H-imidazo[1,5-a]pyrazin-8-one

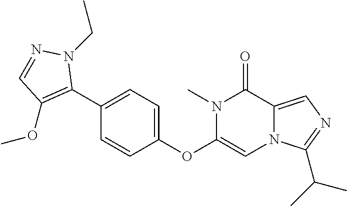 [SMILES: CCn1ncc(OC)c1-c1ccc(Oc2cn3c(C(C)C)ncc3c(=O)n2C)cc1]

¹H NMR (400 MHz, CDCl₃) δ 7.96 (s, 1H), 7.49 (d, J=8.80 Hz, 2H), 7.40 (s, 1H), 7.20 (d, J=8.93 Hz, 2H), 6.74 (s, 1H), 4.13 (q, J=7.21 Hz, 2H), 3.82 (s, 3H), 3.44 (s, 3H), 3.09 (spt, J=6.85 Hz, 1H), 1.37-1.42 (in, 9H). [M+H]=408.2.

### Example 187. 6-{4-[4-Methoxy-1-(propan-2-yl)-1H-pyrazol-3-yl]phenoxy}-7-methyl-3-(propan-2-yl)-7H,8H-imidazo[1,5-a]pyrazin-8-one

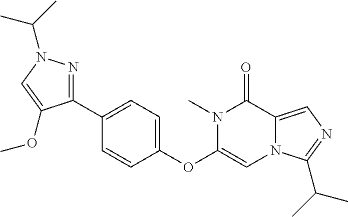 [SMILES: COc1cn(C(C)C)nc1-c1ccc(Oc2cn3c(C(C)C)ncc3c(=O)n2C)cc1]

¹H NMR (400 MHz, CDCl₃) δ 8.06 (d, J=8.93 Hz, 2H), 7.93 (s, 1H), 7.18 (s, 1H), 7.14 (d, J=8.93 Hz, 2H), 6.49 (s, 1H), 4.47 (spt, J=6.70 Hz, 1H), 3.86 (s, 3H), 3.48 (s, 3H), 3.00 (spt, J=6.87 Hz, 1H), 1.55 (d, J=6.72 Hz, 6H), 1.35 (d, J=6.85 Hz, 6H). [M+H]=422.2.

### Example 188. 6-{4-[4-Methoxy-1-(propan-2-yl)-1H-pyrazol-5-yl]phenoxy}-7-methyl-3-(propan-2-yl)-7H,8H-imidazo[1,5-a]pyrazin-8-one

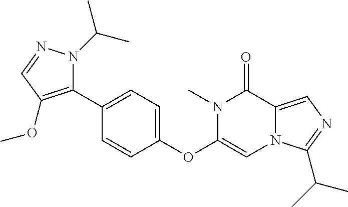 [SMILES: COc1cnn(C(C)C)c1-c1ccc(Oc2cn3c(C(C)C)ncc3c(=O)n2C)cc1]

¹H NMR (400 MHz, CDCl₃) δ 7.96 (s, 1H), 7.45 (d, J=8.93 Hz, 2H), 7.42 (s, 1H), 7.20 (d, J=8.80 Hz, 2H), 6.75 (s, 1H), 4.47 (spt, J=6.60 Hz, 1H), 3.81 (s, 3H), 3.44 (s, 3H), 3.10 (spt, J=6.85 Hz, 1H), 1.48 (d, J=6.60 Hz, 6H), 1.41 (d, J=6.97 Hz, 6H). [M+H]=422.2.

### Example 189. 6-[4-(1-Ethyl-4-methoxy-1H-pyrazol-3-yl)phenoxy]-7-methyl-3-(oxan-4-yl)-7H,8H-imidazo[1,5-a]pyrazin-8-one

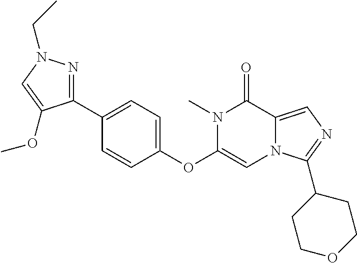 [SMILES: CCn1cc(OC)c(-c2ccc(Oc3cn4c(C5CCOCC5)ncc4c(=O)n3C)cc2)n1]

¹H NMR (400 MHz, CDCl₃) δ 8.04 (d, J=8.93 Hz, 2H), 7.95 (s, 1H), 7.17 (s, 1H), 7.13 (d, J=8.93 Hz, 2H), 6.54 (s, 1H), 4.16 (q, J=7.34 Hz, 2H), 4.04-4.10 (m, 2H), 3.86 (s, 3H), 3.53 (dt, J=2.08, 11.74 Hz, 2H), 3.48 (s, 3H), 2.95 (tt, J=3.88, 11.34 Hz, 1H), 2.04-2.17 (m, 2H), 1.80 (dd, J=1.77, 13.27 Hz, 2H), 1.53 (t, J=7.34 Hz, 3H). [M+H]=450.3.

### Example 190. 6-{4-[4-Methoxy-1-(propan-2-yl)-1H-pyrazol-3-yl]phenoxy}-7-methyl-3-(oxan-4-yl)-7H,8H-imidazo[1,5-a]pyrazin-8-one

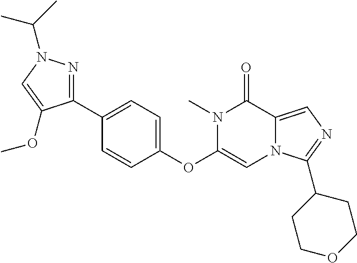 [SMILES: COc1cn(C(C)C)nc1-c1ccc(Oc2cn3c(C4CCOCC4)ncc3c(=O)n2C)cc1]

¹H NMR (400 MHz, CDCl₃) δ 8.05 (d, J=8.80 Hz, 2H), 7.95 (s, 1H), 7.19 (s, 1H), 7.13 (d, J=8.80 Hz, 2H), 6.53 (s, 1H), 4.48 (spt, J=6.70 Hz, 1H), 4.03-4.10 (m, 2H), 3.86 (s, 3H), 3.54 (dt, J=2.02, 11.71 Hz, 2H), 3.48 (s, 3H), 2.94 (tt, J=3.77, 11.26 Hz, 1H), 2.01-2.16 (m, 2H), 1.80 (dd, J=1.77, 13.27 Hz, 2H), 1.55 (d, J=6.72 Hz, 6H). [M+H]=464.3.

### Example 191. 6-[4-(4-Ethyl-1-methyl-1H-pyrazol-5-yl)phenoxy]-7-methyl-3-(oxan-4-yl)-7H,8H-imidazo[1,5-a]pyrazin-8-one

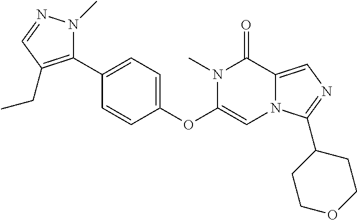 [SMILES: CCc1cnn(C)c1-c1ccc(Oc2cn3c(C4CCOCC4)ncc3c(=O)n2C)cc1]

¹H NMR (400 MHz, CDCl₃) δ 7.97 (s, 1H), 7.46 (s, 1H), 7.38 (d, J=8.80 Hz, 2H), 7.19 (d, J=8.80 Hz, 2H), 6.80 (s, 1H), 4.08-4.16 (m, 2H), 3.78 (s, 3H), 3.53 (dt, J=2.08, 11.68 Hz, 2H), 3.43 (s, 3H), 3.04 (tt, J=3.87, 11.36 Hz, 1H), 2.42 (q, J=7.50 Hz, 2H), 2.07-2.18 (m, 2H), 1.86 (dd, J=1.83, 13.33 Hz, 2H), 1.15 (t, J=7.58 Hz, 3H). [M+H]=434.3.

### Example 192. 6-[4-(1,4-Dimethyl-1H-pyrazol-5-yl)phenoxy]-7-methyl-3-(propan-2-yl)-7H,8H-imidazo[1,5-a]pyrazin-8-one

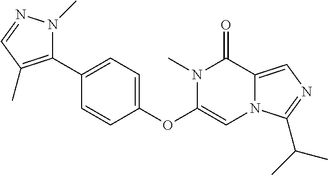 [SMILES: Cc1cnn(C)c1-c1ccc(Oc2cn3c(C(C)C)ncc3c(=O)n2C)cc1]

¹H NMR (400 MHz, CDCl₃) δ 7.97 (s, 1H), 7.41 (s, 1H), 7.39 (d, J=8.80 Hz, 2H), 7.21 (d, J=8.80 Hz, 2H), 6.76 (s, 1H), 3.80 (s, 3H), 3.45 (s, 3H), 3.10 (spt, J=6.89 Hz, 1H), 2.04 (s, 3H), 1.41 (d, J=6.85 Hz, 6H). [M+H]=378.1.

### Example 193. 6-[4-(1,4-Dimethyl-1H-pyrazol-5-yl)phenoxy]-7-methyl-3-(oxan-4-yl)-7H,8H-imidazo[1,5-a]pyrazin-8-one

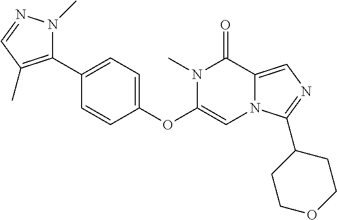 [SMILES: Cc1cnn(C)c1-c1ccc(Oc2cn3c(C4CCOCC4)ncc3c(=O)n2C)cc1]

¹H NMR (400 MHz, CDCl₃) δ 7.98 (s, 1H), 7.41 (s, 1H), 7.39 (d, J=8.80 Hz, 2H), 7.20 (d, J=8.80 Hz, 2H), 6.81 (s, 1H), 4.07-4.14 (m, 2H), 3.80 (s, 3H), 3.53 (dt, J=2.08, 11.74 Hz, 2H), 3.44 (s, 3H), 3.05 (tt, J=3.82, 11.34 Hz, 1H), 2.07-2.20 (m, 3H), 2.04 (s, 3H), 1.86 (dd, J=1.71, 13.33 Hz, 2H). [M+H]=420.2.

### Example 194. 6-[4-(1,4-Dimethyl-1H-pyrazol-3-yl)phenoxy]-7-methyl-3-(propan-2-yl)-7H,8H-imidazo[1,5-a]pyrazin-8-one

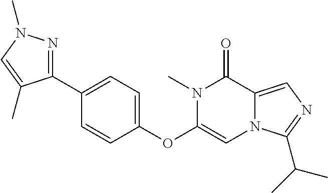 [SMILES: Cc1cn(C)nc1-c1ccc(Oc2cn3c(C(C)C)ncc3c(=O)n2C)cc1]

¹H NMR (400 MHz, CDCl₃) δ 7.94 (s, 1H), 7.76 (d, J=8.80 Hz, 2H), 7.25 (s, 1H), 7.17 (d, J=8.93 Hz, 2H), 6.54 (s, 1H), 3.92 (s, 3H), 3.48 (s, 3H), 3.03 (spt, J=6.91 Hz, 1H), 2.25 (d, J=0.61 Hz, 3H), 1.37 (d, J=6.85 Hz, 6H). [M+H]=378.1.

### Example 195. 6-[4-(1,4-Dimethyl-1H-pyrazol-3-yl)phenoxy]-7-methyl-3-(oxan-4-yl)-7H,8H-imidazo[1,5-a]pyrazin-8-one

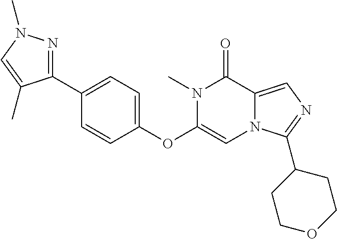 [SMILES: Cc1cn(C)nc1-c1ccc(Oc2cn3c(C4CCOCC4)ncc3c(=O)n2C)cc1]

¹H NMR (400 MHz, CDCl₃) δ 7.96 (s, 1H), 7.75 (d, J=8.80 Hz, 2H), 7.25 (s, 1H), 7.16 (d, J=8.80 Hz, 2H), 6.60 (s, 1H), 4.05-4.12 (m, 2H), 3.93 (s, 3H), 3.54 (dt, J=2.08, 11.68 Hz, 2H), 3.47 (s, 3H), 2.97 (tt, J=3.90, 11.32 Hz, 1H), 2.25 (s, 3H), 2.06-2.17 (m, 2H), 1.81 (dd, J=1.83, 13.33 Hz, 2H). [M+H]=420.2.

### Example 196. 6-[4-(4-Fluoro-1-methyl-1H-pyrazol-3-yl)phenoxy]-7-methyl-3-(propan-2-yl)-7H,8H-imidazo[1,5-a]pyrazin-8-one

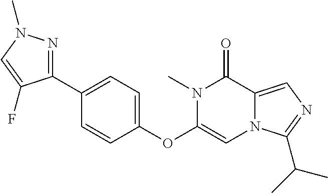 [SMILES: CC(C)c1ncc2c(=O)n(C)c(Oc3ccc(-c4nn(C)cc4F)cc3)cn12]

¹H NMR (400 MHz, CDCl₃) δ 7.94 (d, J=4.40 Hz, 2H), 7.91 (s, 1H), 7.36 (d, J=4.77 Hz, 1H), 7.17 (d, J=8.80 Hz, 2H), 6.56 (s, 1H), 3.91 (s, 3H), 3.47 (s, 3H), 2.96-3.10 (m, 1H), 1.37 (d, J=6.85 Hz, 6H). [M+H]=382.1.

### Example 197. 6-[4-(4-Fluoro-1-methyl-1H-pyrazol-3-yl)phenoxy]-7-methyl-3-(oxan-4-yl)-7H,8H-imidazo[1,5-a]pyrazin-8-one

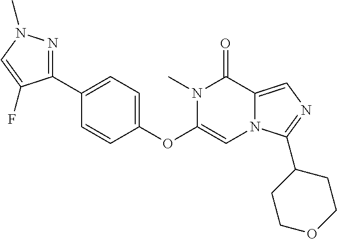 [SMILES: Cn1cc(F)c(-c2ccc(Oc3cn4c(C5CCOCC5)ncc4c(=O)n3C)cc2)n1]

¹H NMR (400 MHz, CDCl₃) δ 7.95 (s, 1H), 7.91 (d, J=8.56 Hz, 2H), 7.37 (d, J=4.65 Hz, 1H), 7.15 (d, J=8.44 Hz, 2H), 6.63 (s, 1H), 4.08 (d, J=11.00 Hz, 2H), 3.91 (s, 3H), 3.53 (t, J=11.31 Hz, 2H), 3.45 (s, 3H), 2.95 (t, J=11.25 Hz, 1H), 2.02-2.17 (m, 2H), 1.78-1.87 (m, 2H). [M+H]=424.2.

### Example 198. 6-[4-(1-Ethyl-4-fluoro-1H-pyrazol-3-yl)phenoxy]-7-methyl-3-(propan-2-yl)-7H,8H-imidazo[1,5-a]pyrazin-8-one

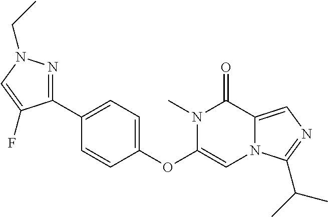 [SMILES: CCn1cc(F)c(-c2ccc(Oc3cn4c(C(C)C)ncc4c(=O)n3C)cc2)n1]

¹H NMR (400 MHz, CDCl₃) δ 7.89-7.96 (m, 3H), 7.39 (d, J=4.77 Hz, 1H), 7.17 (d, J=8.80 Hz, 2H), 6.54 (s, 1H), 4.16 (q, J=7.34 Hz, 2H), 3.47 (s, 3H), 2.97-3.09 (m, 1H), 1.53 (t, J=7.34 Hz, 3H), 1.37 (d, J=6.97 Hz, 6H). [M+H]=396.0.

### Example 199. 6-[4-(1-Ethyl-4-fluoro-1H-pyrazol-3-yl)phenoxy]-7-methyl-3-(oxan-4-yl)-7H,8H-imidazo[1,5-a]pyrazin-8-one

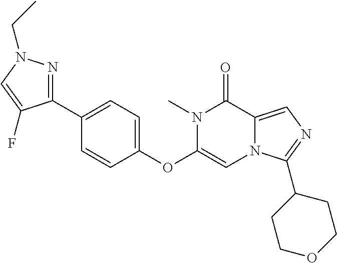 [SMILES: CCn1cc(F)c(-c2ccc(Oc3cn4c(C5CCOCC5)ncc4c(=O)n3C)cc2)n1]

¹H NMR (400 MHz, CDCl₃) δ 7.96 (s, 1H), 7.93 (d, J=8.68 Hz, 2H), 7.40 (d, J=4.77 Hz, 1H), 7.16 (d, J=8.93 Hz, 2H), 6.60 (s, 1H), 4.16 (q, J=7.34 Hz, 2H), 4.03-4.11 (m, 2H), 3.53 (dt, J=2.08, 11.74 Hz, 2H), 3.46 (s, 3H), 2.97 (tt, J=3.90, 11.32 Hz, 1H), 2.07-2.18 (m, 2H), 1.82 (dd, J=1.71, 13.33 Hz, 2H), 1.54 (t, J=7.34 Hz, 3H). [M+H]=438.1.

### Example 200. 6-{4-[4-Fluoro-1-(propan-2-yl)-1H-pyrazol-3-yl]phenoxy}-7-methyl-3-(propan-2-yl)-7H,8H-imidazo[1,5-a]pyrazin-8-one

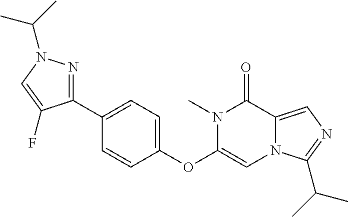 [SMILES: CC(C)c1ncc2c(=O)n(C)c(Oc3ccc(-c4nn(C(C)C)cc4F)cc3)cn12]

¹H NMR (400 MHz, CDCl₃) δ 7.91-7.98 (m, 3H), 7.40 (d, J=4.65 Hz, 1H), 7.17 (d, J=8.44 Hz, 2H), 6.53 (s, 1H), 4.40-4.53 (m, 1H), 3.48 (s, 3H), 2.97-3.11 (m, 1H), 1.54 (d, J=6.60 Hz, 6H), 1.37 (d, J=6.85 Hz, 6H). [M+H]=410.1.

### Example 201. 6-{4-[4-Fluoro-1-(propan-2-yl)-1H-pyrazol-3-yl]phenoxy}-7-methyl-3-(oxan-4-yl)-7H,8H-imidazo[1,5-a]pyrazin-8-one

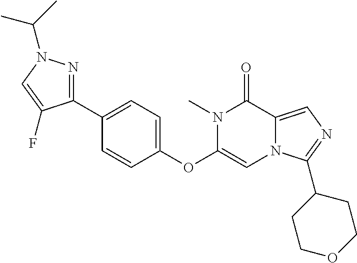 [SMILES: CC(C)n1cc(F)c(-c2ccc(Oc3cn4c(C5CCOCC5)ncc4c(=O)n3C)cc2)n1]

¹H NMR (400 MHz, CDCl₃) δ 7.98-7.89 (m, 3H), 7.41 (d, J=4.8 Hz, 1H), 7.20-7.11 (m, 2H), 6.60 (s, 1H), 4.46 (spt, J=6.7 Hz, 1H), 4.13-4.04 (m, 2H), 3.54 (dt, J=2.1, 11.7 Hz, 2H), 3.46 (s, 3H), 2.95 (tt, J=3.8, 11.3 Hz, 1H), 2.18-2.02 (m, 2H), 1.82 (dd, J=1.9, 13.3 Hz, 2H), 1.55 (d, J=6.7 Hz, 6H). [M+H]=452.4.

Example 202 was prepared in a manner analogous to Example 21, with the appropriate starting material substitutions.

### Example 202. 7-Methyl-3-(oxan-4-yl)-6-{[(1R,4R)-4-(trifluoromethyl)cyclohexyl]methoxy}-7H8H-imidazo[1,5-a]pyrazin-8-one

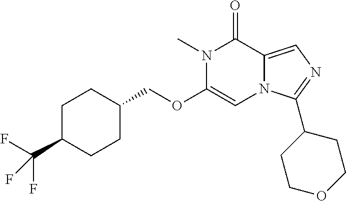 [SMILES: Cn1c(OC[C@H]2CC[C@H](C(F)(F)F)CC2)cn2c(C3CCOCC3)ncc2c1=O]

¹H NMR (400 MHz, CDCl₃) δ 7.87 (s, 1H), 6.33 (s, 1H), 4.19-4.11 (m, 2H), 3.82 (d, J=6.0 Hz, 2H), 3.61 (dt, J=2.1, 11.6 Hz, 2H), 3.42 (s, 3H), 3.07 (tt, J=3.9, 11.3 Hz, 1H), 2.17-2.01 (m, 7H), 1.98-1.83 (m, 3H), 1.52-1.36 (m, 2H), 1.29-1.14 (m, 2H). [M+H]=414.4.

### PHARMACOLOGICAL EXAMPLES

The present disclosure will be further illustrated by the following pharmacological examples. These examples are understood to be exemplary only and are not intended to limit the scope of the invention disclosed herein.

Enzymatic Assay

PDE1B inhibition was determined by an IMAP TR-FRET assay. The IMAP TR-FRET PDE assay was optimized for concentration of enzyme, Calmodulin, cAMP or cGMP substrate, DMSO tolerance, and incubation time.

Into each well of a solid white 1536 well plate (Corning) was dispensed 250 pg full-length recombinant NH-terminal GST tagged human PDE1B enzyme (BPS Bioscience Cat #60011, San Diego, Calif.) in 2.5 μL IMAP BSA reaction buffer (Molecular Devices, Sunnyvale, Calif.) containing 10 U/ml Calmodulin and 2.5 mM CaCl₂ (Sigma Aldrich.) After a brief centrifugation, 30 nL of compound was added by transfer from 1 mM stock in DMSO using a Kalypsys 1536 Pintool. Plates were incubated for 5 minutes at room temperature before dispensing 1.5 μL of 533 nM 5-carboxy fluorescein (FAM)-labeled cAMP (Molecular Devices, Sunnyvale, Calif.) for a final concentration of 200 nM. After a brief centrifugation, the plates were incubated for 30 minutes at room temperature. The assay was terminated by adding 5 μL IMAP binding reagent/Tb complex (Molecular Devices, Sunnyvale, Calif.) to each well.

Plates were incubated 1 hour at room temperature and read on a Viewlux multimode plate reader (Perkin Elmer). The instrument was set to excite using the DUG 11 filter and measure using 490/10 nm and 520/10 nm filters. Ratios of acceptor and donor were then calculated.

Data Analysis

For IC₅₀ calculations, the values of % efficacy versus a series of compound concentrations were then plotted using non-linear regression analysis of sigmoidal dose-response curves generated with the equation Y=B+(T−B)/1+10((Log EC50−X)×Hill Slope), where Y=percent activity, B=minimum percent efficacy, T=maximum percent efficacy, X=logarithm of compound and Hill Slope=slope factor or Hill coefficient. The IC₅₀ value was determined by the concentration causing a half-maximal percent efficacy.

Results

Table 2 presents the negative log of the half-maximal molar inhibitory concentration (pIC₅₀), with respect to PDE1B activity, for Formula I compounds.

TABLE 2

| PDE1b | |
|---|---|
| (pEC₅₀) | Example Numbers |
| >8 | 2, 138, 140, 144, 157, 158, 159, 160, 161, 162, 163, 164, |
| | 165, 166, 167, 169, 171, 176, 177, 178, 180, 181, 182, 184, |
| | 185, 187, 189, 190, 195, 196, 197, 198, 199, 200, 201 |
| 7-8 | 1, 3, 4, 5, 6, 7, 8, 9, 10, 11, 12, 13, 14, 15, 16, 17, 18, 19, 20, |
| | 21, 25, 26, 27, 28, 29, 30, 31, 32, 33, 34, 35, 36, 37, 38, 39, |
| | 40, 41, 42, 43, 44, 45, 46, 47, 48, 49, 50, 51, 52, 53, 54, 55, |
| | 56, 57, 58, 59, 60, 61, 62, 63, 64, 65, 66, 67, 68, 69, 70, 71, |
| | 72, 73, 74, 75, 76, 77, 78, 79, 80, 81, 82, 83, 84, 85, 86, 87, |
| | 88, 89, 90, 91, 92, 93, 94, 95, 97, 99, 100, 101, 103, 105, |
| | 106, 107, 109, 110, 111, 112, 113, 114, 115, 116, 117, 118, |
| | 120, 121, 122, 124, 125, 127, 128, 129, 130, 131, 132, 133, |
| | 134, 135, 139, 146, 147, 148, 151, 152, 153, 154, 155, 156, |
| | 168, 170, 172, 173, 174, 175, 179, 183, 186, 188, 191, |
| | 192, 193, 194, 202 |
| 6-7 | 22, 23, 24, 96, 98, 102, 104, 108, 119, 123, 126, 136, |
| | 137, 141, 142, 143, 145, 149, 150 |

### PDE1 Selectivity of Compounds

Assay Conditions

The selectivity of compounds of the present invention was determined using a panel of recombinant human PDEs and an in vitro enzymatic assay (BPS Bioscience). Series of dilutions of each test compound were prepared with 10% DMSO in assay buffer and 5 μl of the dilution was added to a 50 μl reaction so that the final concentration of DMSO is 1% in all of reactions.

The enzymatic reactions were conducted at room temperature for 60 minutes in a 50 μl mixture containing PDE assay buffer, 100 nM FAM-cAMP, or 100 nM FAM-cGMP, a recombinant PDE enzyme and the test compound.

After the enzymatic reaction, 100 μl of a binding solution (1:100 dilution of the binding agent with the binding agent diluent) was added to each reaction and the reaction was performed at room temperature for 60 minutes.

Fluorescence intensity was measured at an excitation of 485 nm and an emission of 528 nm using a Tecan Infinite M1000 microplate reader.

Data Analysis

PDE activity assays were performed in duplicate at each concentration. Fluorescence intensity is converted to fluorescence polarization using the Tecan Magellan6 software. The fluorescence polarization data were analyzed using the computer software, Graphpad Prism. The fluorescence polarization (FPt) in absence of the compound in each data set was defined as 100% activity. In the absence of PDE and the compound, the value of fluorescent polarization (FPb) in each data set was defined as 0% activity. The percent activity in the presence of the compound was calculated according to the following equation: % activity=(FP−FPb)/(FPt−FPb)×100%, where FP=the fluorescence polarization in the presence of the compound.

For IC₅₀ calculations, the values of % activity versus a series of compound concentrations were then plotted using non-linear regression analysis of Sigmoidal dose-response curve generated with the equation Y=B+(T−B)/1+10((Log EC50−X)×Hill Slope), where Y=percent activity, B=minimum percent activity, T=maximum percent activity, X=logarithm of compound and Hill Slope=slope factor or Hill coefficient. The IC₅₀ value was determined by the concentration causing a half-maximal percent activity.

Results

Exemplary compounds of the present invention displayed selectivity for PDE1 enzymes versus isoforms from many, if not all, other PDE families. In addition, exemplary compounds showed greater specificity for PDE1B compared to PDE1A and PDE1C.

### BIOLOGICAL EXAMPLES

The present disclosure will be further illustrated by the following biological examples. These examples are understood to be exemplary only, and not to limit the scope of the invention disclosed herein.

### Biological Example 1

Effect of Exemplary Compounds on Memory and Catalepsy

The studies here evaluated the effect of exemplary compounds of the present invention on memory and haloperidol induced catalepsy in mice and rats.

Methods

Subjects

Outbred hooded Long Evans rats (400 g average weight, sourced from Taconic Farms or Envigo) were used for rat fear conditioning, object recognition, and catalepsy. Upon arrival, rats were house in standard cages in groups of two. Experiments were always conducted during the light phase of the cycle. The animals received food and water ad libitum except during training and testing. All procedures were consistent with National Institutes of Health (NIH) guidelines and approved by the DNS/Helicon Institutional Animal Care and Use Committee.

Drug Administration

PDE1 inhibitors and positive control were dosed in a Vehicle containing 10% NMP, 40% PEG (MW400) and 50% water, unless specified otherwise. For subcutaneous dosing (s.c.), all drugs were administered at a volume of 10 mL per kg 30 min prior to behavior training unless specified otherwise. For oral dosing (p.o.), animals were dosed at the indicated amount 60 minutes prior to training.

Fear Conditioning

Rationale

Contextual fear conditioning is a form of associative learning in which animals learn to recognize a training environment (conditioned stimulus, CS) that has been previously paired with an aversive stimulus such as foot shock (unconditioned stimulus, US). When exposed to the same context at a later time, conditioned animals show a variety of conditional fear responses, including freezing behavior. See, e.g., Fanselow, 1984, Behav. Neurosci. 98, 269-277; Fanselow, 1984, Behav. Neurosci. 98, 79-95; Phillips and LeDoux, 1992, Behav. Neurosci. 106, 274-285.

Contextual conditioning has been used to investigate the neural substrates mediating fear-motivated learning. See, e.g., Phillips and LeDoux, 1992, Behav. Neurosci. 106, 274-285; Kim et al., 1993, Behav. Neurosci. 107, 1093-1098. Studies in mice and rats have provided evidence for functional interaction between hippocampal and non-hippocampal systems during contextual conditioning training. See, e.g., Maren et al., 1997, Behav. Brain Res. 88, 261-274; Maren et al., 1997, Neurobiol. Learn. Mem. 67, 142-149; Frankland et al., 1998, Behav. Neurosci. 112, 863-874. Specifically, post-training lesions of the hippocampus (but not pre-training lesions) greatly reduced contextual fear, implying that: 1) the hippocampus is essential for contextual memory but not for contextual learning per se and 2) in the absence of the hippocampus during training, non-hippocampal systems can support contextual conditioning.

Contextual conditioning has been extensively used to study the impact of various mutations on hippocampus-dependent learning and memory and strain differences in mice. See, e.g., Bourtchouladze et al., 1994, Cell 79, 59-68; Bourtchouladze et al., 1998, Learn Mem. 5, 365-374; Kogan et al., 1997, Current Biology 7, 1-11; Silva et al., 1996, Current Biology 6, 1509-1518; Abel et al., 1997, Cell 88, 615-626; Giese et al., 1998, Science 279, 870-873; Logue et al., 1997, Neuroscience 80, 1075-1086; Chen et al., 1996, Behav. Neurosci. 110, 1177-1180; Nguyen et al., 2000, Learn Mem. 7, 170-179.

Because robust learning can be triggered with a few minutes training session, contextual conditioning has been especially useful to study the biology of temporally distinct processes of short- and long-term memory. See, e.g., Kim et al., 1993, Behav. Neurosci. 107, 1093-1098; Abel et al., 1997, Cell 88, 615-626; Bourtchouladze et al., 1994, Cell 79, 59-68; Bourtchouladze et al., 1998, Learn. Mem. 5, 365-374. As such, contextual conditioning provides an excellent model to evaluate the role of various novel genes in hippocampal-dependent memory formation.

Protocol

Previous investigations had established that training with 1× or 2× CS-US pairings induces sub-maximal (weak) memory in wild-type mice. See, e.g., U.S.2009/0053140; Tully et al., 2003, Nat. Rev. Drug Discov. 2, 267-77; Bourtchouladze et al. 1998, Learn. Mem. 5, 365-374. Accordingly, contextual conditioning in this study was performed as described by Bourtchouladze et al., 1994, Cell 79, 59-68.

An automated fear conditioning system (Colburn Instruments) was used for contextual conditioning and a manual setup (Med Associates) for trace fear conditioning. Rats were placed in the conditioning chamber and allowed to explore for 2 min. A total of two foot-shocks were delivered (0.4-0.6 mA, 2 s duration) with an inter-trial interval of 1 min. These training conditions generate sub-maximal, or weak, memory in control rats, thereby allowing one to evaluate whether a PDE1b compound of the present invention can enhance memory formation.

Freezing was scored for 30 s after the last foot-shock (immediate freezing). Freezing was scored for 30 s after the last foot-shock (immediate freezing). The rats were then returned to their home-cage. Memory was tested after 24 h (LTM) for 3 min by scoring freezing behavior using automated algorithms (Med Associates).

Object Recognition Memory

Rationale

Novel Object Recognition (NOR) is an assay of recognition learning and memory retrieval, which takes advantage of the spontaneous preference of rodents to investigate a novel object compared with a familiar one.

The NOR test has been employed extensively to assess the potential cognitive-enhancing properties of novel compounds derived from high-throughput screening. Object recognition is an ethologically relevant task that does not result from negative reinforcement (foot shock). This task relies on the natural curiosity of rodents to explore novel objects in their environments more than familiar ones. Obviously, for an object to be “familiar,” the animal must have attended to it before and remembered that experience. Hence, animals with better memory will attend and explore a new object more than an object familiar to them. During testing, the animal is presented with the training object and a second, novel one. Memory of the training object renders it familiar to the animal, and it then spends more time exploring the new novel object rather than the familiar one (See Bourtchouladze et. al., 2003, Proc. Natl. Acad. Sci. USA 100, 10518-10522).

Studies indicate that the NOR procedure involves several brain regions, including the cortex and the hippocampus. Recent neuroimaging studies in humans demonstrated that memory in object recognition depends on prefrontal cortex (PFC). See Delbert et al., 1999, Neurology 52, 1413-1417. Consistent with these findings, rats with the PFC lesions show poor working memory when they are required to discriminate between familiar and novel objects. See Mitchell, 1998, Behav. Brain Res. 97, 107-113. Other studies on monkeys and rodents suggest that the hippocampus is important for novel object recognition. See, e.g., Teng et al., 2000, J. Neurosci 20, 3853-3863; Mumby, 2001, Brain Res. 127, 159-181. Hence, object recognition provides an excellent behavioral model to evaluate drug-compound effects on cognitive task associated with function of the hippocampus and cortex.

Protocol

The novel object recognition task was performed as described by Bevins and Besheer, 2006 (Nat. Protocol. 1, 1306-1311) using a standard novel object recognition system for rats (Stoelting). Objects were placed in the center of the box, testing was carried out in low light, and time exploring objects was assessed using Ethovision Software. All videos were reviewed by trained observers.

For two consecutive days, rats were habituated to the chamber for 5 min with 5 min of handling immediately following exposure to the apparatus. The next day, rats treated with 10% NMP, 40% PEG400, 50% water vehicle or drug 60 min before training were exposed to either two white blocks or two grey balls (˜4 cm in width/diameter) for 3 min. Approximately 24 h after training, rats were exposed to one familiar object and one novel object (grey ball is replaced with a white block and vice versa) and the time exploring each object was measured. Memory was scored by calculation of a discrimination index ((T_{N}−T_{F}/(T_{N}+T_{F}))*100; between group comparison) and by comparison of the time exploring the novel versus familiar object on the test day (within group comparison).

Catalepsy

Rationale

Catalepsy in rats can be defined as a drug-induced state where the animal may be placed in an unnatural body position and will remain in this position for a significantly longer time than vehicle-treated rats (Wadenberg, et al., 1996, Neurosci. Biobehav. Rev., 20, 325-339). The blockade of brain dopamine receptors by classic neuroleptic antipsychotics (e.g., haloperidol) produces extrapyramidal motor side-effects (including catalepsy) in a significant proportion of patients (Baldessarini, et al. “Drugs and the treatment of psychiatric disorders” The pharmacological basis of therapeutics Goodman, et al., (eds.) New York: Pergamon Press, 383-435). The neuroleptic-induced cataleptic state is a generally accepted animal model of the akinesia and rigidity observed in Parkinson's Disease (Sanberg, et al., 1998, Behavioral Neuroscience, 102, 748-759).

Protocol

Catalepsy was assessed with bar test 60 minutes after Haloperidol injection. The fore paws of the rats were placed on a horizontal bar positioned at 10 cm above the floor. Time spent in cataleptic posture, which was defined as an immobile posture while keeping both forelimbs on the bar, was measured with a maximum limit of 180 seconds. Automation of catalepsy scoring was performed using the Kinder Scientific Loco Chambers and data was recorded using Kinder Scientific Motor Monitor software.

Statistical Analyses

All behavioral experiments were designed and performed in a balanced fashion: (i) For each experimental condition (e.g. a specific dose-effect) an equal number of experimental and control animals were used; (ii) Each experimental condition may be replicated several times, and (iii) Replicate days were added to generate final number of subjects. In each experiment, the experimenter was unaware (blind) to the treatment of the subjects during training and testing. Data were analyzed by ANOVA using JMP or Prism software, followed by contrast analysis or Dunnett's multiple comparison tests, the results of which are shown.

Results

Exemplary compounds are also found to enhance contextual memory in the fear conditioning assay. Significant effects were seen at several concentrations, depending on the compound, including 1.0 mg/kg.

Exemplary compounds were also found to reverse haloperidol-induced catalepsy. Significant effects were seen at several concentrations, depending on the compound, ranging from 0.01 to 1.0 mg/kg, p.o.

### Biological Example 2

Effect of Exemplary Compounds on Cardiac Function

Exemplary compounds of the present invention were also evaluated in several models of cardiovascular function, including the telemeterized rat and Beagle dog. Each test compound (or vehicle) was administered by oral gavage, and animals were evaluated after each dose for any abnormal clinical signs. Hemodynamic (Heart rate, systolic, diastolic, and mean arterial pressure) and electrocardiographic parameters (PR interval, QRS duration, QT/QTc interval, RR interval) were recorded following dosing.

Results for Several Exemplary Compounds.

The results showed no notable effects of vehicle administration on systemic blood pressure, heart rate, or arterial pulse pressure in these studies. All parameters were within expected range during the entire monitoring period. In contrast, however, administration of several compounds of the present disclosure led to a reduction in blood pressure, increase in heart rate and in some cases, prolongation of the QTc interval.

It will be understood by one skilled in the art that the described embodiments herein do not limit the scope of the invention. The specification, including the examples, is intended to be exemplary only, and it will be apparent to those skilled in the art that various modifications and variations can be made in the present invention without departing from the scope or spirit of the invention as defined by the appended claims.

Furthermore, while certain details in the present disclosure are provided to convey a thorough understanding of the invention as defined by the appended claims, it will be apparent to those skilled in the art that certain embodiments may be practiced without these details. Moreover, in certain instances, well-known methods, procedures, or other specific details have not been described to avoid unnecessarily obscuring aspects of the invention defined by the appended claims.

1. A compound of Formula (I): 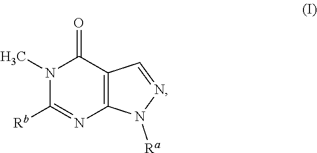
or a pharmaceutically acceptable salt thereof,
wherein,
R^{a} is a 3-6-membered cycloalkyl ring, a 3-6-membered cycloalkoxy ring, or —CHR^{c}R^{d}, where R^{c} and R^{d} are independently —C₁₋₄alkyl;
R^{b} is -L-L², -L¹-L²-L³ or —N(L⁴)L⁵;
L is a member selected from the group consisting of: a bond, —O—, —OCH₂—, —OCH₂CH₂—, and —NH—;
L¹ is a member selected from the group consisting of: a bond, —CH₂—, —CHF—, —CF₂—, —O—, —OCH₂—, —OCH₂CH₂—, and —NH—;
L² is aryl, 5-10-membered heteroaryl, —C₃₋₇cycloalkyl, or 3-12-membered heterocycloalkyl, all optionally substituted with 1 to 3 R^{1A}, where each R^{1A} is independently selected from the group consisting of: halo, —CN, —C₁₋₆alkyl, —C₁₋₆-haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl;
each n is independently 0, 1, or 2;
L³ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1B}, where each R^{1B} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl;
L⁴ and L⁵ are taken together with the nitrogen to which they are attached to form a 3-12-membered heterocycloalkyl ring, optionally substituted with 1 to 3 R^{1C}, where each R^{1C} is independently selected from the group consisting of: L⁶, halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —(CH₂)ₙ-heteroaryl; and
L⁶ is a member selected from the group consisting of: aryl, 5-6-membered heteroaryl, —C₃₋₇cycloalkyl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl, cycloalkyl, and heterocycloalkyl optionally substituted with 1 to 3 R^{1D}, where each R^{1D} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆-alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

2. A compound as in claim 1, having the structure of Formula (Ia): 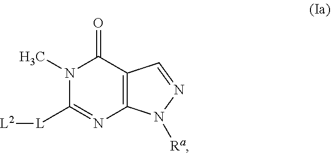
or a pharmaceutically acceptable salt thereof.

3. A compound as in claim 1, having the structure of Formula (Ib): 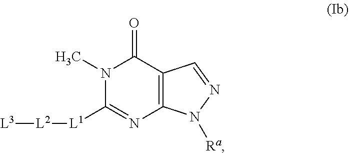
or a pharmaceutically acceptable salt thereof.

4. A compound as in claim 1, having the structure of Formula (Ic): 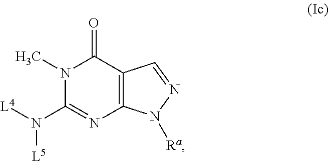
or a pharmaceutically acceptable salt thereof.

5. A compound as in claim 3, having the structure of Formula (Iba): 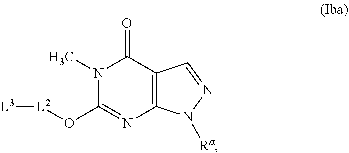
or a pharmaceutically acceptable salt thereof.

6. A compound as in claim 3, having the structure of Formula (Ibb): 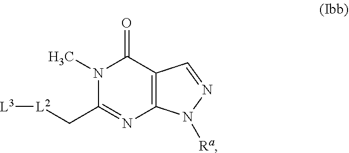
or a pharmaceutically acceptable salt thereof.

7. A compound as in claim 3, having the structure of Formula (Ibc): 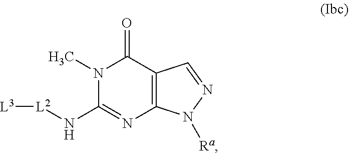
or a pharmaceutically acceptable salt thereof.

8. (canceled)

9. (canceled)

10. (canceled)

11. (canceled)

12. (canceled)

13. (canceled)

14. (canceled)

15. A compound as in claim 1, having the structure of Formula (Id): 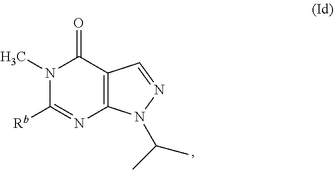
or a pharmaceutically acceptable salt thereof.

16. A compound as in claim 1, having the structure of Formula (Ie): 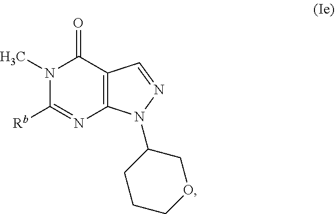
or a pharmaceutically acceptable salt thereof.

17. A compound as in claim 1, having the structure of Formula (If): 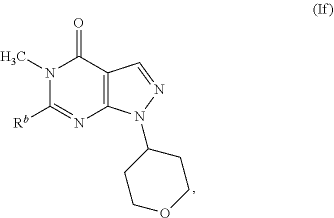
or a pharmaceutically acceptable salt thereof.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. A compound of Formula (II): 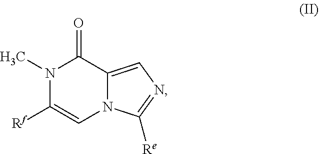
or pharmaceutically acceptable salt thereof,
wherein,
R^{e} is a 3-6-membered cycloalkyl ring, a 3-6-membered cycloalkoxy ring, or —CHR^{g}R^{h}, where R^{g} and R^{h} are independently —C₁₋₄alkyl;
R^{f} is -L⁷-L⁸ or -L⁷-L⁸-L⁹;
L⁷ is —O— or —OCH₂—;
L⁸ is aryl, 5-10-membered heteroaryl, —C₃₋₇cycloalkyl, or 3-12-membered heterocycloalkyl, all optionally substituted with 1 to 3 R^{1E}, where each R^{1E} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-O—C₁₋₆alkyl, —OCH₂CH═CH₂, —O(CH₂)ₙ—C₃₋₆cycloalkyl, —O(CH₂)ₙ-heterocycloalkyl, —NHC₁₋₆alkyl, —N(C₁₋₆alkyl)₂, —SC₁₋₆alkyl, —(CH₂)ₙ-heterocycloalkyl, and —CH₂heteroaryl;
each n is independently 0, 1 or 2; and
L⁹ is a member selected from the group consisting of: aryl, 5-6-membered heteroaryl, 3-10-membered heterocycloalkyl, —O(CH₂)ₙ—C₃₋₆cycloalkyl, —O(CH₂)ₙ-heterocycloalkyl, —(CH₂)ₙ-heterocycloalkyl, and —(CH₂)ₙ-heteroaryl, said aryl, heteroaryl and heterocycloalkyl optionally substituted with 1 to 3 R^{1F}, where each R^{1F} is independently selected from the group consisting of: halo, —CN, —C₁₋₆alkyl, —C₁₋₆haloalkyl, —C₁₋₆alkoxy, —C₁₋₆haloalkoxy, —C₃₋₆cycloalkyl, —C(O)C₁₋₆alkyl, —C₁₋₄alkyl-O—C₁₋₆alkyl, —C₁₋₆alkyl-CN, —OH, ═O, —O—C₁₋₄alkyl-C₃₋₆cycloalkyl, —NHC₁₋₆alkyl, —N(C₁₋₆alkyl)₂, and —SC₁₋₆alkyl.

49. A compound as in claim 48, having the structure of Formula (IIa): 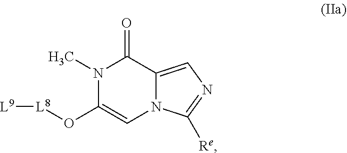
or a pharmaceutically acceptable salt thereof.

50. (canceled)

51. (canceled)

52. (canceled)

53. (canceled)

54. (canceled)

55. (canceled)

56. (canceled)

57. A compound as in claim 48, having the structure of Formula (IIb): 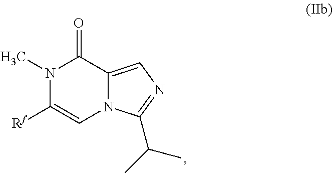
or pharmaceutically acceptable salt thereof.

58. A compound as in claim 48, having the structure of Formula (IIc): 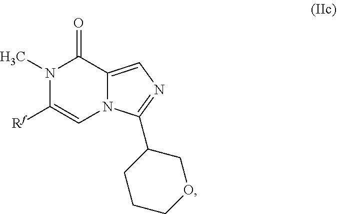
or pharmaceutically acceptable salt thereof.

59. A compound as in claim 48, having the structure of Formula (IId): 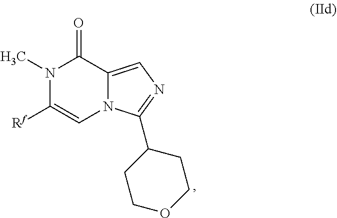
or pharmaceutically acceptable salt thereof.

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. A pharmaceutical composition comprising a compound, or pharmaceutically acceptable salt thereof, of claim 1, and a pharmaceutically acceptable carrier.

78. A method of augmented training to treat a neurological disorder, the method comprising:
(a) providing training to an animal in need of treatment of a neurological impairment associated with the neurological disorder under conditions sufficient to produce an improvement in performance by said animal of a neurological function whose deficit is associated with said neurological impairment;
(b) administering of a compound, or pharmaceutically acceptable salt thereof, of claim 1 to the animal in conjunction with said training;
(c) repeating said providing and administering steps one or more times; and
(d) reducing the number of training sessions sufficient to produce the improvement in performance, relative to the improvement in performance produced by training alone.

79. (canceled)

80. (canceled)

81. (canceled)

82. (canceled)

83. A method of treating a neurological disorder, comprising administering to a subject in need thereof an effective amount of a compound, or pharmaceutically acceptable salt thereof, of claim 1.

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)
